(19) United States
(12) Patent Application Publication
(10) Pub. No.: US 2024/0166593 A1
(43) Pub. Date: May 23, 2024

(54) LIPIDS SUITABLE FOR NUCLEIC ACID DELIVERY

(71) Applicant: RAMOT AT TEL-AVIV UNIVERSITY LTD., Tel Aviv (IL)

(72) Inventors: Srinivas RAMISHETTI, Tel Aviv (IL); Dan PEER, Tel Aviv (IL)

(21) Appl. No.: 18/263,795

(22) PCT Filed: Feb. 2, 2022
(86) PCT No.: PCT/IL2022/050139

§ 371 Date: Aug. 1, 2023

Related U.S. Application Data
(60) Provisional application No. 63/144,500, filed on Feb. 2, 2021.

(51) Int. Cl. C07C 239/22; A61K 9/127; C07C 229/12; C07C 229/16; C07C 233/36; C07D 295/15; C12N 15/88

(52) U.S. Cl. CPC C07C 239/22; A61K 9/1272; C07C 229/12; C07C 229/16; C07C 233/36; C07D 295/15; C12N 15/88

(57) ABSTRACT

The present invention provides lipids and lipid nanoparticle formulations comprising these lipids, alone or in combination with other lipids. These lipid nanoparticles may be formulated with nucleic acids to facilitate their intracellular delivery both in vitro and for therapeutic applications. The present invention also provides methods of chemical synthesis of these lipids, lipid nanoparticle preparation and formulation with nucleic acids.

### FIELD OF THE INVENTION

The present invention provides lipids and lipid nanoparticle formulations comprising these lipids, alone or in combination with other lipids. These lipid nanoparticles may be formulated with nucleic acids to facilitate their intracellular delivery both in vitro and for in vivo therapeutic applications. The present invention also provides methods of chemical synthesis of these lipids, lipid nanoparticle preparation and formulation with nucleic acids.

### BACKGROUND OF THE INVENTION

Therapeutic nucleic acids including small interfering RNA (siRNA), micro RNA (miRNA), antisense oligonucleotides, messenger RNA (mRNA), ribozymes, pDNA and immune stimulating nucleic acids act via a variety of mechanisms. Specific proteins can be downregulated by siRNA or miRNA through RNA interference (RNAi). Hematopoietic cells, such as leukocytes in general, and primary T lymphocytes and B-cells in particular, are notoriously hard to transfect with small interfering RNAs (siRNAs). Modulating immune cells function, such as T cells and B cells, by downregulating specific genes using RNA interference (RNAi) holds tremendous potential in advancing targeted therapies in many immune-related disorders including cancer, inflammation, autoimmunity and viral infections. The therapeutic applications of RNAi are extremely broad, since siRNA and miRNA constructs can be synthesized with any nucleotide sequence directed against a target protein. To date, siRNA constructs have shown the ability to specially silence target proteins in both in vitro and in vivo models. These are currently being evaluated in clinical studies.

Messenger RNA (mRNA) is the family of large RNA molecules which transport the genetic information from DNA to ribosome. Some nucleic acids, such as mRNA or plasmids, can be used to effect expression of specific cellular products. Such nucleic acids would be useful in the treatment to the of diseases related deficiency of a protein or enzyme. However, there are many problems associated with nucleic acids in therapeutic contexts. One of the major problems with therapeutic nucleic acids is the stability of the phosphodiester inter nucleotide link and its susceptibility to nucleases. Apart from that these nucleic acids have limited ability to cross the cell membrane.

Various lipids, e.g., cationic lipids, have proved to be excellent carriers of nucleic acids to treat different diseases in gene therapy applications. Lipid nanoparticles formed from cationic lipids and other co-lipids such as cholesterol, DSPC and PEGylated lipids encapsulated oligonucleotides which protect them from degradation and facilitate the cellular uptake.

WO 2018087753 discloses a cationic lipid comprising a functional group represented by the structure: —W-(T=O)ₘ—X—(CH₂)_{z}—Y, wherein X and Y are each independently O, N or NH, wherein X and Y cannot both be O; W is a bond, O, NH or S; T is C or S; m is 0 or 1; and z is 0 or 2, wherein said functional group is linked to at least one saturated or unsaturated fatty acid residue. Nevertheless, there remains a need in the art for suitable and efficient delivery platforms for delivery of oligonucleotides.

### SUMMARY OF THE INVENTION

The present invention relates to novel lipids which can be used in lipid nanoparticle preparation. These lipid nanoparticles protect nucleic acids from degradation, clearance from circulation and intracellular release. In addition, the nucleic acid encapsulated lipid nanoparticles advantageously are well-tolerated and provide an adequate therapeutic index, such that patient treatment at an effective dose of the nucleic acid is not associated with unacceptable toxicity and/or risk to the patient. The present invention also provides the methods of chemical synthesis of these lipids, lipid nanoparticle preparation and formulations with nucleic acids.

In some embodiments, the present invention relates to novel lipids, and formulations of such lipids with siRNA and pDNA. These lipid nanoparticles (LNPs) were further characterized by DLS and assessed for their in vitro activity in various cancer cell lines.

According to some embodiments, there is provided a lipid represented by the structure of Formula (II):

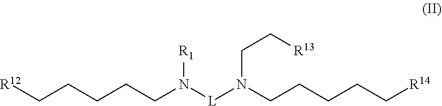

wherein
each one of R¹ and R¹³ is independently selected from the group consisting of: OH, C₁₋₃ alkyl-OH, C₄₋₁₄ alkyl and C₄₋₁₄ alkenyl;
R¹² is selected from the group consisting of: C₁₋₁₃ alkyl; C₂₋₁₅ alkenyl, C₁₋₆ alkyl-CO₂—C₀₋₃ alkylene-N(C₁₋₈ alkyl)₂ and C₁₋₆ alkyl-CO₂—C₀₋₃ alkylene-NH—C₁₋₈ alkyl;
R¹⁴ is selected from the group consisting of: C₁₋₁₃ alkyl; C₂₋₁₅ alkenyl and

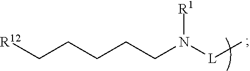

each L is an alkylene ester linker represented by: L^{a}-X^{a}-L^{b};
X^{a} is selected from the group consisting of: —O₂C—, —CO₂—C₂₋₄ alkylene-O₂C—, O₂C—C₂₋₄ alkylene-O₂C—, —CO₂—C₂₋₄ alkylene-CO₂—, and O₂C—C₂₋₄ alkylene-CO₂—;
L^{a} is selected from the group consisting of: C₁₋₃ alkylene, C₄₋₁₂ alkylene, C₂₋₁₀ alkenylene and absent; and
L^{b} is selected from the group consisting of: C₁₋₃ alkylene, C₂₋₁₀ alkenylene and C₄₋₁₂ alkylene.

According to some embodiments, R¹ is selected from the group consisting of: OH, C₂₋₃ alkyl-OH, and C₈₋₁₄ alkyl. Each possibility represents a separate embodiment of the invention.

According to some embodiments, R¹² is selected from the group consisting of: C₉₋₁₅ alkenyl, C₅₋₁₁ alkyl, C₃₋₆ alkyl-CO₂—C₀₋₂ alkylene-N(C₂₋₈ alkyl)₂. Each possibility represents a separate embodiment of the invention.

It is to be understood that the phrase “each L is selected from the group consisting of” refers to the situation wherein R¹⁴ may be

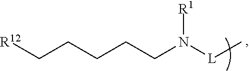

so that two separate L substituents appear in the structure of Formula (II), i.e., one L of the main formula and a second L of R¹⁴. It is intended to mean that in such cases, each of these L, L^{a}, X^{a} and L^{b} substituents may be independently selected from the corresponding selection group of the appropriate embodiment. In other words, in cases that L appears twice, the two sets of L^{a}, X^{a} and L^{b} substituents may be the same or different.

According to some embodiments, R¹³ is selected from the group consisting of: OH, CH₂—OH, and C₄₋₁₂ alkyl. Each possibility represents a separate embodiment of the invention.

According to some embodiments, R¹⁴ is selected from the group consisting of: C₉₋₁₅ alkenyl, C₁₋₉ alkyl and

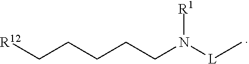

Each possibility represents a separate embodiment of the invention.

According to some embodiments, X^{a} is selected from the group consisting of: —O₂C— and —CO₂—C₂₋₄ alkylene-O₂C—. According to some embodiments, L^{a} is absent or selected from the group consisting of: C₂₋₃ alkylene and C₆₋₁₁ alkylene. According to some embodiments, L^{b} is selected from the group consisting of: C₁₋₃ alkylene and C₄₋₁₂ alkylene.

According to some embodiments, the lipid is selected from the group consisting of: DSL1-1, DSL1-2, DSL1-3, DSL1-4, DSL1-5, DSL3c-1, DSL2-1, DSL2-2, DSL2-49, DSL2-50, DSL4-1 and DSL4-2. Each possibility represents a separate embodiment of the invention. According to some embodiments, the lipid is selected from the group consisting of: DSL1-3 and DSL2-50.

The chemical structures of each of the specific compound are detailed below in the “Exemplary Compounds” Section and in the claims.

According to some embodiments, there is provided a lipid represented by the structure of Formula (I):

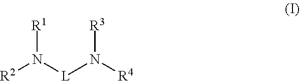

wherein
L is L^{a}-X^{a}-L^{b};
X^{a} is selected from the group consisting of: —O₂C—, —O—, —CO₂-L^{c}-O₂C—, —O₂C-L^{c}-CO₂—, —CO₂-L^{c}-CO₂—, L^{c}-CO₂—, -L^{c}-O₂C—, —CO₂—NH— and —CO—NH—;
L^{a} is selected from the group consisting of: C₄₋₂₀ alkylene, C₄₋₂₀ alkenylene and C₀₋₄ alkylene;
L^{b} is selected from the group consisting of: C₄₋₂₀ alkylene, C₄₋₂₀ alkenylene and C₀₋₄ alkylene;
L^{c} is selected from the group consisting of: C₁₋₆ alkylene-(O—C₁₋₆ alkylene)_{f}-,
C₂₋₆ alkenylene-(O—C₁₋₆ alkylene)_{g}-, C₁₋₆ alkylene-S—C₁₋₆ alkylene, C₁₋₆ alkylene-S—S—C₁₋₆ alkylene, C₀₋₄ alkylene-aryl-C₀₋₄ alkylene, and C₁₋₄ alkylene;
each one off and g is 0, 1, 2, 3, 4 or 5;
R¹ is selected from the group consisting of: C₀₋₁₀ alkylene-Y^{a}, C₂₋₆ alkenylene-Y^{a}, C₁₋₄ alkyl, C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂-C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl;
R² is selected from the group consisting of: C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₂₋₁₅ alkylene-CO₂—C₀₋₄ alkylene-N(C₁₋₁₂ alkyl)₂, C₂₋₁₅ alkylene-CO₂—CH₂CH₂OCH₂CH₂—N(C₁₋₁₂ alkyl)₂, C₂₋₁₅ alkylene-CO₂—C₀₋₄ alkylene-NH—C₁₋₁₂ alkyl, C₂₋₁₅ alkylene-CO₂—C₀₋₄ alkylene-N(C₅₋₂₅ alkenyl)₂, C₂₋₁₅ alkylene-CO₂—C₀₋₄ alkylene-NH—C₅₋₂₅ alkenyl, C₂₋₁₅ alkylene-CO—NH—C₀₋₅ alkylene-N(C₁₋₁₂ alkyl)₂ and C₅₋₁₅ alkylene-CO₂H;
R³ is selected from the group consisting of: H, C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₀₋₆ alkylene-Y^{d}, C₂₋₆ alkenylene-Y^{d} and C₁₋₄ alkyl;
R⁴ is selected from the group consisting of: C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl and C₅₋₁₅ alkylene-CO₂-C₅₋₁₅ alkylene-N(R⁵)R⁶;
R⁵ is selected from the group consisting of: C₁₋₄ alkyl, C₀₋₁₀ alkylene-OH, C₀₋₁₀ alkylene-halogen;
R⁶ is selected from the group consisting of: C₅₋₂₅ alkyl, C₅₋₂₅ alkenyl;
Y^{a} is selected from the group consisting of: O—Y^{b}, halogen, PO₃H₂, PO₃(C₁₋₄ alkyl)₂, PO₃H(C₁₋₄ alkyl), NH₂, pyrrolidinyl, piperidinyl, piperzinyl, SH, NMe₂, NMe₃⁺ and NH—Y^{b};
Y^{b} is selected from the group consisting of: H, C₁₋₄ alkyl, C₁₋₄ alkylene-OH, C₁₋₄ alkylene-(O—C₁₋₄ alkylene)₁₋₃-OH, PO₃H₂, PO₃(C₁₋₄ alkyl)₂, PO₃H(C₁₋₄ alkyl), CO—C₁₋₄ alkyl-Y°, CO-aryl, SO₃H, SO₃—(C₁₋₄ alkyl) and SO₃-aryl;
Y^{c} is selected from the group consisting of: H, NH₂, NH(CH₃), N(CH₃)₂, pyrrolidinyl piperidinyl and piperazinyl each pyrrolidinyl piperidinyl and piperazinyl is optionally substituted with a C₁₋₄ alkyl;
Y^{d} is selected from the group consisting of: O—Y^{e}, halogen, PO₃H₂, PO₃(C₁₋₄ alkyl)₂, PO₃H(C₁₋₄ alkyl), NH₂, SH, pyrrolidinyl, piperidinyl piperazinyl, NMe₂, NMe₃⁺ and NH—Y^{e};
Y^{e} is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl-Y^{f}, CO-aryl, SO₃H, SO₃—(C₁₋₄ alkyl) and SO₃-aryl; and
Y^{f} is selected from the group consisting of: H, NH₂, NH(CH₃), N(CH₃)₂, pyrrolidinyl piperidinyl and piperazinyl, each pyrrolidinyl piperidinyl and piperazinyl is optionally substituted with a C₁₋₄ alkyl; including salts, hydrates, solvates, polymorphs, optical isomers, geometrical isomers, enantiomers, diastereomers, and mixtures thereof.

It is to be understood than when L^{c} is C₁₋₆ alkylene-(O—C₁₋₆ alkylene)_{f}- and f is 0, then L^{c} is C₁₋₆ alkylene-, and when L^{c} is C₂₋₆ alkenylene-(O—C₁₋₆ alkylene)_{g}- and g is 0, then L^{c} is C₂₋₆ alkenylene. Similarly, it is to be understood that recitations of C₀ alkylene (e.g., in C₀₋₄ alkylene as an option of L^{a}) means that this is absent, leaving a single bond between the previous and next moieties. One example is L^{a} of compound DSL2-1, which is described below (Exemplary Compounds section) as absent.

According to some embodiments, R² is selected from the group consisting of: C₈₋₂₅ alkyl, C₈₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₂₋₁₅ alkylene-CO₂—C₀₋₄ alkylene-N(C₁₋₁₂ alkyl)₂, C₂₋₁₅ alkylene-CO₂—C₀₋₄ alkylene-NH—C₁₋₁₂ alkyl, C₂₋₁₅ alkylene-CO₂—C₀₋₄ alkylene-N(C₅₋₂₅ alkenyl)₂, C₂₋₁₅ alkylene-CO₂—C₀₋₄ alkylene-NH—C₅₋₂₅ alkenyl, C₂₋₁₅ alkylene-CO—NH—C₀₋₄ alkylene-N(C₁₋₁₂ alkyl)₂, and C₅₋₁₅ alkylene-CO₂H; and R⁴ is selected from the group consisting of: C₈₋₂₅ alkyl, C₈₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, and C₅₋₁₅ alkylene-CO₂—C₅₋₁₅ alkylene-N(R⁵)R⁶.

According to some embodiments, X^{a} is selected from the group consisting of: —O₂C—, —O—, —CO₂-L^{c}-O₂C—, —O₂C-L^{c}-CO₂—, -L^{c}-O₂C—, —CO₂—NH— and —CO—NH—; L^{a} is selected from the group consisting of: C₄₋₂₀ alkylene, and C₀₋₄ alkylene; L^{b} is selected from the group consisting of: C₄₋₂₀ alkylene, and C₀₋₄ alkylene; L^{c} is selected from the group consisting of: C₁₋₆ alkylene-O—C₁₋₆ alkylene-, C₁₋₆ alkylene-S—S—C₁₋₆ alkylene, and C₁₋₄ alkylene; R¹ is selected from the group consisting of: C₀₋₁₀ alkylene-Y^{a}, C₁₋₄ alkyl, and C₅₋₂₅ alkyl, C₅₋₁₅ alkylene-CO₂—C₅₋₁₅ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₂₋₁₅ alkylene-CO₂—C₀₋₄ alkylene-N(C₁₋₁₂ alkyl)₂, C₂₋₁₅ alkylene-CO₂—C₀₋₄ alkylene-NH—C₁₋₁₂ alkyl, C₂₋₁₅ alkylene-CO₂—C₀₋₄ alkylene-N(C₅₋₂₅ alkenyl)₂, C₂₋₁₅ alkylene-CO₂—C₀₋₄ alkylene-NH—C₅₋₂₅ alkenyl, C₂₋₁₅ alkylene-CO—NH—C₀₋₈ alkylene-N(C₁₋₁₂ alkyl)₂ and C₅₋₁₅ alkylene-CO₂H; R³ is selected from the group consisting of: H, C₅₋₂₅ alkyl, C₅₋₂₅ alkenyl, C₀₋₆ alkylene-Y^{d}, and C₁₋₄ alkyl; R⁴ is selected from the group consisting of: C₅₋₂₅ alkyl, C₅₋₂₅ alkenyl, C₅₋₁₅ alkylene-CO₂—C₅₋₁₅ alkenyl, and C₅₋₁₅ alkylene-CO₂—C₅₋₁₅ alkylene-N(R⁵)R⁶; R⁵ is selected from the group consisting of: C₁₋₄ alkyl, C₀₋₁₀ alkylene-OH, and C₀₋₁₀ alkylene-halogen, R⁶ is selected from the group consisting of: C₅₋₂₅ alkyl, and C₅₋₂₅ alkenyl; Y^{a} is selected from the group consisting of: O—Y^{b}, halogen, PO₃H₂, NH₂, and NMe₂; Y^{b} is selected from the group consisting of: H, C₁₋₄ alkylene-OH, C₁₋₄ alkylene-(O—C₁₋₄ alkylene)₁₋₃-OH, and CO—C₁₋₄ alkyl-Y^{c}; Y^{c} is N(CH₃)₂; Y^{d} is selected from the group consisting of: O—Y^{e}, halogen, and NH₂; Y^{e} is selected from the group consisting of: H, and CO—C₁₋₄ alkyl-Y^{f}; and Y^{f} is selected from the group consisting of: N(CH₃)₂, and piperazinyl, which is optionally substituted with a C₁₋₄ alkyl.

According to some embodiments, X^{a} is selected from the group consisting of: —O₂C—, and —CO₂-L^{c}-O₂C—; L^{a} is selected from the group consisting of: C₄₋₂₀ alkylene, and C₀₋₄ alkylene; L^{b} is selected from the group consisting of: C₄₋₂₀ alkylene, and C₀₋₄ alkylene; L^{c} is C₁₋₄ alkylene; R¹ is selected from the group consisting of: C₀₋₁₀ alkylene-Y^{a}, and C₅₋₂₅ alkyl; R² is selected from the group consisting of: C₅₋₂₅ alkyl, C₅₋₂₅ alkenyl, and C₂₋₁₅ alkylene-CO₂—C₀₋₄ alkylene-N(C₁₋₁₂ alkyl)₂; R³ is selected from the group consisting of: C₅-alkyl, and C₀₋₆ alkylene-Y^{d}; R⁴ is selected from the group consisting of: C₅₋₂₅ alkyl, C₅₋₂₅ alkenyl, and C₅₋₁₅ alkylene-CO₂—C₅₋₁₅ alkylene-N(R⁵)R⁶; R⁵ is C₀₋₁₀ alkylene-OH; R⁶ is C₅₋₂₅ alkenyl; Y^{a} is O—Y^{b}—Y^{b} is H; Y^{d} is O—Y^{e}; and Y^{e} is H.

According to some embodiments, R¹ is OH or CH₂CH₂—R²¹; R² comprises at least 6 carbon atoms and is represented by CH₂CH₂—R²²; R³ is CH₂CH₂—R²³; R⁴ comprises at least 6 carbon atoms and is represented by CH₂CH₂—R²⁴; none of R¹, R², R³, and R⁴ is H; so the lipid is further represented by the structure of Formula (I′):

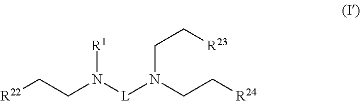

R²¹ is selected from the group consisting of: C₀₋₈ alkylene-Y^{a}, C₂₋₄ alkenylene-Y^{a}, C₁₋₂ alkyl, C₃₋₂₅ alkyl, C₅₋₂₃ alkenyl, C₃₋₂₃ alkynyl, C₃₋₁₃ alkylene-CO₂—C₅₋₁₅ alkyl, C₃₋₁₃ alkylene-CO₂-C₅₋₁₅ alkenyl, C₃₋₁₃ alkylene-O₂C—C₅₋₁₅ alkyl, C₃₋₁₃ alkylene-O₂C—C₅₋₁₅ alkenyl, C₃₋₁₃ alkenylene-CO₂—C₅₋₁₅ alkyl, C₃₋₁₃ alkenylene-CO₂—C₅₋₁₅ alkenyl, C₃₋₁₃ alkenylene-O₂C—C₅₋₁₅ alkyl, and C₃₋₁₃ alkenylene-O₂C—C₅₋₁₅ alkenyl; R²² is selected from the group consisting of: C₄₋₂₃ alkyl, C₄₋₂₃ alkenyl, C₄₋₂₃ alkynyl, C₃₋₁₃ alkylene-CO₂—C₅₋₁₅ alkyl, C₃₋₁₃ alkylene-CO₂—C₅₋₁₅ alkenyl, C₃₋₁₃ alkylene-O₂C—C₅₋₁₅ alkyl, C₃₋₁₃ alkylene-O₂C—C₅₋₁₅ alkenyl, C₃₋₁₃ alkenylene-CO₂—C₅₋₁₅ alkyl, C₃₋₁₃ alkenylene-CO₂—C₅₋₁₅ alkenyl, C₃₋₁₃ alkenylene-O₂C—C₅₋₁₅ alkyl, C₃₋₁₃ alkenylene-O₂C—C₅₋₁₅ alkenyl, C₀₋₁₃ alkylene-CO₂—C₀₋₄ alkylene-N(C₁₋₁₂ alkyl)₂, C₀₋₁₃ alkylene-CO₂—C₀₋₄ alkylene-NH—C₁₋₁₂ alkyl, C₀₋₁₃ alkylene-CO₂—C₀₋₄ alkylene-N(C₅₋₂₅ alkenyl)₂, C₀₋₁₃ alkylene-CO₂—C₀₋₄ alkylene-NH—C₅₋₂₅ alkenyl, C₀₋₁₃ alkylene-CO—NH—C₀₋₈ alkylene-N(C₁₋₁₂ alkyl)₂ and C₃₋₁₃ alkylene-CO₂H; R²³ is selected from the group consisting of: H, C₃₋₂₃ alkyl, C₃₋₂₃ alkenyl, C₃₋₂₃ alkynyl, C₃₋₁₃ alkylene-CO₂—C₅₋₁₅ alkyl, C₃₋₁₃ alkylene-CO₂—C₅₋₁₅ alkenyl, C₃₋₁₃ alkylene-O₂C—C₅₋₁₅ alkyl, C₃₋₁₃ alkylene-O₂C—C₅₋₁₅ alkenyl, C₃₋₁₃ alkenylene-CO₂—C₅₋₁₅ alkyl, C₃₋₁₃ alkenylene-CO₂—C₅₋₁₅ alkenyl, C₃₋₁₃ alkenylene-O₂C—C₅₋₁₅ alkyl, C₃₋₁₃ alkenylene-O₂C—C₅₋₁₅ alkenyl, C₀₋₄ alkylene-Y^{d}, C₀₋₄ alkenylene-Y^{d} and C₁₋₂ alkyl; R²⁴ is selected from the group consisting of: C₃₋₂₃ alkyl, C₃₋₂₃ alkenyl, C₃₋₂₃ alkynyl, C₃₋₁₃ alkylene-CO₂—C₅₋₁₅ alkyl, C₃₋₁₃ alkylene-CO₂—C₅₋₁₅ alkenyl, C₃₋₁₃ alkylene-O₂C—C₅₋₁₅ alkyl, C₃₋₁₃ alkylene-O₂C—C₅₋₁₅ alkenyl, C₃₋₁₃ alkenylene-CO₂—C₅₋₁₅ alkyl, C₃₋₁₃ alkenylene-CO₂—C₅₋₁₅ alkenyl, C₃₋₁₃ alkenylene-O₂C—C₅₋₁₅ alkyl, C₃₋₁₃ alkenylene-O₂C—C₅₋₁₅ alkenyl and C₃₋₁₃ alkylene-CO₂-C₅₋₁₅ alkylene-N(R⁵)R⁶.

According to some embodiments, R²² is represented by CH₂CH₂—R³²; R²⁴ is represented by CH₂CH₂—R³⁴; so the lipid is further represented by the structure of Formula (I″):

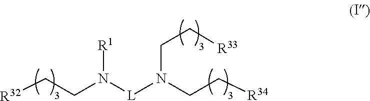

R³² is selected from the group consisting of: C₂₋₂₁ alkyl, C₂₋₂₁ alkenyl, C₂₋₂₁ alkynyl, C₁₋₁₁ alkylene-CO₂—C₅₋₁₅ alkyl, C₁₋ₙ alkylene-CO₂—C₅₋₁₅ alkenyl, C₁₋ₙ alkylene-O₂C—C₅₋₁₅ alkyl, C₁₋ₙ alkylene-O₂C—C₅₋₁₅ alkenyl, C₂₋₁₁ alkenylene-CO₂—C₅₋₁₅ alkyl, C₂₋₁₁ alkenylene-CO₂-C₅₋₁₅ alkenyl, C₂₋₁₁ alkenylene-O₂C—C₅₋₁₅ alkyl, C₂₋₁₁ alkenylene-O₂C—C₅₋₁₅ alkenyl, C₀₋₁₁ alkylene-CO₂—C₀₋₄ alkylene-N(C₁₋₁₂ alkyl)₂, C₀₋₁₁ alkylene-CO₂—C₀₋₄ alkylene-NH—C₁₋₁₂ alkyl, C₀₋₁₃ alkylene-CO₂—C₀₋₄ alkylene-N(C₅₋₂₅ alkenyl)₂, C₀₋₁₁ alkylene-CO₂—C₀₋₄ alkylene-NH—C₅₋₂₅ alkenyl, C₀₋₁₁ alkylene-CO—NH—C₀₋₄ alkylene-N(C₁₋₁₂ alkyl)₂ and C₁₋ₙ alkylene-CO₂H; and R³⁴ is selected from the group consisting of: C₁₋₂₁ alkyl, C₂₋₂₁ alkenyl, C₂₋₂₁ alkynyl, C₁₋₁₁ alkylene-CO₂—C₅₋₁₅ alkyl, C₁₋ₙ alkylene-CO₂—C₅₋₁₅ alkenyl, C₁₋ₙ alkylene-O₂C—C₅₋₁₅ alkyl, —C₁₋₁₁ alkylene-O₂C—C₅₋₁₅ alkenyl, C₂₋₁₁ alkenylene-CO₂—C₅₋₁₅ alkyl, C₂₋₁₁ alkenylene-CO₂-C₅₋₁₅ alkenyl, C₂₋₁₁ alkenylene-O₂C—C₅₋₁₅ alkyl, C₂₋₁₁ alkenylene-O₂C—C₅₋₁₅ alkenyl and C₁₋₁₁ alkylene-CO₂—C₅₋₁₅ alkylene-N(R⁵)R⁶.

According to some embodiments, R³² is represented by CH₂(CH₂)₆—R⁴²; R³⁴ is represented by CH₂(CH₂)—R⁴⁴; so the lipid is further represented by the structure of Formula (I′″):

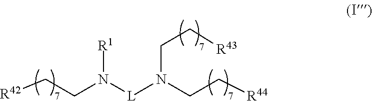

R⁴² is selected from the group consisting of: H, C₁₋₁₇ alkyl, C₂₋₁₇ alkenyl, C₂₋₁₇ alkynyl, C₀₋₇ alkylene-CO₂—C₅₋₁₅ alkyl, C₀₋₇ alkylene-CO₂—C₅₋₁₅ alkenyl, C₀₋₇ alkylene-O₂C—C₅₋₁₅ alkyl, C₀₋₇ alkylene-O₂C—C₅₋₁₅ alkenyl, C₂₋₇ alkenylene-CO₂—C₅₋₁₅ alkyl, C₂₋₇ alkenylene-CO₂—C₅₋₁₅ alkenyl, C₂₋₇ alkenylene-O₂C—C₅₋₁₅ alkyl, C₂₋₇ alkenylene-O₂C—C₅₋₁₅ alkenyl, C₀₋₁₁ alkylene-CO₂—C₀₋₄ alkylene-N(C₁₋₁₂ alkyl)₂, C₀₋₇ alkylene-CO₂—C₀₋₄ alkylene-NH—C₁₋₁₂ alkyl, C₀₋₇ alkylene-CO₂—C₀₋₄ alkylene-N(C₅₋₂₅ alkenyl)₂, C₀₋₇ alkylene-CO₂—C₀₋₄ alkylene-NH—C₅₋₂₅ alkenyl, C₀₋₇ alkylene-CO—NH—C₀₋₄ alkylene-N(C₁₋₁₂ alkyl)₂ and C₀₋₇ alkylene-CO₂H; and R⁴⁴ is selected from the group consisting of: H, C₁₋₁₇ alkyl, C₂₋₁₇ alkenyl, C₂₋₁₇ alkynyl, C₀₋₇ alkylene-CO₂—C₅₋₁₅ alkyl, C₀₋₇ alkylene-CO₂—C₅₋₁₅ alkenyl, C₀₋₇ alkylene-O₂C—C₅₋₁₅ alkyl, C₀₋₇ alkylene-O₂C—C₅₋₁₅ alkenyl, C₂₋₇ alkenylene-CO₂—C₅₋₁₅ alkyl, C₂₋₇ alkenylene-CO₂—C₅₋₁₅ alkenyl, C₂₋₇ alkenylene-O₂C—C₅₋₁₅ alkyl, C₂₋₇ alkenylene-O₂C—C₅₋₁₅ alkenyl and C₀₋₇ alkylene-CO₂—C₅₋₁₅ alkylene-N(R⁵)R⁶.

According to some embodiments, R¹ and R² collectively have at least 9 carbon atoms, and R³ and R⁴ collectively have at least 9 carbon atoms. According to some embodiments, each one of L^{a} and L^{b} is C₀₋₄ alkylene or C₄₋₂₀ alkylene and at least one of L^{a} and L^{b} is C₃₋₁₅ alkylene. According to some embodiments, each one of L^{a} and L^{b} is C₀₋₂₀ alkylene and at least one of L^{a} and L^{b} is C₃₋₁₅ alkylene. According to some embodiments, each one of L^{a} and L^{b} is individually a C₃₋₂₀ alkylene. According to some embodiments, R¹ is selected from the group consisting of: C₈₋₁₈ alkyl, C₈₋₁₈ alkenyl, C₅₋₁₀ alkylene-CO₂—C₅₋₁₀ alkyl, C₁₋₃ alkylene-OH, C₁₋₃ alkylene-O—CH₂CH₂—OH, OH, C₁₋₃ alkylene-halogen, C₁₋₃ alkylene-PO₃H₂, methyl, ethyl, propyl and C₁₋₃ alkylene-NH₂; R² is selected from the group consisting of: C₈₋₁₈ alkyl, C₈₋₁₈ alkenyl, C₂₋₁₂ alkylene-CO₂H, C₅₋₁₀ alkylene-CO₂—C₅₋₁₀ alkenyl, C₅₋₁₀ alkylene-CO₂—C₅₋₁₀ alkyl and L-N(R₃)—R⁴; R³ is selected from the group consisting of: H, C₈₋₁₈ alkyl and C₈₋₁₈ alkenyl, C₁₋₃ alkylene-OH, OH, C₁₋₃ alkylene-halogen, C₁₋₃ alkylene-PO₃H₂, methyl, ethyl, propyl, and C₁₋₃ alkylene-NH₂; and R⁴ is selected from the group consisting of: C₈₋₁₈ alkyl, C₈₋₁₈ alkenyl, and C₅₋₁₀ alkylene-CO₂—C₅₋₁₀ alkenyl.

According to some embodiments, R¹ is selected from the group consisting of: C₁₋₃ alkylene-OH, OH, C₁₋₃ alkylene-halogen, C₁₋₃ alkylene-PO₃H₂, methyl, ethyl, propyl and C₁₋₃ alkylene-NH₂; R² is selected from the group consisting of: C₈₋₁₈ alkyl, C₈₋₁₈ alkenyl, C₅₋₁₀ alkylene-CO₂—C₅₋₁₀ alkenyl and L-N(R₃)—R⁴; R³ is selected from the group consisting of: C₁₋₃ alkylene-OH, OH, C₁₋₃ alkylene-halogen, C₁₋₃ alkylene-PO₃H₂, methyl, ethyl, propyl, and C₁₋₃ alkylene-NH₂; and R⁴ is selected from the group consisting of: C₈₋₁₈ alkyl, C₈₋₁₈ alkenyl, and C₅₋₁₀ alkylene-CO₂-C₅₋₁₀ alkenyl.

According to some embodiments, R¹ is selected from the group consisting of: C₈₋₁₈ alkyl, C₈₋₁₈ alkenyl; C₁₋₃ alkylene-OH, C₁₋₃ alkylene-O—CH₂CH₂—OH, C₁₋₃ alkylene-Cl, C₁₋₃ alkylene-NH₂, OH and C₁₋₃ alkylene-PO₃H₂; R² is selected from the group consisting of: C₈₋₁₈ alkyl, C₈₋₁₈ alkenyl, C₂₋₁₂ alkylene-CO₂H, L-N(R₃)—R₄, and C₅₋₁₀ alkylene-CO₂-C₅₋₁₀ alkenyl; R³ is selected from the group consisting of: H, C₈₋₁₈ alkyl and C₈₋₁₈ alkenyl; and R⁴ is selected from the group consisting of: C₈₋₁₈ alkyl, C₈₋₁₈ alkenyl.

According to some embodiments, R¹ is selected from the group consisting of: C₈₋₁₈ alkyl and C₈₋₁₈ alkenyl and C₅₋₁₀ alkylene-CO₂—C₅₋₁₀ alkyl; R² is selected from the group consisting of: C₈₋₁₈ alkyl, C₈₋₁₈ alkenyl and C₅₋₁₀ alkylene-CO₂—C₅₋₁₀ alkyl; R³ is selected from the group consisting of: H and C₈₋₁₈ alkyl and C₈₋₁₈ alkenyl; and R⁴ is a C₈₋₁₈ alkyl.

According to some embodiments, L is L¹-X¹-L²; X¹ is selected from the group consisting of: —O₂C—, —CO₂-L³-O₂C—, —O₂C-L³-CO₂—, —CO₂-L³-CO₂—, L³-CO₂— and L³-O₂C—; each one of L¹ and L² is independently selected from the group consisting of: C₄₋₂₀ alkylene and C₄₋₂₀ alkenylene; L³ is selected from the group consisting of: C₁₋₆ alkylene-(O—C₁₋₆ alkylene)ₙ-, C₂₋₆ alkenylene-(O—C₁₋₆ alkylene)ₘ-, C₁₋₆ alkylene-S—C₁₋₆ alkylene and C₁₋₆ alkylene-S—S—C₁₋₆ alkylene; each one of n and m is 0, 1, 2, 3, 4 or 5; R¹ is selected from the group consisting of: C₀₋₆ alkylene-Y¹, C₂₋₆ alkenylene-Y¹ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₂₋₁₅ alkylene-CO₂—C₀₋₄ alkylene-N(C₁₋₁₂ alkyl)₂, and C₅₋₁₅ alkylene-O₂C—C₅₋₁₅ alkylene-N(C₁₋₁₂ alkyl)₂; R³ is selected from the group consisting of: C₀₋₆ alkylene-Y¹, C₂₋₆ alkenylene-Y¹, and C₁₋₄ alkyl; R⁴ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl; each Y¹ is selected from the group consisting of: O—Y², halogen, PO₃H₂, PO₃(C₁₋₄ alkyl)₂, PO₃H(C₁₋₄ alkyl), NH₂, NMe₂, and NH—Y²; and Y² is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl;
or
L is L⁴-X²-L⁵; X² is selected from the group consisting of: —O₂C—, —CO₂-L⁶-O₂C—, —O₂C-L⁶-CO₂—, —CO₂-L⁶-CO₂—, L⁶-CO₂—, L⁶-O₂C— and —CO—NH—; L⁴ is a C₀₋₄ alkylene; L⁵ is selected from the group consisting of: C₂₋₂₀ alkylene and C₄₋₂₀ alkenylene; L⁶ is selected from the group consisting of: C₁₋₆ alkylene-(O—C₁₋₆ alkylene)ⱼ-, C₂₋₆ alkenylene-(O—C₁₋₆ alkylene)ₖ-, C₁₋₆ alkylene-S—C₁₋₆ alkylene and C₁₋₆ alkylene-S—S—C₁₋₆ alkylene; each one of j and k is 0, 1, 2, 3, 4 or 5; R¹ is selected from the group consisting of: C₀₋₁₀ alkylene-Y³, C₂₋₆ alkenylene-Y³ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂-C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H, C₂₋₁₅ alkylene-CO₂—C₀₋₄ alkylene-N(C₁₋₁₂ alkyl)₂, C₂₋₁₅ alkylene-CO₂—C₀₋₄ alkylene-NH—C₅₋₂₅ alkenyl, C₂₋₁₅ alkylene-CO₂—C₀₋₄ alkylene-N(C₅₋₂₅ alkenyl)₂, C₂₋₁₅ alkylene-CO—NH—C₀₋₄ alkylene-N(C₁₋₁₂ alkyl)₂ and C₂₋₁₅ alkylene-CO₂—C₀₋₄ alkylene-NH—C₁₋₁₂ alkyl; R³ is selected from the group consisting of: H, C₅₋₂₅ alkyl, C₅₋₂₅ alkenyl, C₅₋₁₅ alkylene-CO₂-C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂-C₅₋₁₅ alkenyl, C₅₋₁₅ alkenylene-O₂C—C₅₋₁₅ alkyl and C₅₋₁₅ alkenylene-O₂C—C₅₋₁₅ alkenyl; R⁴ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; Y³ is selected from the group consisting of: O—Y⁴, halogen, PO₃H₂, PO₃(C₁₋₄ alkyl)₂, PO₃H(C₁₋₄ alkyl), NH₂, and NH—Y⁴; Y⁴ is selected from the group consisting of: H, C₁₋₄ alkyl, C₁₋₄ alkylene-OH, C₁₋₄ alkylene-(O—C₁₋₄ alkylene)₁₋₃-OH, PO₃H₂, PO₃(C₁₋₄ alkyl)₂, PO₃H(C₁₋₄ alkyl), CO—C₁₋₄ alkyl, CO-aryl, CO—C₁₋₄ alkyl-N(CH₃)₂, SO₃H, SO₃—(C₁₋₄ alkyl), NMe₂, CO—C₁₋₄ alkyl-NM₂ and SO₃-aryl;
or
L is L⁷-X³-L⁸; X³ is selected from the group consisting of: —O₂C—, —CO₂—NH—, —CO₂-L⁹-O₂C—, —O₂C-L⁹-CO₂—, —CO₂-L⁹-CO₂— and L⁹-CO₂—, L⁹-O₂C—; L⁷ is a C₀₋₄ alkylene; L⁸ is a C₀₋₄ alkylene or C₄₋₂₀ alkylene; L⁹ is selected from the group consisting of: C₀₋₄ alkylene-aryl-C₀₋₄ alkylene, C₁₋₄ alkylene, C₁₋₆ alkylene-(O—C₁₋₆ alkylene)ₕ-, C₂₋₆ alkenylene-(O—C₁₋₆ alkylene)ᵢ-, C₁₋₆ alkylene-S—C₁₋₆ alkylene, and C₁₋₆ alkylene-S—S—C₁₋₆ alkylene; each one of h and i is 0, 1, 2, 3, 4 or 5; R¹ is selected from the group consisting of: C₅₋₂₅ alkyl, C₅₋₂₅ alkenyl and C₀₋₁₀ alkylene-O—CO—C₁₋₄ alkyl-N(CH₃)₂; each one of R² and R⁴ is independently selected from the group consisting of: C₅₋₂₅ alkyl and C₅₋₂₅ alkenyl; and R³ is selected from the group consisting of: H, C₅₋₂₅ alkyl, and C₅₋₂₅ alkenyl.

According to some embodiments, L is L¹-X¹-L²; X¹ is selected from the group consisting of: —O₂C—, —CO₂-L³-O₂C—, —O₂C-L³-CO₂—, —CO₂-L³-CO₂—, L³-CO₂— and L³-O₂C—; each one of L¹ and L² is selected from the group consisting of: C₄₋₂₀ alkylene and C₄₋₂₀ alkenylene; L³ is selected from the group consisting of: C₁₋₆ alkylene-(O—C₁₋₆ alkylene)ₙ-, C₂₋₆ alkenylene-(O—C₁₋₆ alkylene)ₘ-, C₁₋₆ alkylene-S—C₁₋₆ alkylene and C₁₋₆ alkylene-S—S—C₁₋₆ alkylene; each one of n and m is 0, 1, 2, 3, 4 or 5; R¹ is selected from the group consisting of: C₀₋₆ alkylene-Y¹, C₂₋₆ alkenylene-Y¹ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₂₋₁₅ alkylene-CO₂—C₀₋₄ alkylene-N(C₁₋₁₂ alkyl)₂, and C₅₋₁₅ alkylene-O₂C—C₅₋₁₅ alkylene-N(C₁₋₁₂ alkyl)₂; R³ is selected from the group consisting of: C₀₋₆ alkylene-Y¹, C₂₋₆ alkenylene-Y¹, and C₁₋₄ alkyl; R⁴ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl and C₅₋₁₅ alkylene-CO₂—C₅₋₁₅ alkylene-N(R⁵)R⁶; each Y¹ is selected from the group consisting of: O—Y², halogen, PO₃H₂, PO₃(C₁₋₄ alkyl)₂, PO₃H(C₁₋₄ alkyl), NH₂, NMe₂, and NH—Y²; and Y² is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl. According to some embodiments, X¹ is selected from the group consisting of: —O₂C— and —CO₂-L³-O₂C—; each one of L¹ and L² is C₅₋₁₅ alkylene; L³ is C₁₋₄ alkylene-; R¹ is C₀₋₄ alkylene-Y¹; R² is selected from the group consisting of: C₉₋₂₀ alkyl and C₉₋₂₀ alkenyl; R³ is C₁₋₄ alkylene-Y¹; R⁴ is selected from the group consisting of: C₅₋₂₅ alkyl, C₅₋₂₅ alkenyl, C₅₋₁₅ alkylene-CO₂-C₅₋₁₅ alkylene-N(R⁵)R⁶; and each Y¹ is OH.

It is to be understood that the phrase “each Y¹ is selected from the group consisting of” refers to the situation wherein R¹ may be C₀₋₆ alkylene-Y¹ or C₂₋₆ alkenylene-Y¹, and R³ may be C₀₋₆ alkylene-Y¹, C₂₋₆ alkenylene-Y¹, and it is intended to mean that in cases that Y¹ appears in both R¹ and R³, each of these Y¹ substituents may be independently selected from the Y¹ group. In other words, in cases that Y¹ appears in both R¹ and R³, the two Y¹ substituents may be the same or different.

According to some embodiments, L is L¹-X¹-L², and is selected from the group consisting of: C₆-alkylene-O₂C—C₆₋₁₅ alkylene, C₄₋₁₅ alkylene-CO₂—C₁₋₃ alkylene-(O—C₁₋₃ alkylene)˜-O₂C—C₄₋₁₅ alkylene, and C₄₋₁₅ alkylene-CO₂—C₁₋₄ alkylene-S—S—C₁₋₄ alkylene-O₂C—C₄₋₁₅ alkylene; wherein n is 0 1 or 2.

According to some embodiments, R¹ is selected from the group consisting of: CH₂CH₂PO₃H₂, CH₂CH₂Cl, CH₂CH₂OH, CH₃, OH and C₁₋₃ alkyl. According to some embodiments, R² is selected from the group consisting of: C₈₋₁₆ alkyl, C₈₋₂₀ alkenyl and L-N(R³)—R⁴. According to some embodiments, R² is the same as R⁴. According to some embodiments, R³ is selected from the group consisting of: CH₂CH₂PO₃H₂, CH₂CH₂Cl, CH₂CH₂OH, CH₃, OH and C₁₋₃ alkyl. According to some embodiments, R¹ is the same as R³. According to some embodiments, R⁴ is selected from the group consisting of: C₈₋₁₆ alkyl, C₈₋₂₀ alkenyl, and C₈₋₁₂ alkylene-CO₂—C₈₋₁₂ alkenyl.

According to some embodiments, L is L⁴-X²-L⁵; X² is selected from the group consisting of: —O₂C—, —CO₂-L⁶-O₂C—, —O₂C-L⁶-CO₂—, —CO₂-L⁶-CO₂—, L⁶-CO₂—, L⁶-O₂C— and —CO—NH—; L⁴ is a C₀₋₄ alkylene; L⁵ is selected from the group consisting of: C₂₋₂₀ alkylene and C₄₋₂₀ alkenylene; L⁶ is selected from the group consisting of: C₁₋₆ alkylene-(O—C₁₋₆ alkylene)ⱼ-, C₂₋₆ alkenylene-(O—C₁₋₆ alkylene)ₖ-, C₁₋₆ alkylene-S—C₁₋₆ alkylene and C₁₋₆ alkylene-S—S—C₁₋₆ alkylene; each one of j and k is 0, 1, 2, 3, 4 or 5; R¹ is selected from the group consisting of: C₀₋₁₀ alkylene-Y³, C₂₋₆ alkenylene-Y³ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H, C₂₋₁₅ alkylene-CO₂—C₀₋₄ alkylene-N(C₁₋₁₂ alkyl)₂, C₂₋₁₅ alkylene-CO₂—C₀₋₄ alkylene-NH—C₅₋₂₅ alkenyl, C₂₋₁₅ alkylene-CO₂—C₀₋₄ alkylene-N(C₅₋₂₅ alkenyl)₂, C₂₋₁₅ alkylene-CO—NH—C₀₋₄ alkylene-N(C₁₋₁₂ alkyl)₂ and C₂₋₁₅ alkylene-CO₂—C₀₋₄ alkylene-NH—C₁₋₁₂ alkyl; R³ is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl; R⁴ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; Y³ is selected from the group consisting of: O—Y⁴, halogen, PO₃H₂, PO₃(C₁₋₄ alkyl)₂, PO₃H(C₁₋₄ alkyl), NH₂, and NH—Y⁴; and Y⁴ is selected from the group consisting of: H, C₁₋₄ alkyl, C₁₋₄ alkylene-OH, C₁₋₄ alkylene-(O—C₁₋₄ alkylene)₁₋₃-OH, PO₃H₂, PO₃(C₁₋₄ alkyl)₂, PO₃H(C₁₋₄ alkyl), CO—C₁₋₄ alkyl, CO-aryl, CO—C₁₋₄ alkyl-N(CH₃)₂, SO₃H, SO₃—(C₁₋₄ alkyl), NMe₂, CO—C₁₋₄ alkyl-NM₂ and SO₃-aryl.

According to some embodiments, X² is —O₂C—; L⁴ is a C₀₋₄ alkylene or C₄₋₂₀ alkylene; L⁵ is a C₄₋₂₀ alkylene; R¹ is C₀₋₁₀ alkylene-Y³; R² is selected from the group consisting of: C₅₋₂₅ alkyl, C₅₋₂₅ alkenyl and C₂₋₁₅ alkylene-CO₂—C₀₋₄ alkylene-N(C₁₋₁₂ alkyl)₂; R³ is a C₅₋₂₅ alkyl; R⁴ is a C₅₋₂₅ alkyl; Y³ is O—Y^{b}; and Y⁴ is H.

According to some embodiments, L is L⁴-X²-L⁵; X² is selected from the group consisting of: —O₂C—, —CO₂-L⁶-O₂C—, —O₂C-L⁶-CO₂—, —CO₂-L⁶-CO₂—, L⁶-CO₂—, L⁶-O₂C— and —CO—NH—; L⁴ is a C₀₋₄ alkylene; L⁵ is selected from the group consisting of: C₄₋₂₀ alkylene and C₄₋₂₀ alkenylene; L⁶ is selected from the group consisting of: C₁₋₆ alkylene-(O—C₁₋₆ alkylene)ⱼ-, C₂₋₆ alkenylene-(O—C₁₋₆ alkylene)ₖ-, C₁₋₆ alkylene-S—C₁₋₆ alkylene and C₁₋₆ alkylene-S—S—C₁₋₆ alkylene; each one of j and k is 0, 1, 2, 3, 4 or 5; R¹ is selected from the group consisting of: C₀₋₁₀ alkylene-Y³, C₂₋₆ alkenylene-Y³ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H and L-N(R³)—R⁴; R³ is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl; R⁴ is selected from the group consisting of: C₅₋₂₅ alkyl, C₅-alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; Y³ is selected from the group consisting of: O—Y⁴, halogen, PO₃H₂, PO₃(C₁₋₄ alkyl)₂, PO₃H(C₁₋₄ alkyl), NH₂, and NH—Y⁴; Y⁴ is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl. According to some embodiments, L selected from the group consisting of: —O₂C—C₆₋₁₂ alkylene, C₁₋₃ alkylene-CO₂—C₁₋₃ alkylene-S—S—C₁₋₃ alkylene-O₂C—C₄₋₁₀ alkylene, C₁₋₃ alkylene-CO₂—C₆₋₁₂ alkylene, C₁₋₃ alkylene-O—C₁₋₃ alkylene-O₂C—C₆₋₁₂ alkylene, and C₁₋₃ alkylene-NHCO—C₆₋₁₂ alkylene. According to some embodiments, R¹ is selected from the group consisting of: CH₂CH₂OH, CH₂CH₂OCH₂CH₂OH, CH₂CH₂Cl, OH, CH₂CH₂PO₃H₂, and CH₂CH₂NH₂. According to some embodiments, R² is selected from the group consisting of: C₈₋₁₆ alkyl, C₈₋₂₀ alkenyl, C₆₋₁₂ alkylene-CO₂—C₆₋₁₂ alkenyl, C₆₋₁₂ alkylene-CO₂H, and L-N(R³)—R⁴. According to some embodiments, R³ is selected from the group consisting of: H, C₆₋₁₆ alkyl, and C₆₋₁₆ alkenyl. According to some embodiments, R³ is the same as R⁴. According to some embodiments, R⁴ is selected from the group consisting of: C₆₋₁₆ alkyl, C₆₋₁₆ alkenyl.

According to some embodiments, L is L⁷-X³-L⁸; X³ is selected from the group consisting of: —O₂C—, —CO₂—NH—, —CO₂-L⁹-O₂C—, —O₂C-L⁹-CO₂—, —CO₂-L⁹-CO₂— and L⁹-CO₂—, L⁹-O₂C—; L⁷ is a C₀₋₄ alkylene; L⁸ is a C₀₋₄ alkylene or C₄₋₂₀ alkylene; L⁹ is selected from the group consisting of: C₀₋₄ alkylene-aryl-C₀₋₄ alkylene, C₁₋₄ alkylene, C₁₋₆ alkylene-(O—C₁₋₆ alkylene)ₕ-, C₂₋₆ alkenylene-(O—C₁₋₆ alkylene)ᵢ-, C₁₋₆ alkylene-S—C₁₋₆ alkylene, and C₁₋₆ alkylene-S—S—C₁₋₆ alkylene; each one of h and i is 0, 1, 2, 3, 4 or 5; R¹ is selected from the group consisting of: C₅₋₂₅ alkyl, C₅₋₂₅ alkenyl and C₀₋₁₀ alkylene-O—CO—C₁₋₄ alkyl-N(CH₃)₂; each one of R² and R⁴ is independently selected from the group consisting of: C₅₋₂₅ alkyl and C₅₋₂₅ alkenyl; and R³ is selected from the group consisting of: H, C₅₋₂₅ alkyl, and C₅₋₂₅ alkenyl. According to some embodiments, X³ is-O₂C—; L⁷ is a C₀₋₄ alkylene; L⁸ is a C₀₋₄ alkylene; R¹ is C₅₋₂₅ alkyl; and each one of R¹, R², R³ and R⁴ is a C₅₋₂₅ alkyl.

According to some embodiments, L is L⁷-X³-L⁸; X³ is selected from the group consisting of: —O₂C—, —CO₂-L⁹-O₂C—, —O₂C-L⁹-CO₂—, —CO₂-L⁹-CO₂— and L⁹-CO₂—, L⁹-O₂C—; L⁷ is a C₀₋₄ alkylene; L⁸ is a C₀₋₄ alkylene; L⁹ is selected from the group consisting of: C₀₋₄ alkylene-aryl-C₀₋₄ alkylene, C₁₋₄ alkylene, C₁₋₆ alkylene-(O—C₁₋₆ alkylene)ₕ-, C₂₋₆ alkenylene-(O—C₁₋₆ alkylene)ᵢ-, C₁₋₆ alkylene-S—C₁₋₆ alkylene and C₁₋₆ alkylene-S—S—C₁₋₆ alkylene; each one of h and i is 0, 1, 2, 3, 4 or 5; and each one of R¹, R² and R³ is independently selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; and R⁴ is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl.

According to some embodiments, L is selected from the group consisting of C₁₋₄ alkylene-CO₂—C₁₋₄ alkylene, C₁₋₄ alkylene-CO₂—, O₂C—C₁₋₃ alkylene-C₆H₄-C₀₋₄ alkylene-CO₂, C₁₋₄ alkylene-O—C₁₋₄ alkylene-O₂C—C₁₋₄ alkylene, C₁₋₄ alkylene-O₂C—C₁₋₆ alkylene-CO₂—C₁₋₄ alkylene, O₂C—C₁₋₄ alkylene-S—S—C₁₋₄ alkylene-CO₂, and O₂C—C₁₋₆ alkylene-CO₂.

According to some embodiments, R¹ is selected from the group consisting of: C₈₋₁₅ alkyl, C₈₋₂₀ alkenyl, and C₆₋₁₂ alkylene-O₂C—C₉₋₁₈ alkylene. According to some embodiments, R² is selected from the group consisting of: C₈₋₁₅ alkyl, C₈₋₂₀ alkenyl, and C₆₋₁₂ alkylene-O₂C—C₉₋₁₈ alkylene.

According to some embodiments, R³ is selected from the group consisting of: C₈₋₁₅ alkyl and C₈₋₂₀ alkenyl. According to some embodiments, R⁴ is selected from the group consisting of: H, C₈₋₁₅ alkyl and C₈₋₂₀ alkenyl. According to some embodiments, R⁴ is selected from the group consisting of: C₈₋₁₅ alkyl and C₈₋₂₀ alkenyl.

According to some embodiments, at least two of R¹, R², R³, and R⁴ represent the same substituent.

According to some embodiments, each of R¹, R², R³, and R⁴ represents a different substituent.

According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL1-55, DSL1-56, DSL1-57, DSL1-58, DSL1-59, DSL1-60, DSL3c-1, DSL3c-2, DSL3c-3, DSL3c-4, DSL3c-5, DSL3c-6, DSL3c-7 and DSL3c-8, DSL2-1, DSL2-2, DSL2-3, DSL2-4, DSL2-5, DSL2-6, DSL2-7, DSL2-8, DSL2-9, DSL2-10, DSL2-11, DSL2-12, DSL2-13, DSL2-14, DSL2-15, DSL2-16, DSL2-17, DSL2-18, DSL2-19, DSL2-20, DSL2-21, DSL2-22, DSL2-23, DSL2-24, DSL2-25, DSL2-26, DSL2-27, DSL2-28, DSL2-29, DSL2-30, DSL2-31, DSL2-32, DSL2-33, DSL2-34, DSL2-35, DSL2-36, DSL2-37, DSL2-38, DSL2-39, DSL2-40, DSL2-41, DSL2-42, DSL2-43, DSL2-44, DSL2-45, DSL2-46, DSL2-47, DSL2-48, DSL2-49, DSL2-50, DSL2-51, DSL2-52, DSL2-53, DSL2-54, DSL2-55, DSL2-56, DSL2-57, DSL2-58, DSL2-59, DSL2-60, DSL2-61, DSL2-62, DSL2-63, DSL2-64, DSL2-65, DSL2-66, DSL2-67, DSL2-68, DSL2-69, DSL2-70, DSL2-71, DSL2-72, DSL4-1, DSL4-2, DSL4-3, DSL4-4, DSL4-5, DSL4-6, DSL4-7, DSL4-8, DSL4-9, DSL4-10, DSL4-11, DSL4-12, DSL4-13, DSL4-14 and DSL4-15. The chemical structures of each of the specific compound are detailed below in the “Exemplary Compounds” Section and in the claims.

According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL3c-1, DSL3c-2, DSL3c-3, DSL3c-4, DSL3c-5, DSL3c-6, DSL3c-7 and DSL3c-8, DSL2-1, DSL2-2, DSL2-3, DSL2-4, DSL2-5, DSL2-6, DSL2-7, DSL2-8, DSL2-9, DSL2-10, DSL2-11, DSL2-12, DSL2-13, DSL2-14, DSL2-15, DSL2-16, DSL2-17, DSL2-18, DSL2-19, DSL2-20, DSL2-21, DSL2-22, DSL2-23, DSL2-24, DSL2-25, DSL2-26, DSL2-27, DSL2-28, DSL2-29, DSL2-30, DSL2-31, DSL2-32, DSL2-33, DSL2-34, DSL2-35, DSL2-36, DSL2-37, DSL2-38, DSL2-39, DSL2-40, DSL2-41, DSL2-42, DSL2-43, DSL2-44, DSL2-45, DSL2-46, DSL2-47, DSL2-48, DSL4-1, DSL4-2, DSL4-3, DSL4-4, DSL4-5, DSL4-6, DSL4-7, DSL4-8, DSL4-9, DSL4-10, DSL4-11, DSL4-12, DSL4-13, DSL4-14 and DSL4-15. According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL2-55, DSL2-56, DSL2-57, DSL2-58, DSL2-59, DSL2-60, DSL3c-1, DSL3c-2, DSL3c-3, DSL3c-4, DSL3c-5, DSL3c-6, DSL3c-7 and DSL3c-8. According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL3c-1, DSL3c-2, DSL3c-3, DSL3c-4, DSL3c-5, DSL3c-6, DSL3c-7 and DSL3c-8. According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50 and DSL1-51, DSL1-52, DSL1-53, DSL1-54, DSL1-55, DSL1-56, DSL1-57, DSL1-58, DSL1-59 and DSL1-60. According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50 and DSL1-51, DSL1-52, DSL1-53 and DSL1-54. According to some embodiments, the lipid is selected from the group consisting of: DSL3c-1, DSL3c-2, DSL3c-3, DSL3c-4, DSL3c-5, DSL3c-6, DSL3c-7 and DSL3c-8. According to some embodiments, the lipid is selected is selected from the group consisting of: DSL2-1, DSL2-2, DSL2-3, DSL2-4, DSL2-5, DSL2-6, DSL2-7, DSL2-8, DSL2-9, DSL2-10, DSL2-11, DSL2-12, DSL2-13, DSL2-14, DSL2-15, DSL2-16, DSL2-17, DSL2-18, DSL2-19, DSL2-20, DSL2-21, DSL2-22, DSL2-23, DSL2-24, DSL2-25, DSL2-26, DSL2-27, DSL2-28, DSL2-29, DSL2-30, DSL2-31, DSL2-32, DSL2-33, DSL2-34, DSL2-35, DSL2-36, DSL2-37, DSL2-38, DSL2-39, DSL2-40, DSL2-41, DSL2-42, DSL2-43, DSL2-44, DSL2-45, DSL2-46, DSL2-47, DSL2-48, DSL2-49, DSL2-50, DSL2-51, DSL2-52, DSL2-53, DSL2-54, DSL2-55, DSL2-56, DSL2-57, DSL2-58, DSL2-59, DSL2-60, DSL2-61, DSL2-62, DSL2-63, DSL2-64, DSL2-65, DSL2-66, DSL2-67, DSL2-68, DSL2-69, DSL2-70, DSL2-71, DSL2-72. According to some embodiments, the lipid is selected is selected from the group consisting of: DSL2-1, DSL2-2, DSL2-3, DSL2-4, DSL2-5, DSL2-6, DSL2-7, DSL2-8, DSL2-9, DSL2-10, DSL2-11, DSL2-12, DSL2-13, DSL2-14, DSL2-15, DSL2-16, DSL2-17, DSL2-18, DSL2-19, DSL2-20, DSL2-21, DSL2-22, DSL2-23, DSL2-24, DSL2-25, DSL2-26, DSL2-27, DSL2-28, DSL2-29, DSL2-30, DSL2-31, DSL2-32, DSL2-33, DSL2-34, DSL2-35, DSL2-36, DSL2-37, DSL2-38, DSL2-39, DSL2-40, DSL2-41, DSL2-42, DSL2-43, DSL2-44, DSL2-45, DSL2-46, DSL2-47 and DSL2-48. According to some embodiments, the lipid is selected from the group consisting of: DSL4-1, DSL4-2, DSL4-3, DSL4-4, DSL4-5, DSL4-6, DSL4-7, DSL4-8, DSL4-9, DSL4-10, DSL4-11, DSL4-12, DSL4-13, DSL4-14 and DSL4-15. According to some embodiments, the lipid is selected from the group consisting of: DSL1-1, DSL1-2, DSL1-3, DSL1-4, DSL1-5, DSL2-1, DSL2-2, DSL4-1, DSL4-2, DSL3c-1, DSL2-49 and DSL2-50. According to some embodiments, the lipid is selected from the group consisting of: DSL1-1, DSL1-2, DSL1-3, DSL1-4, DSL1-5, DSL2-1, DSL2-2, DSL4-2, DSL3c-1, DSL2-49 and DSL2-50.

According to some embodiments, the present invention provides a particle comprising the lipid according to the present invention and a membrane stabilizing lipid. According to some embodiments, the particle comprises the membrane stabilizing lipid and a lipid membrane comprising the lipid.

According to some embodiments, the membrane stabilizing lipid is selected from the group consisting of cholesterol, phospholipids, cephalins, sphingolipids and glycoglycerolipids. According to some embodiments, the membrane stabilizing lipid comprises cholesterol. According to some embodiments, the particle further comprising one or more additional components selected from the group consisting of a PEG-lipid conjugate, a neutral lipid and a charged lipid. According to some embodiments, the additional component comprises 1,2-Distearoyl-sn-glycero-3-phosphocholine (DSPC). According to some embodiments, the additional component comprises 1,2-Dimyristoyl-sn-glyceryl-methoxy polyethylene glycol (DMG-PEG). According to some embodiments, the particle comprises the lipid, cholesterol, 1,2-Distearoyl-sn-glycero-3-phosphocholine (DSPC) and 1,2-Dimyristoyl-sn-glyceryl-methoxy polyethylene glycol (DMG-PEG). According to some embodiments, the particle is conjugated to a targeting moiety.

According to some embodiments, the particle further comprises a nucleic acid. According to some embodiments, the nucleic acid is encapsulated within a particle comprising the lipid. According to some embodiments, the nucleic acid is selected from the group consisting of small interfering RNA (siRNA), micro RNA (miRNA), antisense oligo nucleotides, messenger RNA (mRNA), ribozymes, pDNA, CRISPR mRNA, gRNA, circular RNA and immune stimulating nucleic acids.

According to some embodiments, the particle further comprises a therapeutic agent. According to some embodiments, the therapeutic agent is encapsulated within a particle comprising the lipid. According to some embodiments, the therapeutic agent is RNA comprising an open reading frame encoding a polypeptide that comprises a SARS-CoV-2 spike protein or an immunogenic fragment or variant thereof. Each possibility represents a separate embodiment of the invention. According to some embodiments, the therapeutic agent is RNA comprising an open reading frame encoding a polypeptide that comprises a SARS-CoV-2 spike protein, an immunogenic fragment of SARS-CoV-2 or a SARS-CoV-2 variant. Each possibility represents a separate embodiment of the invention. According to some embodiments, the therapeutic agent is RNA comprising an open reading frame encoding a polypeptide that comprises a SARS-CoV-2 spike protein.

According to some embodiments, there is provided a composition comprising a plurality of particles as discloses herein and a pharmaceutically acceptable carrier, diluent or excipient. According to some embodiments, the composition is a liposomal composition.

According to some embodiments, there is provided a method of gene silencing, comprising the step of contacting a cell with a composition according to the present invention.

According to some embodiments, there is provided a method of gene silencing, comprising the step of contacting a cell with a composition comprising a plurality of particles according to the present invention and a pharmaceutically acceptable carrier, diluent or excipient. According to some embodiments, the cell is a cancer cell.

In other embodiments, the compositions of the present invention may be used as a delivery system to administer a therapeutic agent to its target location in the body. According to some embodiments, there is provided method for administering a therapeutic agent, the method comprising the step of preparing a composition comprising a lipid according to the present invention, and a therapeutic agent, and administering the composition to a subject in need thereof. According to some embodiments, the method further comprises encapsulating the therapeutic agent within a particle comprising the lipid. According to some embodiments, the therapeutic agent is RNA comprising an open reading frame encoding a polypeptide that comprises a SARS-CoV-2 spike protein or an immunogenic fragment or variant thereof. Each possibility represents a separate embodiment of the invention. According to some embodiments, the therapeutic agent is RNA comprising an open reading frame encoding a polypeptide that comprises a SARS-CoV-2 spike protein or an immunogenic fragment or variant thereof. Each possibility represents a separate embodiment of the invention. According to some embodiments, the therapeutic agent is RNA comprising an open reading frame encoding a polypeptide that comprises a SARS-CoV-2 spike protein, an immunogenic fragment of SARS-CoV-2 or a SARS-CoV-2 variant. Each possibility represents a separate embodiment of the invention.

The lipids of the present invention can be used alone or in combination with other lipid components such as neutral lipids, charged lipids, steroids (including, for example, sterols) and/or their analogs, and/or polymer conjugated lipids to form lipid nanoparticles for the delivery of therapeutic agents. In some instances the lipid nanoparticles are used to deliver nucleic acids for the treatment of various diseases or conditions, in particular leukocyte associated conditions such as inflammation and/or lack of sufficient protein.

Thus, in some embodiments, the present invention relates to a method of treating a leukocyte associated condition, the method comprising the step of administering to a subject in need thereof a composition according to the present invention. The leukocyte associated condition may be selected from the group consisting of cancer, infection, autoimmune diseases, neurodegenerative diseases and inflammation. According to some embodiments, there is provided a method of treating a leukocyte associated condition, the method comprising the step of administering to a subject in need thereof a composition comprising a plurality of particles according to the present invention and a pharmaceutically acceptable carrier, diluent or excipient.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-B-9A-F represent the 1H NMR spectra (A) and ESI-MS (B) of: DSL1-1 (FIGS. 1A and 1B); DSL1-2 (FIGS. 2A and 2B); DSL1-3 (FIGS. 3A and 3B); DSL1-4 (FIGS. 4A and 4B); DSL1-5 (FIGS. 5A and 5B); DSL2-1 (FIGS. 6A and 6B); DSL2-2 (FIGS. 7A and 7B); DSL4-1 (FIGS. 8A and 8B); DSL4-2 (FIGS. 9A and 9B), DSL3c-1 (FIGS. 9C—1H NMR spectra and 9D—ESI-MS) and DSL1-50 (FIGS. 9E—1H NMR spectra and 9F—ESI-MS).

FIGS. 10A-10G depict transmission electron microscopy (TEM) images of LNPs made of ionizable lipids DSL1-1 (FIG. 10A), DSL1-2 (FIG. 10B), DSL1-5 (FIG. 10C), DSL2-50 (FIG. 10D), DSL1-3, (FIG. 10E), DSL3c-1 (FIG. 10F) or DSL2-49 (FIG. 10G). The images relate to organs from mice after intravenous administration of the lipids. The organs are lung, liver, spleen and kidney in this order (lung—top, and kidney—bottom).

FIGS. 11A-11K depict IVIS images of organs from mice after intravenous administration of mLUC-LNPs comprising DSL1-1 (FIG. 11A), DSL1-2 (FIG. 11B), DSL1-5 (FIG. 11C), DSL1-4 (FIG. 11D), DSL1-3 (FIG. 11E), DSL4-2 (FIG. 11F), DSL3c-1 (FIG. 11G), DSL2-2 (FIG. 111H), DSL2-1 (FIG. 11I), DSL2-49 (FIG. 11J) or DSL2-50 (FIG. 11K) as the ionizable lipid. The organs are lung, liver, spleen and kidney in this order (lung—top, and kidney—bottom).

FIG. 11L depicts IVIS. images of organs from mice administered intravenously with DSL1-3 LNPs comprising either 1.5% (second and third panels) or 2.5% PEG (fourth and fifth panels), untreated mice (first from left panel). The organs are lung, liver, spleen and kidney in this order (lung—top, and kidney—bottom).

FIGS. 11M-11P depict IVIS images of organs from mice administered with LNPs along with either DOPE or DSPC co-lipids. FIG. 11M—DSL1-4 LNPs with DOPE (left panel) or DSPC (right panel); FIG. 11N—DSL1-5 LNPs with DOPE (left panel) or DSPC (right panel); FIG. 11O—DSL2-2 LNPs with DOPE (left panel) or DSPC (right panel); FIG. 11P—DSL2-50 LNPs with DOPE (left panel) or DSPC (right panel). The organs are lung, liver, spleen and kidney in this order (lung—top, and kidney—bottom).

FIG. 11Q depicts IVIS images of organs from mice administered with LNPs at different mole ratios: top left—DSL1-5 at 30% mole ratio; top right—DSL1-5 at 40% mole ratio; bottom left DSL2-50 at 30% mole ratio; bottom right—DSL2-50 at 40% mole ratio. The organs are lung, liver, spleen and kidney in this order (lung—top, and kidney—bottom).

FIG. 11R depicts IVIS images of organs from mice administered with mRNA-LNPs composed of DSL2-50, either intravenously (left panel) or intra-muscularly (right panel). The organs in the left panel are lung, liver, spleen and kidney in this order (lung—top, and kidney—bottom). The organs in the right panel are lung, liver, spleen, kidney and muscle in this order (lung—top, and muscle—bottom).

FIG. 12 is a bar graph showing quantitative analysis of Luciferase expression in the spleen and liver of mice after intravenous administration of LNPs comprising DSL1-1, DSL1-2, DSL1-3, DSL1-4, DSL1-5, DSL2-1, DSL2-2, DSL4-2, DSL3c-1, DSL2-49 and DSL2-50.

FIGS. 13A and 13B are bar graphs demonstrating FACS analysis of tdTomato expression in peritoneal macrophages (FIG. 13A) and splenocytes (FIG. 13B) isolated from untreated (Mock, dotted bar) Ai9 mice or Ai9 mice following intravenous administration of mCRE-LNPs comprising DSL1-1 (black dots on while background), DSL1-2 (while dots on black background) or DSL1-3 (diagonal bar).

FIG. 14 is a bar graph demonstrating FACS analysis of tdTomato expression in different type of immune cells from spleens isolated from untreated (Mock) Ai9 mice or Ai9 mice following intravenous administration of mCRE-LNPs comprising DSL1-3.

FIGS. 15A and 15B are bar graphs showing quantitative analysis of tdTomato expression in the spleen (FIG. 15A) and liver (FIG. 15B) of Ai9 mice following intravenous administration of MC3 mCRE-LNPs LNPs or DSL1-2 mCRE-LNPs, compared to untreated (Mock) mice.

FIGS. 16A-16B show analysis of Luciferase expression as seen in IVIS images of organs from mice administered with DSL2-50 LNPs encapsulated with Luciferase mRNA prepared on day ‘0’ (FIG. 16A, left panel)) and after day ‘210’ (FIG. 16A, right panel). The lung, liver, spleen and kidney are arranged in this order (lung—top, and kidney—bottom). FIG. 16B is a histogram analysis of Luciferase expression on days 0 (vertical bars) or 210 (checkered bars) in mouse lung, liver, spleen and kidney.

FIGS. 16C-16D show analysis of mCherry expression as seen in IVIS images of organs from mice administered with DSL-50 LNPs encapsulated with mCherry mRNA prepared on day ‘0’ (FIG. 16C, middle panel), control (FIG. 16C, left panel) and 90 days after preparation (FIG. 16C, right panel). The organs in FIG. 16C are lung, liver, spleen and kidney in this order (lung—top, and kidney—bottom). FIG. 16D is a histogram analysis of mCherry expression, compared to controls (Mock, hatched bars) on days 0 (dotted bars) or 90 (horizontal bars) in mouse lung, liver, spleen and kidney.

FIG. 17 shows in vivo safety analysis of mRNA-LNPs (DSL2-50 or DSL1-3) after IV administration. After 2 hr and 24 hr blood was collected and serum samples were analyzed for levels of the liver enzymes alkaline phosphatase (Alk Phos), serum glutamic-oxaloacetic transaminase (SGOT) or serum glutamic pyruvic transaminase (SGPT).

FIGS. 18A-18B depict mRNA delivery capability of a single LNP. FIG. 18A shows IVIS images of mice livers following treatment with DSL2-50 LNPs (two right column) or Moderna's lipid SM102 as control (two left columns), encapsulated with luciferase (mLuc, bottom panel) or mCherry (top panel). FIG. 18B shows a histogram analysis of Luciferase and mCherry expression in mice livers.

FIGS. 19A-19B illustrate the evaluation of mRNA-LNPs for COVID-19 vaccine delivery in BALB/c mice, as evaluated by ELISA of SARS-CoV-2 spike-specific antibody titers (FIG. 19A) in sera of mice treated with DSL1-3, 2-2, 2-50 or Moderna's SM102 encapsulated RBD-hFc mRNA. FIG. 19B depicts ELISPOT assay for evaluation of COVID-19 specific T-cell response in splenocytes. Circles denote pre boost markers, triangles—post boost.

FIGS. 20A-20B depict evaluation of mRNA-LNPs for COVID-19 vaccine delivery in C57BL6 mice, as evaluated by ELISA SARS-CoV-2 spike-specific antibody titers (FIG. 20A) in sera of mice DSL1-3, 2-2, 2-50 or Moderna's SM102 encapsulated RBD-hFc mRNA. FIG. 20B depicts ELISPOT assay for evaluation of COVID-19 specific T-cell response in splenocytes. Circles denote pre boost markers, triangles—post boost.

### DETAILED DESCRIPTION OF THE PRESENT INVENTION

The present invention based on the discovery of lipids useful in preparing lipid nanoparticles to deliver active agents in vitro and in vivo. The lipids of the present invention are useful in delivery of nucleic acids such as siRNA, miRNA and mRNA etc.

Lipids

As contemplated herein, the present invention relates to a lipid represented by the structure of Formula (I):

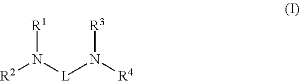

including salts, hydrates, solvates, polymorphs, optical isomers, geometrical isomers, enantiomers, diastereomers, and mixtures thereof.

According to some embodiments, L is L^{a}-X^{a}-L^{b}. It is to be understood that when describing that L^{a}-X^{a}-L^{b}, the order of substituents is only limited to X^{a} being in the center, flanked by L^{a} and L^{b}. it does not intended to mean that L^{a} is bonded to N(R¹)R² and L^{b} is bonded to N(R³)R⁴ or vice versa. Thus, L is selected from the group consisting of:

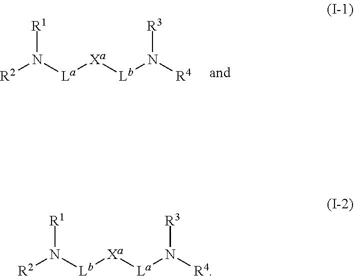

Each possibility represents a separate embodiment of the invention.

According to some embodiments, X^{a} is selected from the group consisting of: —O₂C—, —O—, —CO₂-L^{c}-O₂C—, —O₂C-L^{c}-CO₂—, —CO₂-L^{c}-CO₂—, L^{c}-CO₂—, L^{c}-O₂C—, —CO₂—NH— and —CO—NH—.

According to some embodiments, X^{a} is O₂C—. According to some embodiments, X^{a} is —CO₂-L^{c}-O₂C—. According to some embodiments, X^{a} is —O₂C-L^{c}-CO₂—. According to some embodiments, X^{a} is —CO₂-L^{c}-CO₂—. According to some embodiments, X^{a} is L^{c}-CO₂—. According to some embodiments, X^{a} is L^{c}-O₂C—. According to some embodiments, X^{a} is —CO—NH—. According to some embodiments, X^{a} is —CO₂—NH—.

According to some embodiments, L^{a} is selected from the group consisting of: C₄₋₂₀ alkylene, C₄₋₂₀ alkenylene and C₀₋₄ alkylene. According to some embodiments, L^{a} is C₄₋₂₀ alkylene. According to some embodiments, L^{a} is C₄₋₁₀ alkylene. According to some embodiments, L^{a} is C₁₀₋₂₀ alkylene. According to some embodiments, L^{a} is C₄₋₂₀ alkenylene. According to some embodiments, L^{a} is C₄₋₁₀ alkenylene. According to some embodiments, L^{a} is C₁₀₋₂₀ alkenylene. According to some embodiments, L^{a} is C₀₋₄ alkylene.

According to some embodiments, L^{b} is selected from the group consisting of: C₄₋₂₀ alkylene, C₄₋₂₀ alkenylene and C₀₋₄ alkylene. According to some embodiments, L^{b} is C₄₋₂₀ alkylene. According to some embodiments, L^{b} is C₄₋₁₀ alkylene. According to some embodiments, L^{b} is C₁₀₋₂₀ alkylene. According to some embodiments, L^{b} is C₄₋₂₀ alkenylene. According to some embodiments, L^{b} is C₄₋₁₀ alkenylene. According to some embodiments, L^{b} is C₁₀₋₂₀ alkenylene. According to some embodiments, L^{b} is C₀₋₄ alkylene.

According to some embodiments, L^{c} is selected from the group consisting of: C₁₋₆ alkylene-(O—C₁₋₆ alkylene)_{f}-, C₂₋₆ alkenylene-(O—C₁₋₆ alkylene)_{g}-, C₁₋₆ alkylene-S—C₁₋₆ alkylene, C₁₋₆ alkylene-S—S—C₁₋₆ alkylene, C₀₋₄ alkylene-aryl-C₀₋₄ alkylene, and C₁₋₄ alkylene. According to some embodiments, L^{c} is C₁₋₆ alkylene-(O—C₁₋₆ alkylene)_{f}-. According to some embodiments, L^{c} is C₁₋₆ alkylene. According to some embodiments, L^{c} is C₂₋₆ alkenylene-(O—C₁₋₆ alkylene)_{g}-. According to some embodiments, L^{c} is C₁₋₆ alkylene-S—C₁₋₆ alkylene. According to some embodiments, L^{c} is C₁₋₆ alkylene-S—S—C₁₋₆ alkylene. According to some embodiments, L^{c} is C₁₋₄ alkylene-S—S—C₁₋₄ alkylene. According to some embodiments, L^{c} is C₁₋₂ alkylene-S—S—C₁₋₂ alkylene. According to some embodiments, L^{c} is C₀₋₄ alkylene-aryl-C₀₋₄ alkylene. According to some embodiments, L^{c} is C₁₋₄ alkylene-aryl-C₁₋₄ alkylene. According to some embodiments, L^{c} is C₀₋₂ alkylene-aryl-C₀₋₂ alkylene. According to some embodiments, L^{c} is C₁₋₄ alkylene. According to some embodiments, each one of f and g is 0, 1, 2, 3, 4 or 5. According to some embodiments, f is 0, 1 or 2. According to some embodiments, f is 0. According to some embodiments, f is 1. According to some embodiments, g is 0, 1 or 2. According to some embodiments, g is 0. According to some embodiments, g is 1.

According to some embodiments, R¹ is selected from the group consisting of: C₀₋₁₀ alkylene-Y^{a}, C₂₋₆ alkenylene-Y^{a}, C₁₋₄ alkyl, C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl. According to some embodiments, R¹ is C₀₋₁₀ alkylene-Y^{a}. According to some embodiments, R¹ is Y^{a}. According to some embodiments, R¹ is C₁₋₂ alkylene-Y^{a}. According to some embodiments, R¹ is C₅₋₁₀ alkylene-Y^{a}. According to some embodiments, R¹ is C₂₋₆ alkenylene-Y^{a}. According to some embodiments, R¹ is C₁₋₄ alkyl. According to some embodiments, R¹ is C₅₋₂₅ alkyl. According to some embodiments, R¹ is C₅₋₁₅ alkyl. According to some embodiments, R¹ is C₁₂₋₂₅ alkyl. According to some embodiments, R¹ is C₅₋₂₅ alkenyl. According to some embodiments, R¹ is C₅₋₁₀ alkenyl. According to some embodiments, R¹ is C₁₀₋₂₅ alkenyl. According to some embodiments, R¹ is C₅₋₁₅ alkylene-CO₂—C₅₋₁₅ alkyl. According to some embodiments, R¹ is C₅₋₁₅ alkylene-CO₂-C₅₋₁₅ alkenyl. According to some embodiments, R¹ is C₅₋₁₅ alkylene-O₂C—C₅₋₁₅ alkyl. According to some embodiments, R¹ is C₅₋₁₅ alkylene-O₂C—C₅₋₁₅ alkenyl. According to some embodiments, R¹ is C₅₋₁₅ alkenylene-CO₂—C₅₋₁₅ alkyl. According to some embodiments, R¹ is C₅₋₁₅ alkenylene-CO₂—C₅₋₁₅ alkenyl. According to some embodiments, R¹ is C₅₋₁₅ alkenylene-O₂C—C₅₋₁₅ alkyl. According to some embodiments, R¹ is C₅₋₁₅ alkenylene-O₂C—C₅₋₁₅ alkenyl.

According to some embodiments, R² is selected from the group consisting of: R² is selected from the group consisting of: C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₂₋₁₅ alkylene-CO₂—C₀₋₄ alkylene-N(C₁₋₁₂ alkyl)₂, C₂₋₁₅ alkylene-CO₂—CH₂CH₂OCH₂CH₂—N(C₁₋₁₂ alkyl)₂, C₂₋₁₅ alkylene-CO₂—C₀₋₄ alkylene-NH—C₁₋₁₂ alkyl, C₂₋₁₅ alkylene-CO₂—C₀₋₄ alkylene-N(C₅₋₂₅ alkenyl)₂, C₂₋₁₅ alkylene-CO₂—C₀₋₄ alkylene-NH—C₅₋₂₅ alkenyl, C₂₋₁₅ alkylene-CO—NH—C₀₋₈ alkylene-N(C₁₋₁₂ alkyl)₂ and C₅₋₁₅ alkylene-CO₂H.

According to some embodiments, R² is selected from the group consisting of: C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H and L-N(R₃)—R₄.

According to some embodiments, R² is C₅₋₂₅ alkyl. According to some embodiments, R² is C₅₋₁₀ alkyl. According to some embodiments, R² is C₁₀₋₁₈ alkyl. According to some embodiments, R² is C₁₈₋₂₅ alkyl. According to some embodiments, R² is C₅₋₂₅ alkenyl. According to some embodiments, R² is C₅₋₁₀ alkenyl. According to some embodiments, R² is C₁₀₋₁₈ alkenyl. According to some embodiments, R² is C₁₈₋₂₅ alkenyl. According to some embodiments, R² is C₅₋₁₅ alkylene-CO₂—C₅₋₁₅ alkyl. According to some embodiments, R² is C₅₋₁₅ alkylene-CO₂-C₅₋₁₅ alkenyl. According to some embodiments, R² is C₅₋₁₅ alkylene-O₂C—C₅₋₁₅ alkyl. According to some embodiments, R² is C₅₋₁₅ alkylene-O₂C—C₅₋₁₅ alkenyl. According to some embodiments, R² is C₅₋₁₅ alkenylene-CO₂—C₅₋₁₅ alkyl. According to some embodiments, R² is C₅₋₁₅ alkenylene-CO₂—C₅₋₁₅ alkenyl. According to some embodiments, R² is C₅₋₁₅ alkenylene-O₂C—C₅₋₁₅ alkyl. According to some embodiments, R² is C₅₋₁₅ alkenylene-O₂C—C₅₋₁₅ alkenyl. According to some embodiments, R² is C₅₋₁₅ alkylene-CO₂H. According to some embodiments, R² is L-N(R₃)—R₄. According to some embodiments, R² is C₂₋₁₅ alkylene-CO₂—C₀₋₄ alkylene-N(C₁₋₁₂ alkyl)₂. According to some embodiments, R² is C₂₋₁₅ alkylene-CO₂—CH₂CH₂OCH₂CH₂—N(C₁₋₁₂ alkyl)₂. According to some embodiments, R² is C₂₋₁₅ alkylene-CO₂—C₀₋₄ alkylene-NH—C₁₋₁₂ alkyl. According to some embodiments, R² is C₂₋₁₅ alkylene-CO₂—C₀₋₄ alkylene-N(C₅₋₂₅ alkenyl)₂. According to some embodiments, R² is C₂₋₁₅ alkylene-CO₂—C₀₋₄ alkylene-NH—C₅₋₂₅ alkenyl. According to some embodiments, R² is C₂₋₁₅ alkylene-CO—NH—C₀₋₈ alkylene-N(C₁₋₁₂ alkyl)₂.

According to some embodiments, R³ is selected from the group consisting of: H, C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₀₋₆ alkylene-Y^{d}, C₂₋₆ alkenylene-Y^{d} and C₁₋₄ alkyl.

According to some embodiments, R³ is selected from the group consisting of: H, C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₀₋₆ alkylene-Y^{a}, C₂₋₆ alkenylene-Y^{a} and C₁₋₄ alkyl. According to some embodiments, R³ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂-C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₀₋₆ alkylene-Y^{a}, C₂₋₆ alkenylene-Y^{a} and C₁₋₄ alkyl. According to some embodiments, R³ is H.

According to some embodiments, R³ is C₅₋₂₅ alkyl. According to some embodiments, R³ is C₅₋₁₀ alkyl. According to some embodiments, R³ is C₁₀₋₁₈ alkyl. According to some embodiments, R³ is C₁₈₋₂₅ alkyl. According to some embodiments, R³ is C₅₋₂₅ alkenyl. According to some embodiments, R³ is C₅₋₁₀ alkenyl. According to some embodiments, R³ is C₁₀₋₁₈ alkenyl.

According to some embodiments, R³ is C₁₈₋₂₅ alkenyl. According to some embodiments, R³ is C₅₋₁₅ alkylene-CO₂—C₅₋₁₅ alkyl. According to some embodiments, R³ is C₅₋₁₅ alkylene-CO₂-C₅₋₁₅ alkenyl. According to some embodiments, R³ is C₅₋₁₅ alkylene-O₂C—C₅₋₁₅ alkyl. According to some embodiments, R³ is C₅₋₁₅ alkylene-O₂C—C₅₋₁₅ alkenyl. According to some embodiments, R³ is C₅₋₁₅ alkenylene-CO₂—C₅₋₁₅ alkyl. According to some embodiments, R³ is C₅₋₁₅ alkenylene-CO₂—C₅₋₁₅ alkenyl. According to some embodiments, R³ is C₅₋₁₅ alkenylene-O₂C—C₅₋₁₅ alkyl.

According to some embodiments, R³ is C₅₋₁₅ alkenylene-O₂C—C₅₋₁₅ alkenyl. According to some embodiments, R³ is C₀₋₆ alkylene-Y^{a}. According to some embodiments, R³ is C₀₋₆ alkylene-Y^{d}. According to some embodiments, R³ is C₂₋₆ alkenylene-Y^{d}. According to some embodiments, R³ is C₀₋₁₀ alkylene-Y^{a}. According to some embodiments, R³ is Y^{a}. According to some embodiments, R³ is Y^{d}. According to some embodiments, R³ is C₁₋₂ alkylene-Y^{a}. According to some embodiments, R³ is C₂₋₆ alkenylene-Y^{a}. According to some embodiments, R³ is C₁₋₄ alkyl. According to some embodiments, R³ is not H.

According to some embodiments, R⁴ is selected from the group consisting of: C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl and C₅₋₁₅ alkylene-CO₂—C₅₋₁₅ alkylene-N(R⁵)R⁶.

According to some embodiments, R⁴ is selected from the group consisting of: C₅₋₂₅ alkyl, C₅₋₂₅ alkenyl, C₅₋₂₅ alkynyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl.

According to some embodiments, R⁴ is C₅₋₂₅ alkyl. According to some embodiments, R⁴ is C₅₋₁₀ alkyl. According to some embodiments, R⁴ is C₁₀₋₁₈ alkyl. According to some embodiments, R⁴ is C₁₈₋₂₅ alkyl. According to some embodiments, R⁴ is C₅₋₂₅ alkenyl. According to some embodiments, R⁴ is C₅₋₁₀ alkenyl. According to some embodiments, R⁴ is C₁₀₋₁₈ alkenyl. According to some embodiments, R⁴ is C₁₈₋₂₅ alkenyl. According to some embodiments, R⁴ is C₅₋₁₅ alkylene-CO₂—C₅₋₁₅ alkyl. According to some embodiments, R⁴ is C₅₋₁₅ alkylene-CO₂-C₅₋₁₅ alkenyl. According to some embodiments, R⁴ is C₅₋₁₅ alkylene-O₂C—C₅₋₁₅ alkyl. According to some embodiments, R⁴ is C₅₋₁₅ alkylene-O₂C—C₅₋₁₅ alkenyl. According to some embodiments, R⁴ is C₅₋₁₅ alkenylene-CO₂—C₅₋₁₅ alkyl. According to some embodiments, R⁴ is C₅₋₁₅ alkenylene-CO₂—C₅₋₁₅ alkenyl. According to some embodiments, R⁴ is C₅₋₁₅ alkenylene-O₂C—C₅₋₁₅ alkyl. According to some embodiments, R⁴ is C₅₋₁₅ alkenylene-O₂C—C₅₋₁₅ alkenyl. According to some embodiments, R⁴ is C₅₋₁₅ alkylene-CO₂—C₅₋₁₅ alkylene-N(R⁵)R⁶.

According to some embodiments, R⁵ is selected from the group consisting of: C₁₋₄ alkyl, C₀₋₁₀ alkylene-OH, and C₀₋₁₀ alkylene-halogen. According to some embodiments, R⁵ is C₁₋₄ alkyl. According to some embodiments, R⁵ is C₀₋₁₀ alkylene-OH. According to some embodiments, R⁵ is C₀₋₁₀ alkylene-halogen.

According to some embodiments, R⁶ is selected from the group consisting of: C₅₋₂₅ alkyl, C₅₋₂₅ alkenyl. According to some embodiments, R⁶ is C₅₋₂₅ alkyl. According to some embodiments, R⁶ is C₅₋₂₅ alkenyl. According to some embodiments, Y^{a} is selected from the group consisting of: O—Y^{b}, halogen, PO₃H₂, PO₃(C₁₋₄ alkyl)₂, PO₃H(C₁₋₄ alkyl), pyrrolidinyl, piperidinyl piperazinyl, NH₂, SH, NMe₂, NMe₃⁺ and NH—Y^{b}. According to some embodiments, Y^{a} is O—Y^{b}. According to some embodiments, Y^{a} is a halogen. According to some embodiments, the halogen is selected from the group consisting of: fluorine, chlorine and bromine. According to some embodiments, the halogen is selected from the group consisting of: fluorine and chlorine. According to some embodiments, the halogen is chlorine. According to some embodiments, Y^{a} is selected from the group consisting of PO₃H₂, PO₃(C₁₋₄ alkyl)₂, PO₃H(C₁₋₄ alkyl). According to some embodiments, Y^{a} is PO₃H₂. According to some embodiments, Y^{a} is PO₃(C₁₋₄ alkyl)₂. According to some embodiments, Y^{a} is PO₃H(C₁₋₄ alkyl). According to some embodiments, Y^{a} is SH. According to some embodiments, Y^{a} is NH₂. According to some embodiments, Y^{a} is NH(CH₃). According to some embodiments, Y^{a} is N(CH₃)₂. According to some embodiments, Y^{a} is pyrrolidine. According to some embodiments, Y^{a} is piperidine. According to some embodiments, Y^{a} is piperazine. According to some embodiments, Y^{a} is NH—Y^{b}.

According to some embodiments, Y^{b} is selected from the group consisting of: H, C₁₋₄ alkyl, C₁₋₄ alkylene-OH, C₁₋₄ alkylene-(O—C₁₋₄ alkylene)₁₋₃-OH, PO₃H₂, PO₃(C₁₋₄ alkyl)₂, PO₃H(C₁₋₄ alkyl), CO—C₁₋₄ alkyl-Y^{c}, CO-aryl, SO₃H, SO₃—(C₁₋₄ alkyl) and SO₃-aryl. According to some embodiments, Y^{b} is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl-Y^{c}, CO-aryl, SO₃H, SO₃—(C₁₋₄ alkyl), SO₃-aryl. According to some embodiments, Y^{b} is selected from the group consisting of: C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl.

According to some embodiments, Y^{b} is C₁₋₄ alkyl. According to some embodiments, Y^{b} is C₁₋₄ alkylene-OH. According to some embodiments, Y^{b} is PO₃H₂. According to some embodiments, Y^{b} is PO₃(C₁₋₄ alkyl)₂. According to some embodiments, Y^{b} is PO₃H(C₁₋₄ alkyl). According to some embodiments, Y^{b} is CO—C₁₋₄ alkyl. According to some embodiments, Y^{b} is CO-aryl. According to some embodiments, the aryl is a phenyl, optionally substituted by at least one alkyl, haloalkyl or halogen. According to some embodiments, Y^{b} is SO₃H. According to some embodiments, Y^{b} is SO₃—(C₁₋₄ alkyl). According to some embodiments, Y^{b} is SO₃-aryl. According to some embodiments, Y^{b} is C₁₋₄ alkylene-(O—C₁₋₄ alkylene)₁₋₃-OH.

According to some embodiments, Y^{c} is selected from the group consisting of: H, NH₂, NH(CH₃), N(CH₃)₂, pyrrolidinyl piperidinyl and piperazinyl, each pyrrolidinyl piperidinyl and piperazinyl is optionally substituted with a C₁₋₄ alkyl. Each possibility represents a separate embodiment of the invention.

According to some embodiments, each of the pyrrolidinyl piperidinyl and piperazinyl in either Y^{a} or Y^{c} is bonded through the respective nitrogen atom. ach possibility represents a separate embodiment of the invention.

According to some embodiments, Y^{d} is selected from the group consisting of: O—Y^{e}, halogen, PO₃H₂, PO₃(C₁₋₄ alkyl)₂, PO₃H(C₁₋₄ alkyl), NH₂, SH, pyrrolidinyl, piperidinyl piperazinyl, NMe₂, NMe₃⁺ and NH—Y^{e}. According to some is O—Y^{e}. According to some embodiments, Y^{d} is a halogen. According to embodiments, Y^{d} some embodiments, Y^{d} is PO₃H₂, PO₃(C₁₋₄ alkyl)₂. According to some embodiments, Y^{d} is PO₃H(C₁₋₄ alkyl). According to some embodiments, Y^{d} is NH₂. According to some embodiments, Y^{d} is SH. According to some embodiments, Y^{d} is pyrrolidinyl. According to some embodiments, Y^{d} is piperidinyl. According to some embodiments, Y^{d} is piperazinyl. According to some embodiments, Y^{d} is NMe₂. According to some embodiments, Y^{d} is NMe₃⁺. According to some embodiments, Y^{d} is NH—Y^{e}.

According to some embodiments, Y^{e} is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl-Y^{f}, CO-aryl, SO₃H, SO₃—(C₁₋₄ alkyl) and SO₃-aryl. According to some embodiments, Y^{e} is H. According to some embodiments, Y^{e} is C₁₋₄ alkyl. According to some embodiments, Y^{e} is C₁₋₄ alkylene-OH. According to some embodiments, Y^{e} is PO₃H₂. According to some embodiments, Y^{e} is PO₃(C₁₋₄ alkyl)₂. According to some embodiments, Y^{e} is PO₃H(C₁₋₄ alkyl). According to some embodiments, Y^{e} is CO—C₁₋₄ alkyl-Y^{f}. According to some embodiments, Y^{e} is CO-aryl. According to some embodiments, Y^{e} is SO₃H. According to some embodiments, Y^{e} is SO₃—(C₁₋₄ alkyl). According to some embodiments, Y^{e} is SO₃-aryl.

According to some embodiments, Y^{f} is selected from the group consisting of: H, NH₂, NH(CH₃), N(CH₃)₂, pyrrolidinyl piperidinyl and piperazinyl, each pyrrolidinyl piperidinyl and piperazinyl is optionally substituted with a C₁₋₄ alkyl. Each possibility represents a separate embodiment of the invention.

According to some embodiments, any one of the alkenyl groups of R¹⁻⁴, (e.g., C₅₋₂₅ alkenyl) is a dienyl (e.g., C₅₋₂₅ dienyl). According to some embodiments, any one of the alkenyl groups of R¹⁻⁴, (e.g., C₅₋₂₅ alkenyl) is a mono-enyl (e.g., C₅₋₂₅ mono-enyl).

According to some embodiments, R² is selected from the group consisting of: C₈₋₂₅ alkyl, C₈₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₂₋₁₅ alkylene-CO₂—C₀₋₄ alkylene-N(C₁₋₁₂ alkyl)₂, C₂₋₁₅ alkylene-CO₂—C₀₋₄ alkylene-NH—C₁₋₁₂ alkyl, C₂₋₁₅ alkylene-CO₂—C₀₋₄ alkylene-N(C₅₋₂₅ alkenyl)₂, C₂₋₁₅ alkylene-CO₂—C₀₋₄ alkylene-NH—C₅₋₂₅ alkenyl, C₂₋₁₅ alkylene-CO—NH—C₀₋₄ alkylene-N(C₁₋₁₂ alkyl)₂, and C₅₋₁₅ alkylene-CO₂H; and R⁴ is selected from the group consisting of: C₈₋₂₅ alkyl, C₈₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂-C₅₋₁₅ alkenyl, C₅₋₁₅ alkenylene-O₂C—C₅₋₁₅ alkyl, C₅₋₁₅ alkenylene-O₂C—C₅₋₁₅ alkenyl, and C₅₋₁₅ alkylene-CO₂—C₅₋₁₅ alkylene-N(R⁵)R⁶. According to some embodiments, R² is selected from the group consisting of: C₈₋₂₅ alkyl, C₈₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H and L-N(R₃)—R₄; and R⁴ is selected from the group consisting of: C₈₋₂₅ alkyl, C₈₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl.

One of the features of the lipids of the present invention is that each of the nitrogen atoms in Formula I includes at least one lipid moiety. According to some embodiments, R¹ and R² collectively have at least 9 carbon atoms. According to some embodiments, R³ and R⁴ collectively have at least 9 carbon atoms. According to some embodiments, R¹ and R² collectively have at least 9 carbon atoms, and R³ and R⁴ collectively have at least 9 carbon atoms. According to some embodiments, R¹ and R² collectively have at least 10 carbon atoms. According to some embodiments, R³ and R⁴ collectively have at least 10 carbon atoms. According to some embodiments, R¹ and R² collectively have at least 10 carbon atoms, and R³ and R⁴ collectively have at least 10 carbon atoms. According to some embodiments, R¹ and R² collectively have at least 11 carbon atoms. According to some embodiments, R³ and R⁴ collectively have at least 11 carbon atoms. According to some embodiments, R¹ and R² collectively have at least 11 carbon atoms, and R³ and R⁴ collectively have at least 11 carbon atoms. According to some embodiments, R¹ and R² collectively have at least 12 carbon atoms. According to some embodiments, R³ and R⁴ collectively have at least 12 carbon atoms. According to some embodiments, R¹ and R² collectively have at least 12 carbon atoms, and R³ and R⁴ collectively have at least 12 carbon atoms. According to some embodiments, R¹ and R² collectively have at least 13 carbon atoms. According to some embodiments, R³ and R⁴ collectively have at least 13 carbon atoms. According to some embodiments, R¹ and R² collectively have at least 13 carbon atoms, and R³ and R⁴ collectively have at 13 carbon atoms.

According to some embodiments, each one of L^{a} and L^{b} is C₀₋₄ alkylene or C₄₋₂₀ alkylene and at least one of L^{a} and L^{b} is C₃₋₁₅ alkylene. According to some embodiments, each one of L^{a} and L^{b} is C₀₋₂₀ alkylene and at least one of L^{a} and L^{b} is C₃₋₁₅ alkylene. According to some embodiments, each one of L^{a} and L^{b} is C₀₋₂₀ alkylene. According to some embodiments, at least one of L^{a} and L^{b} is C₃₋₁₅ alkylene. According to some embodiments, each one of L^{a} and L^{b} is C₀₋₂₀ alkylene and at least one of L^{a} and L^{b} is C₃₋₁₅ alkylene. According to some embodiments, each one of L^{a} and L^{b} is C₀₋₁₅ alkylene. According to some embodiments, at least one of L^{a} and L^{b} is C₅₋₁₅ alkylene. According to some embodiments, each one of L^{a} and L^{b} is C₀₋₁₅ alkylene and at least one of L^{a} and L^{b} is C₅₋₁₅ alkylene.

According to some embodiments, each one of L^{a} and L^{b} is C₃₋₂₀ alkylene. According to some embodiments, each one of L^{a} and L^{b} is C₃₋₁₅ alkylene.

According to some embodiments, L^{a} and L^{b} collectively have at least 5 carbon atoms. According to some embodiments, L^{a} and L^{b} collectively have at least 6 carbon atoms. According to some embodiments, L^{a} and L^{b} collectively have at least 7 carbon atoms. According to some embodiments, L^{a} and L^{b} collectively have at least 8 carbon atoms. According to some embodiments, L^{a} and L^{b} collectively have at least 9 carbon atoms. According to some embodiments, L^{a} and L^{b} collectively have at least 10 carbon atoms. According to some embodiments, L^{a} and L^{b} collectively have at least 11 carbon atoms. According to some embodiments, L^{a} and L^{b} collectively have at least 12 carbon atoms.

According to some embodiments, R¹ is selected from the group consisting of: C₈₋₁₈ alkyl, C₈₋₁₈ alkenyl, C₅₋₁₀ alkylene-CO₂—C₅₋₁₀ alkyl, C₁₋₃ alkylene-OH, C₁₋₃ alkylene-O—CH₂CH₂—OH, OH, C₁₋₃ alkylene-halogen, C₁₋₃ alkylene-PO₃H₂, methyl, ethyl, propyl and C₁₋₃ alkylene-NH₂; R² is selected from the group consisting of: C₈₋₁₈ alkyl, C₈₋₁₈ alkenyl, C₂₋₁₂ alkylene-CO₂H, C₅₋₁₀ alkylene-CO₂—C₅₋₁₀ alkenyl, C₅₋₁₀ alkylene-CO₂—C₅₋₁₀ alkyl and L-N(R₃)—R⁴; R³ is selected from the group consisting of: H, C₈₋₁₈ alkyl and C₈₋₁₈ alkenyl, C₁₋₃ alkylene-OH, OH, C₁₋₃ alkylene-halogen, C₁₋₃ alkylene-PO₃H₂, methyl, ethyl, propyl, and C₁₋₃ alkylene-NH₂; and R⁴ is selected from the group consisting of: C₈₋₁₈ alkyl, C₈₋₁₈ alkenyl, and C₅₋₁₀ alkylene-CO₂—C₅₋₁₀ alkenyl.

According to some embodiments, X^{a} is selected from the group consisting of: —O₂C—, —O—, —CO₂-L^{c}-O₂C—, —O₂C-L^{c}-CO₂—, -L^{c}-O₂C—, —CO₂—NH— and —CO—NH—; L^{a} is selected from the group consisting of: C₄₋₂₀ alkylene, and C₀₋₄ alkylene; L^{b} is selected from the group consisting of: C₄₋₂₀ alkylene, and C₀₋₄ alkylene; L^{c} is selected from the group consisting of: C₁₋₆ alkylene-O—C₁₋₆ alkylene-, C₁₋₆ alkylene-S—S—C₁₋₆ alkylene, and C₁₋₄ alkylene; R¹ is selected from the group consisting of: C₀₋₁₀ alkylene-Y^{a}, C₁₋₄ alkyl, and C₅₋₂₅ alkyl, C₅₋₁₅ alkylene-CO₂—C₅₋₁₅ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₂₋₁₅ alkylene-CO₂—C₀₋₄ alkylene-N(C₁₋₁₂ alkyl)₂, C₂₋₁₅ alkylene-CO₂—C₀₋₄ alkylene-NH—C₁₋₁₂ alkyl, C₂₋₁₅ alkylene-CO₂—C₀₋₄ alkylene-N(C₅₋₂₅ alkenyl)₂, C₂₋₁₅ alkylene-CO₂—C₀₋₄ alkylene-NH—C₅₋₂₅ alkenyl, C₂₋₁₅ alkylene-CO—NH—C₀₋₈ alkylene-N(C₁₋₁₂ alkyl)₂ and C₅₋₁₅ alkylene-CO₂H; R³ is selected from the group consisting of: H, C₅₋₂₅ alkyl, C₅₋₂₅ alkenyl, C₀₋₆ alkylene-Y^{d}, and C₁₋₄ alkyl; R⁴ is selected from the group consisting of: C₅₋₂₅ alkyl, C₅₋₂₅ alkenyl, C₅₋₁₅ alkylene-CO₂—C₅₋₁₅ alkenyl, and C₅₋₁₅ alkylene-CO₂-C₅₋₁₅ alkylene-N(R⁵)R⁶; R⁵ is selected from the group consisting of: C₁₋₄ alkyl, C₀₋₁₀ alkylene-OH, and C₀₋₁₀ alkylene-halogen, R⁶ is selected from the group consisting of: C₅₋₂₅ alkyl, and C₅₋₂₅ alkenyl; Y^{a} is selected from the group consisting of: O—Y^{b}, halogen, PO₃H₂, NH₂, and NMe₂; Y^{b} is selected from the group consisting of: H, C₁₋₄ alkylene-OH, C₁₋₄ alkylene-(O—C₁₋₄ alkylene)₁₋₃-OH, and CO—C₁₋₄ alkyl-Y^{c}; Y^{c} is N(CH₃)₂; Y^{d} is selected from the group consisting of: O—Y^{e}, halogen, and NH₂; Y^{e} is selected from the group consisting of: H, and CO—C₁₋₄ alkyl-Y^{f}; and Y^{f} is selected from the group consisting of: N(CH₃)₂, and piperazinyl, which is optionally substituted with a C₁₋₄ alkyl.

According to some embodiments, X^{a} is selected from the group consisting of: —O₂C—, and —CO₂-L^{c}-O₂C—; L^{a} is selected from the group consisting of: C₄₋₂₀ alkylene, and C₀₋₄ alkylene; L^{b} is selected from the group consisting of: C₄₋₂₀ alkylene, and C₀₋₄ alkylene; L^{c} is C₁₋₄ alkylene; R¹ is selected from the group consisting of: C₀₋₁₀ alkylene-Y^{a}, and C₅₋₂₅ alkyl; R² is selected from the group consisting of: C₅₋₂₅ alkyl, C₅₋₂₅ alkenyl, and C₂₋₁₅ alkylene-CO₂—C₀₋₄ alkylene-N(C₁₋₁₂ alkyl)₂; R³ is selected from the group consisting of: C₅₋₂₅ alkyl, and C₀₋₆ alkylene-Y^{d}; R⁴ is selected from the group consisting of: C₅₋₂₅ alkyl, C₅₋₂₅ alkenyl, and C₅₋₁₅ alkylene-CO₂—C₅₋₁₅ alkylene-N(R⁵)R⁶; R⁵ is C₀₋₁₀ alkylene-OH; R⁶ is C₅₋₂₅ alkenyl; Y^{a} is O—Y^{b}; Y^{b} is H; Y^{d} is O—Y^{e}; and Y^{e} is H.

Formula (I′)

According to some embodiments, R² is represented by CH₂CH₂—R²²; R³ is represented by CH₂CH₂—R²³ and R⁴ is represented by CH₂CH₂—R²⁴; so the lipid is further represented by the structure of Formula (I′). According to some embodiments, R¹ is OH or CH₂CH₂—R²¹; R² comprises at least 6 carbon atoms and is represented by CH₂CH₂—R²²; R³ is CH₂CH₂—R²³; R⁴ comprises at least 6 carbon atoms and is represented by CH₂CH₂-R²⁴; none of R¹, R², R³, and R⁴ is H; so the lipid is further represented by the structure of Formula (I′).

The structure of Formula (I′) is presented below:

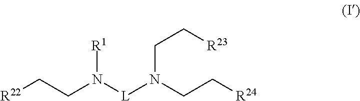

As can be understood by the person having ordinary skill in the art Formula (I′) is a substructure of formula (I) which is presented herein. Thus, embodiments relating to L and to R¹ as presented with respect to Formula (I) may similarly be used to describe Formula (I′). Also, one of the options of R¹ is CH₂CH₂—R²¹, as described above, thus as understood by the person having ordinary skill in the art, each of the options presented above for R¹, which comprise a terminal ethylene group, may be converted into a respective set of CH₂CH₂—R²¹ and R²¹. For example, R¹ is described above as optionally C₅₋₂₅ alkyl—this embodiment may be converted to R²¹ described as C₃₋₂₂ alkyl (given that the first two carbon atoms of the R¹ are each CH₂). Also, R¹ is described above as optionally C₀₋₁₀ alkylene-Y^{a}—this embodiment may be converted to R²¹ described as C₀₋₈ alkylene-Y^{a} (given that the first two carbon atoms of the R¹ are each CH₂). Similarly, R² is represented by CH₂CH₂—R²²; R³ is represented by CH₂CH₂—R²³ and R⁴ is represented by CH₂CH₂—R²⁴. So, similar conversions may be made to interpret R²²⁻²⁴, in view of the embodiments previously described for R²⁻⁴.

According to some embodiments, R¹ is OH or CH₂CH₂—R²¹. According to some embodiments, R¹ is OH. According to some embodiments, R¹ is CH₂CH₂—R²¹. According to some embodiments, R² comprises at least 6 carbon atoms. According to some embodiments, R² comprises at least 7, 8, 9, 10, 11 or 12 carbon atoms. Each possibility represents a separate embodiment of the invention. According to some embodiments, R²² comprises at least 4 carbon atoms. According to some embodiments, R²² comprises at least 5, 6, 7, 8, 9, or 10 carbon atoms. Each possibility represents a separate embodiment of the invention. According to some embodiments, R⁴ comprises at least 6 carbon atoms. According to some embodiments, R⁴ comprises at least 7, 8, 9, 10, 11 or 12 carbon atoms. Each possibility represents a separate embodiment of the invention. According to some embodiments, R²⁴ comprises at least 4 carbon atoms. According to some embodiments, R²⁴ comprises at least 5, 6, 7, 8, 9, or 10 carbon atoms. According to some embodiments, none of R¹, R², R³, and R⁴ is H.

According to some embodiments, R²¹ is selected from the group consisting of: C₀₋₈ alkylene-Y^{a}, C₂₋₄ alkenylene-Y^{a}, C₁₋₂ alkyl, C₃₋₂₅ alkyl, C₅₋₂₃ alkenyl, C₃₋₂₃ alkynyl, C₃₋₁₃ alkylene-CO₂—C₅₋₁₅ alkyl, C₃₋₁₃ alkylene-CO₂—C₅₋₁₅ alkenyl, C₃₋₁₃ alkylene-O₂C—C₅₋₁₅ alkyl, C₃₋₁₃ alkylene-O₂C—C₅₋₁₅ alkenyl, C₃₋₁₃ alkenylene-CO₂—C₅₋₁₅ alkyl, C₃₋₁₃ alkenylene-CO₂—C₅₋₁₅ alkenyl, C₃₋₁₃ alkenylene-O₂C—C₅₋₁₅ alkyl, and C₃₋₁₃ alkenylene-O₂C—C₅₋₁₅ alkenyl. According to some embodiments, R²¹ is C₀₋₈ alkylene-Y^{a}. According to some embodiments, R²¹ is C₂₋₄ alkenylene-Y^{a}. According to some embodiments, R²¹ is C₁₋₂ alkyl. According to some embodiments, R²¹ is C₃₋₂₅ alkyl. According to some embodiments, R²¹ is C₅₋₂₃ alkenyl. According to some embodiments, R²¹ is C₃₋₂₃ alkynyl. According to some embodiments, R²¹ is C₃₋₁₃ alkylene-CO₂-C₅₋₁₅ alkyl. According to some embodiments, R²¹ is C₃₋₁₃ alkylene-CO₂—C₅₋₁₅ alkenyl.

According to some embodiments, R²¹ is C₃₋₁₃ alkylene-O₂C—C₅₋₁₅ alkyl. According to some embodiments, R²¹ is C₃₋₁₃ alkylene-O₂C—C₅₋₁₅ alkenyl. According to some embodiments, R²¹ is C₃₋₁₃ alkenylene-CO₂—C₅₋₁₅ alkyl. According to some embodiments, R²¹ is C₃₋₁₃ alkenylene-CO₂-C₅₋₁₅ alkenyl. According to some embodiments, R²¹ is C₃₋₁₃ alkenylene-O₂C—C₅₋₁₅ alkyl. According to some embodiments, R²¹ is and C₃₋₁₃ alkenylene-O₂C—C₅₋₁₅ alkenyl.

According to some embodiments, R²² is selected from the group consisting of: C₄₋₂₃ alkyl, C₄₋₂₃ alkenyl, C₄₋₂₃ alkynyl, C₃₋₁₃ alkylene-CO₂—C₅₋₁₅ alkyl, C₃₋₁₃ alkylene-CO₂—C₅₋₁₅ alkenyl, C₃₋₁₃ alkylene-O₂C—C₅₋₁₅ alkyl, C₃₋₁₃ alkylene-O₂C—C₅₋₁₅ alkenyl, C₃₋₁₃ alkenylene-CO₂—C₅₋₁₅ alkyl, C₃₋₁₃ alkenylene-CO₂—C₅₋₁₅ alkenyl, C₃₋₁₃ alkenylene-O₂C—C₅₋₁₅ alkyl, C₃₋₁₃ alkenylene-O₂C—C₅₋₁₅ alkenyl, C₀₋₁₃ alkylene-CO₂—C₀₋₄ alkylene-N(C₁₋₁₂ alkyl)₂, C₀₋₁₃ alkylene-CO₂—C₀₋₄ alkylene-NH—C₁₋₁₂ alkyl, C₀₋₁₃ alkylene-CO₂—C₀₋₄ alkylene-N(C₅₋₂₅ alkenyl)₂, C₀₋₁₃ alkylene-CO₂—C₀₋₄ alkylene-NH—C₅₋₂₅ alkenyl, C₀₋₁₃ alkylene-CO—NH—C₀₋₈ alkylene-N(C₁₋₁₂ alkyl)₂ and C₃₋₁₃ alkylene-CO₂H. According to some embodiments, R²² is C₄₋₂₃ alkyl. According to some embodiments, R²² is C₄₋₂₃ alkenyl. According to some embodiments, R²² is C₄₋₂₃ alkynyl. According to some embodiments, R²² is C₃₋₁₃ alkylene-CO₂—C₅₋₁₅ alkyl. According to some embodiments, R²² is C₃₋₁₃ alkylene-CO₂—C₅₋₁₅ alkenyl. According to some embodiments, R²² is C₃₋₁₃ alkylene-O₂C—C₅₋₁₅ alkyl. According to some embodiments, R²² is C₃₋₁₃ alkylene-O₂C—C₅₋₁₅ alkenyl. According to some embodiments, R²² is C₃₋₁₃ alkenylene-CO₂—C₅₋₁₅ alkyl. According to some embodiments, R²² is C₃₋₁₃ alkenylene-CO₂—C₅₋₁₅ alkenyl. According to some embodiments, R²² is C₃₋₁₃ alkenylene-O₂C—C₅₋₁₅ alkyl. According to some embodiments, R²² is C₃₋₁₃ alkenylene-O₂C—C₅₋₁₅ alkenyl. According to some embodiments, R²² is C₀₋₁₃ alkylene-CO₂-C₀₋₄ alkylene-N(C₁₋₁₂ alkyl)₂. According to some embodiments, R²² is C₀₋₁₃ alkylene-CO₂-C₀₋₄ alkylene-NH—C₁₋₁₂ alkyl. According to some embodiments, R²² is C₀₋₁₃ alkylene-CO₂-C₀₋₄ alkylene-N(C₅₋₂₅ alkenyl)₂. According to some embodiments, R²² is C₀₋₁₃ alkylene-CO₂-C₀₋₄ alkylene-NH—C₅₋₂₅ alkenyl. According to some embodiments, R²² is C₀₋₁₃ alkylene-CO—NH—C₀₋ₛ alkylene-N(C₁₋₁₂ alkyl)₂. According to some embodiments, R²² is C₃₋₁₃ alkylene-CO₂H. R²³ is selected from the group consisting of: H, C₃₋₂₃ alkyl, C₃₋₂₃ alkenyl, C₃₋₂₃ alkynyl, C₃₋₁₃ alkylene-CO₂—C₅₋₁₅ alkyl, C₃₋₁₃ alkylene-CO₂—C₅₋₁₅ alkenyl, C₃₋₁₃ alkylene-O₂C—C₅₋₁₅ alkyl, C₃₋₁₃ alkylene-O₂C—C₅₋₁₅ alkenyl, C₃₋₁₃ alkenylene-CO₂—C₅₋₁₅ alkyl, C₃₋₁₃ alkenylene-CO₂-C₅₋₁₅ alkenyl, C₃₋₁₃ alkenylene-O₂C—C₅₋₁₅ alkyl, C₃₋₁₃ alkenylene-O₂C—C₅₋₁₅ alkenyl, C₀₋₄ alkylene-Y^{d}, C₀₋₄ alkenylene-Y^{d} and C₁₋₂ alkyl. According to some embodiments, R²³ is H. According to some embodiments, R²³ is C₃₋₂₃ alkyl. According to some embodiments, R²³ is C₃₋₂₃ alkenyl. According to some embodiments, R²³ is C₃₋₂₃ alkynyl. According to some embodiments, R²³ is C₃₋₁₃ alkylene-CO₂—C₅₋₁₅ alkyl. According to some embodiments, R²³ is C₃₋₁₃ alkylene-CO₂—C₅₋₁₅ alkenyl. According to some embodiments, R²³ is C₃₋₁₃ alkylene-O₂C—C₅₋₁₅ alkyl. According to some embodiments, R²³ is C₃₋₁₃ alkylene-O₂C—C₅₋₁₅ alkenyl. According to some embodiments, R²³ is C₃₋₁₃ alkenylene-CO₂—C₅₋₁₅ alkyl. According to some embodiments, R²³ is C₃₋₁₃ alkenylene-CO₂—C₅₋₁₅ alkenyl. According to some embodiments, R²³ is C₃₋₁₃ alkenylene-O₂C—C₅₋₁₅ alkyl. According to some embodiments, R²³ is C₃₋₁₃ alkenylene-O₂C—C₅₋₁₅ alkenyl. According to some embodiments, R²³ is C₀₋₄ alkylene-Y^{d}. According to some embodiments, R²³ is C₀₋₄ alkenylene-Y^{d}. According to some embodiments, R²³ is C₁₋₂ alkyl. Y^{d} is as described in various embodiments of the present invention, e.g., when relating to Formula (I). According to some embodiments, R²⁴ is selected from the group consisting of: C₃₋₂₃ alkyl, C₃₋₂₃ alkenyl, C₃₋₂₃ alkynyl, C₃₋₁₃ alkylene-CO₂—C₅₋₁₅ alkyl, C₃₋₁₃ alkylene-CO₂—C₅₋₁₅ alkenyl, C₃₋₁₃ alkylene-O₂C—C₅₋₁₅ alkyl, C₃₋₁₃ alkylene-O₂C—C₅₋₁₅ alkenyl, C₃₋₁₃ alkenylene-CO₂—C₅₋₁₅ alkyl, C₃₋₁₃ alkenylene-CO₂—C₅₋₁₅ alkenyl, C₃₋₁₃ alkenylene-O₂C—C₅₋₁₅ alkyl, C₃₋₁₃ alkenylene-O₂C—C₅₋₁₅ alkenyl and C₃₋₁₃ alkylene-CO₂—C₅₋₁₅ alkylene-N(R⁵)R⁶.

According to some embodiments. According to some embodiments, R²⁴ is R²⁴ is C₃₋₂₃ alkyl. According to some embodiments, R²⁴ is C₃₋₂₃ alkenyl. According to some embodiments, R²⁴ is C₃₋₂₃ alkynyl. According to some embodiments, R²⁴ is C₃₋₁₃ alkylene-CO₂—C₅₋₁₅ alkyl. According to some embodiments, R²⁴ is C₃₋₁₃ alkylene-CO₂—C₅₋₁₅ alkenyl. According to some embodiments, R²⁴ is C₃₋₁₃ alkylene-O₂C—C₅₋₁₅ alkyl. According to some embodiments, R²⁴ is C₃₋₁₃ alkylene-O₂C—C₅₋₁₅ alkenyl. According to some embodiments, R²⁴ is C₃₋₁₃ alkenylene-CO₂—C₅₋₁₅ alkyl. According to some embodiments, R²⁴ is C₃₋₁₃ alkenylene-CO₂—C₅₋₁₅ alkenyl. According to some embodiments, R²⁴ is C₃₋₁₃ alkenylene-O₂C—C₅₋₁₅ alkyl. According to some embodiments, R²⁴ is C₃₋₁₃ alkenylene-O₂C—C₅₋₁₅ alkenyl. According to some embodiments, R²⁴ is C₃₋₁₃ alkylene-CO₂-C₅₋₁₅ alkylene-N(R⁵)R⁶.

Formula (I″)

According to some embodiments, R²² is represented by CH₂CH₂—R³². According to some embodiments, R²⁴ is represented by CH₂CH₂—R³⁴. According to some embodiments, R²² is represented by CH₂CH₂—R³² and R²⁴ is represented by CH₂CH₂—R³⁴, so the lipid is further represented by the structure of Formula (I″). According to some embodiments, R² is represented by CH₂(CH₂)₃—R³². According to some embodiments, R⁴ is represented by CH₂(CH₂)₃—R³⁴. According to some embodiments, R² is represented by CH₂(CH₂)₃—R³² and R⁴ is represented by CH₂(CH₂)₃—R³⁴, so the lipid is further represented by the structure of Formula (I″). In addition, as detailed above for Formula (I′), R¹ may be OH or CH₂CH₂—R²¹ and R³ may be CH₂CH₂—R²³, according to some embodiments. Thus, any reference to R¹, R²¹, R³ and R²³, may similarly apply to the respective substituent in Formula (I″).

The structure of Formula (I″) is presented below:

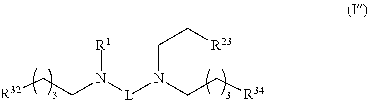

As can be understood by the person having ordinary skill in the art Formula (I″) is a substructure of Formulas (I) and (I′) which are presented herein. Thus, embodiments relating to L and to R¹ as presented with respect to Formulas (I) and/or (I′) may similarly be used to describe Formula (I″). Also, one of the options of R¹ is CH₂CH₂—R²¹, as described above, thus as understood by the person having ordinary skill in the art, each of the options presented above for R¹, which comprise a terminal ethylene group, may be converted into a respective set of CH₂CH₂—R²¹ and R²¹. For example, R¹ is described above as optionally C₅₋₂₅ alkyl—this embodiment may be converted to R²¹ described as C₃₋₂₂ alkyl (given that the first two carbon atoms of the R¹ are each CH₂). Also, R¹ is described above as optionally C₀₋₁₀ alkylene-Y^{a}—this embodiment may be converted to R²¹ described as C₀₋₈ alkylene-Y^{a} (given that the first two carbon atoms of the R¹ are each CH₂). Likewise, according to some embodiments, R²² is represented by CH₂CH₂—R³² and/or R² is represented by CH₂(CH₂)₃—R³²—thus, similar conversions may be made to interpret R³², in view of the embodiments previously described for R² (e.g., when relating to Formula (I)) and/or R²² (e.g., when relating to Formula (I′)). For example, R² is described above as optionally C₅₋₁₅ alkylene-CO₂—C₅₋₁₅ alkyl—this embodiment may be converted to (a) R²² described as C₃₋₁₃ alkylene-CO₂—C₅₋₁₅ alkyl (given that the first two carbon atoms of the R² are each CH₂); and/or (b) R³² described as C₁₋ₙ alkylene-CO₂—C₅₋₁₅ alkyl (given that the first two carbon atoms of the R²² are each CH₂, i.e., the first four carbon atoms of the R² are each CH₂). Similarly, according to some embodiments, R²⁴ is represented by CH₂CH₂—R³⁴ and/or R⁴ is represented by CH₂(CH₂)₃—R³⁴—thus, similar conversions may be made to interpret R³⁴, in view of the embodiments previously described for R⁴ and/or R²⁴. Lastly, R²³ of Formula (I″) is as described for R²³ of Formula (I′). According to some embodiments, R¹ is OH or CH₂CH₂—R²¹. According to some embodiments, R¹ is OH. According to some embodiments, R¹ is CH₂CH₂-R²¹. According to some embodiments, R² comprises at least 6 carbon atoms. According to some embodiments, R² comprises at least 7, 8, 9, 10, 11 or 12 carbon atoms. Each possibility represents a separate embodiment of the invention. According to some embodiments, R³² comprises at least 2 carbon atoms. According to some embodiments, R³² comprises at least 3, 4, 5, 6, 7 or 8 carbon atoms. Each possibility represents a separate embodiment of the invention. According to some embodiments, R⁴ comprises at least 6 carbon atoms. According to some embodiments, R⁴ comprises at least 7, 8, 9, 10, 11 or 12 carbon atoms. Each possibility represents a separate embodiment of the invention. According to some embodiments, R³⁴ comprises at least 2 carbon atoms. According to some embodiments, R³⁴ comprises at least 3, 4, 5, 6, 7 or 8 carbon atoms. According to some embodiments, none of R¹, R², R³, and R⁴ is H.

According to some embodiments, R³² is selected from the group consisting of: C₂₋₂₁ alkyl, C₂₋₂₁ alkenyl, C₂₋₂₁ alkynyl, C₁₋₁₁ alkylene-CO₂—C₅₋₁₅ alkyl, C₁₋₁₁ alkylene-CO₂—C₅₋₁₅ alkenyl, C₁₋₁₁ alkylene-O₂C—C₅₋₁₅ alkyl, C₁₋₁₁ alkylene-O₂C—C₅₋₁₅ alkenyl, C₂₋₁₁ alkenylene-CO₂—C₅₋₁₅ alkyl, C₂₋₁₁ alkenylene-CO₂—C₅₋₁₅ alkenyl, C₂₋₁₁ alkenylene-O₂C—C₅₋₁₅ alkyl, C₂₋₁₁ alkenylene-O₂C—C₅₋₁₅ alkenyl, C₀₋₁₁ alkylene-CO₂—C₀₋₄ alkylene-N(C₁₋₁₂ alkyl)₂, C₀₋₁₁ alkylene-CO₂—C₀₋₄ alkylene-NH—C₁₋₁₂ alkyl, C₀₋₁₃ alkylene-CO₂—C₀₋₄ alkylene-N(C₅₋₂₅ alkenyl)₂, C₀₋₁₁ alkylene-CO₂—C₀₋₄ alkylene-NH—C₅₋₂₅ alkenyl, C₀₋₁₁ alkylene-CO—NH—C₀₋₄ alkylene-N(C₁₋₁₂ alkyl)₂ and C₁₋₁₁ alkylene-CO₂H. According to some embodiments, R³² is C₂₋₂₁ alkyl. According to some embodiments, R³² is C₂₋₂₁ alkenyl. According to some embodiments, R³² is C₂₋₂₁ alkynyl. According to some embodiments, R³² is C₁₋₁₁ alkylene-CO₂—C₅₋₁₅ alkyl. According to some embodiments, R³² is C₁₋₁₁ alkylene-CO₂—C₅₋₁₅ alkenyl. According to some embodiments, R³² is C₁₋₁₁ alkylene-O₂C—C₅₋₁₅ alkyl. According to some embodiments, R³² is C₁₋₁₁ alkylene-O₂C—C₅₋₁₅ alkenyl. According to some embodiments, R³² is C₂₋₁₁ alkenylene-CO₂—C₅₋₁₅ alkyl. According to some embodiments, R³² is C₂₋₁₁ alkenylene-CO₂—C₅₋₁₅ alkenyl. According to some embodiments, R³² is C₂₋₁₁ alkenylene-O₂C—C₅₋₁₅ alkyl. According to some embodiments, R³² is C₂₋₁₁ alkenylene-O₂C—C₅₋₁₅ alkenyl. According to some embodiments, R³² is C₀₋₁₁ alkylene-CO₂-C₀₋₄ alkylene-N(C₁₋₁₂ alkyl)₂. According to some embodiments, R³² is C₀₋₁₁ alkylene-CO₂—C₀₋₄ alkylene-NH—C₁₋₁₂ alkyl. According to some embodiments, R³² is C₀₋₁₃ alkylene-CO₂—C₀₋₄ alkylene-N(C₅₋₂₅ alkenyl)₂. According to some embodiments, R³² is C₀₋₁₁ alkylene-CO₂—C₀₋₄ alkylene-NH—C₅₋₂₅ alkenyl. According to some embodiments, R³² is C₀₋₁₁ alkylene-CO—NH—C₀₋₄ alkylene-N(C₁₋₁₂ alkyl)₂. According to some embodiments, R³² is C₁-ii alkylene-CO₂H. R³⁴ is selected from the group consisting of: C₁₋₂₁ alkyl, C₂₋₂₁ alkenyl, C₂₋₂₁ alkynyl, C₁₋₁₁ alkylene-CO₂—C₅₋₁₅ alkyl, C₁₋ₙ alkylene-CO₂—C₅₋₁₅ alkenyl, C₁₋ₙ alkylene-O₂C—C₅₋₁₅ alkyl, C₁₋ₙ alkylene-O₂C—C₅₋₁₅ alkenyl, C₂₋₁₁ alkenylene-CO₂—C₅₋₁₅ alkyl, C₂₋₁₁ alkenylene-CO₂—C₅-alkenyl, C₂₋₁₁ alkenylene-O₂C—C₅₋₁₅ alkyl, C₂₋₁₁ alkenylene-O₂C—C₅₋₁₅ alkenyl and C₁₋₁₁ alkylene-CO₂—C₅₋₁₅ alkylene-N(R⁵)R⁶. According to some embodiments, R³⁴ is C₁₋₂₁ alkyl. According to some embodiments, R³⁴ is C₂₋₂₁ alkenyl. According to some embodiments, R³⁴ is C₂₋₂₁ alkynyl. According to some embodiments, R³⁴ is C₁₋ₙ alkylene-CO₂—C₅₋₁₅ alkyl. According to some embodiments, R³⁴ is C₁₋₁₁ alkylene-CO₂—C₅₋₁₅ alkenyl. According to some embodiments, R³⁴ is C₁₋ₙ alkylene-O₂C—C₅₋₁₅ alkyl. According to some embodiments, R³⁴ is C₁₋₁₁ alkylene-O₂C—C₅₋₁₅ alkenyl. According to some embodiments, R³⁴ is C₂₋₁₁ alkenylene-CO₂—C₅₋₁₅ alkyl. According to some embodiments, R³⁴ is C₂₋₁₁ alkenylene-CO₂—C₅₋₁₅ alkenyl. According to some embodiments, R³⁴ is C₂₋₁₁ alkenylene-O₂C—C₅₋₁₅ alkyl. According to some embodiments, R³⁴ is C₂₋₁₁ alkenylene-O₂C—C₅₋₁₅ alkenyl and C₁₋ₙ alkylene-CO₂—C₅₋₁₅ alkylene-N(R⁵)R⁶.

Formula (I′″)

According to some embodiments, R³² is represented by CH₂(CH₂)₃—R⁴². According to some embodiments, R³⁴ is represented by CH₂(CH₂)₃—R⁴⁴. According to some embodiments, R³² is represented by CH₂(CH₂)₃—R⁴² and R³⁴ is represented by CH₂(CH₂)₃—R⁴⁴, so the lipid is further represented by the structure of Formula (I′″). According to some embodiments, R² is represented by CH₂(CH₂)₇—R⁴². According to some embodiments, R⁴ is represented by CH₂(CH₂)₇—R⁴⁴. According to some embodiments, R² is represented by CH₂(CH₂)₇—R⁴² and R⁴ is represented by CH₂(CH₂)₇—R⁴⁴, so the lipid is further represented by the structure of Formula (I′″). In addition, as detailed above for Formulas (I′) and (I″), R¹ may be OH or CH₂CH₂—R²¹ and R³ may be CH₂CH₂—R²³, according to some embodiments. Thus, any reference to R¹, R²¹, R³ and R²³, may similarly apply to the respective substituent in Formula (I′″).

The structure of Formula (I′″) is presented below

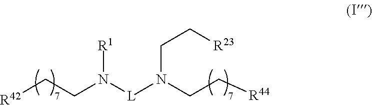

As can be understood by the person having ordinary skill in the art Formula (I′″) is a substructure of Formulas (I), (I′) and (I″), which are presented herein. Thus, embodiments relating to L and to R¹ as presented with respect to Formulas (I), (I′) and/or (I″) may similarly be used to describe Formula (I′″). Also, one of the options of R¹ is CH₂CH₂—R²¹, as described above, and it may be interpreted as also described above.

Likewise, according to some embodiments, R³² is represented by CH₂(CH₂)₃—R⁴² and/or R² is represented by CH₂(CH₂)₇—R³²—thus, similar conversions may be made to interpret R⁴², in view of the embodiments previously described for R² (e.g., when relating to Formula (I)) and/or R³² (e.g., when relating to Formula (I″)). For example, R² is described above as optionally C₅₋₁₅ alkylene-CO₂—C₅₋₁₅ alkyl—this embodiment may be converted to (a) R²² described as C₃₋₁₃ alkylene-CO₂—C₅₋₁₅ alkyl (given that the first two carbon atoms of the R² are each CH₂); (b) R³² described as C₁₋ₙ alkylene-CO₂—C₅₋₁₅ alkyl (given that the first two carbon atoms of the R²² are each CH₂, i.e., the first four carbon atoms of the R² are each CH₂); and (c) R⁴² described as C₀₋₇ alkylene-CO₂—C₅₋₁₅ alkyl (given that R²² is at least C₄ alkylene-CO₂—C₅₋₁₅ alkyl and its first four carbon atoms are each CH₂, i.e., R² is also at least C₄ alkylene-CO₂—C₅₋₁₅ alkyl and its the first eight carbon atoms of the are each CH₂). Similarly, according to some embodiments, R³⁴ is represented by CH₂(CH₂)₃—R⁴⁴ and/or R⁴ is represented by CH₂(CH₂)₈—R⁴⁴—thus, similar conversions may be made to interpret R⁴⁴, in view of the embodiments previously described for R⁴ and/or R³⁴. Lastly, R²³ of Formula (I′″) is as described for R²³ of Formulas (I′) and/or (I″). According to some embodiments, R⁴² is selected from the group consisting of: H, C₁₋₁₇ alkyl, C₂₋₁₇ alkenyl, C₂₋₁₇ alkynyl, C₀₋₇ alkylene-CO₂—C₅₋₁₅ alkyl, C₀₋₇ alkylene-CO₂—C₅₋₁₅ alkenyl, C₀₋₇ alkylene-O₂C—C₅₋₁₅ alkyl, C₀₋₇ alkylene-O₂C—C₅₋₁₅ alkenyl, C₂₋₇ alkenylene-CO₂—C₅₋₁₅ alkyl, C₂₋₇ alkenylene-CO₂—C₅₋₁₅ alkenyl, C₂₋₇ alkenylene-O₂C—C₅₋₁₅ alkyl, C₂₋₇ alkenylene-O₂C—C₅₋₁₅ alkenyl, C₁₋₁₁ alkylene-CO₂—C₀₋₄ alkylene-N(C₁₋₁₂ alkyl)₂, C₀₋₇ alkylene-CO₂—C₀₋₄ alkylene-NH—C₁₋₁₂ alkyl, C₀₋₇ alkylene-CO₂—C₀₋₄ alkylene-N(C₅₋₂₅ alkenyl)₂, C₀₋₇ alkylene-CO₂—C₀₋₄ alkylene-NH—C₅₋₂₅ alkenyl, C₀₋₇ alkylene-CO—NH—C₀₋₄ alkylene-N(C₁₋₁₂ alkyl)₂ and C₀₋₇ alkylene-CO₂H. According to some embodiments, R⁴² is H. According to some embodiments, R⁴² is C₁₋₁₇ alkyl. According to some embodiments, R⁴² is C₂₋₁₇ alkenyl. According to some embodiments, R⁴² is C₂₋₁₇ alkynyl. According to some embodiments, R⁴² is C₀₋₇ alkylene-CO₂—C₅₋₁₅ alkyl. According to some embodiments, R⁴² is C₀₋₇ alkylene-CO₂—C₅₋₁₅ alkenyl. According to some embodiments, R⁴² is C₀₋₇ alkylene-O₂C—C₅₋₁₅ alkyl. According to some embodiments, R⁴² is C₀₋₇ alkylene-O₂C—C₅₋₁₅ alkenyl. According to some embodiments, R⁴² is C₂₋₇ alkenylene-CO₂—C₅₋₁₅ alkyl. According to some embodiments, R⁴² is C₂₋₇ alkenylene-CO₂-C₅₋₁₅ alkenyl. According to some embodiments, R⁴² is C₂₋₇ alkenylene-O₂C—C₅₋₁₅ alkyl. According to some embodiments, R⁴² is C₂₋₇ alkenylene-O₂C—C₅₋₁₅ alkenyl. According to some embodiments, R⁴² is C₀₋₁₁ alkylene-CO₂—C₀₋₄ alkylene-N(C₁₋₁₂ alkyl)₂. According to some embodiments, R⁴² is C₀₋₇ alkylene-CO₂—C₀₋₄ alkylene-NH—C₁₋₁₂ alkyl. According to some embodiments, R⁴² is C₀₋₇ alkylene-CO₂—C₀₋₄ alkylene-N(C₅₋₂₅ alkenyl)₂. According to some embodiments, R⁴² is C₀₋₇ alkylene-CO₂—C₀₋₄ alkylene-NH—C₅₋₂₅ alkenyl. According to some embodiments, R⁴² is C₀₋₇ alkylene-CO—NH—C₀₋₄ alkylene-N(C₁₋₁₂ alkyl)₂. According to some embodiments, R⁴² is C₀₋₇ alkylene-CO₂H.

According to some embodiments, R⁴⁴ is selected from the group consisting of: H, C₁₋₁₇ alkyl, C₂₋₁₇ alkenyl, C₂₋₁₇ alkynyl, C₀₋₇ alkylene-CO₂—C₅₋₁₅ alkyl, C₀₋₇ alkylene-CO₂—C₅₋₁₅ alkenyl, C₀₋₇ alkylene-O₂C—C₅₋₁₅ alkyl, C₀₋₇ alkylene-O₂C—C₅₋₁₅ alkenyl, C₂₋₇ alkenylene-CO₂—C₅₋₁₅ alkyl, C₂₋₇ alkenylene-CO₂—C₅₋₁₅ alkenyl, C₂₋₇ alkenylene-O₂C—C₅₋₁₅ alkyl, C₂₋₇ alkenylene-O₂C—C₅₋₁₅ alkenyl and C₀₋₇ alkylene-CO₂—C₅₋₁₅ alkylene-N(R⁵)R⁶. According to some embodiments, R⁴⁴ is H. According to some embodiments, R⁴⁴ is C₁₋₁₇ alkyl. According to some embodiments, R⁴⁴ is C₂₋₁₇ alkenyl. According to some embodiments, R⁴⁴ is C₂₋₁₇ alkynyl. According to some embodiments, R⁴⁴ is C₀₋₇ alkylene-CO₂—C₅₋₁₅ alkyl. According to some embodiments, R⁴⁴ is C₀₋₇ alkylene-CO₂—C₅₋₁₅ alkenyl. According to some embodiments, R⁴⁴ is C₀₋₇ alkylene-O₂C—C₅₋₁₅ alkyl. According to some embodiments, R⁴⁴ is C₀₋₇ alkylene-O₂C—C₅₋₁₅ alkenyl. According to some embodiments, R⁴⁴ is C₂₋₇ alkenylene-CO₂—C₅₋₁₅ alkyl. According to some embodiments, R⁴⁴ is C₂₋₇ alkenylene-CO₂—C₅₋₁₅ alkenyl. According to some embodiments, R⁴⁴ is C₂₋₇ alkenylene-O₂C—C₅₋₁₅ alkyl. According to some embodiments, R⁴⁴ is C₂₋₇ alkenylene-O₂C—C₅₋₁₅ alkenyl and C₀₋₇ alkylene-CO₂—C₅₋₁₅ alkylene-N(R⁵)R⁶.

Formulas (Ia-c)

L, according to some embodiments, is selected from the group consisting of: L¹-X¹-L², L⁴-X²-L³, and L⁷-X³-L⁸. Thus, according to some embodiments, the structure of Formula (I) is represented by the structure of Formula (Ia), the structure of Formula (Ib), or the structure of Formula (Ic):

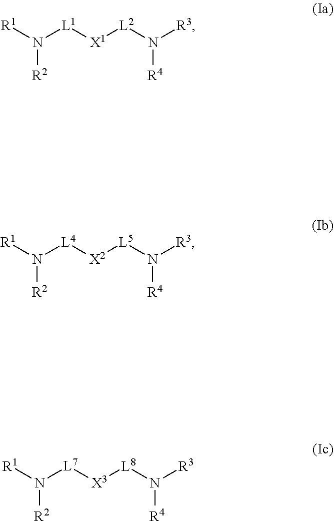

Each one of L¹, X¹, L², L⁴, X², L³, L⁷, X³, and L⁸ is described herein.

According to some embodiments, L is L¹-X¹-L² (i.e., Formula (Ia)), wherein
X¹ is selected from the group consisting of: —O₂C—, —CO₂-L³-O₂C—, —O₂C-L³-CO₂—, —CO₂-L³-CO₂—, L³-CO₂— and L³-O₂C—; each one of L¹ and L² is independently selected from the group consisting of: C₄₋₂₀ alkylene and C₄₋₂₀ alkenylene; L³ is selected from the group consisting of: C₁₋₆ alkylene-(O—C₁₋₆ alkylene)ₙ-, C₂₋₆ alkenylene-(O—C₁₋₆ alkylene)ₘ-, C₁₋₆ alkylene-S—C₁₋₆ alkylene and C₁₋₆ alkylene-S—S—C₁₋₆ alkylene; each one of n and m is 0, 1, 2, 3, 4 or 5; R¹ is selected from the group consisting of: C₀₋₆ alkylene-Y¹, C₂₋₆ alkenylene-Y¹ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl and L-N(R³)—R⁴; or R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₂₋₁₅ alkylene-CO₂—C₀₋₄ alkylene-N(C₁₋₁₂ alkyl)₂, and C₅₋₅ alkylene-O₂C—C₅₋₁₅ alkylene-N(C₁₋₁₂ alkyl)₂; R³ is selected from the group consisting of: C₀₋₆ alkylene-Y¹, C₂₋₆ alkenylene-Y¹, and C₁₋₄ alkyl;
R⁴ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl; each Y¹ is selected from the group consisting of: O—Y², halogen, PO₃H₂, PO₃(C₁₋₄ alkyl)₂, PO₃H(C₁₋₄ alkyl), NH₂, NMe₂, and NH—Y²; and Y² is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃ aryl; or
L is L⁴-X²-L⁵ (i.e. Formula (Tb)), wherein;
X² is selected from the group consisting of: —O₂C—, —CO₂-L⁶-O₂C—, —O₂C-L⁶-CO₂—, —CO₂-L⁶-CO₂—, L⁶-CO₂—, L⁶-O₂C— and —CO—NH—; L⁴ is a C₀₋₄ alkylene; L⁵ is selected from the group consisting of: C₂₋₂₀ alkylene and C₄₋₂₀ alkenylene; L⁶ is selected from the group consisting of: C₁₋₆ alkylene-(O—C₁₋₆ alkylene)ⱼ-, C₂₋₆ alkenylene-(O—C₁₋₆ alkylene)ₖ-, C₁₋₆ alkylene-S—C₁₋₆ alkylene and C₁₋₆ alkylene-S—S—C₁₋₆ alkylene; each one of j and k is 0, 1, 2, 3, 4 or 5; R¹ is selected from the group consisting of: C₀₋₁₀ alkylene-Y³, C₂₋₆ alkenylene-Y³ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H and L-N(R³)—R⁴; or R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H, C₂₋₁₅ alkylene-CO₂—C₀₋₄ alkylene-N(C₁₋₁₂ alkyl)₂, C₂₋₁₅ alkylene-CO₂—C₀₋₄ alkylene-NH—C₅₋₂₅ alkenyl, C₂₋₁₅ alkylene-CO₂—C₀₋₄ alkylene-N(C₅₋₂₅ alkenyl)₂, C₂₋₁₅ alkylene-CO—NH—C₀₋₄ alkylene-N(C₁₋₁₂ alkyl)₂ and C₂₋₁₅ alkylene-CO₂—C₀₋₄ alkylene-NH—C₁₋₁₂ alkyl; R³ is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl; R⁴ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; Y³ is selected from the group consisting of: O—Y⁴, halogen, PO₃H₂, PO₃(C₁₋₄ alkyl)₂, PO₃H(C₁₋₄ alkyl), NH₂, and NH—Y⁴; Y⁴ is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl; or Y⁴ is selected from the group consisting of: H, C₁₋₄ alkyl, C₁₋₄ alkylene-OH, C₁₋₄ alkylene-(O—C₁₋₄ alkylene)₁₋₃-OH, PO₃H₂, PO₃(C₁₋₄ alkyl)₂, PO₃H(C₁₋₄ alkyl), CO—C₁₋₄ alkyl, CO-aryl, CO—C₁₋₄ alkyl-N(CH₃)₂, SO₃H, SO₃—(C₁₋₄ alkyl), NMe₂, CO—C₁₋₄ alkyl-NM₂ and SO₃-aryl; or
L is L⁷-X³-L⁸ (i.e. Formula (Ia)), wherein
X³ is selected from the group consisting of: —O₂C—, —CO₂-L⁹-O₂C—, —O₂C-L⁹-CO₂—, —CO₂-L⁹-CO₂— and L⁹-CO₂—, L⁹-O₂C—; L⁷ is a C₀₋₄ alkylene; L⁸ is a C₀₋₄ alkylene or C₄₋₂₀ alkylene; L⁹ is selected from the group consisting of: C₀₋₄ alkylene-aryl-C₀₋₄ alkylene, C₁₋₄ alkylene, C₁₋₆ alkylene-(O—C₁₋₆ alkylene)ₕ-, C₂₋₆ alkenylene-(O—C₁₋₆ alkylene)ᵢ-, C₁₋₆ alkylene-S—C₁₋₆ alkylene and C₁₋₆ alkylene-S—S—C₁₋₆ alkylene; each one of h and i is 0, 1, 2, 3, 4 or 5; each one of R¹, R² and R³ is independently selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl and R⁴ is selected from the group consisting of: H, C₅₋₂₅ alkyl, C₅₋₂₅ alkenyl, C₅₋₁₅ alkylene-CO₂-C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; or R¹ is selected from the group consisting of: C₅₋₂₅ alkyl, C₅₋₂₅ alkenyl and C₀₋₁₀ alkylene-O—CO—C₁₋₄ alkyl-N(CH₃)₂; each one of R² and R⁴ is independently selected from the group consisting of: C₅₋₂₅ alkyl and C₅₋₂₅ alkenyl; and R³ is selected from the group consisting of: H, C₅₋₂₅ alkyl, and C₅₋₂₅ alkenyl.

Formula (Ia)

According to some embodiments, L is L¹-X¹-L². Thus, according to some embodiments, the structure of Formula (I) is represented by the structure of Formula (Ia):

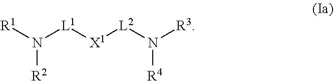

According to some embodiments, L^{a} is L¹ or L². According to some embodiments, L^{a} is L¹. According to some embodiments, L^{a} is L². According to some embodiments, L^{b} is L¹ or L². According to some embodiments, L^{b} is L¹. According to some embodiments, L^{b} is L². According to some embodiments, X^{a} is X¹. Thus, it is to be understood that the definitions detailed above with respect to L^{a}, L^{b} and X^{a} may similarly apply for L¹, L² and X¹, when appropriate.

According to some embodiments, X¹ is selected from the group consisting of: —O₂C—, —CO₂-L³-O₂C—, —O₂C-L³-CO₂—, —CO₂-L³-CO₂—, L³-CO₂— and L³-O₂C—. Each possibility represents a separate embodiment.

According to some embodiments, each one of L¹ and L² is selected from the group consisting of: C₄₋₂₀ alkylene and C₄₋₂₀ alkenylene. Each possibility represents a separate embodiment.

According to some embodiments, L³ is selected from the group consisting of: C₁₋₆ alkylene-(O—C₁₋₆ alkylene)ₙ-, C₂₋₆ alkenylene-(O—C₁₋₆ alkylene)ₘ-, C₁₋₆ alkylene-S—C₁₋₆ alkylene and C₁₋₆ alkylene-S—S—C₁₋₆ alkylene. Each possibility represents a separate embodiment. According to some embodiments, each one of n and m in Formula (Ia) is 0, 1, 2, 3, 4 or 5. Each possibility represents a separate embodiment.

According to some embodiments, R¹ in Formula (Ia) is selected from the group consisting of: C₀₋₆ alkylene-Y¹, C₂₋₆ alkenylene-Y¹ and C₁₋₄ alkyl. Each possibility represents a separate embodiment.

According to some embodiments, R² in Formula (Ia) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl and L-N(R³)—R⁴. Each possibility represents a separate embodiment. According to some embodiments, R² in Formula (Ia) is selected from the group consisting of: R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₂₋₁₅ alkylene-CO₂—C₀₋₄ alkylene-N(C₁₋₁₂ alkyl)₂, and C₅₋₁₅ alkylene-O₂C—C₅₋₁₅ alkylene-N(C₁₋₁₂ alkyl)₂. Each possibility represents a separate embodiment.

According to some embodiments, R³ in Formula (Ia) is selected from the group consisting of: C₀₋₆ alkylene-Y¹, C₂₋₆ alkenylene-Y¹, and C₁₋₄ alkyl. Each possibility represents a separate embodiment.

According to some embodiments, R⁴ in Formula (Ia) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl. Each possibility represents a separate embodiment. According to some embodiments, R⁴ in Formula (Ia) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl and C₅₋₁₅ alkylene-CO₂—C₅₋₁₅ alkylene-N(R⁵)R⁶.

According to some embodiments, each Y¹ is selected from the group consisting of: O—Y², halogen, PO₃H₂, PO₃(C₁₋₄ alkyl)₂, PO₃H(C₁₋₄ alkyl), NH₂, and NH—Y². Each possibility represents a separate embodiment. According to some embodiments, each Y¹ is selected from the group consisting of: O—Y², halogen, PO₃H₂, PO₃(C₁₋₄ alkyl)₂, PO₃H(C₁₋₄ alkyl), NH₂, NMe₂, and NH—Y². Each possibility represents a separate embodiment of the invention.

According to some embodiments, Y² is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl. Each possibility represents a separate embodiment. According to some embodiments, Y² is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl.

It is to be understood that the definitions detailed above with respect to Y^{a} and Y^{b} may similarly apply for Y¹ and Y², when appropriate.

According to some embodiments, X¹ is selected from the group consisting of: —O₂C—and —CO₂-L³-O₂C—; each one of L¹ and L² is C₅₋₁₅ alkylene; L³ is C₁₋₄ alkylene-; R¹ is C₀₋₄ alkylene-Y¹; R² is selected from the group consisting of: C₉₋₂₀ alkyl and C₉₋₂₀ alkenyl; R³ is C₁₋₄ alkylene-Y¹; R⁴ is selected from the group consisting of: C₅₋₂₅ alkyl, C₅₋₂₅ alkenyl, C₅₋₁₅ alkylene-CO₂—C₅₋₁₅ alkylene-N(R⁵)R⁶; and each Y¹ is OH.

According to some embodiments, X¹ is selected from the group consisting of: —O₂C—, —CO₂-L³-O₂C—, —O₂C-L³-CO₂—, —CO₂-L³-CO₂—, L³-CO₂— and L³-O₂C—; each one of L¹ and L² is selected from the group consisting of: C₄₋₂₀ alkylene and C₄₋₂₀ alkenylene; L³ is selected from the group consisting of: C₁₋₆ alkylene-(O—C₁₋₆ alkylene)ₙ-, C₂₋₆ alkenylene-(O—C₁₋₆ alkylene)ₘ-, C₁₋₆ alkylene-S—C₁₋₆ alkylene and C₁₋₆ alkylene-S—S—C₁₋₆ alkylene; each one of n and m is 0, 1, 2, 3, 4 or 5; R¹ is selected from the group consisting of: C₀₋₆ alkylene-Y¹, C₂₋₆ alkenylene-Y¹ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl and L-N(R³)—R⁴; R³ is selected from the group consisting of: C₀₋₆ alkylene-Y¹, C₂₋₆ alkenylene-Y¹, and C₁₋₄ alkyl; R⁴ is selected from the group consisting of: C₅₋₂₅ alkyl, C₅₋₂₅ alkenyl, C₅₋₁₅ alkylene-CO₂-C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl; Y¹ is selected from the group consisting of: O—Y², halogen, PO₃H₂, PO₃(C₁₋₄ alkyl)₂, PO₃H(C₁₋₄ alkyl), NH₂, and —NH—Y²; and Y² is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl.

According to some embodiments, R¹ is selected from the group consisting of: C₁₋₃ alkylene-OH, OH, C₁₋₃ alkylene-halogen, C₁₋₃ alkylene-PO₃H₂, methyl, ethyl, propyl and C₁₋₃ alkylene-NH₂; R² is selected from the group consisting of: C₈₋₁₈ alkyl, C₈₋₁₈ alkenyl, C₅₋₁₀ alkylene-CO₂—C₅₋₁₀ alkenyl and L-N(R³)—R⁴; R³ is selected from the group consisting of: C₁₋₃ alkylene-OH, OH, C₁₋₃ alkylene-halogen, C₁₋₃ alkylene-PO₃H₂, methyl, ethyl, propyl, and C₁₋₃ alkylene-NH₂; and R⁴ is selected from the group consisting of: C₈₋₁₈ alkyl, C₈₋₁₈ alkenyl, and C₅₋₁₀ alkylene-CO₂-C₅₋₁₀ alkenyl.

According to some embodiments, L is L¹-X¹-L², and is selected from the group consisting of: C₆₋₁₅ alkylene-O₂C—C₆₋₁₅ alkylene, C₄₋₁₅ alkylene-CO₂—C₁₋₃ alkylene-(O—C₁₋₃ alkylene)ₙ-O₂C—C₄₋₁₅ alkylene, and C₄₋₁₅ alkylene-CO₂—C₁₋₄ alkylene-S—S—C₁₋₄ alkylene-O₂C—C₄₋₁₅ alkylene; and n is 0 1 or 2; According to some embodiments, R¹ is selected from the group consisting of: CH₂CH₂PO₃H₂, CH₂CH₂Cl, CH₂CH₂OH, CH₃, OH and C₁₋₃ alkyl.

According to some embodiments, R² is selected from the group consisting of: C₈₋₁₆ alkyl, C₈₋₂₀ alkenyl and L-N(R³)—R⁴. According to some embodiments, R³ is selected from the group consisting of: CH₂CH₂PO₃H₂, CH₂CH₂Cl, CH₂CH₂OH, CH₃, OH and C₁₋₃ alkyl. According to some embodiments, R⁴ is selected from the group consisting of: C₈₋₁₆ alkyl, C₈₋₂₀ alkenyl, and C₈₋₁₂ alkylene-CO₂—C₈₋₁₂ alkenyl. According to some embodiments, R² is the same as R⁴.

According to some embodiments, R¹ is the same as R³. According to some embodiments, R² is the same as R⁴ and R¹ is the same as R³.

It is the be understood that the term “the same” in the previous paragraph means that the two specified R substituents have the same chemical definition. For example, compound DSL1-1 as shown below has the same R² and R⁴, each of which is —(CH₂)₈—CH═CH—CH₂—CH═CH—(CH₂)₄—CH₃. This compound also has the same R¹ and R³, each of which is 2-hydroxyethyl, —(CH₂)₂—OH. According to some embodiments, the lipid of Formula (Ia) is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54 DSL1-55, DSL1-56, DSL1-57, DSL1-58, DSL1-59, DSL1-60, DSL3c-1, DSL3c-2, DSL3c-3, DSL3c-4, DSL3c-5, DSL3c-6, DSL3c-7 and DSL3c-8, including salts, hydrates, solvates, polymorphs, optical isomers, geometrical isomers, enantiomers, diastereomers, and mixtures thereof. Each possibility represents a separate embodiment of the invention. According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL1-55, DSL1-56, DSL1-57, DSL1-58, DSL1-59, DSL1-60, including salts, hydrates, solvates, polymorphs, optical isomers, geometrical isomers, enantiomers, diastereomers, and mixtures thereof. According to some embodiments, the lipid is selected from the group consisting of: DSL3c-1, DSL3c-2, DSL3c-3, DSL3c-4, DSL3c-5, DSL3c-6, DSL3c-7 and DSL3c-8. According to some embodiments, the lipid is selected from the group consisting of: DSL1-1, DSL1-2, DSL1-3, DSL1-4, DSL1-5 and DSL3c-1. The chemical structures of each of said compounds is provided below, including salts, hydrates, solvates, polymorphs, optical isomers, geometrical isomers, enantiomers, diastereomers, and mixtures thereof.

Formula (Ib)

According to some embodiments, L is L⁴-X²-L⁵. Thus, according to some embodiments, the structure of Formula (I) is represented by the structure of Formula (Ib):

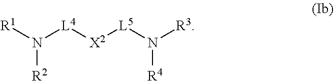

According to some embodiments, L^{a} is L⁴ or L⁵. According to some embodiments, L^{a} is L⁴.

According to some embodiments, L^{a} is L⁵. According to some embodiments, L^{b} is L⁴ or L⁵.

According to some embodiments, L^{b} is L⁴. According to some embodiments, L^{b} is L⁵. According to some embodiments, X^{a} is X². Thus, it is to be understood that the definitions detailed above with respect to L^{a}, L^{b} and X^{a} may similarly apply for L⁴, L⁵ and X², when appropriate. According to some embodiments, X² is selected from the group consisting of: —O₂C—, —CO₂-L⁶-O₂C—, —O₂C-L⁶-CO₂—, —CO₂-L⁶-CO₂—, L⁶-CO₂—, L⁶-O₂C— and —CO—NH—. Each possibility represents a separate embodiment.

According to some embodiments, L⁴ is a C₀₋₄ alkylene.

According to some embodiments, L⁵ is selected from the group consisting of: C₂₋₂₀ alkylene and C₄₋₂₀ alkenylene. Each possibility represents a separate embodiment.

According to some embodiments, L⁶ is selected from the group consisting of: C₁₋₆ alkylene-(O—C₁₋₆ alkylene)ⱼ-, C₂₋₆ alkenylene-(O—C₁₋₆ alkylene)ₖ-, C₁₋₆ alkylene-S—C₁₋₆ alkylene and C₁₋₆ alkylene-S—S—C₁₋₆ alkylene. Each possibility represents a separate embodiment.

According to some embodiments, each one of j and k in Formula (Ib) is 0, 1, 2, 3, 4 or 5. Each possibility represents a separate embodiment.

According to some embodiments, R¹ in Formula (Ib) is selected from the group consisting of: C₀-alkylene-Y³, C₂₋₆ alkenylene-Y³ and C₁₋₄ alkyl. Each possibility represents a separate embodiment.

According to some embodiments, R² in Formula (Ib) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H and L-N(R³)—R⁴. Each possibility represents a separate embodiment. According to some embodiments, R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H, C₂₋₁₅ alkylene-CO₂—C₀₋₄ alkylene-N(C₁₋₁₂ alkyl)₂, C₂₋₁₅ alkylene-CO₂—C₀₋₄ alkylene-NH—C₅₋₂₅ alkenyl, C₂₋₁₅ alkylene-CO₂—C₀₋₄ alkylene-N(C₅₋₂₅ alkenyl)₂, C₂₋₁₅ alkylene-CO—NH—C₀₋₄ alkylene-N(C₁₋₁₂ alkyl)₂ and C₂₋₁₅ alkylene-CO₂—C₀₋₄ alkylene-NH—C₁₋₁₂ alkyl. Each possibility represents a separate embodiment of the invention.

According to some embodiments, R³ in Formula (Ib) is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl. Each possibility represents a separate embodiment.

According to some embodiments, R⁴ in Formula (Ib) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl. Each possibility represents a separate embodiment.

According to some embodiments, Y³ is selected from the group consisting of: O—Y⁴, halogen, PO₃H₂, PO₃(C₁₋₄ alkyl)₂, PO₃H(C₁₋₄ alkyl), NH₂, and NH—Y⁴. Each possibility represents a separate embodiment.

According to some embodiments, Y⁴ is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl. Each possibility represents a separate embodiment. According to some embodiments, Y⁴ is selected from the group consisting of: H, C₁₋₄ alkyl, C₁₋₄ alkylene-OH, C₁₋₄ alkylene-(O—C₁₋₄ alkylene)₁₋₃-OH, PO₃H₂, PO₃(C₁₋₄ alkyl)₂, PO₃H(C₁₋₄ alkyl), CO—C₁₋₄ alkyl, CO-aryl, CO—C₁₋₄ alkyl-N(CH₃)₂, SO₃H, SO₃—(C₁₋₄ alkyl), NMe₂, CO—C₁₋₄ alkyl-NM₂ and SO₃-aryl.

It is to be understood that the definitions detailed above with respect to Y^{a} and Y^{b} may similarly apply for Y³ and Y⁴, when appropriate.

According to some embodiments, X² is —O₂C—; L⁴ is a C₀₋₄ alkylene or C₄₋₂₀ alkylene; L⁵ is a C₄₋₂₀ alkylene; R¹ is C₀₋₁₀ alkylene-Y³; R² is selected from the group consisting of: C₅₋₂₅ alkyl, C₅₋₂₅ alkenyl and C₂₋₁₅ alkylene-CO₂—C₀₋₄ alkylene-N(C₁₋₁₂ alkyl)₂; R³ is a C₅₋₂₅ alkyl; R⁴ is a C₅₋₂₅ alkyl; Y³ is O—Y^{b}; and Y⁴ is H.

According to some embodiments, L is L⁴-X²-L⁵; X² is selected from the group consisting of: —O₂C—, —CO₂-L⁶-O₂C—, —O₂C-L⁶-CO₂—, —CO₂-L⁶-CO₂—, L⁶-CO₂—, L⁶-O₂C— and —CO—NH—; L⁴ is a C₀₋₄ alkylene; L⁵ is selected from the group consisting of: C₄₋₂₀ alkylene and C₄₋₂₀ alkenylene; L⁶ is selected from the group consisting of: C₁₋₆ alkylene-(O—C₁₋₆ alkylene)ⱼ-, C₂₋₆ alkenylene-(O—C₁₋₆ alkylene)ₖ-, C₁₋₆ alkylene-S—C₁₋₆ alkylene and C₁₋₆ alkylene-S—S—C₁₋₆ alkylene; each one of j and k is 0, 1, 2, 3, 4 or 5; R¹ is selected from the group consisting of: C₀₋₁₀ alkylene-Y³, C₂₋₆ alkenylene-Y³ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H and L-N(R³)—R⁴; R³ is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl; R⁴ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; Y³ is selected from the group consisting of: O—Y⁴, halogen, PO₃H₂, PO₃(C₁₋₄ alkyl)₂, PO₃H(C₁₋₄ alkyl), NH₂, and NH—Y⁴; Y⁴ is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl. According to some embodiments, L selected from the group consisting of: —O₂C—C₆₋₁₂ alkylene, C₁₋₃ alkylene-CO₂—C₁₋₃ alkylene-S—S—C₁₋₃ alkylene-O₂C—C₄₋₁₀ alkylene, C₁₋₃ alkylene-CO₂—C₆₋₁₂ alkylene, C₁₋₃ alkylene-O—C₁₋₃ alkylene-O₂C—C₆₋₁₂ alkylene, and C₁₋₃ alkylene-NHCO—C₆₋₁₂ alkylene.

According to some embodiments, X² is selected from the group consisting of: —O₂C—, —CO₂-L⁶-O₂C—, —O₂C-L⁶-CO₂—, —CO₂-L⁶-CO₂—, L⁶-CO₂—, L⁶-O₂C— and —CO—NH—; L⁴ is a C₀₋₄ alkylene; L⁵ is selected from the group consisting of: C₄₋₂₀ alkylene and C₄₋₂₀ alkenylene; L⁶ is selected from the group consisting of: C₁₋₆ alkylene-(O—C₁₋₆ alkylene)ⱼ-, C₂₋₆ alkenylene-(O—C₁₋₆ alkylene)ₖ-, and C₁₋₆ alkylene-S—C₁₋₆ alkylene and C₁₋₆ alkylene-S—S—C₁₋₆ alkylene; each one of j and k is 0, 1, 2, 3, 4 or 5; R¹ is selected from the group consisting of: C₀₋₁₀ alkylene-Y³, C₂₋₆ alkenylene-Y³ and C₁₋₄ alkyl; R² is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, C₅₋₁₅ alkenylene-O₂C—C₅₋₁₅ alkenyl, C₅₋₁₅ alkylene-CO₂H and L-N(R³)—R⁴.

According to some embodiments, R³ is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl and C₅₋₁₅ alkenylene-O₂C—C₅₋₁₅ alkenyl; R⁴ is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂-C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; Y³ is selected from the group consisting of: O—Y⁴, halogen, PO₃H₂, PO₃(C₁₋₄ alkyl)₂, PO₃H(C₁₋₄ alkyl), NH₂, and NH—Y⁴; and Y⁴ is selected from the group consisting of: H, C₁₋₄ alkyl, C₁₋₄ alkylene-OH, PO₃H₂, PO₃(C₁₋₄ alkyl)₂, PO₃H(C₁₋₄ alkyl), CO—C₁₋₄ alkyl, CO-aryl, SO₃H, SO₃—(C₁₋₄ alkyl) and SO₃-aryl.

According to some embodiments, R¹ is selected from the group consisting of: C₈₋₁₈ alkyl, C₈₋₁₈ alkenyl; C₁₋₃ alkylene-OH, C₁₋₃ alkylene-O—CH₂CH₂—OH, C₁₋₃ alkylene-C₁, C₁₋₃ alkylene-NH₂, OH and C₁₋₃ alkylene-PO₃H₂; R² is selected from the group consisting of: C₈₋₁₈ alkyl, C₈₋₁₈ alkenyl, C₂₋₁₂ alkylene-CO₂H, L-N(R³)—R⁴, and C₅₋₁₀ alkylene-CO₂—C₅₋₁₀ alkenyl; R³ is selected from the group consisting of: H, C₈₋₁₈ alkyl and C₈₋₁₈ alkenyl; and R⁴ is selected from the group consisting of: C₈₋₁₈ alkyl, C₈₋₁₈ alkenyl.

According to some embodiments, L is L⁴-X²-L⁵ is selected from the group consisting of: —O₂C—C₆₋₁₂ alkylene, C₁₋₃ alkylene-CO₂—C₆₋₁₂ alkylene, C₁₋₃ alkylene-CO₂—C₁₋₃ alkylene-S—S—C₁₋₃ alkylene-O₂C—C₄₋₁₀ alkylene, C₁₋₃ alkylene-O—C₁₋₃ alkylene-O₂C—C₆₋₁₂ alkylene, and C₁₋₃ alkylene-NHCO—C₆₋₁₂ alkylene.

According to some embodiments, R¹ in Formula (Tb) is selected from the group consisting of: CH₂CH₂OH, CH₂CH₂OCH₂CH₂OH, CH₂CH₂Cl, OH, CH₂CH₂PO₃H₂, and CH₂CH₂NH₂. According to some embodiments, R² in Formula (Tb) is selected from the group consisting of: C₈₋₁₆ alkyl, C₈₋₂₀ alkenyl, C₆₋₁₂ alkylene-CO₂—C₆₋₁₂ alkenyl, C₆₋₁₂ alkylene-CO₂H, and L-N(R³)—R⁴. According to some embodiments, R³ in Formula (Ib) is selected from the group consisting of: H, C₆₋₁₆ alkyl, and C₆₋₁₆ alkenyl. According to some embodiments, R⁴ in Formula (Tb) is selected from the group consisting of: C₆₋₁₆ alkyl, C₆₋₁₆ alkenyl. According to some embodiments, R³ in Formula (Tb) is the same as R⁴. The term “the same” as used herein is as defined with respect to the parallel substituents of Formula (Ia).

According to some embodiments, the lipid of Formula (Ib) is selected is selected from the group consisting of: DSL2-1, DSL2-2, DSL2-3, DSL2-4, DSL2-5, DSL2-6, DSL2-7, DSL2-8, DSL2-9, DSL2-10, DSL2-11, DSL2-12, DSL2-13, DSL2-14, DSL2-15, DSL2-16, DSL2-17, DSL2-18, DSL2-19, DSL2-20, DSL2-21, DSL2-22, DSL2-23, DSL2-24, DSL2-25, DSL2-26, DSL2-27, DSL2-28, DSL2-29, DSL2-30, DSL2-31, DSL2-32, DSL2-33, DSL2-34, DSL2-35, DSL2-36, DSL2-37, DSL2-38, DSL2-39, DSL2-40, DSL2-41, DSL2-42, DSL2-43, DSL2-44, DSL2-45, DSL2-46, DSL2-47, DSL2-48, DSL2-49, DSL2-50, DSL2-51, DSL2-52, DSL2-53, DSL2-54, DSL2-55, DSL2-56, DSL2-57, DSL2-58, DSL2-59, DSL2-60, DSL2-61, DSL2-62, DSL2-63, DSL2-64, DSL2-65, DSL2-66, DSL2-67, DSL2-68, DSL2-69, DSL2-70, DSL2-71, DSL2-72 including salts, hydrates, solvates, polymorphs, optical isomers, geometrical isomers, enantiomers, diastereomers, and mixtures thereof. The chemical structures of each of said compounds is provided below.

According to some exemplary embodiments, the lipid is selected from the group consisting of: DSL2-1, DSL2-2, DSL2-49 and DSL2-50 including salts, hydrates, solvates, polymorphs, optical isomers, geometrical isomers, enantiomers, diastereomers, and mixtures thereof.

Formula (Ic)

According to some embodiments, L is L⁷-X³-L⁸. Thus, according to some embodiments, the structure of Formula (I) is represented by the structure of Formula (Ic):

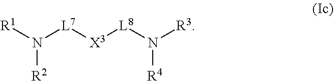

According to some embodiments, L^{a} is L⁷ or L^{g}. According to some embodiments, L^{a} is L⁷.

According to some embodiments, L^{a} is L^{g}. According to some embodiments, L^{b} is L⁷ or L^{g}.

According to some embodiments, L^{b} is L⁷. According to some embodiments, L^{b} is L⁸. According to some embodiments, X^{a} is X³. Thus, it is to be understood that the definitions detailed above with respect to L^{a}, L^{b} and X^{a} may similarly apply for L⁷, L⁸ and X³, when appropriate.

According to some embodiments, X³ is selected from the group consisting of: —O₂C—, —CO₂-L⁹-O₂C—, —O₂C-L⁹-CO₂—, —CO₂-L⁹-CO₂— and L⁹-CO₂—, L⁹-O₂C—. Each possibility represents a separate embodiment. According to some embodiments, X³ is selected from the group consisting of: —O₂C—, —CO₂—NH—, —CO₂-L⁹-O₂C—, —O₂C-L⁹-CO₂—, —CO₂-L⁹-CO₂— and L⁹-CO₂—, L⁹-O₂C—. Each possibility represents a separate embodiment of the invention.

According to some embodiments, L⁷ is a C₀₋₄ alkylene. According to some embodiments, L⁸ is a C₀₋₄ alkylene. According to some embodiments, L⁸ is a C₀₋₄ alkylene or C₄₋₂₀ alkylene. According to some embodiments, L⁹ is selected from the group consisting of: C₀₋₄ alkylene-aryl-C₀₋₄ alkylene, C₁₋₄ alkylene, C₁₋₆ alkylene-(O—C₁₋₆ alkylene)ₕ-, C₂₋₆ alkenylene-(O—C₁₋₆ alkylene)ᵢ-, C₁₋₆ alkylene-S—C₁₋₆ alkylene and C₁₋₆ alkylene-S—S—C₁₋₆ alkylene. Each possibility represents a separate embodiment. According to some embodiments, each one of h and i is 0, 1, 2, 3, 4 or 5.

Each possibility represents a separate embodiment.

According to some embodiments, R¹ is selected from the group consisting of: C₅₋₂₅ alkyl, C₅₋₂₅ alkenyl and C₀₋₁₀ alkylene-O—CO—C₁₋₄ alkyl-N(CH₃)₂; each one of R² and R⁴ is independently selected from the group consisting of: C₅₋₂₅ alkyl and C₅₋₂₅ alkenyl; and R³ is selected from the group consisting of: H, C₅₋₂₅ alkyl, and C₅₋₂₅ alkenyl.

According to some embodiments, X³ is-O₂C—; L⁷ is a C₀₋₄ alkylene; L⁸ is a C₀₋₄ alkylene; R¹ is C₅₋₂₅ alkyl; and each one of R¹, R², R³ and R⁴ is a C₅₋₂₅ alkyl.

According to some embodiments, each one of R¹, R² and R³ in Formula (Ic) is independently selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl. According to some embodiments, R¹ in Formula (Ic) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl. According to some embodiments, R² in Formula (Ic) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂-C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl. According to some embodiments, R³ in Formula (Ic) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl.

According to some embodiments, R⁴ in Formula (Ic) is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl. According to some embodiments, R⁴ in Formula (Ic) is selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl.

According to some embodiments, each one of R¹, R², R³ and R⁴ in Formula (Ic) is independently selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl.

According to some embodiments, L is L⁷-X³-L⁸; X³ is selected from the group consisting of: —O₂C—, —CO₂-L⁹-O₂C—, —O₂C-L⁹-CO₂—, —CO₂-L⁹-CO₂— and L⁹-CO₂—, L⁹-O₂C—; L⁷ is a C₀₋₄ alkylene; L⁸ is a C₀₋₄ alkylene; L⁹ is selected from the group consisting of: C₀₋₄ alkylene-aryl-C₀₋₄ alkylene, C₁₋₄ alkylene, C₁₋₆ alkylene-(O—C₁₋₆ alkylene)ₕ-, C₂₋₆ alkenylene-(O—C₁₋₆ alkylene)ᵢ-, C₁₋₆ alkylene-S—C₁₋₆ alkylene and C₁₋₆ alkylene-S—S—C₁₋₆ alkylene; each one of h and i is 0, 1, 2, 3, 4 or 5; and each one of R¹, R² and R³ is independently selected from the group consisting of: C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl; and R⁴ is selected from the group consisting of: H, C₅₋₂₅ alkyl, C₅₋₂₅ alkenyl, C₅₋₁₅ alkylene-CO₂—C₅₋₁₅ alkyl, C₅₋₁₅ alkylene-CO₂—C₅₋₁₅ alkenyl, C₅₋₁₅ alkylene-O₂C—C₅₋₁₅ alkyl, C₅₋₁₅ alkylene-O₂C—C₅₋₁₅ alkenyl, C₅₋₁₅ alkenylene-CO₂—C₅₋₁₅ alkyl, C₅₋₁₅ alkenylene-CO₂—C₅₋₁₅ alkenyl, C₅₋₁₅ alkenylene-O₂C—C₅₋₁₅ alkyl, and C₅₋₁₅ alkenylene-O₂C—C₅₋₁₅ alkenyl.

According to some embodiments, in Formula (Ic), R¹ is selected from the group consisting of: C₈₋₁₈ alkyl and C₈₋₁₈ alkenyl and C₅₋₁₀ alkylene-CO₂—C₅₋₁₀ alkyl; R² is selected from the group consisting of: C₈₋₁₈ alkyl, C₈₋₁₈ alkenyl and C₅₋₁₀ alkylene-CO₂—C₅₋₁₀ alkyl; R³ is selected from the group consisting of: H and C₈₋₁₈ alkyl and C₈₋₁₈ alkenyl; and R⁴ is a C₈₋₁₈ alkyl.

According to some embodiments, L in Formula (Ic) is selected from the group consisting of C₁₋₄ alkylene-CO₂—C₁₋₄ alkylene, C₁₋₄ alkylene-CO₂—, O₂C—C₁₋₃ alkylene-C₆H₄-C₀₋₄ alkylene-CO₂, C₁₋₄ alkylene-O—C₁₋₄ alkylene-O₂C—C₁₋₄ alkylene, C₁₋₄ alkylene-O₂C—C₁₋₆ alkylene-CO₂—C₁₋₄ alkylene, O₂C—C₁₋₄ alkylene-S—S—C₁₋₄ alkylene-CO₂, and O₂C—C₁₋₆ alkylene-CO₂. It is to be understood that “C₆H₄” refers to a phenylene group, which is a divalent benzene. Depending on the bonding positions, phenylenes are selected from para-phenylene, wherein the two substituents are positions in a 1-4 relation; meta-phenylene, wherein the two substituents are positions in a 1-3 relation and ortho-phenylene, wherein the two substituents are positions in a 1-2 relation. According to some embodiments, the C₆14 is para-phenylene, which is shown below.

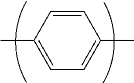

According to some embodiments, R¹ in Formula (Ic) is selected from the group consisting of: C₈₋₁₅ alkyl, C₈₋₂₀ alkenyl, and C₆₋₁₂ alkylene-O₂C—C₉₋₁₈ alkylene. According to some embodiments, R² in Formula (Ic) is selected from the group consisting of: C₈₋₁₅ alkyl, C₈₋₂₀ alkenyl, and C₆₋₁₂ alkylene-O₂C—C₉₋₁₈ alkylene. According to some embodiments, R³ in Formula (Ic) is selected from the group consisting of: C₈₋₁₅ alkyl and C₈₋₂₀ alkenyl. According to some embodiments, R⁴ in Formula (Ic) is selected from the group consisting of: H, C₈₋₁₅ alkyl and C₈-alkenyl. According to some embodiments, R⁴ in Formula (Ic) is selected from the group consisting of: C₈₋₁₅ alkyl and C₈₋₂₀ alkenyl.

According to some embodiments, at least two of R¹, R², R³, and R⁴ represent the same substituent.

According to some embodiments, at least three of R¹, R², R³, and R⁴ represent the same substituent.

According to some embodiments, each of R¹, R², R³, and R⁴ represent the same substituent.

According to some embodiments, no more than two of R¹, R², R³, and R⁴ represent the same substituent. According to some embodiments, no more than three of R¹, R², R³, and R⁴ represent the same substituent. According to some embodiments, each of R¹, R², R³, and R⁴ represents a different substituent.

The term “the same” as used herein is as defined with respect to the parallel substituents of Formula (Ta). The term “different” refer to a substituent, which is not the same. For example, Compounds DSL 11, 12 and 14 are compounds in which each of R¹, R², R³, and R⁴ represents a different substituent. So, these compounds fall under the definitions “no more than two/three of R¹, R², R³, and R⁴ represent the same substituent” and “each of R¹, R², R³, and R⁴ represents a different substituent”.

According to some embodiments, the lipid of Formula (Ic) is selected from the group consisting of: DSL4-1, DSL4-2, DSL4-3, DSL4-4, DSL4-5, DSL4-6, DSL4-7, DSL4-8, DSL4-9, DSL4-10, DSL4-11, DSL4-12, DSL4-13, DSL4-14 and DSL4-15, including salts, hydrates, solvates, polymorphs, optical isomers, geometrical isomers, enantiomers, diastereomers, and mixtures thereof. The chemical structures of each of said compounds is provided below.

According to some exemplary embodiments, the lipid is selected from the group consisting of: DSL4-1 and DSL4-2, including salts, hydrates, solvates, polymorphs, optical isomers, geometrical isomers, enantiomers, diastereomers, and mixtures thereof. According to some exemplary embodiments, the lipid is DSL4-2.

Formula (II)

According to some embodiments, R² is represented by CH₂(CH₂)₄—R²²; R³ is represented by CH₂CH₂—R¹³ and R⁴ is represented by CH₂(CH₂)₄—R¹⁴, so the lipid is further represented by the structure of Formula (II). The structure of Formula (II) is presented below:

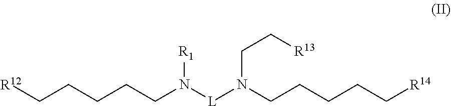

As can be understood by the person having ordinary skill in the art Formula (I′) is a substructure of formula (I) which is presented herein. Thus, embodiments relating to L and to R¹ as presented with respect to Formula (I) may similarly be used to describe Formula (II). Also, R² is represented by CH₂(CH₂)₄—R¹², as described above, thus as understood by the person having ordinary skill in the art, each of the options presented above for R², which comprise a terminal CH₂(CH₂)₄ group, may be converted into a respective set of CH₂(CH₂)₄—R¹² and/or R¹². For example, R² is described above as optionally C₅₋₂₅ alkenyl—this embodiment may be converted to R¹² described as C₂₋₂₀ alkyl (given that the first five carbon atoms of the R² are each CH₂, and given that alkenyls include at least two carbon atoms). Similarly, R³ is represented by CH₂CH₂—R¹³ and R⁴ is represented by CH₂(CH₂)₄—R¹⁴. So, similar conversions may be made to interpret R¹³⁻¹⁴, in view of the embodiments previously described for R³⁻⁴.

According to some embodiments, the lipid of the present invention is represented by the structure of Formula (II), wherein
each one of R¹ and R¹³ is independently selected from the group consisting of: OH, C₁₋₃ alkyl-OH, C₄₋₁₄ alkyl and C₄₋₁₄ alkenyl;
R¹² is selected from the group consisting of: C₁₋₁₃ alkyl; C₂₋₁₅ alkenyl, C₁₋₆ alkyl-CO₂—C₀₋₃ alkylene-N(C₁₋₈ alkyl)₂ and C₁₋₆ alkyl-CO₂— C₀₋₃ alkylene-NH—C₁₋₈ alkyl;
R¹⁴ is selected from the group consisting of: C₁₋₁₃ alkyl; C₂₋₁₅ alkenyl and

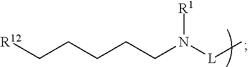

each L is an alkylene ester linker represented by: L^{a}-X^{a}-L^{b};
X^{a} is selected from the group consisting of: —O₂C—, —CO₂—C₂₋₄ alkylene-O₂C—, O₂C—C₂₋₄ alkylene-O₂C—, —CO₂—C₂₋₄ alkylene-CO₂—, and O₂C—C₂₋₄ alkylene-CO₂—;
L^{a} is selected from the group consisting of: C₁₋₃ alkylene, C₄₁₂ alkylene, C₂₋₁₀ alkenylene and absent; and
L^{b} is selected from the group consisting of: C₁₋₃ alkylene, C₂₋₁₀ alkenylene and C₄₁₂ alkylene.

According to some embodiments, R¹ is selected from the group consisting of: OH, C₁₋₃ alkyl-OH, C₄₋₁₄ alkyl and C₄₋₁₄ alkenyl. According to some embodiments, R¹ is selected from the group consisting of: OH, C₂₋₃ alkyl-OH, and C₈₋₁₄ alkyl. According to some embodiments, R¹ is OH. According to some embodiments, R¹ is C₁₋₃ alkyl-OH. According to some embodiments, R¹ is C₂₋₃ alkyl-OH. According to some embodiments, R¹ is C₄₋₁₄ alkyl. According to some embodiments, R¹ is C₈₋₁₄ alkyl. According to some embodiments, R¹ is C₄₋₁₄ alkenyl.

According to some embodiments, R¹² is C₁₋₁₃ alkyl. According to some embodiments, R¹² is C₂₋₁₅ alkenyl. According to some embodiments, R¹² is C₁₋₆ alkyl-CO₂—C₀₋₃ alkylene-N(C₁₋₈ alkyl)₂. According to some embodiments, R¹² is C₁₋₆ alkyl-CO₂—C₀₋₃ alkylene-NH—C₁₋₈ alkyl. According to some embodiments, R¹² is selected from the group consisting of: C₉₋₁₅ alkenyl, C₅₋₁₁ alkyl, C₃₋₆ alkyl-CO₂—C₀₋₂ alkylene-N(C₂₋₈ alkyl)₂. According to some embodiments R¹² is C₉₋₁₅ alkenyl.

According to some embodiments R¹² is C₅₁₁ alkyl. According to some embodiments R¹² is C₃₋₆ alkyl-CO₂—C₀₋₂ alkylene-N(C₂₋₈ alkyl)₂.

According to some embodiments, R¹³ is selected from the group consisting of: OH, C₁₋₃ alkyl-OH, C₄₋₁₄ alkyl and C₄₋₁₄ alkenyl. According to some embodiments, R¹³ is OH. According to some embodiments, R¹³ is C₁₋₃ alkyl-OH. According to some embodiments, R¹³ is C₄₋₁₄ alkyl. According to some embodiments, R¹ is C₄₋₁₄ alkenyl. According to some embodiments, R¹³ is selected from the group consisting of: OH, CH₂—OH, and C₄₋₂ alkyl. According to some embodiments, R¹³ is CH₂—OH. According to some embodiments, R¹³ is C₄₋₂ alkyl.

According to some embodiments, R¹⁴ is selected from the group consisting of: C₁₋₁₃ alkyl; C₂₋₁₅ alkenyl and

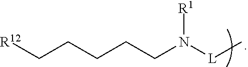

According to some embodiments, C₁₋₁₃ alkyl. According to some embodiments, R¹⁴ is C₂₋₁₅ alkenyl. According to some embodiments, R¹⁴ is:

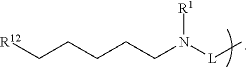

According to some embodiments, R¹⁴ is selected from the group consisting of: C₉₋₁₅ alkenyl, C₁₋₉ alkyl and

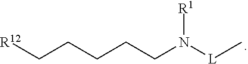

According to some embodiments, R¹⁴ is C₉₋₁₅ alkenyl. According to some embodiments, R¹⁴ is C₁₋₉ alkyl

According to some embodiments, each L (i.e., the L of Formula (II), the L of R¹⁴, or both) is an alkylene ester linker represented by: L^{a}-X^{a}-L^{b}. each one of L^{a}, X^{a}, and L^{b} is described above when describing the various substructures (e.g., Formula (I).

According to some embodiments, X^{a} is selected from the group consisting of: —O₂C—, —CO₂—C₂₋₄ alkylene-O₂C—, O₂C—C₂₋₄ alkylene-O₂C—, —CO₂—C₂₋₄ alkylene-CO₂— and O₂C—C₂₋₄ alkylene-CO₂—. According to some embodiments X^{a} is-O₂C—. According to some embodiments, X^{a} is —CO₂—C₂₋₄ alkylene-O₂C—. According to some embodiments, X^{a} is O₂C—C₂₋₄ alkylene-O₂C—. According to some embodiments, X^{a} is —CO₂—C₂₋₄ alkylene-CO₂—. According to some embodiments, X^{a} is O₂C—C₂₋₄ alkylene-CO₂—. According to some embodiments, X^{a} is selected from the group consisting of: —O₂C—and —CO₂—C₂₋₄ alkylene-O₂C—.

According to some embodiments, L^{a} is selected from the group consisting of: C₁₋₃ alkylene, C₄₋₁₂ alkylene, C₂₋₁₀ alkenylene and absent. According to some embodiments L^{a} is C₁₋₃ alkylene. According to some embodiments, L^{a} is C₄₋₁₂ alkylene. According to some embodiments, L^{a} is C₂₋₁₀ alkenylene. According to some embodiments, L^{a} is absent. According to some embodiments, L^{a} is absent or selected from the group consisting of: C₂₋₃ alkylene and C₆₋₁₁ alkylene. According to some embodiments, L^{a} is C₂₋₃ alkylene. According to some embodiments, L^{a} is C₆₋₁₁ alkylene.

According to some embodiments, L^{b} is selected from the group consisting of: C₁₋₃ alkylene, C₂₋₁₀ alkenylene and C₄₋₁₂ alkylene. According to some embodiments, L^{b} is C₁₋₃ alkylene. According to some embodiments, L^{b} is C₂₋₁₀ alkenylene. According to some embodiments, L^{b} is C₄₋₁₂ alkylene.

According to some embodiments, L^{b} is selected from the group consisting of: C₁₋₃ alkylene and C₄₋₁₂ alkylene. According to some embodiments, L^{b} is C₁₋₃ alkylene. According to some embodiments, L^{b} is C₄₋₁₂ alkylene.

According to some embodiments, the lipid of Formula (II) is selected from the group consisting of: DSL1-1, DSL1-2, DSL1-3, DSL1-4, DSL1-5, DSL1-13, DSL1-14, DSL1-15, DSL1-20, DSL1-58, DSL3c-1, DSL3c-4, DSL2-1, DSL2-2, DSL2-13, DSL2-14, DSL2-16, DSL2-29, DSL2-49, DSL2-50, DSL2-54, DSL2-63, DSL2-65, DSL4-1, DSL4-2, DSL2-5 and DSL2-7. Each possibility represents a separate embodiment of the invention. According to some embodiments, the lipid of Formula (II) is selected from the group consisting of: DSL1-1, DSL1-2, DSL1-3, DSL1-4, DSL1-5, DSL3c-1, DSL2-1, DSL2-2, DSL2-49, DSL2-50, DSL4-1 and DSL4-2.

According to some embodiments, the lipid of Formula (II) is selected from the group consisting of: DSL1-1, DSL1-2, DSL1-3, DSL1-4, DSL1-5, DSL3c-1, DSL2-1, DSL2-2, DSL2-49, DSL2-50, and DSL4-2. According to some embodiments, the lipid is selected from the group consisting of: DSL1-3 and DSL2-50.

The chemical structures of each of the specific compound are detailed below in the “Exemplary Compounds” Section and in the claims.

Exemplary Compounds

Exemplary compounds according to Formula (I), Formula (I′), Formula (I″), Formula (I′″), Formula (Ia), Formula (Ib), Formula (Ic) and Formula (II) of the present invention are shown below. It is to be understood that, according to some embodiments, the invention is not limited to any one or more of the following exemplary compounds.

According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL1-55, DSL1-56, DSL1-57, DSL1-58, DSL1-59, DSL1-60, DSL3c-1, DSL3c-2, DSL3c-3, DSL3c-4, DSL3c-5, DSL3c-6, DSL3c-7 and DSL3c-8, DSL2-1, DSL2-2, DSL2-3, DSL2-4, DSL2-5, DSL2-6, DSL2-7, DSL2-8, DSL2-9, DSL2-10, DSL2-11, DSL2-12, DSL2-13, DSL2-14, DSL2-15, DSL2-16, DSL2-17, DSL2-18, DSL2-19, DSL2-20, DSL2-21, DSL2-22, DSL2-23, DSL2-24, DSL2-25, DSL2-26, DSL2-27, DSL2-28, DSL2-29, DSL2-30, DSL2-31, DSL2-32, DSL2-33, DSL2-34, DSL2-35, DSL2-36, DSL2-37, DSL2-38, DSL2-39, DSL2-40, DSL2-41, DSL2-42, DSL2-43, DSL2-44, DSL2-45, DSL2-46, DSL2-47, DSL2-48, DSL2-49, DSL2-50, DSL2-51, DSL2-52, DSL2-53, DSL2-54, DSL2-55, DSL2-56, DSL2-57, DSL2-58, DSL2-59, DSL2-60, DSL2-61, DSL2-62, DSL2-63, DSL2-64, DSL2-65, DSL2-66, DSL2-67, DSL2-68, DSL2-69, DSL2-70, DSL2-71, DSL2-72, DSL4-1, DSL4-2, DSL4-3, DSL4-4, DSL4-5, DSL4-6, DSL4-7, DSL4-8, DSL4-9, DSL4-10, DSL4-11, DSL4-12, DSL4-13, DSL4-14 and DSL4-15, including salts, hydrates, solvates, polymorphs, optical isomers, geometrical isomers, enantiomers, diastereomers, and mixtures thereof. Each possibility represents a separate embodiment of the present invention.

The following exemplary compounds are portrayed as non-limiting examples of the lipids of the present invention. The substituents of each lipid are identified below, sometimes within curly brackets, { }, for identification of most specific embodiments.

Specifically, the compound DSL1-1 is shown below,

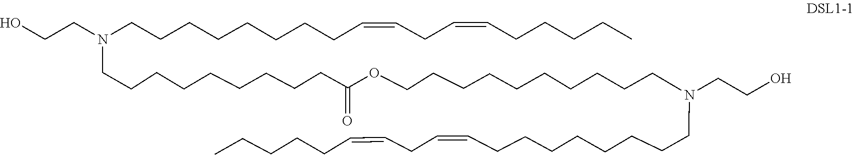 [SMILES: CCCCC/C=C\C/C=C\CCCCCCCCN(CCO)CCCCCCCCCCOC(=O)CCCCCCCCCN(CCO)CCCCCCCC/C=C\C/C=C\CCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is O—Y^{b}, wherein Y^{b} is H; R² is C₅₋₂₅ alkenyl {C₁₈ diene chain}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkenyl {C₁₈ diene chain}. As may be understood, DSL1-1 can also be represented by other formulas described herein, e.g., Formula (II).

The compound DSL1-2 is shown below,

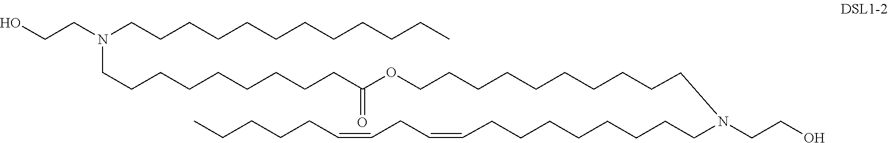 [SMILES: CCCCC/C=C\C/C=C\CCCCCCCCN(CCO)CCCCCCCCCCOC(=O)CCCCCCCCCN(CCO)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is O—Y^{b}, wherein Y^{b} is H; R² is C₅₋₂₅ alkyl {C₁₂ alkyl}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkenyl {C₁₈ diene chain}. As may be understood, DSL1-2 can also be represented by other formulas described herein, e.g., Formula (II).

The compound DSL1-3 is shown below,

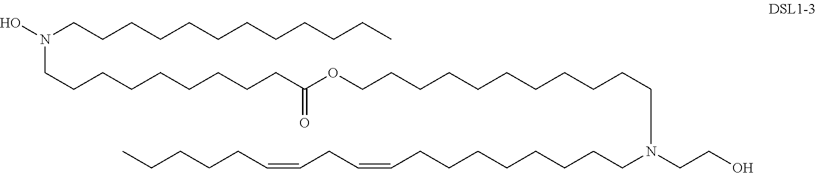 [SMILES: CCCCC/C=C\C/C=C\CCCCCCCCN(CCO)CCCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is C₀₋₁₀ alkylene-Y^{a} {C₀ alkylene-Y^{a}, i.e., only Y^{a}}, wherein Y^{a} is O—Y^{b}, wherein Y^{b} is H; R² is C₅₋₂₅ alkyl {C₁₂ alkyl}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkenyl {C₁₈ diene chain}. As may be understood, DSL1-3 can also be represented by other formulas described herein, e.g., Formula (II).

The compound DSL1-4 is shown below,

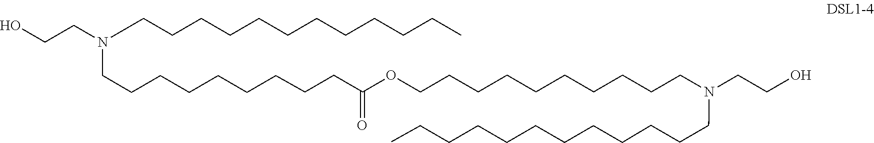 [SMILES: CCCCCCCCCCCCN(CCO)CCCCCCCCCCOC(=O)CCCCCCCCCN(CCO)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is O—Y^{b}, wherein Y^{b} is H; R² is C₅₋₂₅ alkyl {C₁₂ alkyl}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkyl {C₁₂ alkyl}. As may be understood, DSL1-4 can also be represented by other formulas described herein, e.g., Formula (II).

The compound DSL1-5 is shown below,

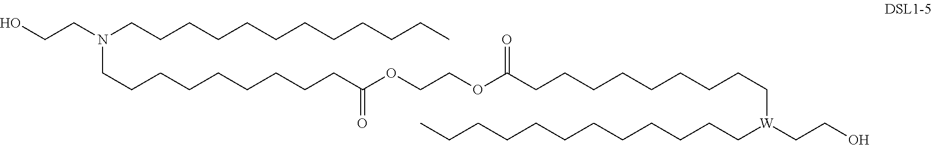

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is O—Y^{b}, wherein Y^{b} is H; R² is C₅₋₂₅ alkyl {C₁₂ alkyl}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkyl {C₁₂ alkyl}. As may be understood, DSL1-5 can also be represented by other formulas described herein, e.g., Formula (II).

The compound DSL1-6 is shown below,

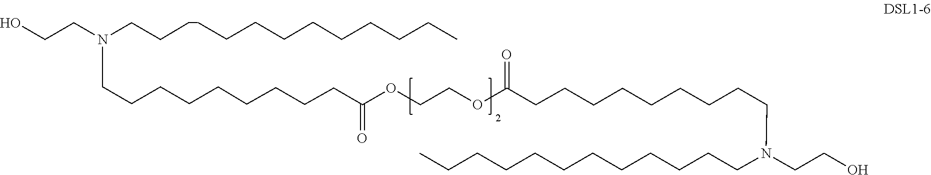

It is represented by Formula (I), wherein L is L^{a}-X^{a}-L^{b}, X^{a} is —CO₂-L^{c}-O₂C—, wherein L^{c} is C₁₋₆ alkylene-(O—C₁₋₆ alkylene)_{f} {i.e., C₂ alkylene-(O—C₂ alkylene)_{f}}, wherein f is 1; L^{a} is C₄₋₂₀ alkylene {C₉ alkylene}, L^{b} is C₄₋₂₀ alkylene {C₉ alkylene}; R¹ is C₀₋₁₀ alkylene-Y^{a} {i.e., C₂ alkylene-Y^{a}}, wherein Y^{a} is O—Y^{b}, wherein Y^{b} is H; R² is C₅₋₂₅ alkyl {C₁₂ alkyl}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkyl {C₁₂ alkyl}.

The compound DSL1-7 is shown below,

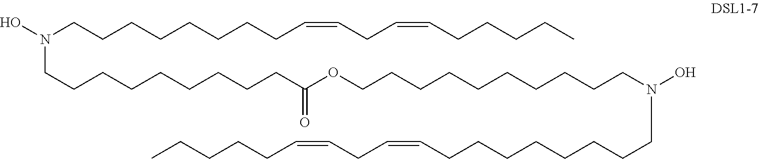 [SMILES: CCCCC/C=C\C/C=C\CCCCCCCCN(O)CCCCCCCCCCOC(=O)CCCCCCCCCN(O)CCCCCCCC/C=C\C/C=C\CCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₀ alkylene-Y^{a}, i.e., only Y^{a}}, wherein Y^{a} is O—Y^{b}, wherein Y^{b} is H; R² is C₅₋₂₅ alkenyl {C₁₈ diene chain}; R³ is C₀₋₆ alkylene-Y^{d} {C₀ alkylene-Y^{d}, i.e., only Y^{d}}, wherein Y^{d} is O—Y^{e}, wherein Y^{e} is H; and R⁴ is C₅-alkenyl {C₁₈ diene chain}.

The compound DSL1-8 is shown below,

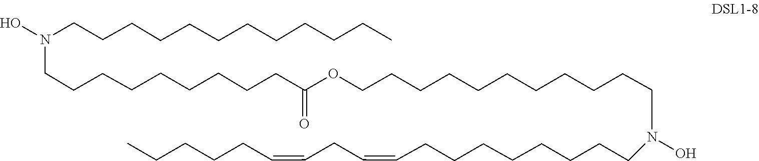 [SMILES: CCCCC/C=C\C/C=C\CCCCCCCCN(O)CCCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is C₀₋₁₀ alkylene-Y^{a} {C₀ alkylene-Y^{a}, i.e., only Y^{a}}, wherein Y^{a} is O—Y^{b}, wherein Y^{b} is H; R² is C₅₋₂₅ alkyl {C₁₂ alkyl}; R³ is C₀₋₆ alkylene-Y^{d}{C₀ alkylene-Y^{d}, i.e., only Y^{d}}, wherein Y^{d} is O—Y^{e}, wherein Y^{e} is H; and R⁴ is C₅₋₂₅ alkenyl IC₁₈ diene chain}.

The compound DSL1-9 is shown below,

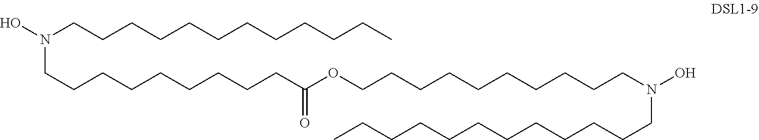 [SMILES: CCCCCCCCCCCCN(O)CCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₀ alkylene-Y^{a}, i.e., only Y^{a}}, wherein Y^{a} is O—Y^{b}, wherein Y^{b} is H; R² is C₅₋₂₅ alkyl {C₁₂ alkyl}; R³ is C₀₋₆ alkylene-Y^{d} {C₀ alkylene-Y^{d}, i.e., only Y^{d}}, wherein Y^{d} is O—Y^{e}, wherein Y^{e} is H; and R⁴ is C₅₋₂₅ alkyl IC₁₂ alkyl}.

The compound DSL1-10 is shown below,

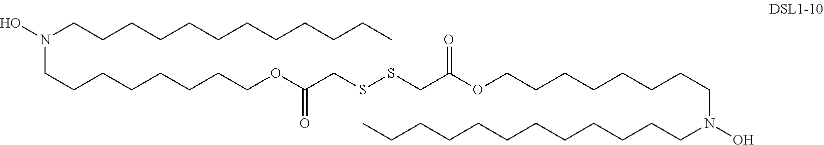 [SMILES: CCCCCCCCCCCCN(O)CCCCCCCCOC(=O)CSSCC(=O)OCCCCCCCCN(O)CCCCCCCCCCCC]

It is represented by Formula (I), wherein L is L^{a}-X^{a}-L^{b}, X^{a} is —O₂C-L^{c}-CO₂—, L^{c} is C₁₋₆ alkylene-S—S—C₁₋₆ alkylene {C₁ alkylene-S—S—C₁ alkylene}, L^{a} is C₄₋₂₀ alkylene {C₈ alkylene}, L^{b} is C₄₋₂₀ alkylene {C₈ alkylene}; R¹ is OH as detailed for DSL1-7; R² is C₅₋₂₅ alkyl {C₁₂ alkyl}; R³ is OH as detailed for DSL1-7; and R⁴ is C₅₋₂₅ alkyl {C₁₂ alkyl}.

The compound DSL1-11 is shown below,

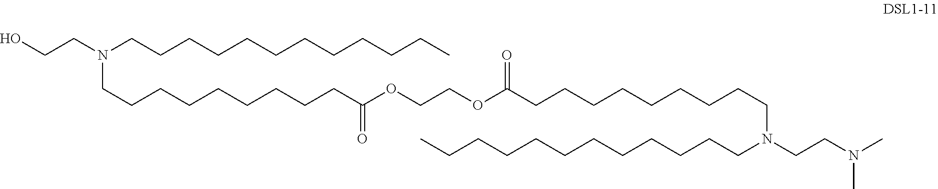 [SMILES: CCCCCCCCCCCCN(CCO)CCCCCCCCCC(=O)OCCOC(=O)CCCCCCCCCN(CCCCCCCCCCCC)CCN(C)C]

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is hydroxyethyl as detailed for DSL1-5; R² is C₅₋₂₅ alkyl {C₁₂ alkyl}; R³ is C₀₋₁₀ alkylene-Y^{d}, wherein Y^{d} is NMe₂; and R⁴ is C₅₋₂₅ alkyl {C₁₂ alkyl}.

The compound DSL1-12 is shown below,

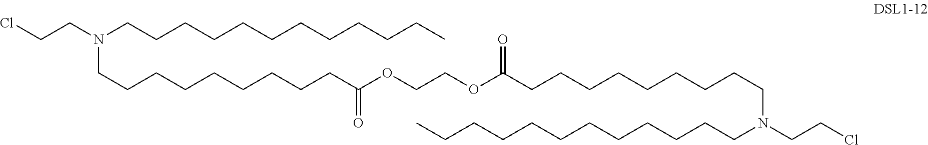 [SMILES: CCCCCCCCCCCCN(CCCl)CCCCCCCCCC(=O)OCCOC(=O)CCCCCCCCCN(CCCl)CCCCCCCCCCCC]

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is C₀₋₁₀ alkylene-Y^{a} {i.e., C₂ alkylene-Y^{a}}, wherein Y^{a} is halogen {Chlorine}; R² is C₅₋₂₅ alkyl IC₁₂ alkyl}; R³ is C₀₋₆ alkylene-Y^{d} {i.e., C₂ alkylene-Y^{d}}, wherein Y^{d} is halogen {Chlorine}; and R⁴ is C₅₋₂₅ alkyl {C₁₂ alkyl}.

The compound DSL1-13 is shown below,

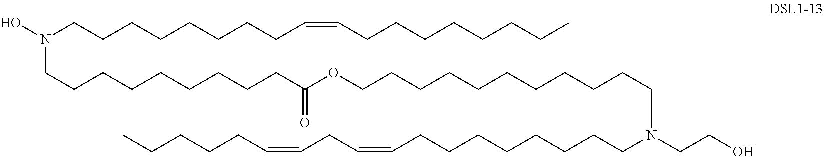 [SMILES: CCCCC/C=C\C/C=C\CCCCCCCCN(CCO)CCCCCCCCCCCOC(=O)CCCCCCCCCN(O)CCCCCCCC/C=C\CCCCCCCC]

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is C₀₋₁₀ alkylene-Y^{a} {C₀ alkylene-Y^{a}, i.e., only Y^{a}}, wherein Y^{a} is O—Y^{b}, wherein Y^{b} is H; R² is C₅₋₂₅ alkenyl {C₁₈ alkenyl}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkenyl {C₁₈ diene chain}. As may be understood, DSL1-13 can also be represented by other formulas described herein, e.g., Formula (II).

The compound DSL1-14 is shown below,

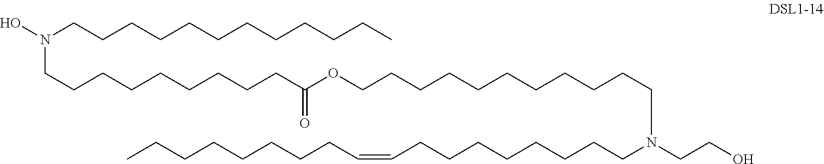 [SMILES: CCCCCCCC/C=C\CCCCCCCCN(CCO)CCCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is OH, as detailed for DSL1-3; R² is C₅₋₂₅ alkyl {C₁₂ alkyl}; R³ is hydroxyethyl as detailed for DSL1-3; and R⁴ is C₅₋₂₅ alkenyl {C₁₈ alkenyl}. As may be understood, DSL1-14 can also be represented by other formulas described herein, e.g., Formula (II).

The compound DSL1-15 is shown below,

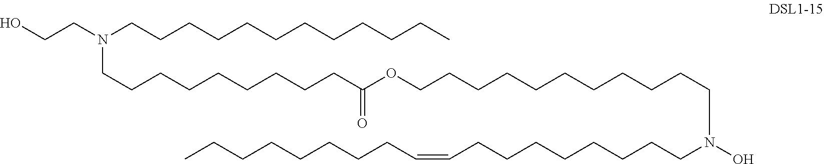 [SMILES: CCCCCCCC/C=C\CCCCCCCCN(O)CCCCCCCCCCCOC(=O)CCCCCCCCCN(CCO)CCCCCCCCCCCC]

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is O—Y^{b}, wherein Y^{b} is H; R² is C₅₋₂₅ alkyl {C₁₂ alkyl}; R³ is C₀₋₆ alkylene-Y^{d} {C₀ alkylene-Y^{d}, i.e., only Y^{d}}, wherein Y^{d} is O—Y^{e}, wherein Y^{e} is H; and R⁴ is C₅₋₂₅ alkenyl {C₁₈ alkenyl}. As may be understood, DSL1-15 can also be represented by other formulas described herein, e.g., Formula (II).

The compound DSL1-16 is shown below,

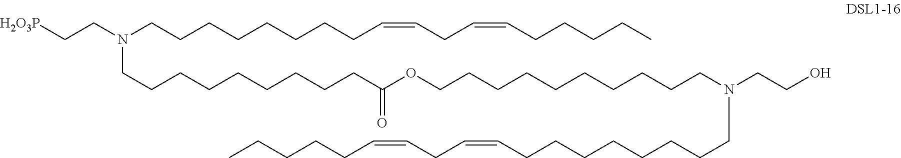

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is PO₃H₂; R² is C₅₋₂₅ alkenyl {C₁₈ diene chain}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkenyl {C₁₈ diene chain}.

The compound DSL1-17 is shown below,

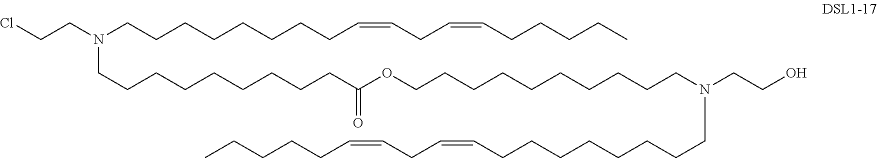 [SMILES: CCCCC/C=C\C/C=C\CCCCCCCCN(CCO)CCCCCCCCCCOC(=O)CCCCCCCCCN(CCCl)CCCCCCCC/C=C\C/C=C\CCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is halogen {chlorine}; R² is C₅₋₂₅ alkenyl {C₁₈ diene chain}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkenyl {C₁₈ diene chain}.

The compound DSL1-18 is shown below,

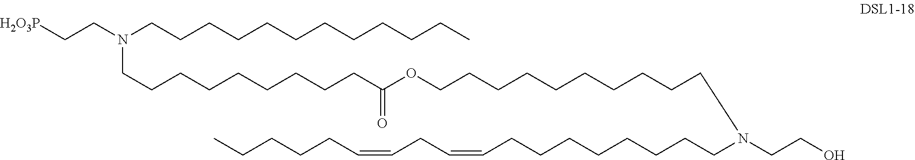

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is PO₃H₂; R² is C₅₋₂₅ alkyl {C₁₂ alkyl}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkenyl {C₁₈ diene chain}.

The compound DSL1-19 is shown below,

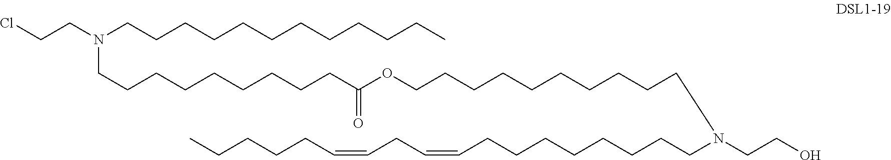 [SMILES: CCCCC/C=C\C/C=C\CCCCCCCCN(CCO)CCCCCCCCCCOC(=O)CCCCCCCCCN(CCCl)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is halogen {chlorine}; R² is C₅₋₂₅ alkyl {C₁₂ alkyl}; R³ is C₀₋₆ alkylene-Y^{d} {C₂ alkylene-Y^{d}}, wherein Y^{d} is O—Y^{e}, wherein Y^{e} is H; and R⁴ is C₅₋₂₅ alkenyl {C₁₈ diene chain}.

The compound DSL1-20 is shown below,

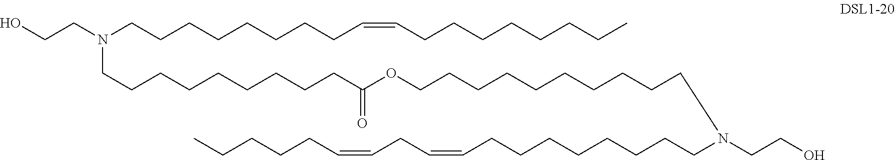 [SMILES: CCCCC/C=C\C/C=C\CCCCCCCCN(CCO)CCCCCCCCCCOC(=O)CCCCCCCCCN(CCO)CCCCCCCC/C=C\CCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is hydroxyethyl as shown for DSl1-1; R² is C₅₋₂₅ alkenyl {C₁₈ alkenyl}; R³ is hydroxyethyl as shown for DSl1-1; and R⁴ is C₅₋₂₅ alkenyl {C₁₈ diene chain}. As may be understood, DSL1-20 can also be represented by other formulas described herein, e.g., Formula (II).

The compound DSL1-21 is shown below,

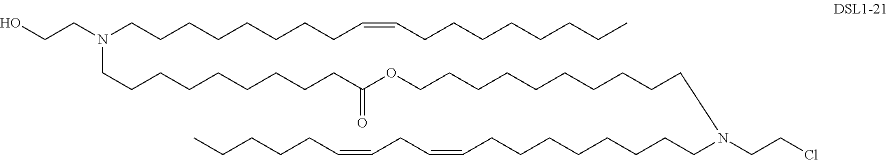 [SMILES: CCCCC/C=C\C/C=C\CCCCCCCCN(CCCl)CCCCCCCCCCOC(=O)CCCCCCCCCN(CCO)CCCCCCCC/C=C\CCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹C₀₋₆ alkylene-Y^{a} {C₂ alkylene-Y^{a}}, wherein Y^{a} is O—Y^{b}, wherein Y^{b} is H; R² is C₅₋₂₅ alkenyl {C₁₈ alkenyl}; R³ is C₀₋₁₀ alkylene-Y^{d} {C₂ alkylene-Y^{d}}, wherein Y^{d} is halogen {chlorine}; and R⁴ is C₅₋₂₅ alkenyl {C₁₈ diene chain}.

The compound DSL1-22 is shown below,

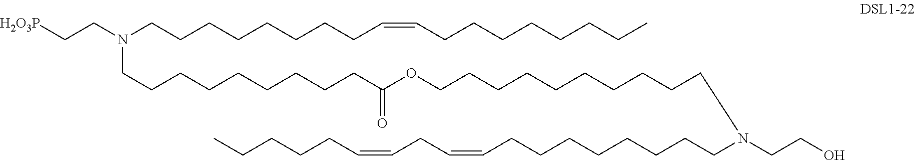

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is CH₂CH₂—PO₃H₂ as detailed for DSL1-18; R² is C₅₋₂₅ alkenyl {C₁₈ alkenyl}; R³ is hydroxyethyl as detailed for DSL1-18; and R⁴ is C₅₋₂₅ alkenyl {C₁₈ diene chain}.

The compound DSL1-23 is shown below,

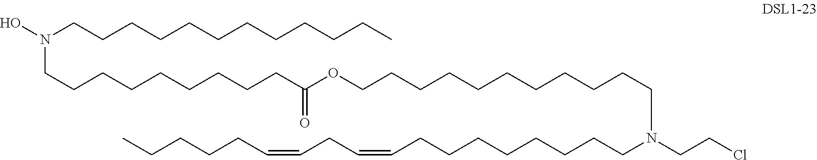 [SMILES: CCCCC/C=C\C/C=C\CCCCCCCCN(CCCl)CCCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is OH as detailed for DSL1-7; R² is C₅₋₂₅ alkyl {C₁₂ alkyl}; R³ is chloroethyl as detailed for DSL1-12; and R⁴ is C₅₋₂₅ alkenyl {C₁₈ diene chain}.

The compound DSL1-24 is shown below,

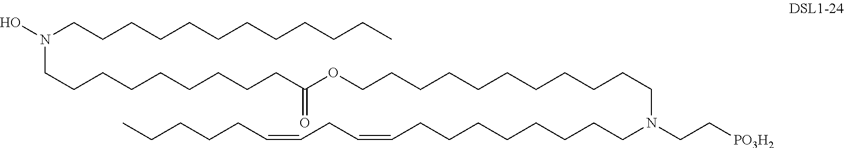

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is OH as detailed for DSL1-7; R² is C₅₋₂₅ alkyl {C₁₂ alkyl}; R³ is C₀₋₁₀ alkylene-Y^{d} {C₂ alkylene-Y^{d}}, wherein Y^{d} is PO₃H₂; and R⁴ is C₅₋₂₅ alkenyl {C₁₈ diene chain}.

The compound DSL1-25 is shown below,

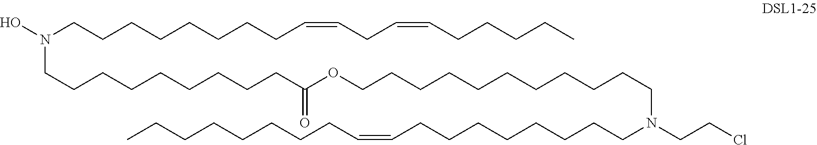 [SMILES: CCCCC/C=C\C/C=C\CCCCCCCCN(O)CCCCCCCCCC(=O)OCCCCCCCCCCCN(CCCl)CCCCCCCC/C=C\CCCCCCCC]

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is OH as detailed for DSL1-7; R² is C₅₋₂₅ alkenyl {C₁₈ dienyl}; R³ is chloroethyl as detailed for DSL1-12; and R⁴ is C₅₋₂₅ alkenyl {C₁₈ alkenyl}.

The compound DSL1-26 is shown below,

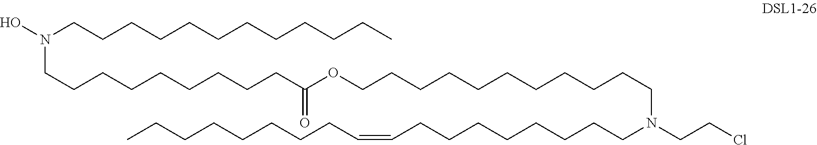 [SMILES: CCCCCCCC/C=C\CCCCCCCCN(CCCl)CCCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is OH, as detailed for DSL1-3; R² is C₅₋₂₅ alkyl {C₁₂ alkyl}; R³ is chloroethyl as detailed for DSL1-12; and R⁴ is C₅₋₂₅ alkenyl {C₁₈ alkenyl}.

The compound DSL1-27 is shown below

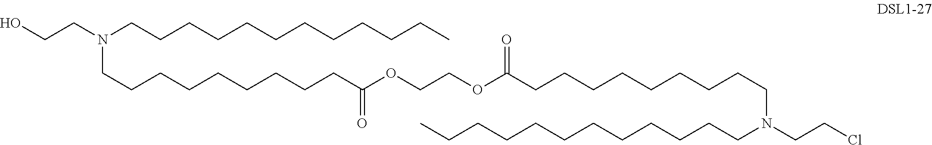 [SMILES: CCCCCCCCCCCCN(CCO)CCCCCCCCCC(=O)OCCOC(=O)CCCCCCCCCN(CCCl)CCCCCCCCCCCC]

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is hydroxyethyl as detailed for DSL1-1; R² is C₅₋₂₅ alkyl {C₁₂ alkyl}; R³ is chloroethyl as detailed for DSL1-12; and R⁴ is C₅₋₂₅ alkyl {C₁₂ alkyl}.

The compound DSL1-28 is shown below,

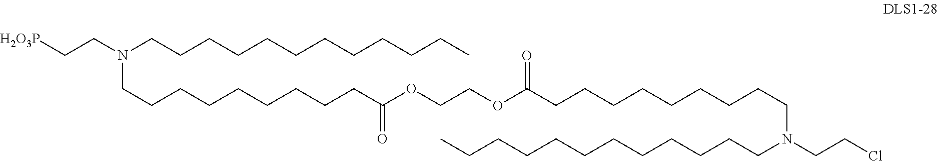

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is CH₂CH₂—PO₃H₂ as detailed for DSL1-16; R² is C₅₋₂₅ alkyl {C₁₂ alkyl}; R³ is chloroethyl as detailed for DSL1-12; and R⁴ is C₅₋₂₅ alkyl {C₁₂ alkyl}.

The compound DSL1-29 is shown below,

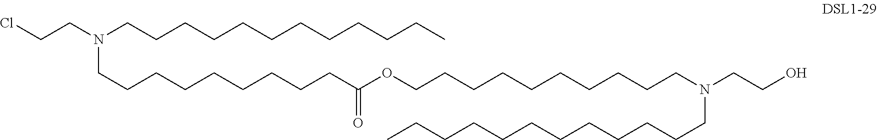 [SMILES: CCCCCCCCCCCCN(CCO)CCCCCCCCCCOC(=O)CCCCCCCCCN(CCCl)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is chloroethyl as detailed for DSL1-12; R² is C₅₋₂₅ alkyl {C₁₂ alkyl}; R³ hydroxyethyl as detailed for DSL1-1; and R⁴ is C₅₋₂₅ alkyl {C₁₂ alkyl}.

The compound DSL1-30 is shown below,

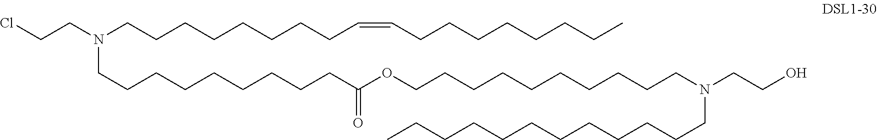 [SMILES: CCCCCCCC/C=C\CCCCCCCCN(CCCl)CCCCCCCCCC(=O)OCCCCCCCCCCN(CCO)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is chloroethyl as detailed for DSL1-12; R² is C₅-alkenyl {C₁₈ alkenyl}; R³ hydroxyethyl as detailed for DSL1-1; and R⁴ is C₅₋₂₅ alkyl {C₁₂ alkyl}. The compound DSL1-31 is shown below,

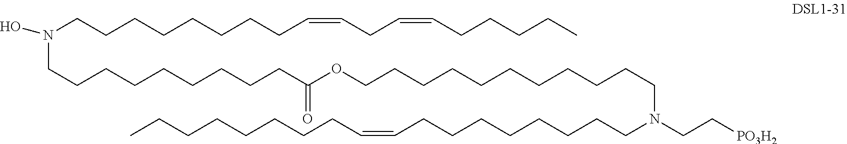

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is OH as detailed for DSL1-7; R² is C₅₋₂₅ alkenyl {C₁₈ dienyl}; R³ is CH₂CH₂—PO₃H₂ as detailed for DSL1-24; and R⁴ is C₅₋₂₅ alkenyl {C₁₈ alkenyl}. The compound DSL1-32 is shown below,

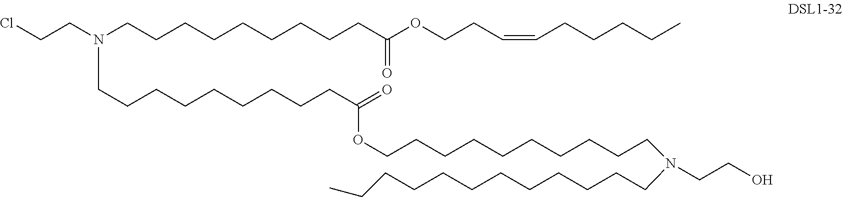 [SMILES: CCCCC/C=C\CCOC(=O)CCCCCCCCCN(CCCl)CCCCCCCCCC(=O)OCCCCCCCCCCN(CCO)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is chloroethyl as detailed for DSL1-12; R² is C₅-alkylene-CO₂—C₅₋₁₅ alkenyl {C₉ alkylene-CO₂—C₉ alkenyl}; R³ is hydroxyethyl as detailed for DSL1-1; and R⁴ is C₅₋₂₅ alkyl {C₁₂ alkyl}.

The compound DSL1-33 is shown below,

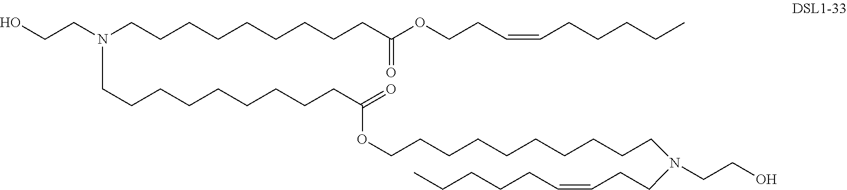 [SMILES: CCCCC/C=C\CCOC(=O)CCCCCCCCCN(CCO)CCCCCCCCCC(=O)OCCCCCCCCCCN(CCO)CC/C=C\CCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is hydroxyethyl as detailed for DSL1-1; R² is C₅-alkylene-CO₂—C₅₋₁₅ alkenyl {C₉ alkylene-CO₂—C₉ alkenyl}; R³ is hydroxyethyl as detailed for DSL1-1; and R⁴ is C₅₋₂₅ alkenyl {C₉ alkenyl}.

The compound DSL1-34 is shown below,

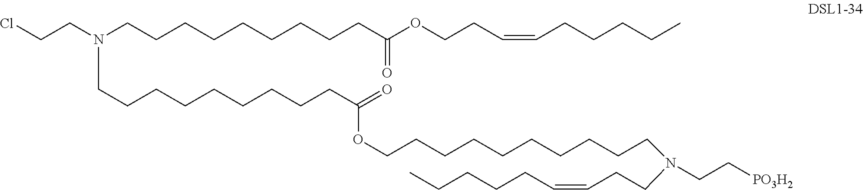

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is chloroethyl as detailed for DSL1-12; R² is C₅₋₁₅ alkylene-CO₂—C₅₋₁₅ alkenyl {C₉ alkylene-CO₂—C₉ alkenyl}; R³ is CH₂CH₂—PO₃H₂ as detailed for DSL1-24; and R⁴ is C₅₋₂₅ alkenyl {C₉ alkenyl}.

The compound DSL1-35 is shown below,

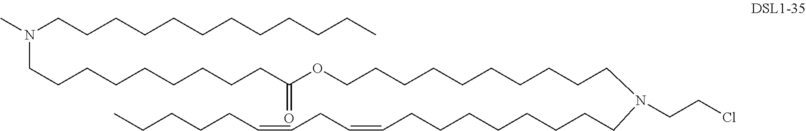 [SMILES: CCCCC/C=C\C/C=C\CCCCCCCCN(CCCl)CCCCCCCCCCOC(=O)CCCCCCCCCN(C)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₁₋₄ alkyl {methyl}; R² is C₅₋₂₅ alkyl {C₁₂ alkyl}; R³ is chloroethyl as detailed for DSL1-12; and R⁴ is C₅₋₂₅ alkenyl {C₁₈ diene chain}.

The compound DSL1-36 is shown below,

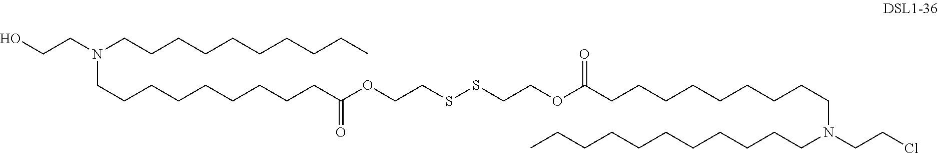 [SMILES: CCCCCCCCCCCN(CCCl)CCCCCCCCCC(=O)OCCSSCCOC(=O)CCCCCCCCCN(CCO)CCCCCCCCCC]

It is represented by Formula (I), wherein L is L^{a}-X^{a}-L^{b}, X^{a} is —CO₂-L^{c}-O₂C—, L^{c} is C₁₋₆ alkylene-S—S—C₁₋₆ alkylene {C₂ alkylene-S—S—C₂ alkylene}, L^{a} is C₄₋₂₀ alkylene {C₉ alkylene}, L^{b} is C₄₋₂₀ alkylene {C₉ alkylene}; R¹ is hydroxyethyl as detailed for DSL1-1; R² is C₅₋₂₅ alkyl {C₁₀ alkyl}; R³ is chloroethyl as detailed for DSL1-12; and R⁴ is C₅₋₂₅ alkyl {C₁₁ alkyl}.

The compound DSL1-37 is shown below,

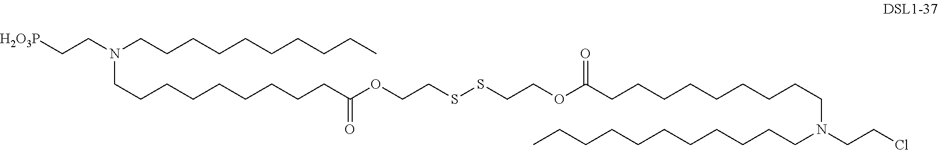

It is represented by Formula (I), wherein L is L^{a}-X^{a}-L^{b}, X^{a} is —CO₂-L^{c}-O₂C—, L^{c} is C₁₋₆ alkylene-S—S—C₁₋₆ alkylene {C₂ alkylene-S—S—C₂ alkylene}, L^{a} is C₄₋₂₀ alkylene {C₉ alkylene}, L^{b} is C₄₋₂₀ alkylene {C₉ alkylene}; R¹ is CH₂CH₂—PO₃H₂ as detailed for DSL1-16; R² is C₅₋₂₅ alkyl {C₁₀ alkyl}; R³ is chloroethyl as detailed for DSL1-12; and R⁴ is C₅₋₂₅ alkyl {C₁₁ alkyl}.

The compound DSL1-38 is shown below,

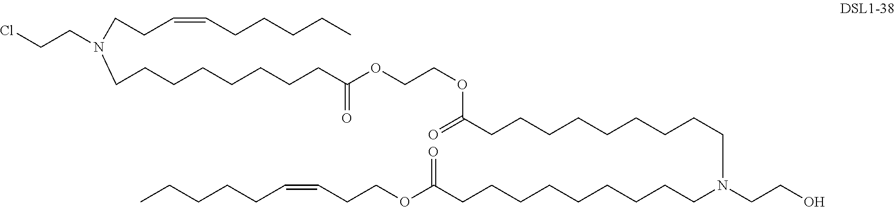 [SMILES: CCCCC/C=C\CCOC(=O)CCCCCCCCCN(CCO)CCCCCCCCCC(=O)OCCOC(=O)CCCCCCCCN(CCCl)CC/C=C\CCCCC]

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₈ alkylene}; R¹ is chloroethyl as detailed for DSL1-12; R² is C₅₋₂₅ alkenyl {C₉ alkenyl}; R³ is hydroxyethyl as detailed for DSL1-1; and R⁴ is C₅₋₁₅ alkylene-CO₂—C₅₋₁₅ alkenyl {C₉ alkylene-CO₂—C₉ alkenyl}.

The compound DSL1-39 is shown below,

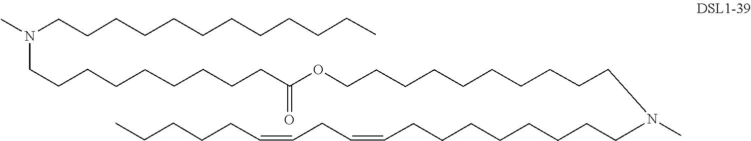

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₁₋₄ alkyl {methyl}; R² is C₅₋₂₅ alkyl {C₁₂ alkyl}; R³ is C₁₋₄ alkyl {methyl}; and R⁴ is C₅₋₂₅ alkenyl {C₁₈ diene chain}.

The compound DSL1-40 is shown below,

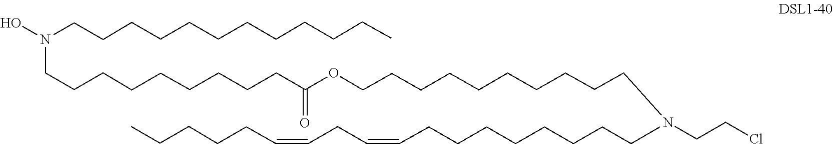 [SMILES: CCCCC/C=C\C/C=C\CCCCCCCCN(CCCl)CCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R² is C₅₋₂₅ alkyl {C₁₂ alkyl}; R³ is chloroethyl as detailed for DSL1-12; and R⁴ is C₅₋₂₅ alkenyl {C₁₈ diene chain}.

The compound DSL1-41 is shown below,

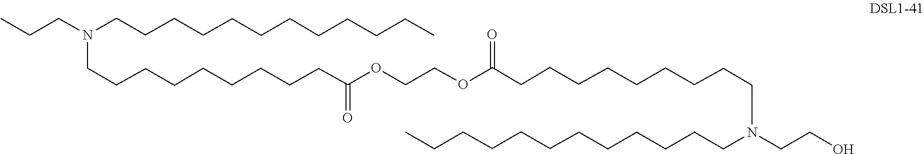 [SMILES: CCCCCCCCCCCCN(CCC)CCCCCCCCCC(=O)OCCOC(=O)CCCCCCCCCN(CCO)CCCCCCCCCCCC]

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is C₁₋₄ alkyl {propyl}; R² is C₅₋₂₅ alkyl {C₁₂ alkyl}; R³ is hydroxyethyl as detailed for DSL1-1; and R⁴ is C₅₋₂₅ alkyl {C₁₂ alkyl}.

The compound DSL1-42 is shown below,

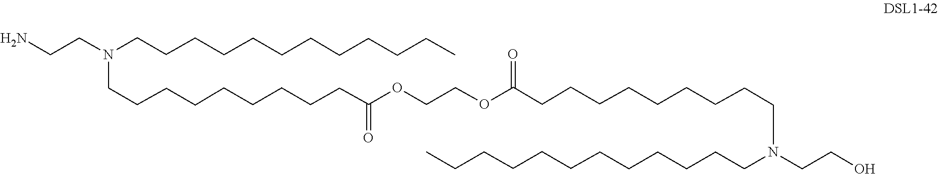 [SMILES: CCCCCCCCCCCCN(CCN)CCCCCCCCCC(=O)OCCOC(=O)CCCCCCCCCN(CCO)CCCCCCCCCCCC]

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is C₀₋₁₀ alkylene-Y^{a}, wherein Y^{a} is NH₂; R² is C₅₋₂₅ alkyl {C₁₂ alkyl}; R³ is hydroxyethyl as detailed for DSL1-1; and R⁴ is C₅₋₂₅ alkyl {C₁₂ alkyl}.

The compound DSL1-43 is shown below,

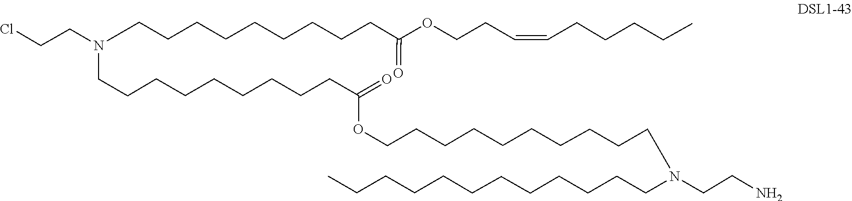

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is chloroethyl as detailed for DSL1-12; R² is C₅-alkylene-CO₂—C₅₋₁₅ alkenyl {C₉ alkylene-CO₂—C₉ alkenyl}; R³ is C₀₋₁₀ alkylene-Y^{d}, wherein Y^{d} is NH₂; and R⁴ is C₅₋₂₅ alkyl {C₁₂ alkyl}.

The compound DSL1-44 is shown below,

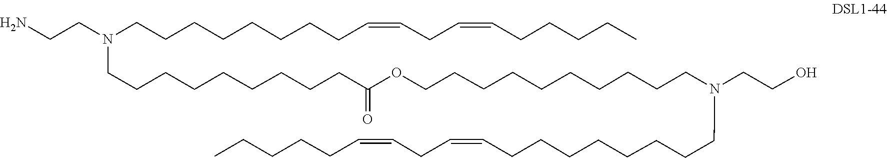 [SMILES: CCCCC/C=C\C/C=C\CCCCCCCCN(CCN)CCCCCCCCCC(=O)OCCCCCCCCCCN(CCO)CCCCCCCC/C=C\C/C=C\CCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹R¹ is aminoethyl as detailed for DSL1-42; R² is C₅₋₂₅ alkenyl {C₁₈ diene chain}; R³ is hydroxyethyl as detailed for DSL1-1; and R⁴ is C₅₋₂₅ alkenyl {C₁₈ diene chain}.

The compound DSL1-45 is shown below,

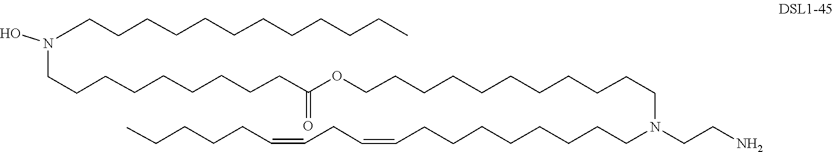 [SMILES: CCCCC/C=C\C/C=C\CCCCCCCCN(CCN)CCCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₁ alkylene}, L^{b} is C₄₋₂₀ alkylene {C₉ alkylene}; R¹ is OH as detailed for DSL1-3; R² is C₅₋₂₅ alkyl {C₁₂ alkyl}; R³ is aminoethyl as detailed for DSL1-43; and R⁴ is C₅₋₂₅ alkenyl {C₁₈ diene chain}.

The compound DSL1-46 is shown below,

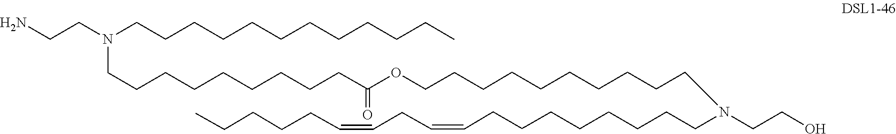 [SMILES: CCCCC/C=C\C/C=C\CCCCCCCCN(CCO)CCCCCCCCCCOC(=O)CCCCCCCCCN(CCN)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is aminoethyl as detailed for DSL1-42; R² is C₅₋₂₅ alkyl {C₁₂ alkyl}; R³ is hydroxyethyl as detailed for DSL1-1; and R⁴ is C₅₋₂₅ alkenyl {C₁₈ diene chain}.

The compound DSL1-47 is shown below,

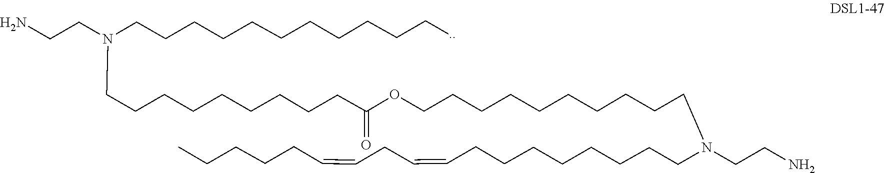

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is aminoethyl as detailed for DSL1-42; R² is C₅₋₂₅ alkyl {C₁₂ alkyl}; R³ is aminoethyl as detailed for DSL1-43; and R⁴ is C₅₋₂₅ alkenyl {C₁₈ diene chain}.

The compound DSL1-48 is shown below,

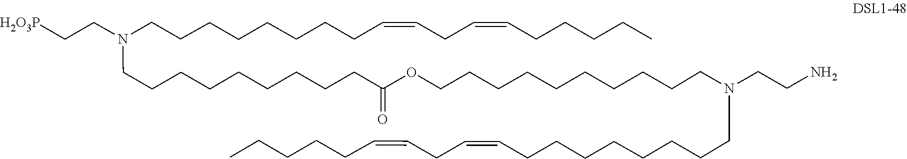

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is CH₂CH₂—PO₃H₂ as detailed for DSL1-16; R² is C₅₋₂₅ alkenyl {C₁₈ diene chain}; R³ is aminoethyl as detailed for DSL1-43; and R⁴ is C₅₋₂₅ alkenyl {C₁₈ diene chain}.

The compound DSL1-49 is shown below,

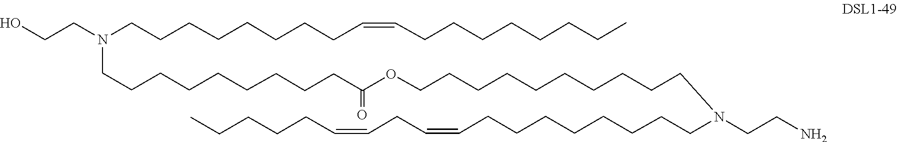 [SMILES: CCCCC/C=C\C/C=C\CCCCCCCCN(CCN)CCCCCCCCCCOC(=O)CCCCCCCCCN(CCO)CCCCCCCC/C=C\CCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is hydroxyethyl as detailed for DSL1-1; R² is C₅₋₂₅ alkenyl {C₁₈ alkenyl}; R³ is aminoethyl as detailed for DSL1-43; and R⁴ is C₅₋₂₅ alkenyl {C₁₈ diene chain}.

The compound DSL1-50 is shown below,

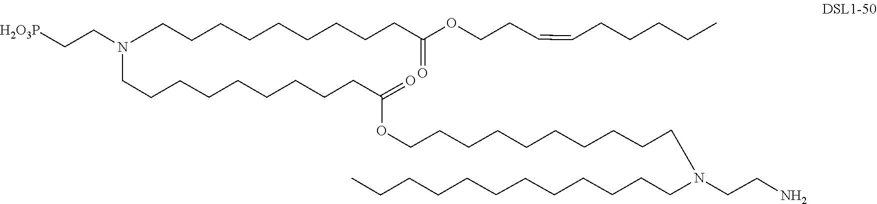

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is CH₂CH₂—PO₃H₂ as detailed for DSL1-16; R² is C₅₋₁₅ alkylene-CO₂—C₅₋₁₅ alkenyl {C₉ alkylene-CO₂—C₉ alkenyl}; R³ is aminoethyl as detailed for DSL1-43; and R⁴ is C₅₋₂₅ alkyl {C₁₂ alkyl}.

The compound DSL1-51 is shown below,

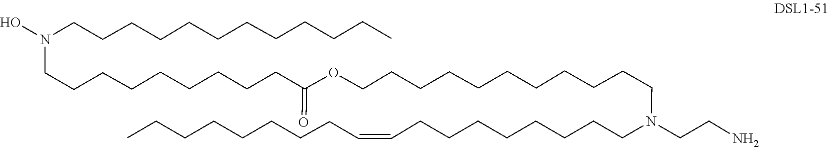 [SMILES: CCCCCCCC/C=C\CCCCCCCCN(CCN)CCCCCCCCCCCOC(=O)CCCCCCCCCN(O)CCCCCCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is OH as detailed for DSL1-3; R² is C₅₋₂₅ alkyl {C₁₂ alkyl}; R³ is aminoethyl as detailed for DSL1-43; and R⁴ is C₅₋₂₅ alkenyl {C₁₈ alkenyl}.

The compound DSL1-52 is shown below,

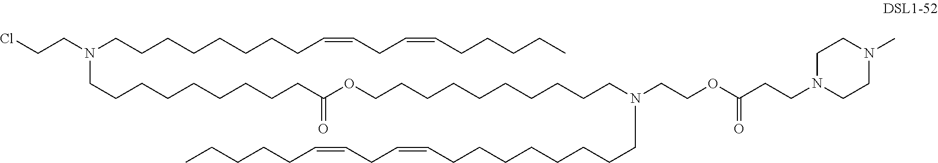 [SMILES: CCCCC/C=C\C/C=C\CCCCCCCCN(CCCl)CCCCCCCCCC(=O)OCCCCCCCCCCN(CCCCCCCC/C=C\C/C=C\CCCCC)CCOC(=O)CCN1CCN(C)CC1]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ chloroethyl as detailed for DSL1-12; R² is C₅₋₂₅ alkenyl {C₁₈ diene chain}; R³ is C₀₋₆ alkylene-Y^{d} IC₂ alkylene-Y^{d}} wherein Y^{d} is CO—C₁₋₄ alkyl-Y^{f} {CO—C₂ alkyl-Y^{f}}, wherein Y^{f} is piperazinyl substituted with a C₁₋₄ alkyl {1-piperazinyl substituted with methyl at the 4 position}; and R⁴ is C₅₋₂₅ alkenyl {C₁₈ diene chain}.

The compound DSL1-53 is shown below,

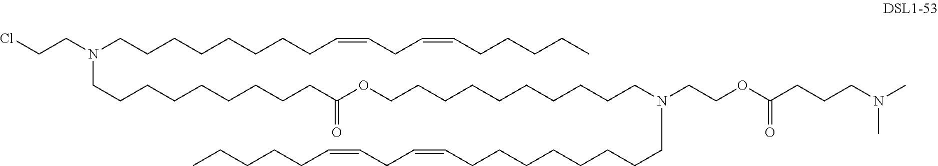 [SMILES: CCCCC/C=C\C/C=C\CCCCCCCCN(CCCl)CCCCCCCCCC(=O)OCCCCCCCCCCN(CCCCCCCC/C=C\C/C=C\CCCCC)CCOC(=O)CCCN(C)C]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ chloroethyl as detailed for DSL1-12; R² is C₅₋₂₅ alkenyl {C₁₈ diene chain}; R³ is C₀₋₆ alkylene-Y^{d} {C₂ alkylene-Y^{d}} wherein Y^{d} is CO—C₁₋₄ alkyl-Y^{f} {CO—C₃ alkyl-Y^{f}}, wherein Y^{f} is N(CH₃)₂; and R⁴ is C₅₋₂₅ alkenyl {C₁₈ diene chain}.

The compound DSL1-54 is shown below,

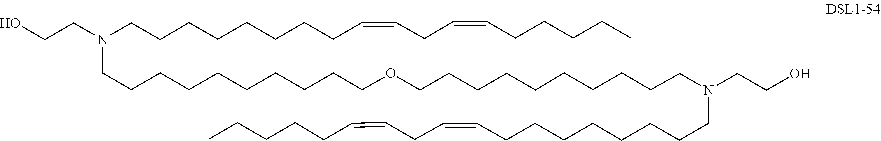 [SMILES: CCCCC/C=C\C/C=C\CCCCCCCCN(CCO)CCCCCCCCCCOCCCCCCCCCCN(CCO)CCCCCCCC/C=C\C/C=C\CCCCC]

It is represented by Formula (I), wherein L is L^{a}-X^{a}-L^{b}, X^{a} is —O—, L^{a} is C₄₋₂₀ alkylene {C₁₀ alkylene}, L^{b} is C₄₋₂₀ alkylene {C₁₀ alkylene}; R¹ is hydroxyethyl as detailed for DSL1-1; R² is C₅₋₂₅ alkenyl {C₁₈ diene chain}; R³ is hydroxyethyl as detailed for DSL1-1; and R⁴ is C₅₋₂₅ alkenyl {C₁₈ diene chain}.

The compound DSL1-55 is shown below,

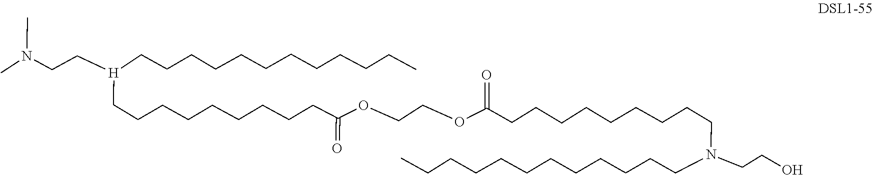

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is NMe₂; R² is C₅₋₂₅ alkyl {C₁₂ alkyl}; R³ is hydroxyethyl as detailed for DSL1-1; and R⁴ is C₅₋₂₅ alkyl {C₁₂ alkyl}.

The compound DSL1-56 is shown below,

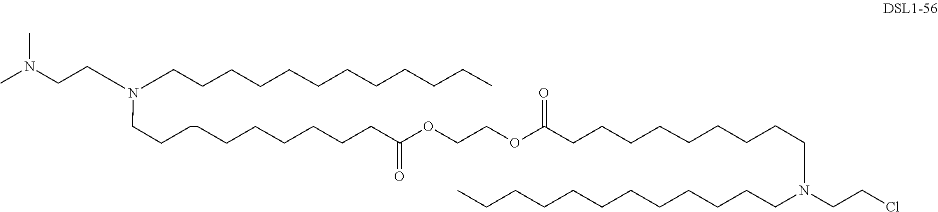 [SMILES: CCCCCCCCCCCCN(CCCl)CCCCCCCCCC(=O)OCCOC(=O)CCCCCCCCCCN(CCCCCCCCCCCC)CCCN(C)C]

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is dimethylaminoethyl as detailed for DSL1-55; R² is C₅₋₂₅ alkyl {C₁₂ alkyl}; R³ is chloroethyl as detailed for DSL1-12; and R⁴ is C₅₋₂₅ alkyl {C₁₂ alkyl}.

The compound DSL1-57 is shown below,

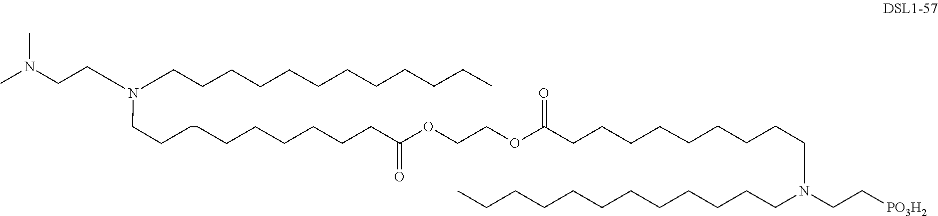

It is represented by Formula (I), wherein L is L^{a}-X^{a}-L^{b}, X^{a} is —CO₂-L^{c}-O₂C—, wherein L^{c} is C₁₋₄ alkylene {C₂ alkylene}, L^{a} is C₄₋₂₀ alkylene {C₉ alkylene}, L^{b} is C₄₋₂₀ alkylene {C₉ alkylene}; R¹ is dimethylaminoethyl as detailed for DSL1-55; R² is C₅₋₂₅ alkyl {C₁₂ alkyl}; R³ is CH₂CH₂—PO₃H₂ as detailed for DSL1-24; and R⁴ is C₅₋₂₅ alkyl {C₁₂ alkyl}.

The compound DSL1-58 is shown below,

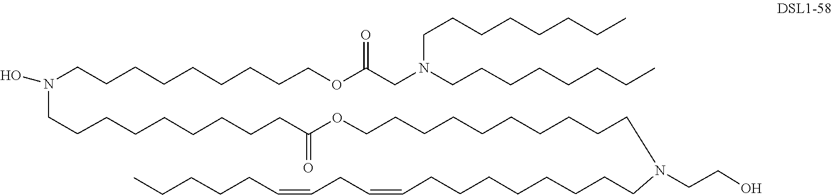 [SMILES: CCCCC/C=C\C/C=C\CCCCCCCCN(CCO)CCCCCCCCCCOC(=O)CCCCCCCCCN(O)CCCCCCCCCOC(=O)CN(CCCCCCCC)CCCCCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is OH as detailed for DSL1-3; R² is C₅₋₁₅ alkylene-O₂C—C₅₋₁₅ alkylene-N(C₁₋₁₂ alkyl)₂ {C₉ alkylene-O₂C—C₁ alkylene-N(C₈ alkyl)₂}; R³ is hydroxyethyl as detailed for DSL1-1; and R⁴ is C₅₋₂₅ alkenyl {C₁₈ diene chain}. As may be understood, DSL1-58 can also be represented by other formulas described herein, e.g., Formula (II).

The compound DSL1-59 is shown below,

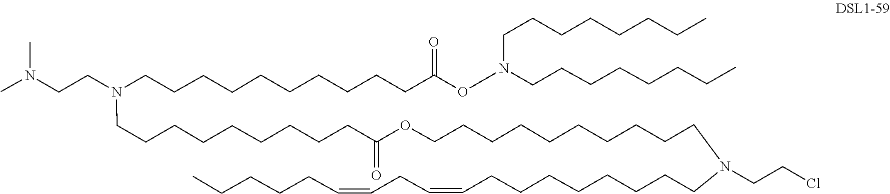 [SMILES: CCCCC/C=C\C/C=C\CCCCCCCCN(CCCl)CCCCCCCCCCOC(=O)CCCCCCCCCN(CCCCCCCCCCC(=O)ON(CCCCCCCC)CCCCCCCC)CCN(C)C]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is dimethylaminoethyl as detailed for DSL1-55; R² is C₂₋₁₅ alkylene-CO₂—C₀₋₈ alkylene-N(C₁₋₁₂ alkyl)₂ {C₁₀ alkylene-CO₂—C₀ alkylene-N(C₈ alkyl)₂}; R³ is chloroethyl as detailed for DSL1-12; and R⁴ is C₅₋₂₅ alkenyl {C₁₈ diene chain}.

The compound DSL1-60 is shown below,

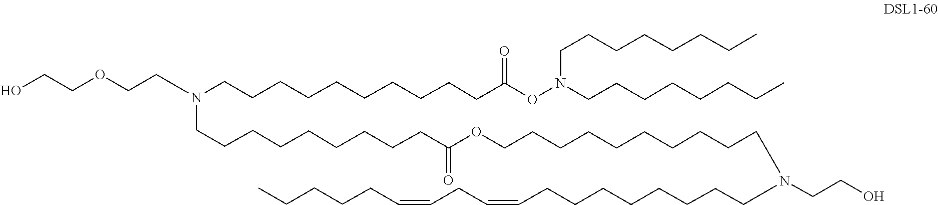 [SMILES: CCCCC/C=C\C/C=C\CCCCCCCCN(CCO)CCCCCCCCCCOC(=O)CCCCCCCCCN(CCCCCCCCCCC(=O)ON(CCCCCCCC)CCCCCCCC)CCOCCO]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is C₀₋₁₀ alkylene-Y^{a} {C₂ alkylene-Y^{a}}, wherein Y^{a} is O—Y^{b}, wherein Y^{b} is C₁₋₄ alkylene-OH {C₂ alkylene-OH}; R² is as detailed for DSL1-59; R³ is hydroxyethyl as detailed for DSL1-1; and R⁴ is C₅₋₂₅ alkenyl {C₁₈ diene chain}.

The compound DSL3C-1 is shown below,

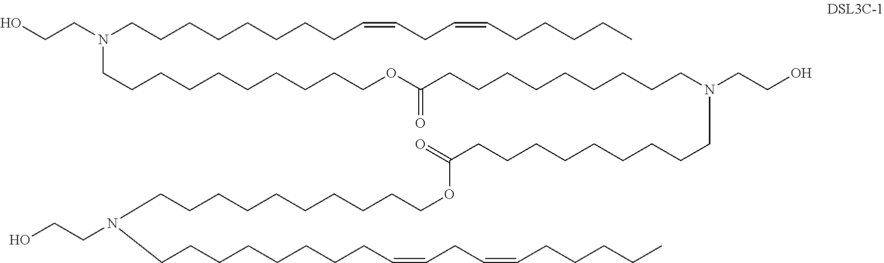 [SMILES: CCCCC/C=C\C/C=C\CCCCCCCCN(CCO)CCCCCCCCCCOC(=O)CCCCCCCCCN(CCO)CCCCCCCCCC(=O)OCCCCCCCCCCN(CCO)CCCCCCCC/C=C\C/C=C\CCCCC]

It is represented by Formula (I), wherein L is L^{a}-X^{a}-L^{b}, X^{a} is —O₂C—, L^{a} is C₄₋₂₀ alkylene {C₁₀ alkylene}, L^{b} is C₄₋₂₀ alkylene {C₉ alkylene}; R¹ is hydroxyethyl as detailed for DSL1-1; R² is C₅₋₂₅ alkenyl {C₁₈ diene chain}; R³ is hydroxyethyl as detailed for DSL1-1; R⁴ is C₅₋₁₅ alkylene-CO₂—C₅₋₁₅ alkylene-N(R⁵)R⁶ {C₉ alkylene-CO₂—C₁₀ alkylene-N(R⁵)R⁶}; R⁵ is C₀₋₁₀ alkylene-OH {C₂ alkylene-OH}; and R⁶ is C₅₋₂₅ alkenyl {C₁₈ diene chain}. As may be understood, DSL3C-1 can also be represented by other formulas described herein, e.g., Formula (II).

The compound DSL3C-2 is shown below,

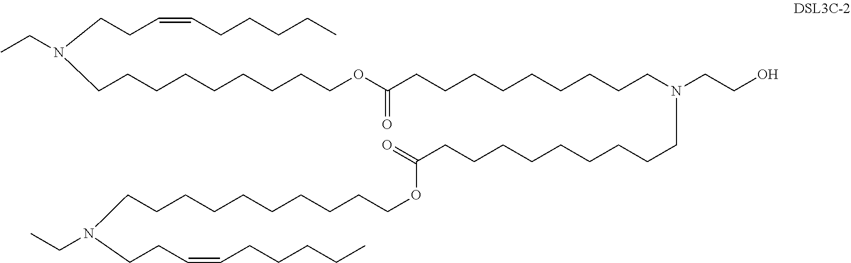 [SMILES: CCCCC/C=C\CCN(CC)CCCCCCCCCCOC(=O)CCCCCCCCCN(CCO)CCCCCCCCCC(=O)OCCCCCCCCCN(CC)CC/C=C\CCCCC]

It is represented by Formula (I), wherein L is L^{a}-X^{a}-L^{b}, X^{a} is —O₂C—, L^{a} is C₄₋₂₀ alkylene {C₉ alkylene}, L^{b} is C₄₋₂₀ alkylene {C₉ alkylene}; R¹ is C₁₋₄ alkyl {ethyl}; R² is C₅₋₂₅ alkenyl {C₉ alkenyl}; R³ is hydroxyethyl as detailed for DSL1-1; R⁴ is C₅₋₁₅ alkylene-CO₂—C₅₋₁₅ alkylene-N(R⁵)R⁶ {C₉ alkylene-CO₂—C₁₀ alkylene-N(R⁵)R⁶}; R⁵ is C₁₋₄ alkyl {ethyl}; and R⁶ is C₅₋₂₅ alkenyl {C₉ alkenyl}.

The compound DSL3C-3 is shown below,

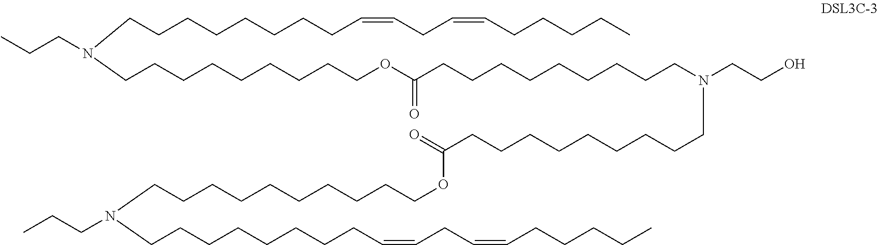 [SMILES: CCCCC/C=C\C/C=C\CCCCCCCCN(CCC)CCCCCCCCCCOC(=O)CCCCCCCCCN(CCO)CCCCCCCCCC(=O)OCCCCCCCCCN(CCC)CCCCCCCC/C=C\C/C=C\CCCCC]

It is represented by Formula (I), wherein L is L^{a}-X^{a}-L^{b}, X^{a} is —O₂C—, L^{a} is C₄₋₂₀ alkylene {C₉ alkylene}, L^{b} is C₄₋₂₀ alkylene {C₉ alkylene}; R¹ is C₁₋₄ alkyl {n-propyl}; R² is C₅₋₂₅ alkenyl {C₁₈ dienyl}; R³ is hydroxyethyl as detailed for DSL1-1; R⁴ is C₅₋₁₅ alkylene-CO₂—C₅₋₁₅ alkylene-N(R⁵)R⁶ {C₉ alkylene-CO₂—C₁₀ alkylene-N(R⁵)R⁶}; R⁵ is C₁₋₄ alkyl {n-propyl}; and R⁶ is C₅₋₂₅ alkenyl {C₁₈ dienyl}.

The compound DSL3C-4 is shown below,

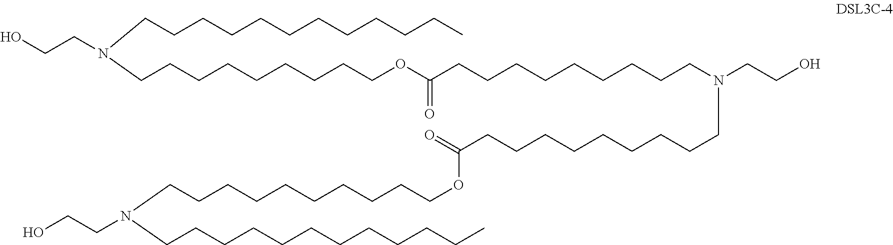 [SMILES: CCCCCCCCCCCCN(CCO)CCCCCCCCCCOC(=O)CCCCCCCCCN(CCO)CCCCCCCCCC(=O)OCCCCCCCCCN(CCO)CCCCCCCCCCCC]

It is represented by Formula (I), wherein L is L^{a}-X^{a}-L^{b}, X^{a} is —O₂C—, L^{a} is C₄₋₂₀ alkylene {C₉ alkylene}, L^{b} is C₄₋₂₀ alkylene {C₉ alkylene}; R¹ is hydroxyethyl as detailed for DSL1-1; R² is C₅₋₂₅ alkyl {C₁₂ alkyl; R³ is hydroxyethyl as detailed for DSL1-1; R⁴ is C₅₋₁₅ alkylene-CO₂—C₅₋₁₅ alkylene-N(R⁵)R⁶ {C₁₀ alkylene-CO₂—C₁₀ alkylene-N(R⁵)R⁶}; R⁵ is C₀₋₁₀ alkylene-OH {C₂ alkylene-OH}; and R⁶ is C₅₋₂₅ alkyl {C₁₈ alkyl}. As may be understood, DSL3C-4 can also be represented by other formulas described herein, e.g., Formula (II).

The compound DSL3C-5 is shown below,

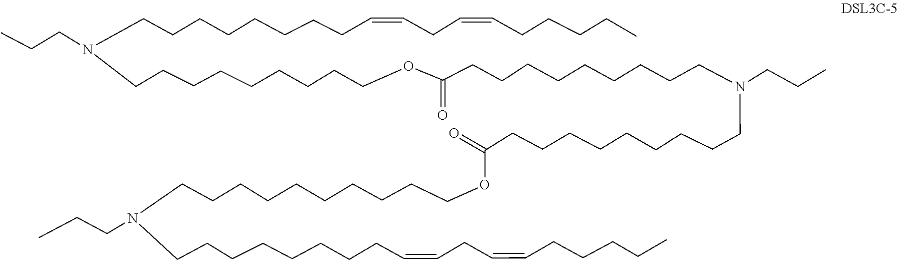 [SMILES: CCCCC/C=C\C/C=C\CCCCCCCCN(CCC)CCCCCCCCCCOC(=O)CCCCCCCCCN(CCC)CCCCCCCCCC(=O)OCCCCCCCCCN(CCC)CCCCCCCC/C=C\C/C=C\CCCCC]

It is represented by Formula (I), wherein L is L^{a}-X^{a}-L^{b}, X^{a} is —O₂C—, L^{a} is C₄₋₂₀ alkylene {C₉ alkylene}, L^{b} is C₄₋₂₀ alkylene {C₉ alkylene}; R¹ is C₁₋₄ alkyl {n-propyl}; R² is C₅₋₂₅ alkenyl {C₁₈ dienyl}; R³ is C₁₋₄ alkyl {n-propyl}; R⁴ is C₅₋₁₅ alkylene-CO₂—C₅₋₁₅ alkylene-N(R⁵)R⁶ {C₉ alkylene-CO₂—C₁₀ alkylene-N(R⁵)R⁶}; R⁵ is C₁₋₄ alkyl {n-propyl}; and R⁶ is C₅₋₂₅ alkenyl {C₁₈ dienyl}.

The compound DSL3C-6 is shown below,

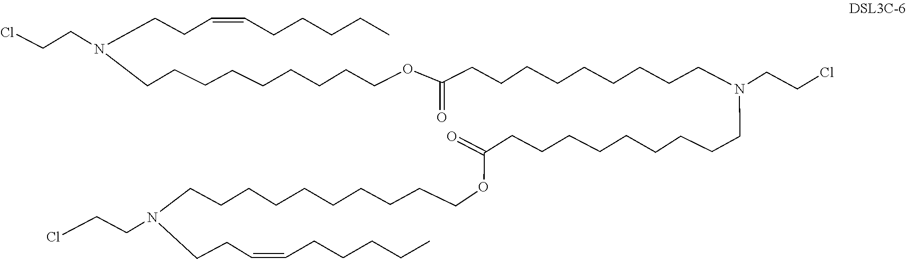 [SMILES: CCCCC/C=C\CCN(CCCl)CCCCCCCCCCOC(=O)CCCCCCCCCN(CCCl)CCCCCCCCCC(=O)OCCCCCCCCCN(CCCl)CC/C=C\CCCCC]

It is represented by Formula (I), wherein L is L^{a}-X^{a}-L^{b}, X^{a} is —O₂C—, L^{a} is C₄₋₂₀ alkylene {C₉ alkylene}, L^{b} is C₄₋₂₀ alkylene {C₉ alkylene}; R¹ is chloroethyl as detailed for DSL1-12; R² is C₅-alkenyl {C₉ alkenyl}; R³ is chloroethyl as detailed for DSL1-12; R⁴ is C₅₋₁₅ alkylene-CO₂—C₅₋₁₅ alkylene-N(R⁵)R⁶ {C₉ alkylene-CO₂—C₁₀ alkylene-N(R⁵)R⁶}; R⁵ is C₀₋₁₀ alkylene-halogen {chloroethyl}; and R⁶ is C₅₋₂₅ alkenyl {C₉ alkenyl}.

The compound DSL3C-7 is shown below,

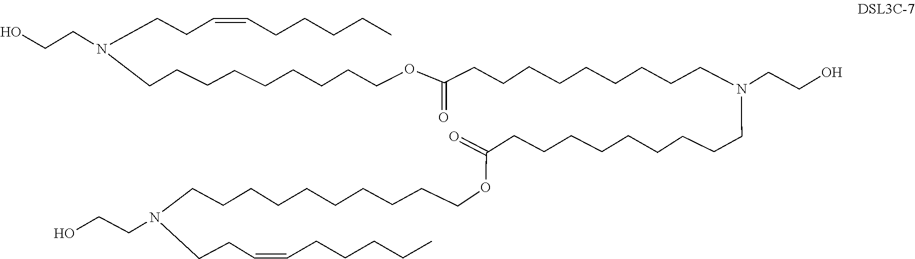 [SMILES: CCCCC/C=C\CCN(CCO)CCCCCCCCCCOC(=O)CCCCCCCCCN(CCO)CCCCCCCCCC(=O)OCCCCCCCCCN(CCO)CC/C=C\CCCCC]

It is represented by Formula (I), wherein L is L^{a}-X^{a}-L^{b}, X^{a} is —O₂C—, L^{a} is C₄₋₂₀ alkylene {C₉ alkylene}, L^{b} is C₄₋₂₀ alkylene {C₉ alkylene}; R¹ is hydroxyethyl as detailed for DSL1-1; R² is C₅₋₂₅ alkenyl {C₉ alkenyl}; R³ is hydroxyethyl as detailed for DSL1-1; R⁴ is C₅₋₁₅ alkylene-CO₂—C₅₋₁₅ alkylene-N(R⁵)R⁶ {C₉ alkylene-CO₂—C₁₀ alkylene-N(R⁵)R⁶}; R⁵ is C₀₋₁₀ alkylene-OH {C₂ alkylene-OH}; and R⁶ is C₅₋₂₅ alkenyl {C₉ alkenyl}.

The compound DSL3C-8 is shown below,

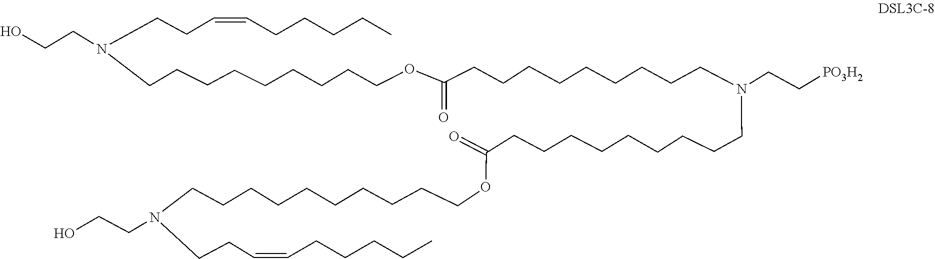

It is represented by Formula (I), wherein L is L^{a}-X^{a}-L^{b}, X^{a} is —O₂C—, L^{a} is C₄₋₂₀ alkylene {C₉ alkylene}, L^{b} is C₄₋₂₀ alkylene {C₉ alkylene}; R¹ is hydroxyethyl as detailed for DSL1-1; R² is C₅₋₂₅ alkenyl {C₉ alkenyl}; R³ is CH₂CH₂—PO₃H₂ as detailed for DSL1-24; R⁴ is C₅₋₁₅ alkylene-CO₂-C₅₋₁₅ alkylene-N(R⁵)R⁶ {C₉ alkylene-CO₂—C₁₀ alkylene-N(R⁵)R⁶}; R⁵ is C₀₋₁₀ alkylene-OH {C₂ alkylene-OH}; and R⁶ is C₅₋₂₅ alkenyl {C₉ alkenyl}.

The compound DSL2-1 is shown below,

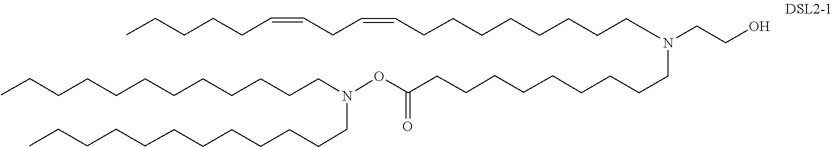 [SMILES: CCCCC/C=C\C/C=C\CCCCCCCCN(CCO)CCCCCCCCCC(=O)ON(CCCCCCCCCCCC)CCCCCCCCCCCC]

It is represented by Formula (I), wherein L is L^{a}-X^{a}-L^{b}, X^{a} is —O₂C—, L^{a} is C₀₋₄ alkylene {C₀ alkylene, i.e., absent}, L^{b} is C₄₋₂₀ alkylene {C₉ alkylene}; R¹ is hydroxyethyl as detailed for DSL1-1; R² is C₅₋₂₅ alkenyl {C₁₈ diene chain}; R³ is C₅₋₂₅ alkyl {C₁₂ alkyl}; and R⁴ is C₅₋₂₅ alkyl {C₁₂ alkyl}. As may be understood, DSL2-1 can also be represented by other formulas described herein, e.g., Formula (II).

The compound DSL2-2 is shown below,

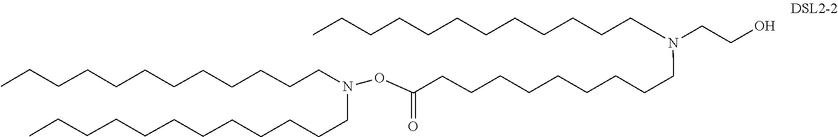 [SMILES: CCCCCCCCCCCCN(CCO)CCCCCCCCCC(=O)ON(CCCCCCCCCCCC)CCCCCCCCCCCC]

It is represented by Formula (I), wherein L is L^{a}-X^{a}-L^{b}, X^{a} is —O₂C—, L^{a} is C₀₋₄ alkylene {C₀ alkylene, i.e., absent}, L^{b} is C₄₋₂₀ alkylene {C₉ alkylene}; R¹ is hydroxyethyl as detailed for DSL1-1; R² is C₅₋₂₅ alkyl {C₁₂ alkyl}; R³ is C₅₋₂₅ alkyl {C₁₂ alkyl}; and R⁴ is C₅₋₂₅ alkyl {C₁₂ alkyl}. As may be understood, DSL2-2 can also be represented by other formulas described herein, e.g., Formula (II).

The compound DSL2-3 is shown below,

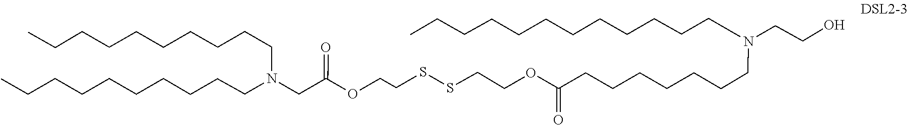 [SMILES: CCCCCCCCCCCCN(CCO)CCCCCCCC(=O)OCCSSCCOC(=O)CN(CCCCCCCCCC)CCCCCCCCCC]

It is represented by Formula (I), wherein L is L^{a}-X^{a}-L^{b}, X^{a} is —CO₂-L^{c}-O₂C—, L^{c} is C₁₋₆ alkylene-S—S—C₁₋₆ alkylene {C₂ alkylene-S—S—C₂ alkylene}, L^{a} is C₀₋₄ alkylene {C₁ alkylene}, L^{b} is C₄₋₂₀ alkylene {C₇ alkylene}; R¹ is hydroxyethyl as detailed for DSL1-1; R² is C₅₋₂₅ alkyl {C₁₂ alkyl}; R³ is C₅₋₂₅ alkyl {C₁₀ alkyl}; and R⁴ is C₅₋₂₅ alkyl {C₁₀ alkyl}

The compound DSL2-4 is shown below,

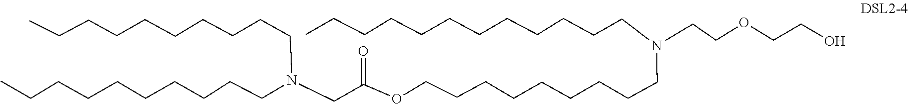 [SMILES: CCCCCCCCCCCCN(CCCCCCCCCOC(=O)CN(CCCCCCCCCC)CCCCCCCCCC)CCOCCO]

It is represented by Formula (I), wherein L is L^{a}-X^{a}-L^{b}, X^{a} is —O₂C—, L^{a} is C₄₋₂₀ alkylene {C₉ alkylene}; L^{b} is C₀₋₄ alkylene {C₁ alkylene}; R¹ is C₀₋₁₀ alkylene-Y^{a} {C₂ alkylene-Y^{a}}, wherein Y^{a} is O—Y^{b}, wherein Y^{b} is C₁₋₄ alkylene-OH {C₂ alkylene-OH}; R² is C₅₋₂₅ alkyl {C₁₂ alkyl}; R³ is C₅₋₂₅ alkyl {C₁₀ alkyl}; and R⁴ is C₅₋₂₅ alkyl {C₁₀ alkyl}.

The compound DSL2-5 is shown below,

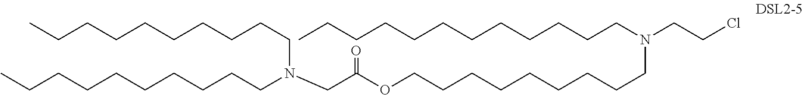 [SMILES: CCCCCCCCCCCCN(CCCl)CCCCCCCCCOC(=O)CN(CCCCCCCCCC)CCCCCCCCCC]

It is represented by Formula (I), wherein L is L^{a}-X^{a}-L^{b}, X^{a} is —O₂C—, L^{a} is C₄₋₂₀ alkylene {C₉ alkylene}; L^{b} is C₀₋₄ alkylene {C₁ alkylene}; R¹ is chloroethyl as detailed for DSL1-12; R² is C₅-alkyl {C₁₂ alkyl}; R³ is C₅₋₂₅ alkyl {C₁₀ alkyl}; and R⁴ is C₅₋₂₅ alkyl {C₁₀ alkyl}.

The compound DSL2-6 is shown below,

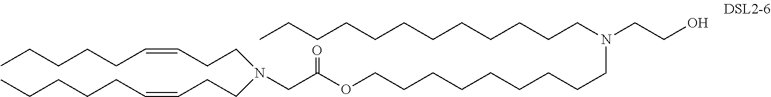 [SMILES: CCCCC/C=C\CCN(CC/C=C\CCCCC)CC(=O)OCCCCCCCCCN(CCO)CCCCCCCCCCCC]

It is represented by Formula (I), wherein L is L^{a}-X^{a}-L^{b}, X^{a} is —O₂C—, L^{a} is C₄₋₂₀ alkylene {C₉ alkylene}; L^{b} is C₀₋₄ alkylene {C₁ alkylene}; R¹ is hydroxyethyl as detailed for DSL1-1; R² is C₅-alkyl {C₁₂ alkyl}; R³ is C₅₋₂₅ alkenyl {C₉ alkenyl}; and R⁴ is C₅₋₂₅ alkenyl {C₉ alkenyl}.

The compound DSL2-7 is shown below,

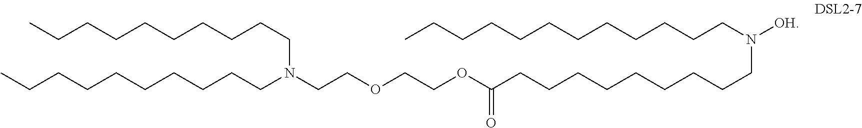 [SMILES: CCCCCCCCCCCCN(O)CCCCCCCCCC(=O)OCCOCCN(CCCCCCCCCC)CCCCCCCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₄₋₂₀ alkylene {C₉ alkylene}; R¹ is OH as detailed for DSL1-3; R² is C₅₋₂₅ alkyl {C₁₂ alkyl}; R³ is C₅₋₂₅ alkyl {C₁₀ alkyl}; and R⁴ is C₅₋₂₅ alkyl {C₁₀ alkyl}.

The compound DSL2-8 is shown below,

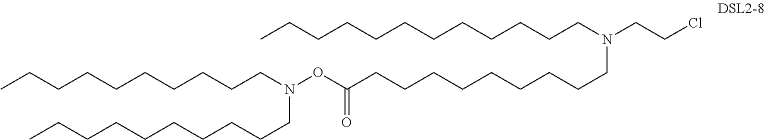 [SMILES: CCCCCCCCCCCCN(CCCl)CCCCCCCCCC(=O)ON(CCCCCCCCCC)CCCCCCCCCC]

It is represented by Formula (I), wherein L is L^{a}-X^{a}-L^{b}, X^{a} is —O₂C—, L^{a} is C₀₋₄ alkylene {C₀ alkylene, i.e., absent}, L^{b} is C₄₋₂₀ alkylene {C₉ alkylene}; R¹ is chloroethyl as detailed for DSL1-121; R² is C₅₋₂₅ alkyl {C₁₂ alkyl}; R³ is C₅₋₂₅ alkyl {C₁₀ alkyl}; and R⁴ is C₅₋₂₅ alkyl {C₁₀ alkyl}.

The compound DSL2-9 is shown below,

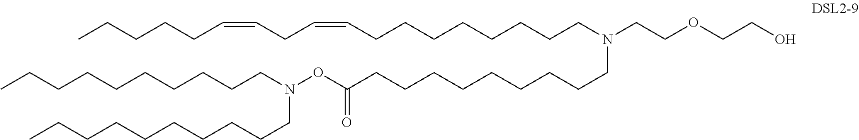 [SMILES: CCCCC/C=C\C/C=C\CCCCCCCCN(CCCCCCCCCC(=O)ON(CCCCCCCCCC)CCCCCCCCCC)CCOCCO]

It is represented by Formula (I), wherein L is L^{a}-X^{a}-L^{b}, X^{a} is —O₂C—, L^{a} is C₀₋₄ alkylene {C₀ alkylene, i.e., absent}; L^{b} is C₀₋₄ alkylene {C₁ alkylene}; R¹ is C₀₋₁₀ alkylene-Y^{a} {C₂ alkylene-Y^{a}}, wherein Y^{a} is O—Y^{b}, wherein Y^{b} is C₁₋₄ alkylene-OH {C₂ alkylene-OH}; R² is C₅₋₂₅ alkenyl {C₁₈ dienyl}; R³ is C₅₋₂₅ alkyl {C₁₀ alkyl}; and R⁴ is C₅₋₂₅ alkyl {C₁₀ alkyl}.

The compound DSL2-10 is shown below,

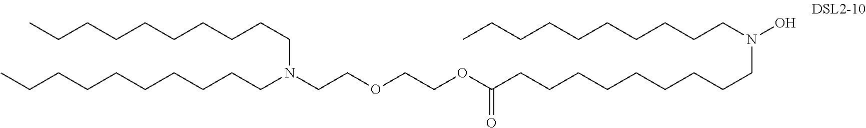 [SMILES: CCCCCCCCCCN(O)CCCCCCCCCC(=O)OCCOCCN(CCCCCCCCCC)CCCCCCCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₄₋₂₀ alkylene {C₉ alkylene}; R¹ is OH as detailed for DSL1-3; R² is C₅₋₂₅ alkyl {C₁₀ alkyl}; R³ is C₅₋₂₅ alkyl {C₁₀ alkyl}; and R⁴ is C₅₋₂₅ alkyl {C₁₀ alkyl}.

The compound DSL2-11 is shown below,

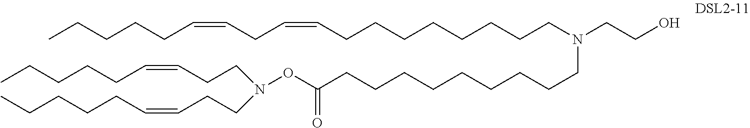 [SMILES: CCCCC/C=C\C/C=C\CCCCCCCCN(CCO)CCCCCCCCCC(=O)ON(CC/C=C\CCCCC)CC/C=C\CCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₅₋₂₅ alkenyl {C₁₈ dienyl}; R³ is C₅₋₂₅ alkenyl {C₉ alkenyl}; and R⁴ is alkenyl {C₉ alkenyl}.

The compound DSL2-12 is shown below,

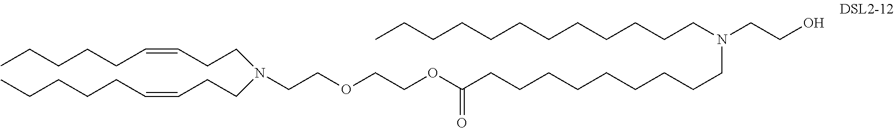 [SMILES: CCCCC/C=C\CCN(CC/C=C\CCCCC)CCOCCOC(=O)CCCCCCCCCN(CCO)CCCCCCCCCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₅₋₂₅ alkyl {C₁₂ alkyl}; R³ is C₅₋₂₅ alkenyl {C₉ alkenyl}; and R⁴ is C₅₋₂₅ alkenyl {C₉ alkenyl}.

The compound DSL2-13 is shown below,

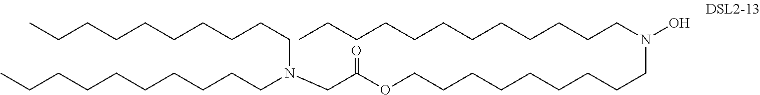 [SMILES: CCCCCCCCCCCCN(O)CCCCCCCCCOC(=O)CN(CCCCCCCCCC)CCCCCCCCCC]

It is represented by Formula (I), wherein L is L^{a}-X^{a}-L^{b}; X^{a} is —O₂C—, L^{a} is C₄₋₂₀ alkylene {C₉ alkylene}; L^{b} is C₀₋₄ alkylene {C₁ alkylene}; R¹ is OH as detailed for DSL1-3; R² is C₅₋₂₅ alkyl {C₁₂ alkyl}; R³ is C₅₋₂₅ alkyl {C₁₀ alkyl}; and R⁴ is C₅₋₂₅ alkyl {C₁₀ alkyl}. As may be understood, DSL2-13 can also be represented by other formulas described herein, e.g., Formula (II).

The compound DSL2-14 is shown below,

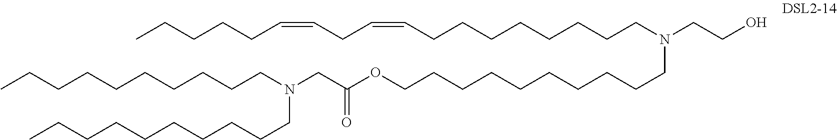 [SMILES: CCCCC/C=C\C/C=C\CCCCCCCCN(CCO)CCCCCCCCCCOC(=O)CN(CCCCCCCCCC)CCCCCCCCCC]

It is represented by Formula (I), wherein L is L^{a}-X^{a}-L^{b}; X^{a} is —O₂C—, L^{a} is C₄₋₂₀ alkylene {C₁₀ alkylene}; L^{b} is C₀₋₄ alkylene {C₁ alkylene}; R¹ is hydroxyethyl as detailed for DSL1-1; R² is C₅₋₂₅ alkenyl {C₁₈ dienyl}; R³ is C₅₋₂₅ alkyl {C₁₀ alkyl}; and R⁴ is C₅₋₂₅ alkyl {C₁₀ alkyl}. As may be understood, DSL2-14 can also be represented by other formulas described herein, e.g., Formula (II).

The compound DSL2-15 is shown below,

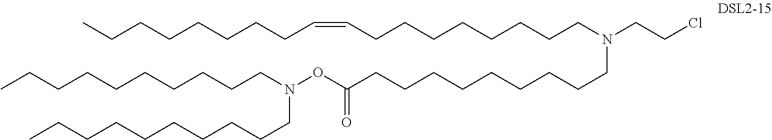 [SMILES: CCCCCCCC/C=C\CCCCCCCCN(CCCl)CCCCCCCCCC(=O)ON(CCCCCCCCCC)CCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is chloroethyl as detailed for DSL1-12; R² is C₅₋₂₅ alkenyl {C₁₈ alkenyl}; R³ is C₅₋₂₅ alkyl {C₁₀ alkyl}; and R⁴ is C₅₋₂₅ alkyl {C₁₀ alkyl}.

The compound DSL2-16 is shown below,

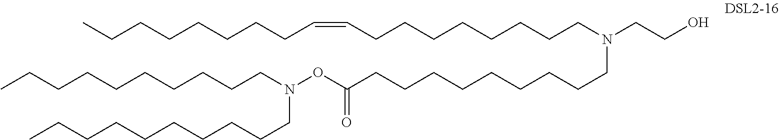 [SMILES: CCCCCCCC/C=C\CCCCCCCCN(CCO)CCCCCCCCCC(=O)ON(CCCCCCCCCC)CCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₅₋₂₅ alkenyl {C₁₈ alkenyl}; R³ is C₅₋₂₅ alkyl {C₁₀ alkyl}; and R⁴ is C₅₋₂₅ alkyl {C₁₀ alkyl}. As may be understood, DSL2-16 can also be represented by other formulas described herein, e.g., Formula (II).

The compound DSL2-17 is shown below,

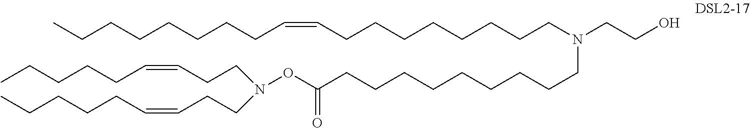 [SMILES: CCCCC/C=C\CCN(CC/C=C\CCCCC)OC(=O)CCCCCCCCCN(CCO)CCCCCCCC/C=C\CCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₅₋₂₅ alkenyl {C₁₈ alkenyl}; R³ is C₅₋₂₅ alkenyl {C₉ alkenyl}; and R⁴ is C₅₋₂₅ alkenyl {C₉ alkenyl}.

The compound DSL2-18 is shown below,

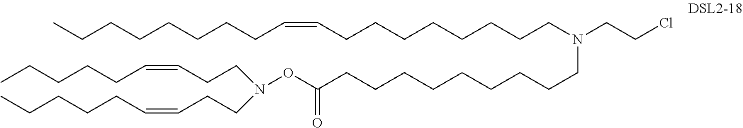 [SMILES: CCCCC/C=C\CCN(CC/C=C\CCCCC)OC(=O)CCCCCCCCCN(CCCl)CCCCCCCC/C=C\CCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₅₋₂₅ alkenyl {C₁₈ alkenyl}; R³ is C₅₋₂₅ alkenyl {C₉ alkenyl}; and R⁴ is C₅₋₂₅ alkenyl {C₉ alkenyl}.

The compound DSL2-19 is shown below,

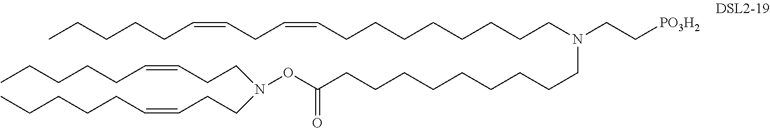

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₅₋₂₅ alkenyl {C₁₈ dienyl}; R³ is C₅₋₂₅ alkenyl {C₉ alkenyl}; and R⁴ is C₅₋₂₅ alkenyl {C₉ alkenyl}.

The compound DSL2-20 is shown below,

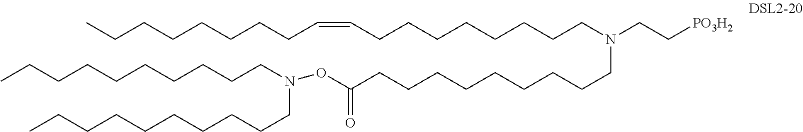

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₅₋₂₅ alkenyl {C₁₈ alkenyl}; R³ is C₅₋₂₅ alkenyl {C₉ alkenyl}; and R⁴ is C₅₋₂₅ alkenyl {C₉ alkenyl}.

The compound DSL2-21 is shown below,

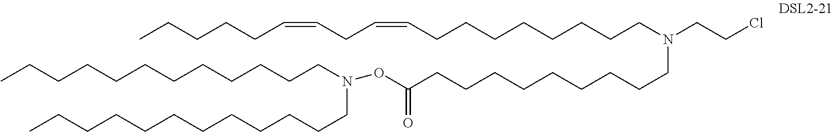 [SMILES: CCCCC/C=C\C/C=C\CCCCCCCCN(CCCl)CCCCCCCCCC(=O)ON(CCCCCCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is chloroethyl as detailed for DSL1-12; R² is C₅₋₂₅ alkenyl {C₁₈ dienyl}; R³ is C₅₋₂₅ alkyl {C₁₂ alkyl}; and R⁴ is C₅₋₂₅ alkyl {C₁₂ alkyl}.

The compound DSL2-22 is shown below,

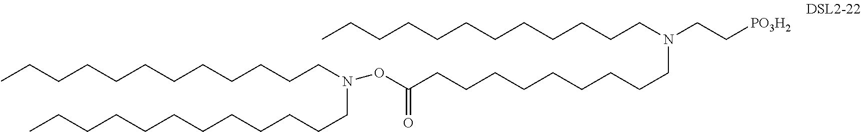

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₅₋₂₅ alkyl {C₁₂ alkyl}; R³ is C₅₋₂₅ alkyl {C₁₂ alkyl}; and R⁴ is C₅₋₂₅ alkyl {C₁₂ alkyl}.

The compound DSL2-23 is shown below,

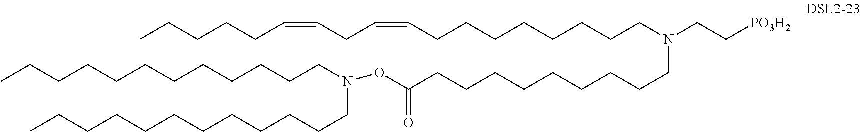

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₅₋₂₅ alkenyl {C₁₈ dienyl}; R³ is C₅₋₂₅ alkyl {C₁₂ alkyl}; and R⁴ is C₅₋₂₅ alkyl {C₁₂ alkyl}.

The compound DSL2-24 is shown below,

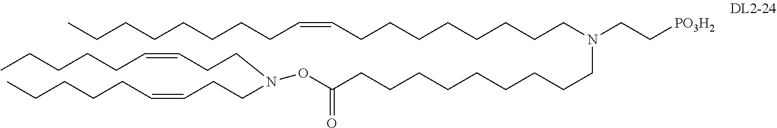

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₅₋₂₅ alkenyl {C₁₈ alkenyl}; R³ is C₅₋₂₅ alkenyl {C₉ alkenyl}; and R⁴ is C₅₋₂₅ alkenyl {C₉ alkenyl}.

The compound DSL2-25 is shown below,

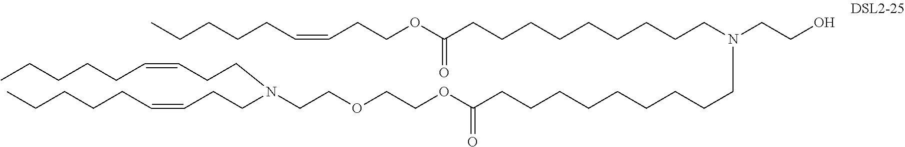 [SMILES: CCCCC/C=C\CCOC(=O)CCCCCCCCCN(CCO)CCCCCCCCCC(=O)OCCOCCN(CC/C=C\CCCCC)CC/C=C\CCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₅₋₁₅ alkylene-CO₂—C₅₋₁₅ alkenyl {C₉ alkylene-CO₂—C₉ alkenyl}; R³ is C₅₋₂₅ alkenyl {C₉ alkenyl}; and R⁴ is C₅₋₂₅ alkenyl {C₉ alkenyl}.

The compound DSL2-26 is shown below,

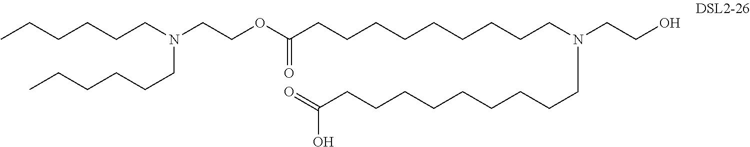 [SMILES: CCCCCCN(CCCCCC)CCOC(=O)CCCCCCCCCN(CCO)CCCCCCCCCC(=O)O]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is hydroxyethyl as detailed for DSL1-1; R² is C₅₋₁₅ alkylene-CO₂H {C₉ alkylene-CO₂H}; R³ is C₅₋₂₅ alkyl {C₆ alkyl}; and R⁴ is C₅₋₂₅ alkyl {C₆ alkyl}.

The compound DSL2-27 is shown below,

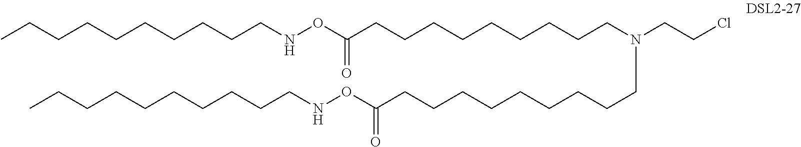 [SMILES: CCCCCCCCCCNOC(=O)CCCCCCCCCN(CCCl)CCCCCCCCCC(=O)ONCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is chloroethyl as detailed for DSL1-12; R² is C₂₋₁₅ alkylene-CO₂—C₀₋₄ alkylene-NH—C₁₋₁₂ alkyl {C₉ alkylene-CO₂—C₀ alkylene-NH—C₁₀ alkyl}; R³ is H; and R⁴ is C₅₋₂₅ alkyl {C₁₀ alkyl}.

The compound DSL2-28 is shown below,

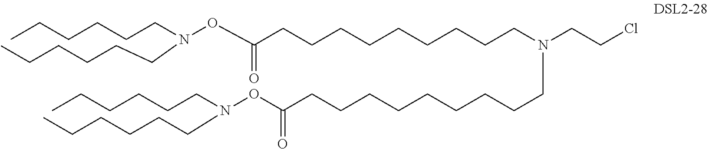 [SMILES: CCCCCCN(CCCCCC)OC(=O)CCCCCCCCCN(CCCl)CCCCCCCCCC(=O)ON(CCCCCC)CCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is chloroethyl as detailed for DSL1-12; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-29 is shown below,

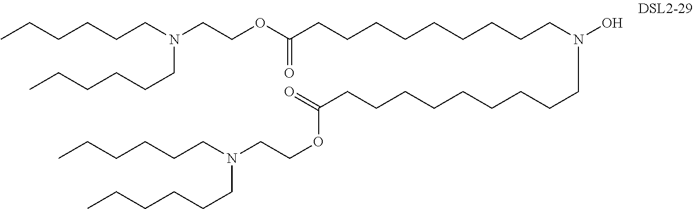 [SMILES: CCCCCCN(CCCCCC)CCOC(=O)CCCCCCCCCN(O)CCCCCCCCCC(=O)OCCN(CCCCCC)CCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is OH as detailed for DSL1-3; R² is C₂₋₁₅ alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}. As may be understood, DSL2-29 can also be represented by other formulas described herein, e.g., Formula (II).

The compound DSL2-30 is shown below,

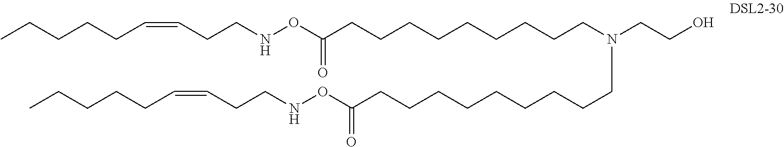 [SMILES: CCCCC/C=C\CCNOC(=O)CCCCCCCCCN(CCO)CCCCCCCCCC(=O)ONCC/C=C\CCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₂₋₁₅ alkylene-CO₂—C₀₋₄ alkylene-NH—C₅₋₂₅ alkenyl {C₉ alkylene-CO₂—C₀ alkylene-NH—C₉ alkenyl}; R³ is H; and R⁴ is C₅₋₂₅ alkenyl {C₉ alkenyl}.

The compound DSL2-31 is shown below,

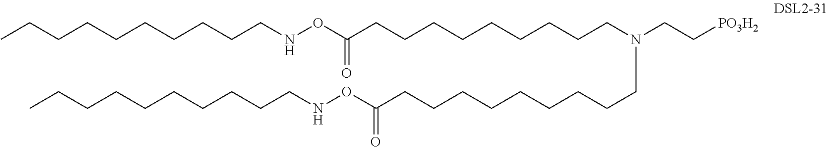

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₂₋₁₅ alkylene-CO₂—C₀₋₄ alkylene-NH—C₁₋₁₂ alkyl {C₉ alkylene-CO₂—C₀ alkylene-NH—C₁₀ alkyl}; R³ is H; and R⁴ is C₅₋₂₅ alkyl {C₁₀ alkyl}.

The compound DSL2-32 is shown below,

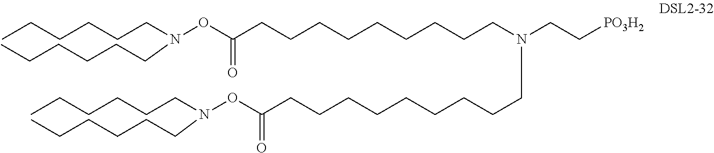

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-33 is shown below,

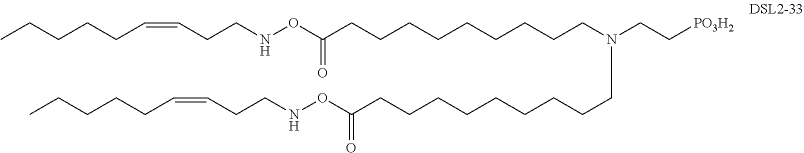

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₂₋₁₅ alkylene-CO₂—C₀₋₄ alkylene-NH—C₅₋₂₅ alkenyl {C₉ alkylene-CO₂—C₀ alkylene-NH—C₉ alkenyl}; R³ is H; and R⁴ is C₅₋₂₅ alkenyl {C₉ alkenyl}.

The compound DSL2-34 is shown below,

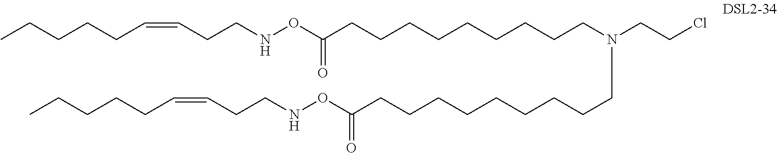 [SMILES: CCCCC/C=C\CCNOC(=O)CCCCCCCCCN(CCCl)CCCCCCCCCC(=O)ONCC/C=C\CCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is chloroethyl as detailed for DSL1-12; R² is C₂₋₁₅ alkylene-CO₂—C₀₋₄ alkylene-NH—C₅₋₂₅ alkenyl {C₉ alkylene-CO₂—C₀ alkylene-NH—C₉ alkenyl}; R³ is H; and R⁴ is C₅₋₂₅ alkenyl {C₉ alkenyl}.

The compound DSL2-35 is shown below,

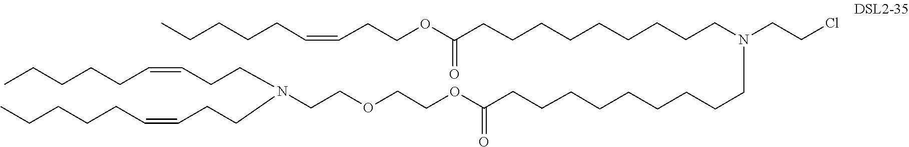 [SMILES: CCCCC/C=C\CCOC(=O)CCCCCCCCCN(CCCl)CCCCCCCCCC(=O)OCCOCCN(CC/C=C\CCCCC)CC/C=C\CCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₅₋₁₅ alkylene-CO₂—C₅₋₁₅ alkenyl {C₉ alkylene-CO₂—C₉ alkenyl}; R³ is C₅₋₂₅ alkenyl {C₉ alkenyl}; and R⁴ is C₅₋₂₅ alkenyl {C₉ alkenyl}.

The compound DSL2-36 is shown below,

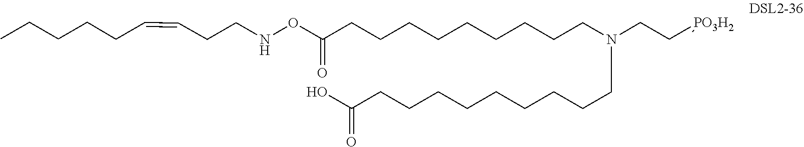

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₅₋₁₅ alkylene-CO₂H {C₉ alkylene-CO₂H}; R³ is H; and R⁴ is C₅₋₂₅ alkenyl {C₉ alkenyl}.

The compound DSL2-37 is shown below,

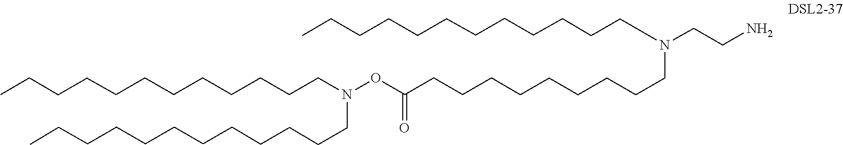 [SMILES: CCCCCCCCCCCCN(CCN)CCCCCCCCCC(=O)ON(CCCCCCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is aminoethyl as detailed for DSL1-42; R² is C₅₋₂₅ alkyl {C₁₂ alkyl}; R³ is C₅₋₂₅ alkyl {C₁₂ alkyl}; and R⁴ is C₅₋₂₅ alkyl {C₁₂ alkyl}.

The compound DSL2-38 is shown below,

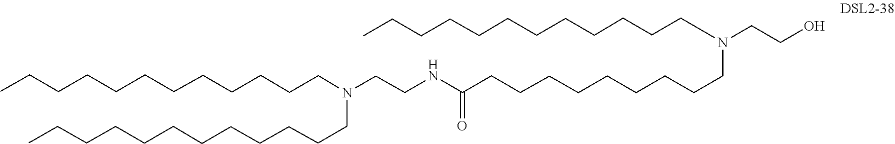 [SMILES: CCCCCCCCCCCCN(CCO)CCCCCCCCCC(=O)NCCN(CCCCCCCCCCCC)CCCCCCCCCCCC]

It is represented by Formula (I), wherein L is L^{a}-X^{a}-L^{b}; X^{a} is —CO—NH—, L^{a} is C₄₋₂₀ alkylene {C₉ alkylene}; L^{b} is C₀₋₄ alkylene {C₂ alkylene}; R¹ is hydroxyethyl as detailed for DSL1-1; R² is C₅-alkyl {C₁₂ alkyl}; R³ is C₅₋₂₅ alkyl {C₁₂ alkyl}; and R⁴ is C₅₋₂₅ alkyl {C₁₂ alkyl}.

The compound DSL2-39 is shown below,

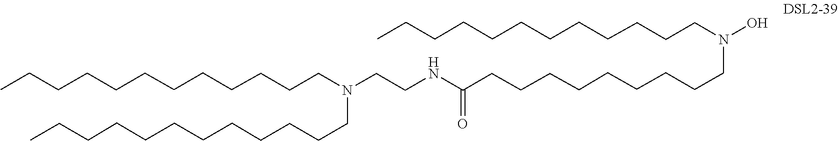 [SMILES: CCCCCCCCCCCCN(O)CCCCCCCCCC(=O)NCCN(CCCCCCCCCCCC)CCCCCCCCCCCC]

It is represented by Formula (I), wherein L is L^{a}-X^{a}-L^{b}; X^{a} is —CO—NH—, L^{a} is C₄₋₂₀ alkylene {C₉ alkylene}; L^{b} is C₀₋₄ alkylene {C₂ alkylene}; R¹ is OH as detailed for DSL1-3; R² is C₅₋₂₅ alkyl {C₁₂ alkyl}; R³ is C₅₋₂₅ alkyl {C₁₂ alkyl}; and R⁴ is C₅₋₂₅ alkyl {C₁₂ alkyl}.

The compound DSL2-40 is shown below,

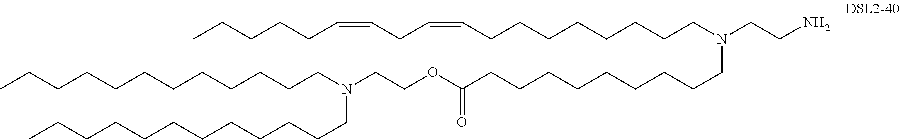 [SMILES: CCCCC/C=C\C/C=C\CCCCCCCCN(CCN)CCCCCCCCCC(=O)OCCN(CCCCCCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is aminoethyl as detailed for DSL1-42; R² is C₅-alkenyl {C₁₈ dienyl}; R³ is C₅₋₂₅ alkyl {C₁₂ alkyl}; and R⁴ is C₅₋₂₅ alkyl {C₁₂ alkyl}.

The compound DSL2-41 is shown below,

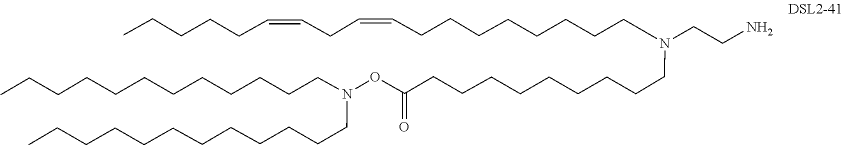 [SMILES: CCCCC/C=C\C/C=C\CCCCCCCCN(CCN)CCCCCCCCCC(=O)ON(CCCCCCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is aminoethyl as detailed for DSL1-42; R² is C₅₋₂₅ alkenyl {C₁₈ dienyl}; R³ is C₅₋₂₅ alkyl {C₁₂ alkyl}; and R⁴ is C₅₋₂₅ alkyl {C₁₂ alkyl}.

The compound DSL2-42 is shown below,

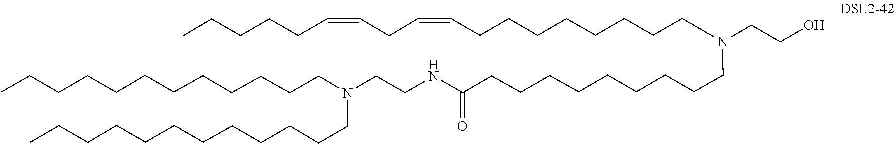 [SMILES: CCCCC/C=C\C/C=C\CCCCCCCCN(CCO)CCCCCCCCCC(=O)NCCN(CCCCCCCCCCCC)CCCCCCCCCCCC]

It is represented by Formula (I), wherein L is L^{a}-X^{a}-L^{b}; X^{a} is —CO—NH—, L^{a} is C₄₋₂₀ alkylene {C₉ alkylene}; L^{b} is C₀₋₄ alkylene IC₂ alkylene}; R¹ is hydroxyethyl as detailed for DSL1-1; R² is C₅-alkenyl {C₁₈ dienyl}; R³ is C₅₋₂₅ alkyl {C₁₂ alkyl}; and R⁴ is C₅₋₂₅ alkyl {C₁₂ alkyl}.

The compound DSL2-43 is shown below,

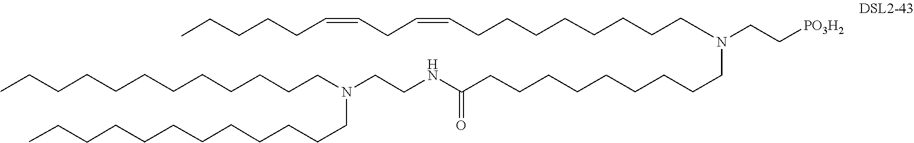

It is represented by Formula (I), wherein L is L^{a}-X^{a}-L^{b}; X^{a} is —CO—NH—, L^{a} is C₄₋₂₀ alkylene {C₉ alkylene}; L^{b} is C₀₋₄ alkylene {C₂ alkylene}; R¹ is CH₂CH₂—PO₃H₂ as detailed for DSL1-16; R² is C₅₋₂₅ alkenyl {C₁₈ dienyl}; R³ is C₅₋₂₅ alkyl {C₁₂ alkyl}; and R⁴ is C₅₋₂₅ alkyl {C₁₂ alkyl}.

The compound DSL2-44 is shown below,

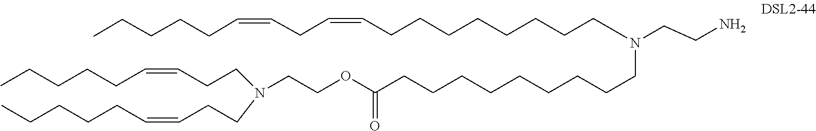 [SMILES: CCCCC/C=C\C/C=C\CCCCCCCCN(CCN)CCCCCCCCCC(=O)OCCN(CC/C=C\CCCCC)CC/C=C\CCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is aminoethyl as detailed for DSL1-42; R² is C₅-alkenyl {C₁₈ dienyl}; R³ is C₅₋₂₅ alkenyl {C₉ alkenyl}; and R⁴ is C₅₋₂₅ alkenyl {C₉ alkenyl}.

The compound DSL2-45 is shown below,

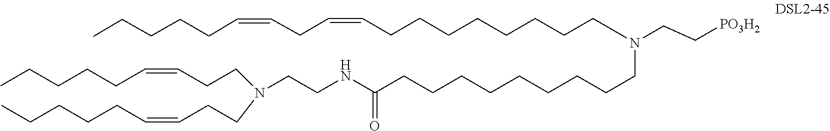

It is represented by Formula (I), wherein L is L^{a}-X^{a}-L^{b}; X^{a} is —CO—NH—, L^{a} is C₄₋₂₀ alkylene {C₉ alkylene}; L^{b} is C₀₋₄ alkylene {C₂ alkylene}; R¹ is CH₂CH₂—PO₃H₂ as detailed for DSL1-16; R² is C₅₋₂₅ alkenyl {C₁₈ dienyl}; R³ is C₅₋₂₅ alkenyl {C₉ alkenyl}; and R⁴ is C₅₋₂₅ alkenyl {C₉ alkenyl}.

The compound DSL2-46 is shown below,

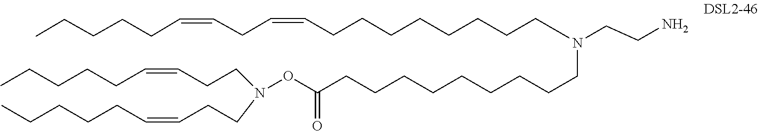 [SMILES: CCCCC/C=C\C/C=C\CCCCCCCCN(CCN)CCCCCCCCCC(=O)ON(CC/C=C\CCCCC)CC/C=C\CCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is aminoethyl as detailed for DSL1-42; R² is C₅₋₂₅ alkenyl {C₁₈ dienyl}; R³ is C₅₋₂₅ alkenyl {C₉ alkenyl}; and R⁴ is C₅₋₂₅ alkenyl {C₉ alkenyl}.

The compound DSL2-47 is shown below,

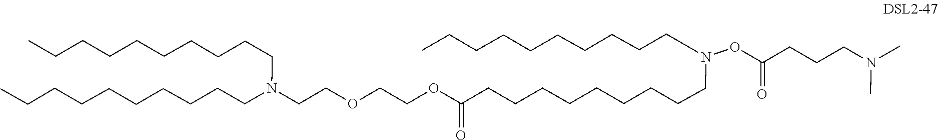

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₄₋₂₀ alkylene {C₉ alkylene}; R¹ is C₀₋₁₀ alkylene-Y^{a} {C₀ alkylene-Y^{a}, i.e., R¹ is Y^{a}}, wherein Y^{a} is O—Y^{b}, Y^{b} is CO—C₁₋₄ alkyl-Y^{c} {CO—C₃ alkyl-Y^{c}}, wherein Y^{c} is N(CH₃)₂; R² is C₅₋₂₅ alkyl {C₁₀ alkyl}; R³ is C₅₋₂₅ alkyl {C₁₀ alkyl}; and R⁴ is C₅₋₂₅ alkyl {C₁₀ alkyl}.

The compound DSL2-48 is shown below,

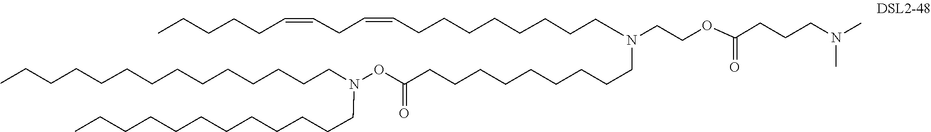 [SMILES: CCCCC/C=C\C/C=C\CCCCCCCCN(CCCCCCCCCC(=O)ON(CCCCCCCCCCCC)CCCCCCCCCCCCCC)CCOC(=O)CCCN(C)C]

It is represented by Formula (I), wherein L is L^{a}-X^{a}-L^{b}; X^{a} is-O₂C—; L^{a} is C₀₋₄ alkylene {C₀ alkylene, i.e., absent}; L^{b} is C₄₋₂₀ alkylene {C₉ alkylene}; R¹ is C₀₋₁₀ alkylene-Y^{a} {C₂ alkylene-Y^{a}}, wherein Y^{a} is O—Y^{b}, Y^{b} is CO—C₁₋₄ alkyl-Y^{c} {CO—C₃ alkyl-Y^{c}}, wherein Y^{c} is N(CH₃)₂; R² is C₅₋₂₅ alkenyl {C₁₈ dienyl}; R³ is C₅₋₂₅ alkyl {C₁₂ alkyl}; and R⁴ is C₅₋₂₅ alkyl {C₁₂ alkyl}.

The compound DSL2-49 is shown below,

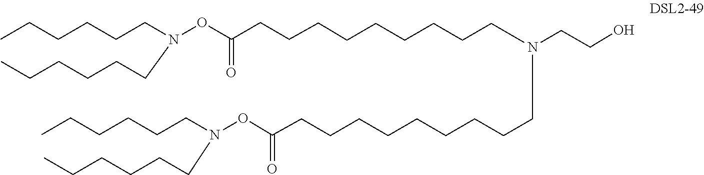 [SMILES: CCCCCCN(CCCCCC)OC(=O)CCCCCCCCCN(CCO)CCCCCCCCCC(=O)ON(CCCCCC)CCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}. As may be understood, DSL2-49 can also be represented by other formulas described herein, e.g., Formula (II).

The compound DSL2-50 is shown below,

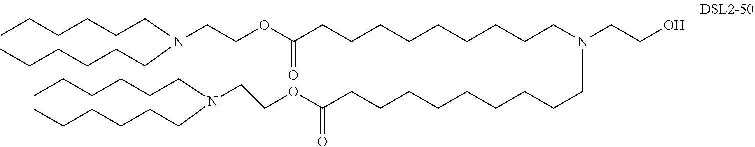 [SMILES: CCCCCCN(CCCCCC)CCOC(=O)CCCCCCCCCN(CCO)CCCCCCCCCC(=O)OCCN(CCCCCC)CCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is hydroxyethyl as detailed for DSL1-1; R² is C₂-alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}. As may be understood, DSL2-50 can also be represented by other formulas described herein, e.g., Formula (II).

The compound DSL2-51 is shown below,

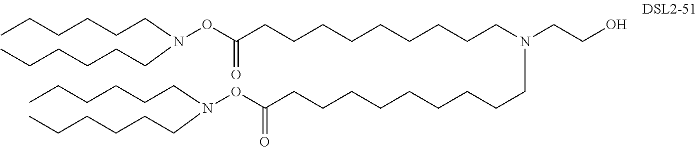 [SMILES: CCCCCCN(CCCCCC)OC(=O)CCCCCCCCCN(CCO)CCCCCCCCCC(=O)ON(CCCCCC)CCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is C₀₋₁₀ alkylene-Y^{a} {C₄ alkylene-Y^{a}}, wherein Y^{a} is O—Y^{b}, wherein Y^{b} is H; R² is C₂₋₁₅ alkylene-CO₂-C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-52 is shown below,

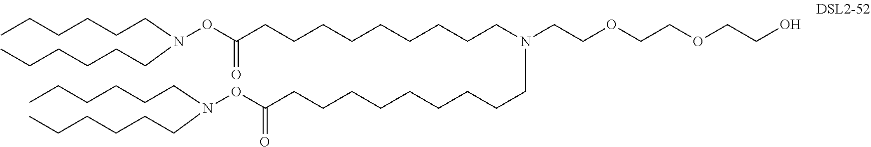 [SMILES: CCCCCCN(CCCCCC)OC(=O)CCCCCCCCCN(CCCCCCCCCC(=O)ON(CCCCCC)CCCCCC)CCOCCOCCO]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is C₀₋₁₀ alkylene-Y^{a}, wherein Y^{a} is O—Y^{b}, wherein Y^{b} is C₁₋₄ alkylene-(O—C₁₋₄ alkylene)ᵢ_₃—OH {C₂ alkylene-(O—C₂ alkylene)ᵢ-OH}; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-53 is shown below,

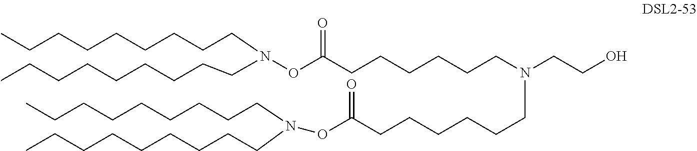 [SMILES: CCCCCCCCCN(CCCCCCCCC)OC(=O)CCCCCCN(CCO)CCCCCCC(=O)ON(CCCCCCCCC)CCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₆ alkylene}; R¹ is hydroxyethyl as detailed for DSL1-1; R² is C₂₋₁₅ alkylene-CO₂—C₀₋₈ alkylene-N(C₁₋₁₂ alkyl)₂ {C₆ alkylene-CO₂—C₀ alkylene-N(C₉ alkyl)₂}; R³ is C₅₋₂₅ alkyl {C₉ alkyl}; and R⁴ is C₅₋₂₅ alkyl {C₉ alkyl}.

The compound DSL2-54 is shown below,

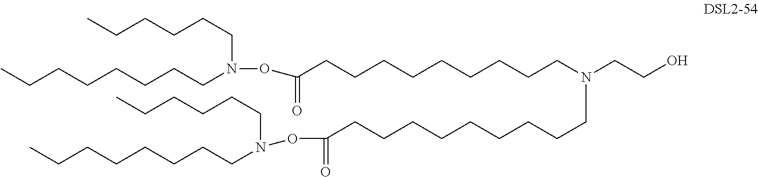 [SMILES: CCCCCCCCN(CCCCCC)OC(=O)CCCCCCCCCN(CCO)CCCCCCCCCC(=O)ON(CCCCCC)CCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)(C₈ alkyl)}; R³ is C₅₋₂₅ alkyl {C₆ alkyl}; and R⁴ is C₅₋₂₅ alkyl {C₈ alkyl}. As may be understood, DSL2-54 can also be represented by other formulas described herein, e.g., Formula (II).

The compound DSL2-55 is shown below,

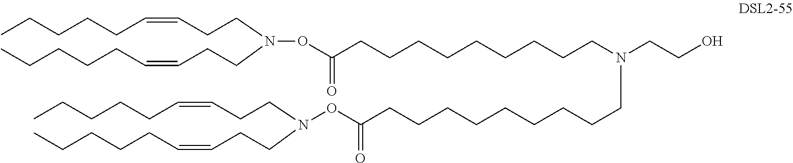 [SMILES: CCCCC/C=C\CCN(CC/C=C\CCCCC)OC(=O)CCCCCCCCCN(CCO)CCCCCCCCCC(=O)ON(CC/C=C\CCCCC)CC/C=C\CCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₂₋₁₅ alkylene-CO₂—C₀₋₄ alkylene-N(C₅₋₂₅ alkenyl)₂ {C₉ alkylene-CO₂—C₀ alkylene-N(C₉ alkenyl)₂}; R³ is C₅₋₂₅ alkenyl {C₉ alkenyl}; and R⁴ is C₅₋₂₅ alkenyl {C₉ alkenyl}.

The compound DSL2-56 is shown below,

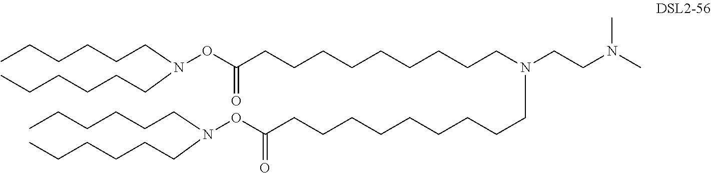 [SMILES: CCCCCCN(CCCCCC)OC(=O)CCCCCCCCCN(CCCCCCCCCC(=O)ON(CCCCCC)CCCCCC)CCN(C)C]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is C₀₋₁₀ alkylene-Y^{a}, wherein Y^{a} is NMe₂; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-57 is shown below,

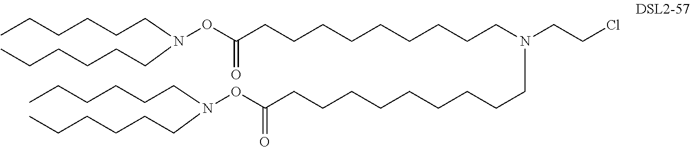 [SMILES: CCCCCCN(CCCCCC)OC(=O)CCCCCCCCCN(CCCl)CCCCCCCCCC(=O)ON(CCCCCC)CCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is chloroethyl as detailed for DSL1-12; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-58 is shown below,

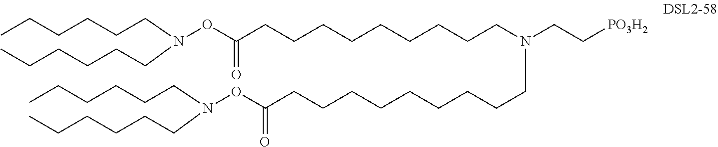

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is CH₂CH₂—PO₃H₂ as detailed for DSL1-16; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-59 is shown below,

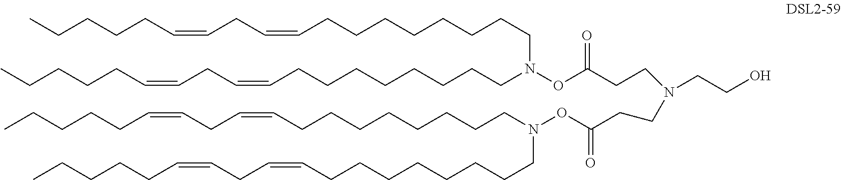 [SMILES: CCCCC/C=C\C/C=C\CCCCCCCCN(CCCCCCCC/C=C\C/C=C\CCCCC)OC(=O)CCN(CCO)CCC(=O)ON(CCCCCCCC/C=C\C/C=C\CCCCC)CCCCCCCC/C=C\C/C=C\CCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₀₋₄ alkylene {C₂ alkylene}; R¹ is hydroxyethyl as detailed for DSL1-1; R² is C₂₋₁₅ alkylene-CO₂—C₀₋₄ alkylene-N(C₅₋₂₅ alkenyl)₂{C₂ alkylene-CO₂—C₀ alkylene-N(C₁₈ dienyl)₂}; R³ is C₅₋₂₅ alkenyl {C₁₈ dienyl}; and R⁴ is C₅₋₂₅ alkenyl {C₁₈ dienyl}.

The compound DSL2-60 is shown below,

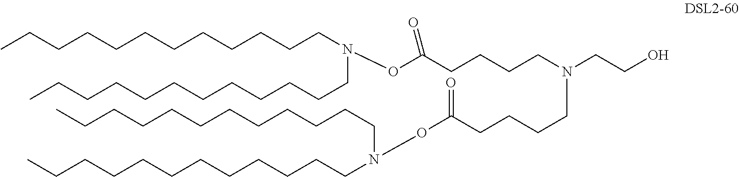 [SMILES: CCCCCCCCCCCCN(CCCCCCCCCCCC)OC(=O)CCCCN(CCO)CCCCC(=O)ON(CCCCCCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₄ alkylene}; R¹ is hydroxyethyl as detailed for DSL1-1; R² is C₂₋₁₅ alkylene-CO₂—C₀₋₈ alkylene-N(C₁₋₁₂ alkyl)₂ {C₄ alkylene-CO₂—C₀ alkylene-N(C₁₂ alkyl)₂}; R³ is C₅₋₂₅ alkyl {C₁₂ alkyl}; and R⁴ is C₅₋₂₅ alkyl {C₁₂ alkyl}.

The compound DSL2-61 is shown below,

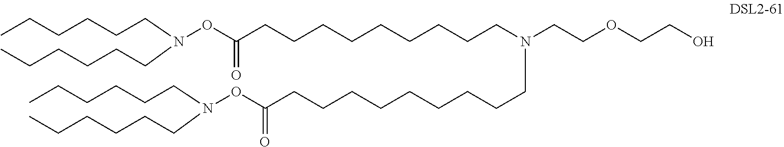 [SMILES: CCCCCCN(CCCCCC)OC(=O)CCCCCCCCCN(CCCCCCCCCC(=O)ON(CCCCCC)CCCCCC)CCOCCO]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₄₋₂₀ alkylene {C₉ alkylene}; R¹ is C₀₋₁₀ alkylene-Y^{a}, wherein Y^{a} is O—Y^{b}, wherein Y^{b} is C₁₋₄ alkylene-OH {C₂ alkylene-OH}; R² is C₂₋₁₅ alkylene-CO₂—C₀₋₈ alkylene-N(C₁₋₁₂ alkyl)₂ {C₉ alkylene-CO₂—C₀ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-62 is shown below,

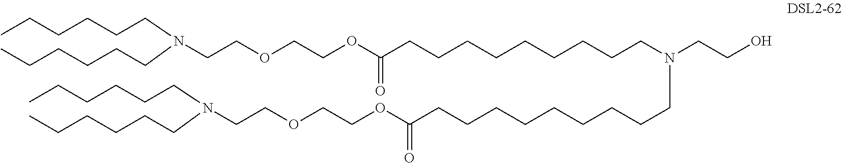 [SMILES: CCCCCCN(CCCCCC)CCOCCOC(=O)CCCCCCCCCN(CCO)CCCCCCCCCC(=O)OCCOCCN(CCCCCC)CCCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f}-{C₂ alkylene-(O—C₂ alkylene)_{f}}, wherein f is 1; L^{a} is C₀₋₄ alkylene {C₀ alkylene, i.e., absent}; L^{b} is C₄₋₂₀ alkylene {C₉ alkylene}; R¹ is hydroxyethyl as detailed for DSL1-1; R² is C₂₋₁₅ alkylene-CO₂—CH₂CH₂OCH₂CH₂—N(C₁₋₁₂ alkyl)₂ {C₉ alkylene-CO₂—CH₂CH₂OCH₂CH₂—N(C₆₂ alkyl)₂}; R³ is C₅₋₂₅ alkyl {C₆ alkyl}; and R⁴ is C₆ alkyl {C₁₂ alkyl}.

The compound DSL2-63 is shown below,

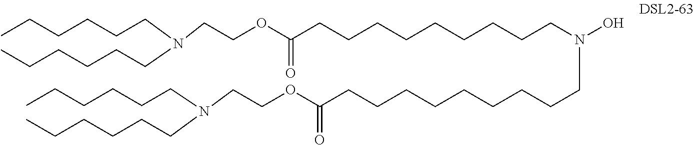 [SMILES: CCCCCCN(CCCCCC)CCOC(=O)CCCCCCCCCN(O)CCCCCCCCCC(=O)OCCN(CCCCCC)CCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is OH as detailed for DSL1-3; R² is C₂₋₁₅ alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}. As may be understood, DSL2-63 can also be represented by other formulas described herein, e.g., Formula (II).

The compound DSL2-64 is shown below,

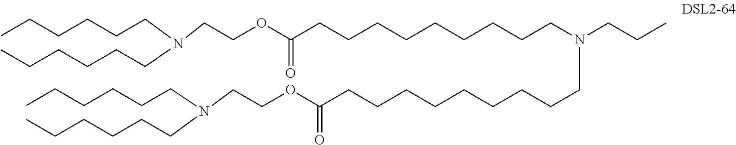 [SMILES: CCCCCCN(CCCCCC)CCOC(=O)CCCCCCCCCN(CCC)CCCCCCCCCC(=O)OCCN(CCCCCC)CCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is C₁₋₄ alkyl (propyl); R² is C₂₋₁₅ alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-65 is shown below,

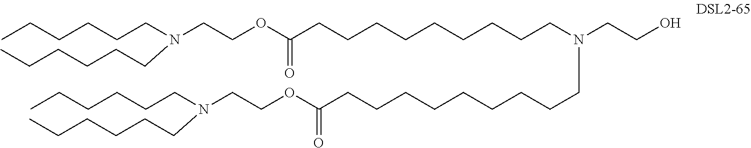 [SMILES: CCCCCCN(CCCCCC)CCOC(=O)CCCCCCCCCN(CCO)CCCCCCCCCC(=O)OCCN(CCCCCC)CCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is hydroxyethyl as detailed for DSL1-1; R² is C₂₋₁₅ alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}. As may be understood, DSL2-65 can also be represented by other formulas described herein, e.g., Formula (II).

The compound DSL2-66 is shown below,

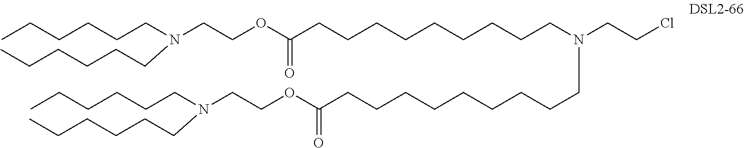 [SMILES: CCCCCCN(CCCCCC)CCOC(=O)CCCCCCCCCN(CCCl)CCCCCCCCCC(=O)OCCN(CCCCCC)CCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is chloroethyl as detailed for DSL1-12; R² is C₂-alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-67 is shown below,

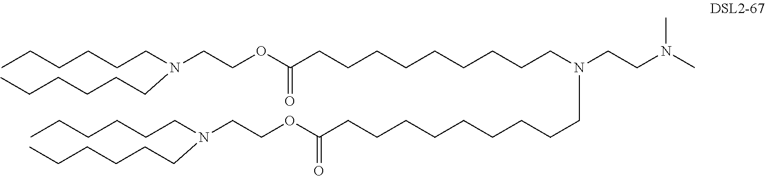 [SMILES: CCCCCCN(CCCCCC)CCOC(=O)CCCCCCCCCN(CCCCCCCCCC(=O)OCCN(CCCCCC)CCCCCC)CCN(C)C]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is C₀₋₁₀ alkylene-Y^{a} {C₂ alkylene-Y^{a}}, wherein Y^{a} is NMe₂; R² is C₂₋₁₅ alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-68 is shown below,

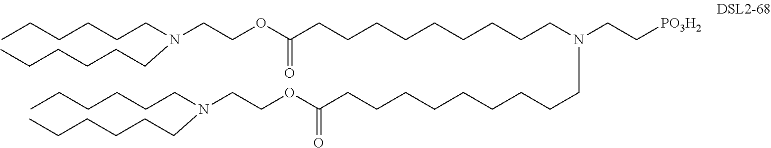

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is CH₂CH₂—PO₃H₂ as detailed for DSL1-16; R² is C₂₋₁₅ alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-69 is shown below,

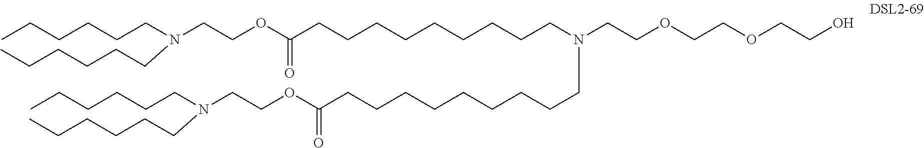 [SMILES: CCCCCCN(CCCCCC)CCOC(=O)CCCCCCCCCN(CCCCCCCCCC(=O)OCCN(CCCCCC)CCCCCC)CCOCCOCCO]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is C₀₋₁₀ alkylene-Y^{a} {C₂ alkylene} wherein Y^{a} is O—Y^{b}, wherein Y^{b} is C₁₋₄ alkylene-(O—C₁₋₄ alkylene)ᵢ₋₃-OH {C₂ alkylene-(O—C₂ alkylene)ᵢ-OH}; R² is C₂₋₁₅ alkylene-CO₂—C₀₋₈ alkylene-N(C₁₋₁₂ alkyl)₂ {C₉ alkylene-CO₂—C₂ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-70 is shown below,

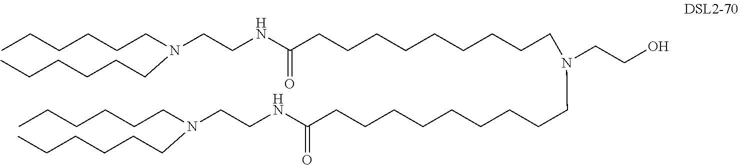 [SMILES: CCCCCCN(CCCCCC)CCNC(=O)CCCCCCCCCN(CCO)CCCCCCCCCC(=O)NCCN(CCCCCC)CCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene {C₉ alkylene}; R¹ is hydroxyethyl as detailed for DSL1-1; R² is C₂₋₁₅ alkylene-CO—NH—C₀₋₈ alkylene-N(C₁₋₁₂ alkyl)₂ {C₉ alkylene-CO—NH—C₂ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-71 is shown below,

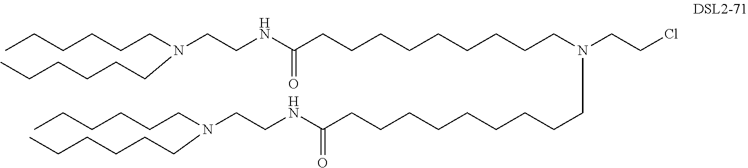 [SMILES: CCCCCCN(CCCCCC)CCNC(=O)CCCCCCCCCN(CCCl)CCCCCCCCCC(=O)NCCN(CCCCCC)CCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₄₋₂₀ alkylene IC₉ alkylene}; R¹ is chloroethyl as detailed for DSL1-12; R² is C₂₋₁₅ alkylene-CO—NH—C₀₋₄ alkylene-N(C₁₋₁₂ alkyl)₂ {C₉ alkylene-CO—NH—C₂ alkylene-N(C₆ alkyl)₂}; R³ is C₅₋₂₅ alkyl {C₆ alkyl}; and R⁴ is C₅₋₂₅ alkyl {C₆ alkyl}.

The compound DSL2-72 is shown below,

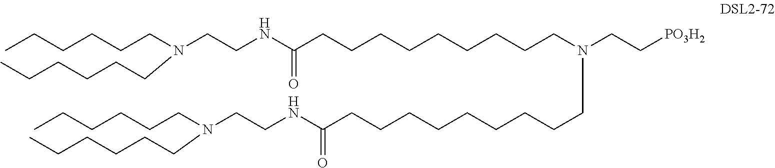

It is represented by Formula (I), wherein L is L^{a}-X^{a}-L^{b}; X^{a} is —CO—NH—, L^{a} is C₀₋₄ alkylene {C₂ alkylene}; L^{b} is C₄₋₂₀ alkylene {C₉ alkylene}; R¹ is CH₂CH₂—PO₃H₂ as detailed for DSL1-16; R² is C₂₋₁₅ alkylene-CO—NH—C₀₋₄ alkylene-N(C₁₋₁₂ alkyl)₂ {C₉ alkylene-CO—NH—C₂ alkylene-N(C₆ alkyl)₂}; R³ is C₅₋₂₅ alkyl IC₆ alkyl}; and R⁴ is C₅₋₂₅ alkyl IC₆ alkyl}.

The compound DSL4-1 is shown below,

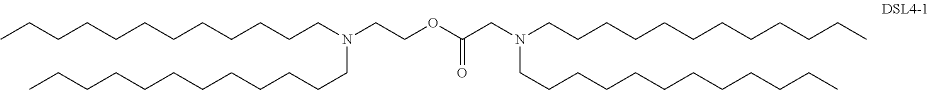 [SMILES: CCCCCCCCCCCCN(CCCCCCCCCCCC)CCOC(=O)CN(CCCCCCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₀₋₄ alkylene {C₂ alkylene}; R¹ is C₅₋₂₅ alkyl {C₁₂ alkyl}; R² is C₅₋₂₅ alkyl {C₁₂ alkyl}; R³ is C₅₋₂₅ alkyl {C₁₂ alkyl}; and R⁴ is C₅₋₂₅ alkyl {C₁₂ alkyl}. As may be understood, DSL4-1 can also be represented by other formulas described herein, e.g., Formula (II).

The compound DSL4-2 is shown below,

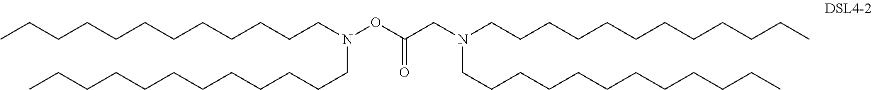 [SMILES: CCCCCCCCCCCCN(CCCCCCCCCCCC)CC(=O)ON(CCCCCCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₀₋₄ alkylene {C₁ alkylene}; R¹ is C₅₋₂₅ alkyl {C₁₂ alkyl}; R² is C₅₋₂₅ alkyl {C₁₂ alkyl}; R³ is C₅₋₂₅ alkyl {C₁₂ alkyl}; and R⁴ is C₅₋₂₅ alkyl {C₁₂ alkyl}. As may be understood, DSL4-2 can also be represented by other formulas described herein, e.g., Formula (II).

The compound DSL4-3 is shown below,

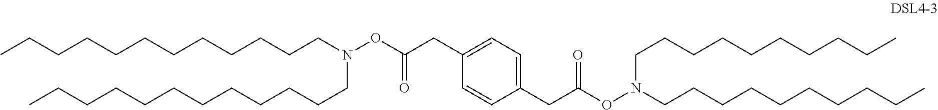 [SMILES: CCCCCCCCCCCCN(CCCCCCCCCCCC)OC(=O)Cc1ccc(CC(=O)ON(CCCCCCCCCC)CCCCCCCCCC)cc1]

It is represented by Formula (I), wherein L is L^{a}-X^{a}-L^{b}, X^{a} is —O₂C-L^{c}-CO₂—, L^{c} is C₀₋₄ alkylene-aryl-C₀₋₄ alkylene {C₁ alkylene-para-C₆H₄-C₁ alkylene}, L^{a} is C₀₋₄ alkylene {C₀ alkylene, i.e., absent}, L^{b} is C₀₋₄ alkylene {C₀ alkylene, i.e., absent}; R¹ is C₅₋₂₅ alkyl {C₁₀ alkyl}; R² is C₅₋₂₅ alkyl {C₁₀ alkyl}; R³ is C₅₋₂₅ alkyl {C₁₀ alkyl}; and R⁴ is C₅₋₂₅ alkyl {C₁₀ alkyl}.

The compound DSL4-4 is shown below,

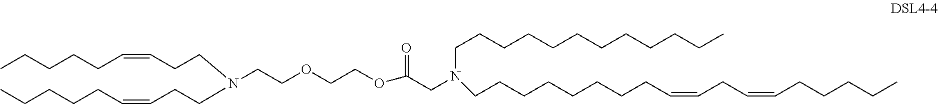 [SMILES: CCCCC/C=C\C/C=C\CCCCCCCCN(CCCCCCCCCCCC)CC(=O)OCCOCCN(CC/C=C\CCCCC)CC/C=C\CCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₀₋₄ alkylene {C₁ alkylene}; R¹ is C₅₋₂₅ alkyl {C₁₂ alkyl}; R² is C₅₋₂₅ alkenyl {C₁₈ dienyl}; R³ is C₅₋₂₅ alkenyl {C₉ alkenyl}; and R⁴ is C₉ alkenyl {C₉ alkenyl}.

The compound DSL4-5 is shown below,

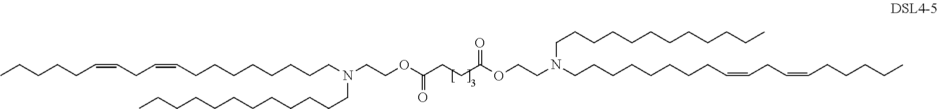

It is represented by Formula (I), wherein L is L^{a}-X^{a}-L^{b}, X^{a} is —O₂C-L^{c}-CO₂—, L^{c} is C₁₋₄ alkylene {C₄ alkylene}, L^{a} is C₀₋₄ alkylene {C₂ alkylene}, L^{b} is C₀₋₄ alkylene {C₂ alkylene}; R¹ is C₁₂ alkyl {C₁₂ alkyl}; R² is C₅₋₂₅ alkenyl {C₁₈ dienyl}; R³ is C₅₋₂₅ alkenyl {C₁₈ dienyl}; and R⁴ is C₅₋₂₅ alkyl {C₁₂ alkyl}. As may be understood, DSL4-5 can also be represented by other formulas described herein, e.g., Formula (II).

The compound DSL4-6 is shown below,

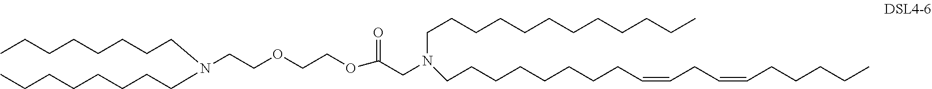 [SMILES: CCCCC/C=C\C/C=C\CCCCCCCCN(CCCCCCCCCCCC)CC(=O)OCCOCCN(CCCCCCCC)CCCCCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₀₋₄ alkylene {C₁ alkylene}; R¹ is C₅₋₂₅ alkyl {C₁₂ alkyl}; R² is C₅₋₂₅ alkenyl {C₁₈ dienyl}; R³ is C₅₋₂₅ alkyl {C₈ alkenyl}; and R⁴ is C₈ alkyl {C₉ alkenyl}.

The compound DSL4-7 is shown below,

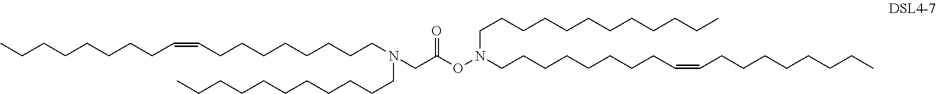 [SMILES: CCCCCCCC/C=C\CCCCCCCCN(CCCCCCCCCCC)CC(=O)ON(CCCCCCCC/C=C\CCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₀₋₄ alkylene {C₁ alkylene}; R¹ is C₅₋₂₅ alkyl {C₁₂ alkyl}; R² is C₅₋₂₅ alkenyl {C₁₈ alkenyl}; R³ is C₅₋₂₅ alkenyl {C₁₈ alkenyl}; and R⁴ is C₅₋₂₅ alkyl {C₁₁ alkyl}. As may be understood, DSL4-7 can also be represented by other formulas described herein, e.g., Formula (II).

The compound DSL4-8 is shown below,

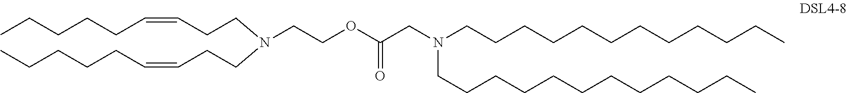 [SMILES: CCCCC/C=C\CCN(CC/C=C\CCCCC)CCOC(=O)CN(CCCCCCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₂ alkylene}; L^{b} is C₀₋₄ alkylene {C₁ alkylene}; R¹ is C₅₋₂₅ alkyl {C₁₂ alkyl}; R² is C₅₋₂₅ alkyl {C₁₂ alkyl}; R³ is C₅₋₂₅ alkenyl {C₉ alkenyl}; and R⁴ is C₅₋₂₅ alkenyl {C₉ alkenyl}.

The compound DSL4-9 is shown below,

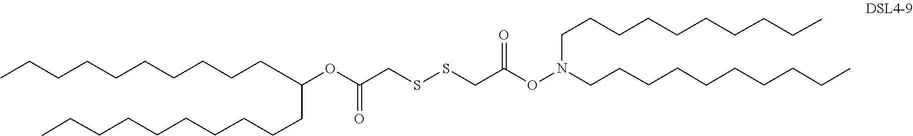 [SMILES: CCCCCCCCCCC(CCCCCCCCCC)OC(=O)CSSCC(=O)ON(CCCCCCCCCC)CCCCCCCCCC]

It is represented by Formula (I), wherein L is L^{a}-X^{a}-L^{b}, X^{a} is —O₂C-L^{c}-CO₂—, L^{c} is C₁₋₆ alkylene-S—S—C₁₋₆ alkylene {C₁ alkylene-S—S—C₁ alkylene}; L^{a} is C₀₋₄ alkylene {C₀ alkylene, i.e., absent}; L^{b} is C₀₋₄ alkylene {C₀ alkylene, i.e., absent}; R¹ is C₅₋₂₅ alkyl {C₁₀ alkyl};
R² is C₅₋₂₅ alkyl {C₁₀ alkyl}; R³ is C₅₋₂₅ alkyl {C₁₀ alkyl}; and R⁴ is C₅₋₂₅ alkyl {C₁₀ alkyl}

The compound DSL4-10 is shown below,

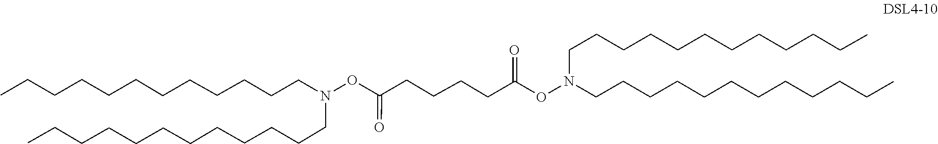 [SMILES: CCCCCCCCCCCCN(CCCCCCCCCCCC)OC(=O)CCCCC(=O)ON(CCCCCCCCCCCC)CCCCCCCCCCCC]

It is represented by Formula (I), wherein L is L^{a}-X^{a}-L^{b}, X^{a} is —O₂C-L^{c}-CO₂—, L^{c} is C₁₋₄ alkylene {C₄ alkylene}; L^{a} is C₀₋₄ alkylene {C₀ alkylene, i.e., absent}; L^{b} is C₀₋₄ alkylene {C₀ alkylene, i.e., absent}; R¹ is C₅₋₂₅ alkyl {C₁₂ alkyl}; R² is C₅₋₂₅ alkyl {C₁₂ alkyl}; R³ is C₅₋₂₅ alkyl {C₁₂ alkyl}; and R⁴ is C₅₋₂₅ alkyl {C₁₂ alkyl}

The compound DSL4-11 is shown below,

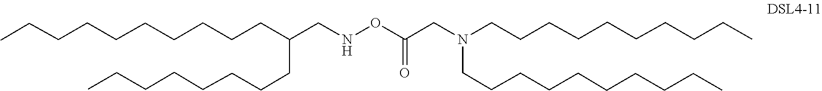 [SMILES: CCCCCCCCCCC(CCCCCCCC)CNOC(=O)CN(CCCCCCCCCC)CCCCCCCCCC]

It is represented by Formula (I), wherein L is L^{a}-X^{a}-L^{b}; X^{a} is —CO₂—NH—; L^{a} is C₀₋₄ alkylene {C₁ alkylene}; L^{b} is C₀₋₄ alkylene {C₁ alkylene}; R¹ is C₅₋₂₅ alkyl {C₁₀ alkyl}; R² is C₅₋₂₅ alkyl {C₁₀ alkyl}; R³ is H; and R⁴ is C₅₋₂₅ alkyl {C₂₀ alkyl}.

The compound DSL4-12 is shown below,

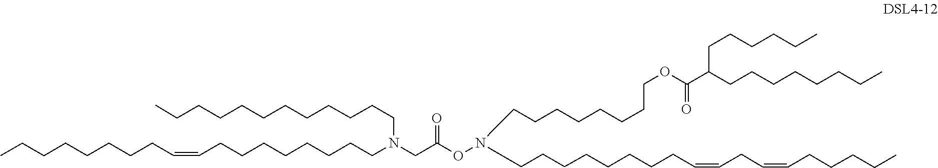 [SMILES: CCCCC/C=C\C/C=C\CCCCCCCCN(CCCCCCCCOC(=O)C(CCCCCC)CCCCCCCC)OC(=O)CN(CCCCCCCC/C=C\CCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₀₋₄ alkylene {C₁ alkylene}; R¹ is C₅₋₁₅ alkylene-CO₂—C₅₋₁₅ alkyl {C₈ alkylene-CO₂—C₁₈ alkyl}; R² is C₅₋₂₅ alkenyl {C₁₈ dienyl}; R³ is C₅₋₂₅ alkyl {C₁₂ alkyl}; and R⁴ is C₅₋₂₅ alkenyl {C₁₈ alkenyl}. It is noted that the although the variables are similar for DSL4-12 and DSL4-15, the difference is the orientation of L, which is not limited by Formula (I)—in DSL4-12, L^{a} is adjacent to R¹ and R², and L^{b} is adjacent to R³ and R⁴, while in DSL4-15, L^{a} is adjacent to R³ and R⁴, and L^{b} is adjacent to R¹ and R².

The compound DSL4-13 is shown below,

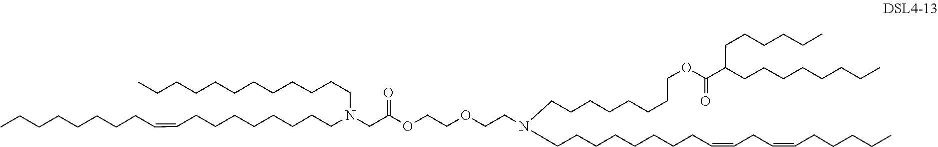 [SMILES: CCCCC/C=C\C/C=C\CCCCCCCCN(CCCCCCCCOC(=O)C(CCCCCC)CCCCCCCC)CCOCCOC(=O)CN(CCCCCCCC/C=C\CCCCCCCC)CCCCCCCCCCCC]

It is represented by Formula (I), wherein L is L^{a}-X^{a}-L^{b}; X^{a} is L^{c}-O₂C—, wherein L^{c} is C₁₋₆ alkylene-(O—C₁₋₆ alkylene)_{f} {C₂ alkylene-(O—C₂ alkylene)_{f}}, wherein f is 1; L^{a} is C₀₋₄ alkylene {C₀ alkylene, i.e., absent}; L^{b} is C₀₋₄ alkylene {C₁ alkylene}; R¹ is C₅₋₁₅ alkylene-CO₂—C₅₋₁₅ alkyl {C₈ alkylene-CO₂—C₁₅ alkyl}; R² is C₅₋₂₅ alkenyl {C₁₈ dienyl}; R³ is C₅₋₂₅ alkyl {C₁₂ alkyl}; and R⁴ is C₅₋₂₅ alkenyl {C₁₈ alkenyl}

The compound DSL4-14 is shown below,

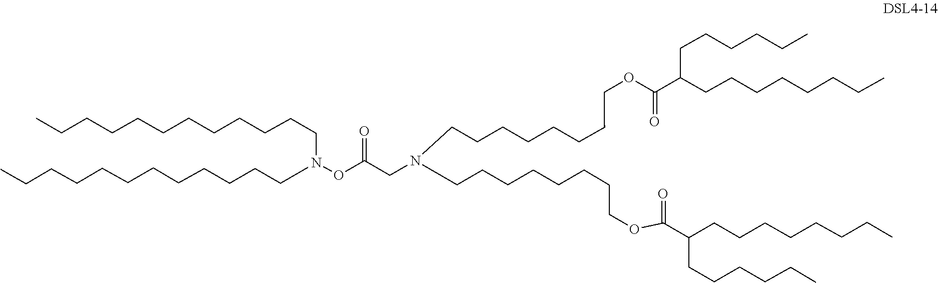

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₀₋₄ alkylene {C₁ alkylene}; R¹ is C₅₋₁₅ alkylene-CO₂—C₅₋₁₅ alkyl {C₈ alkylene-CO₂—C₁₈ alkyl}; R² is C₅₋₁₅ alkylene-CO₂—C₅₋₁₅ alkyl {C₈ alkylene-CO₂—C₁₈ alkyl}; R³ is C₅₋₂₅ alkyl {C₁₂ alkyl}; and R⁴ is C₅₋₂₅ alkyl {C₁₂ alkyl}.

The compound DSL4-15 is shown below,

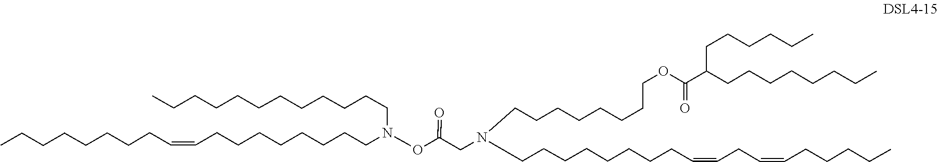 [SMILES: CCCCC/C=C\C/C=C\CCCCCCCCN(CCCCCCCCOC(=O)C(CCCCCC)CCCCCCCC)CC(=O)ON(CCCCCCCC/C=C\CCCCCCCC)CCCCCCCCCCCC]

It is represented by Formula (I), wherein L is L^{a}-X^{a}-L^{b}; X^{a} is —O₂C—, L^{a} is C₀₋₄ alkylene {C₀ alkylene, i.e., absent}; L^{b} is C₀₋₄ alkylene {C₁ alkylene}; R¹ is C₅₋₁₅ alkylene-CO₂—C₅₋₁₅ alkyl {C₈ alkylene-CO₂—C₁₈ alkyl}; R² is C₅₋₂₅ alkenyl {C₁₈ dienyl}; R³ is C₅₋₂₅ alkyl {C₁₂ alkyl}; and R⁴ is C₅₋₂₅ alkenyl {C₁₈ alkenyl}. It is noted that the although the variables are similar for DSL4-12 and DSL4-15, the difference is the orientation of L, which is not limited by Formula (I)—in DSL4-12, L^{a} is adjacent to R¹ and R², and L^{b} is adjacent to R³ and R⁴, while in DSL4-15, L^{a} is adjacent to R³ and R⁴, and L^{b} is adjacent to R¹ and R².

According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL1-55, DSL1-56, DSL1-57, DSL1-58, DSL1-59, DSL1-60, DSL3c-1, DSL3c-2, DSL3c-3, DSL3c-4, DSL3c-5, DSL3c-6, DSL3c-7 and DSL3c-8, including salts, hydrates, solvates, polymorphs, optical isomers, geometrical isomers, enantiomers, diastereomers, and mixtures thereof. According to some embodiments, the lipid is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL1-55, DSL1-56, DSL1-57, DSL1-58, DSL1-59, DSL1-60 including salts, hydrates, solvates, polymorphs, optical isomers, geometrical isomers, enantiomers, diastereomers, and mixtures thereof. According to some embodiments, the lipid is selected from the group consisting of: DSL3c-1, DSL3c-2, DSL3c-3, DSL3c-4, DSL3c-5, DSL3c-6, DSL3c-7 and DSL3c-8, including salts, hydrates, solvates, polymorphs, optical isomers, geometrical isomers, enantiomers, diastereomers, and mixtures thereof. According to some embodiments, the lipid is selected is selected from the group consisting of: DSL2-1, DSL2-2, DSL2-3, DSL2-4, DSL2-5, DSL2-6, DSL2-7, DSL2-8, DSL2-9, DSL2-10, DSL2-11, DSL2-12, DSL2-13, DSL2-14, DSL2-15, DSL2-16, DSL2-17, DSL2-18, DSL2-19, DSL2-20, DSL2-21, DSL2-22, DSL2-23, DSL2-24, DSL2-25, DSL2-26, DSL2-27, DSL2-28, DSL2-29, DSL2-30, DSL2-31, DSL2-32, DSL2-33, DSL2-34, DSL2-35, DSL2-36, DSL2-37, DSL2-38, DSL2-39, DSL2-40, DSL2-41, DSL2-42, DSL2-43, DSL2-44, DSL2-45, DSL2-46, DSL2-47, DSL2-48. DSL2-49, DSL2-50, DSL2-51, DSL2-52, DSL2-53, DSL2-54, DSL2-55, DSL2-56, DSL2-57, DSL2-58, DSL2-59, DSL2-60, DSL2-61, DSL2-62, DSL2-63, DSL2-64, DSL2-65, DSL2-66, DSL2-67, DSL2-68, DSL2-69, DSL2-70, DSL2-71 and DSL2-72 including salts, hydrates, solvates, polymorphs, optical isomers, geometrical isomers, enantiomers, diastereomers, and mixtures thereof. According to some embodiments, the lipid is selected from the group consisting of: DSL4-1, DSL4-2, DSL4-3, DSL4-4, DSL4-5, DSL4-6, DSL4-7, DSL4-8, DSL4-9, DSL4-10, DSL4-11, DSL4-12, DSL4-13, DSL4-14 and DSL4-15, including salts, hydrates, solvates, polymorphs, optical isomers, geometrical isomers, enantiomers, diastereomers, and mixtures thereof. According to some exemplary embodiments, the lipid is selected from the group consisting of: DSL1-1, DSL1-2, DSL1-3, DSL1-4, DSL1-5, DSL3C-1, DSL2-1, DSL2-2, DSL4-1 and DSL4-2, including salts, hydrates, solvates, polymorphs, optical isomers, geometrical isomers, enantiomers, diastereomers, and mixtures thereof. According to some exemplary embodiments, the lipid is selected from the group consisting of: DSL1-1, DSL1-2, DSL1-3, DSL1-4, DSL1-5, DSL3C-1, DSL2-1, DSL2-2, DSL2-49, DSL2-50, DSL4-1 and DSL4-2, including salts, hydrates, solvates, polymorphs, optical isomers, geometrical isomers, enantiomers, diastereomers, and mixtures thereof.

### Chemical Definitions

The term “cationic lipid”, as used herein refers to lipid species that carries a net positive charge at a selected pH. Selected pH values include, but not limited to physiological pH, pH=7 and the like. It is to be understood by the person having ordinary skill in the art that the lipids of the present invention may be considered as cationic lipids, since they bear 2 or more nitrogen atom, where these atoms are typically basic and protonizable at the selected pH, so that the compound may carry a net positive charge. According to some embodiments, the lipid of the present invention is a cationic lipid.

An “alkyl” group refers to any saturated aliphatic hydrocarbon, including straight-chain and branched-chain alkyl groups. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, 3-pentyl, hexyl, 1,2,2-trimethylpropyl and the like. The term “alkylene,” employed alone or in combination with other terms, refers to a divalent alkyl linking group. An alkylene group formally corresponds to an alkane with two C—H bonds replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like. It is to be understood that C₀-alkylene means that the specified substituent is absent. For example, when referring to the substituent C₀₋₄ alkylene-aryl-C₀₋₄ alkylene, if both numerals are 0, the substituent is divalent aryl (e.g., phenylene, C₆H₄). Also, when referring to L is L^{a}-X^{a}-L^{b}; and each one of L^{a} and L^{b} is C₀ alkylene, then L is X^{a}. In various section of the present application ranges of alkyl chains are presented, e.g., C₀₋₄ alkyl, C₄₋₂₀ alkyl, C₄₋₁₄ alkyl etc. It is to be understood that such ranges include any sub range thereof. for example, C₄₋₁₄ alkyl may include and/or be directed to: C₄₋₈ alkyl, C₈₋₁₄ alkyl, C₆₋₁₂ alkyl, C₉ alkyl etc.

An “alkenyl” group refers to an aliphatic hydrocarbon group containing at least one carbon-carbon double bond including straight-chain, branched-chain and cyclic alkenyl groups. Exemplary alkenyl groups include ethenyl, propenyl, n-butenyl, i-butenyl, 3-methylbut-2-enyl, n-pentenyl, heptenyl, octenyl, cyclohexyl-butenyl and decenyl. The alkenyl group can be unsubstituted or substituted through available carbon atoms with one or more groups defined hereinabove for alkyl. Alkenyls according to the present invention may include more than one carbon-carbon double bond. Thus, dienes (see e.g., compound DSL1-1, substituents R² and R⁴) and trienes are within the definition of alkenyl. According to some embodiments, the alkenyl is a dienyl. The term “Cₙ₋ₘ alkenyl”, refers to an alkyl group having n to m carbon atoms. An alkenyl group formally corresponds to an alkene with one C—H bond replaced by the point of attachment of the alkenyl group to the remainder of the compound. Examples of alkenyl moieties include, but are not limited to, chemical groups such as ethenyl, propenyl, isopropenyl, n-butenyl, sec-butenyl the like. The term “alkenylene,” employed alone or in combination with other terms, refers to a divalent alkenyl linking group. An alkenylene group formally corresponds to an alkane with two C—H bonds replaced by points of attachment of the alkenylene group to the remainder of the compound. The term “Cₙ₋ₘ alkenylene” refers to an alkenylene group having n to m carbon atoms. In various section of the present application ranges of alkenyl chains are presented, e.g., C₂₋₈ alkenyl, C₄₋₂₀ alkenyl etc. It is to be understood that such ranges include any sub range thereof. for example, C₄₋₁₄ alkenyl may include and/or be directed to: C₄₋₈ alkenyl, C₈₋₁₄ alkenyl, C₆₋₁₂ alkenyl, C₉ alkenyl etc.

One or more of the lipids of the invention, may be present as a salt. The term “salt” encompasses both basic and acid addition salts, including but not limited to, carboxylate salts or salts with amine nitrogen atoms, and include salts formed with the organic and inorganic anions and cations discussed below. Furthermore, the term includes salts that form by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids. Each possibility represents a separate embodiment of the invention. The term “organic or inorganic cation” refers to counter-ions for the anion of a salt. The counter-ions include, but are not limited to, alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, Berge et al., J. Pharm. Sci. (1977), 66:1-19, which is incorporated herein by reference.

Particles, Compositions and Uses

According to some embodiments, the present invention provides a particle comprising the lipid according to the present invention and a membrane stabilizing lipid. Thus, in some aspects, the present invention provides a composition comprising a lipid according to any one of formulae (I), (I′), (I″), (I′″), (Ia), (Ib), (Ic) and (II), e.g., any one of compounds DSL1-1 to DSL1-60, DSL3c-1 to DSL3c-8, DSL2-1 to DSL2-72, or DSL4-1 to DSL4-15, and a pharmaceutically acceptable excipient.

According to some embodiments, there is provided a composition comprising a plurality of particles as discloses herein and a pharmaceutically acceptable carrier, diluent or excipient. According to some embodiments, the composition is a liposomal composition. According to some embodiments, the particles of the present invention are in the form of liposomes. In other embodiments, the composition further comprises one or more components selected from the group consisting of a neutral lipid, a charged lipid, a steroid, and a polymer-conjugated lipid. Each possibility represents a separate embodiment of the present invention.

According to some embodiments, the particle comprises the membrane stabilizing lipid and a lipid membrane comprising the lipid. According to some embodiments, the membrane stabilizing lipid is selected from the group consisting of cholesterol, phospholipids, cephalins, sphingolipids and glycoglycerolipids. According to some embodiments, the membrane stabilizing lipid comprises cholesterol.

In some embodiments, the membrane stabilizing lipids may be selected from, but not limited to: cholesterol, phospholipids (such as, for example, phosphatidylcholine, phosphatidylethanolamine, phosphatidylinositol, phosphatidylserine, phosphatidylglycerol, diphosphatidylglycerols), cephalins, sphingolipids (sphingomyelins and glycosphingolipids), glycoglycerolipids, and combinations thereof. Each possibility represents a separate embodiment of the present invention. In some embodiments, the phosphatidylethanolamines may be selected from, but not limited to: 1,2-dilauroyl-L-phosphatidyl-ethanolamine (DLPE), 1,2-Dioleoyl-sn-glycero-3-phosphoethanolamine (DOPE), 1,2-Diphytanoyl-sn-glycero-3-phosphoethanolamine (DPhPE) 1,3-Dipalmitoyl-sn-glycero-2-phosphoethanolamine (1,3-DPPE), 1-Palmitoyl-3-oleoyl-sn-glycero-2-phosphoethanolamine (1,3-POPE), Biotin-Phosphatidylethanolamine, 1,2-Dimyristoyl-sn-glycero-3-phosphoethanolamine (DMPE), Dipalmitoylphosphatidylethanolamine (DPPE), 1,2-Distearoyl-sn-glycero-3-phosphoethanolamine (DSPE) or combinations thereof. In some embodiments, the Phosphatidylethanolamines may be conjugated to a PEG-Amine derivative. Each possibility represents a separate embodiment of the present invention.

According to some embodiments, “neutral lipid” refers to any of a number of lipid species that exist either in an uncharged or neutral zwitterionic form at physiological pH, such lipids include, but are not limited to, phosphotidylcholines such as 1,2-Distearoyl-sn-glycero-3-phosphocholine (DSPC), 1,2-Dipalmitoyl-sn-glyccro-3-phosphocholine (DPPC), 1,2-Dimyristoyl-sn-glyccro-3-phosphocholine (DMPC), 1-Palmitoyl-2-olcoyl-sn-glyccro-3-phosphocholine (POPC), 1,2-dioleoyl-sn-glycero-3-phosphocholine (DOPC), phophatidyl ethanolamines such as 1,2-Diolcoyl-sn-glyccro-3-phosphoethanolamine (DOPE), sphingomyelins (SM), ceramides, steroids such as sterols and their derivatives. Neutral lipids may be synthetic or naturally derived.

According to some embodiments, the particle further comprising one or more additional components selected from the group consisting of a PEG-lipid conjugate, a neutral lipid and a charged lipid. According to some embodiments, the additional component comprises 1,2-Distearoyl-sn-glycero-3-phosphocholine (DSPC). According to some embodiments, the additional component comprises 1,2-Dimyristoyl-sn-glyceryl-methoxy polyethylene glycol (DMG-PEG). According to some embodiments, the particles (lipid phase thereof), may further include one or more PEG derivatives. In some embodiments, the PEG derivatives may be conjugated to one or more additional molecules, such as, a lipid. In some embodiments, the PEG derivative is selected from, but not limited to: PEG-DMG 3-N-(-methoxy poly(ethylene glycol)2000)carbamoyl-1,2-dimyrisyl glycerol, PEG-cDMA 3-N-(-methoxy poly(ethylene glycol)2000)carbamoyl-1,2-dimyristyloxy-propylamine; PEG-cDSA, 3-N-(-methoxy poly(ethylene glycol)2000)carbamoyl-1,2-distearyloxy-propylamine, DSPE-PEG, PEG-maleimide, DSPE-PEG-maleimide, or combinations thereof. Each possibility represents a separate embodiment of the present invention. According to some embodiments, the particle comprises the lipid, cholesterol, 1,2-Distearoyl-sn-glycero-3-phosphocholine (DSPC) and 1,2-Dimyristoyl-sn-glyceryl-methoxy polyethylene glycol (DMG-PEG). According to some embodiments, particle comprises the lipid according to the present invention, a membrane stabilizing lipid, an additional phospholipid and PEG-lipid conjugate.

According to some embodiments, the ratio between the various lipids in the particle may vary. In some embodiments, the ratio is a molar ratio. In some embodiments, the ratio is a weight ratio. In some embodiments, each of the lipid groups may be at molar ratio/a weight ratio of about 1%-99%. According to some embodiments, particle comprises 10-70% mol % of the lipid according to the present invention, 20-80% mol % of the membrane stabilizing lipid, 5-50% of the additional phospholipid and 0.5-10% of the PEG-lipid conjugate. According to some embodiments, the molar percentage of the lipid is at least 10 mol % of the particle. According to some embodiments, the molar percentage of the lipid is at least 15 mol % of the particle. According to some embodiments, the molar percentage of the lipid is at least 20 mol % of the particle. According to some embodiments, the molar percentage of the lipid is at least 25 mol % of the particle. According to some embodiments, the molar percentage of the lipid is no more than 50 mol % of the particle. According to some embodiments, the molar percentage of the lipid is no more than 45 mol % of the particle. According to some embodiments, the molar percentage of the lipid is no more than mol % of the particle. According to some embodiments, the molar percentage of the lipid is no more than 35 mol % of the particle.

It is to be understood that by the phrase “the molar percentage of the lipid is at least x mol % of the particle” it is meant that at least x % of the particle molecules are of the lipid. The same terminology is reflected with other components of the present particle. Similarly, the phrase “the molar percentage of the lipid is no more than x mol % of the particle” it is meant that no more than x % of the particle molecules are of the lipid. The unit “mol %” is also sometimes referred as “mol:mol” or “% mol:mol”.

According to some embodiments, the molar percentage of the additional phospholipid is at least 5 mol % of the particle. According to some embodiments, the molar percentage of the additional phospholipid is at least 10 mol % of the particle. According to some embodiments, the molar percentage of the additional phospholipid is no more than 35 mol % of the particle. According to some embodiments, the molar percentage of the additional phospholipid is no more than 25 mol % of the particle. According to some embodiments, the molar percentage of the membrane stabilizing lipid is at least 30 mol % of the particle. According to some embodiments, the molar percentage of the membrane stabilizing lipid is at least 40 mol % of the particle. According to some embodiments, the molar percentage of the membrane stabilizing lipid is no more than 70 mol % of the particle. According to some embodiments, the molar percentage of the membrane stabilizing lipid is no more than 60 mol % of the particle. According to some embodiments, the molar percentage of the PEG-lipid conjugate is at least 1 mol % of the particle. According to some embodiments, the molar percentage of the PEG-lipid conjugate is at least 1.5 mol % of the particle. According to some embodiments, the molar percentage of the PEG-lipid conjugate is no more than 5 mol % of the particle. According to some embodiments, the molar percentage of the PEG-lipid conjugate is no more than 3.5 mol % of the particle.

According to some embodiments, the particles of the present intention are nanoparticles. According to some embodiments, the lipidic particles of the present intention are lipid nanoparticles.

In some embodiments, the particles (including any nucleic acid, therapeutic agent and the like encapsulated within and any targeting moiety conjugated thereto) have a particle size (diameter) in the range of about 10 to about 500 nm. In some embodiments, the particles have a particle size (diameter) in the range of about 10 to about 350 nm. In some embodiments, the particles have a particle size (diameter) in the range of about 40 to about 270 nm. In some embodiments, the particles have a particle size (diameter) in the range of over about 10 nm. In some embodiments, the particles have a particle size (diameter) of over about 20 nm. In some embodiments, the particles have a particle size (diameter) of over about 30 nm. In some embodiments, the particles have a particle size (diameter) of over about 40 nm. In some embodiments, the particles have a particle size (diameter) of over about 45 nm. In some embodiments, the particles have a particle size (diameter) of over about 50 nm. In some embodiments, the particles have a particle size (diameter) of over about 60 nm. In some embodiments, the particles have a particle size (diameter) of over about 70 nm. In some embodiments, the particles have a particle size (diameter) of over about 80 nm. In some embodiments, the particles have a particle size (diameter) of over about 90 nm. In some embodiments, the particles have a particle size (diameter) of over about 100 nm. In some embodiments, the particles have a particle size (diameter) of over about 150 nm. In some embodiments, the particles have a particle size (diameter) of not more than about 500 nm. In some embodiments, the particles have a particle size (diameter) of not more than about 400 nm. In some embodiments, the particles have a particle size (diameter) of not more than about 300 nm. In some embodiments, the size is a hydrodynamic diameter.

According to some embodiments, the particle further comprises a nucleic acid. According to some embodiments, the nucleic acid is encapsulated within a particle comprising the lipid. According to some embodiments, the nucleic acid is selected from the group consisting of small interfering RNA (siRNA), micro RNA (miRNA), antisense oligo nucleotides, messenger RNA (mRNA), ribozymes, pDNA, CRISPR mRNA, gRNA, circular RNA and immune stimulating nucleic acids. In some embodiments, the composition may further comprise a nucleic acid. Examples of nucleic acids include small interfering RNA (siRNA), micro RNA (miRNA), antisense oligo nucleotides, messenger RNA (mRNA), ribozymes, pDNA, CRISPR mRNA, gRNA, circular RNA and immune stimulating nucleic acids. Each possibility represents a separate embodiment of the present invention.

According to some embodiments, the weight ratio between the nucleic acid and the lipid mixture may be adjusted so as to achieve maximal biological effect by the nucleic acid on the target site. In some embodiments, the ratio between the nucleic acid and the lipid phase may be 1:1. For example, the weight ratio between the nucleic acid and the lipid phase may be 1:2. For example, the weight ratio between the nucleic acid and the lipid phase may be 1:5. For example, the weight ratio between the nucleic acid and the lipid phase may be 1:10. For example, the weight ratio between the nucleic acid and the lipids phase may be 1:16. For example, the weight ratio between the nucleic acid and the lipid phase may be 1:20. In some embodiments, the weight ratio between the nucleic acid and the lipid phase is about 1:1 to 1:20 (w:w).

According to some embodiments, the particle further comprises a therapeutic agent. According to some embodiments, the therapeutic agent is encapsulated within a particle comprising the lipid. According to some embodiments, the therapeutic agent is RNA comprising an open reading frame encoding a polypeptide that comprises a SARS-CoV-2 spike protein or an immunogenic fragment or variant thereof. Each possibility represents a separate embodiment of the invention. According to some embodiments, the therapeutic agent is RNA comprising an open reading frame encoding a polypeptide that comprises a SARS-CoV-2 spike protein, an immunogenic fragment of SARS-CoV-2 or a SARS-CoV-2 variant. Each possibility represents a separate embodiment of the invention. According to some embodiments, the therapeutic agent is RNA comprising an open reading frame encoding a polypeptide that comprises a SARS-CoV-2 spike protein. According to some embodiments, there is provided a method of gene silencing, comprising the step of contacting a cell with a composition comprising a plurality of particles according to the present invention and a pharmaceutically acceptable carrier, diluent or excipient. In some embodiments, the present invention provides a method of gene silencing, comprising the step of contacting a cell with a composition comprising a lipid of the present invention. In some embodiments, the cell is a cancer cell.

In other embodiments, the compositions of the present invention may be used as a delivery system to administer a therapeutic agent to its target location in the body. Thus, in some embodiments, the present invention relates to a method for administering a therapeutic agent, by preparing a composition comprising a lipid as described herein and a therapeutic agent, and administering the composition to a subject in need thereof. According to some embodiments, the present invention relates to a method for administering a therapeutic agent, by preparing a particle as described herein comprising a therapeutic agent, and administering the composition to a subject in need thereof. According to some embodiments, the method further comprises encapsulating the therapeutic agent within a particle comprising the lipid. According to some embodiments, the therapeutic agent is RNA comprising an open reading frame encoding a polypeptide that comprises a SARS-CoV-2 spike protein or an immunogenic fragment or variant thereof. Each possibility represents a separate embodiment of the invention. According to some embodiments, the therapeutic agent is RNA comprising an open reading frame encoding a polypeptide that comprises a SARS-CoV-2 spike protein or an immunogenic fragment or variant thereof. Each possibility represents a separate embodiment of the invention. According to some embodiments, the therapeutic agent is RNA comprising an open reading frame encoding a polypeptide that comprises a SARS-CoV-2 spike protein, an immunogenic fragment of SARS-CoV-2 or a SARS-CoV-2 variant. Each possibility represents a separate embodiment of the invention.

In particular embodiments, the present invention provides novel lipids that enable the formulation of improved compositions for the in vitro and in vivo delivery of IVT-mRNA and/or other oligonucleotides.

In some embodiments, these lipid nanoparticle compositions are useful for expression of protein encoded by mRNA.

In other embodiments, these improved lipid nanoparticles compositions are useful for upregulation of endogenous protein expression by delivering miRNA inhibitors targeting one specific miRNA or a group of miRNA regulating one target mRNA or several mRNA.

In other embodiments, these improved lipid nanoparticle compositions are useful for down-regulating (e.g., silencing) the protein levels and/or mRNA levels of target genes.

In some other embodiments, the lipid nanoparticles are also useful for delivery of mRNA and plasmids for expression of transgenes.

In yet other embodiments, the lipid nanoparticle compositions are useful for inducing a pharmacological effect resulting from expression of a protein, e.g., increased production of red blood cells through the delivery of a suitable erythropoietin mRNA, or protection against infection through delivery of mRNA encoding for a suitable antibody.

According to some embodiments, the lipid may be in the form of nanoparticles and administered as is. In some embodiments, the nanoparticles may be administered in a solution. In some embodiments, the nanoparticles may be formulated to a suitable pharmaceutical composition to be administered by any desired route of administration. Exemplary routes of administration include such routes as, but not limited to: topical, oral or parenteral. Depending on the intended mode of administration, the compositions used may be in the form of solid, semi-solid or liquid dosage forms, such, as for example, tablets, suppositories, pills, capsules, powders, liquids, suspensions, or the like, preferably in unit dosage forms suitable for single administration of precise dosages. The pharmaceutical compositions may include the particles, a pharmaceutical acceptable excipient, and, optionally, may include other medicinal agents, pharmaceutical agents, carriers, adjuvants, and the like. It is preferred that the pharmaceutically acceptable carrier be one which is inert to the nucleic acid encapsulated within the particles and which has no detrimental side effects or toxicity under the conditions of use. In some embodiments, the administration is localized. In some embodiments, the administration is systemic.

In some embodiments, injectable formulations for parenteral administration can be prepared as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, and the like. Aqueous injection suspensions may also contain substances that increase the viscosity of the suspension, including, for example, sodium carboxymethylcellulose, sorbitol, and/or dextran. Optionally, the suspension may also contain stabilizers. The parenteral formulations can be present in unit dose or multiple dose sealed containers, such as ampules and vials, and can be stored in a freeze-dried (lyophilized) condition requiring only the addition of the sterile liquid carrier, such as, for example, water, for injections immediately prior to use. In some embodiments, parenteral administration includes intravenous administration.

In other embodiments, for oral administration, a pharmaceutically acceptable, non-toxic composition may be formed by the incorporation of any of the normally employed excipients, such as, for example, mannitol, lactose, starch, magnesium stearate, sodium saccharine, talcum, cellulose, sodium crosscarmellose, glucose, gelatin, sucrose, magnesium carbonate, and the like. Such compositions include solutions, suspensions, tablets, dispersible tablets, pills, capsules, powders, sustained release formulations and the like. Formulations suitable for oral administration can consist of liquid solutions such as effective amounts of the compound(s) dissolved in diluents such as water, saline, or orange juice; sachets, lozenges, and troches, each containing a predetermined amount of the active ingredient as solids or granules; powders, suspensions in an appropriate liquid; and suitable emulsions. Liquid formulations may include diluents such as water and alcohols, (such as, for example ethanol, benzyl alcohol, and the polyethylene alcohols), either with or without the addition of a pharmaceutically acceptable surfactant, suspending agents, or emulsifying agents.

In determining the dosages of the particles to be administered, the dosage and frequency of administration may be selected in relation to the pharmacological properties of the specific nucleic acids encapsulated within the particles.

The lipids of the present invention can be used alone or in combination with other lipid components such as neutral lipids, charged lipids, steroids (including, for example, sterols) and/or their analogs, and/or polymer conjugated lipids to form lipid nanoparticles for the delivery of therapeutic agents.

In some instances the lipid nanoparticles are used to deliver nucleic acids for the treatment of various diseases or conditions, in particular leukocyte associated conditions such as inflammation and/or lack of sufficient protein.

Thus, in some embodiments, the present invention relates to a method of treating a leukocyte associated condition, the method comprising the step of administering to a subject in need thereof a composition according to the present invention. The leukocyte associated condition may be selected from the group consisting of cancer, infection, autoimmune diseases, neurodegenerative diseases and inflammation.

In some representative embodiments, the particle comprises a nucleic acid, such as, for example, siRNA, miRNA, shRNA, anti-sense RNA, and the like, may be used in the treatment of various leukocyte-associated conditions, depending on the identity of the nucleic acid, the specific target leukocyte, and the like. In some embodiments, the nucleic acid encapsulated within the particles may be a nucleic acid capable of inducing silencing of a target gene. In some embodiments, the target gene may be any gene, the expression of which is related to the condition to be treated. In some embodiments, the target gene may be a gene selected from, but not limited to: growth factors (such as EGFR, PDGFR), genes related to angiogenesis pathways (such as VEGF, Integrins), genes involved in intracellular signaling pathways and cell cycle regulation (such as PI3K/AKT/mTOR, Ras/Raf/MAPK, PDK1, CHK1, PLK1, Cyclins). In some embodiments, a combination of nucleic acids, each having one or more targets may be encapsulated within the particles.

According to some embodiments, exemplary leukocyte-associated conditions that may be treated by the targeted particles may be selected from, but not limited to: various types of cancer, various infections (such as, for example, viral infection, bacterial infection, fungal infection, and the like), autoimmune diseases, neurodegenerative diseases, inflammations, and the like.

In some representative embodiments, the targeted particles comprising a nucleic acid (such as, siRNA or miRNA, shRNA, anti-sense RNA, or the like), may be used for the treatment of cancer. In some embodiments, cancer is a disorder in which a population of cells has become, in varying degrees, unresponsive to the control mechanisms that normally govern proliferation and differentiation. In some embodiments, the cancer is a blood cancer. Non-limiting examples of blood cancers are lymphoma, leukemia and myloma. Lymphomas may be divided into two categories: Hodgkin lymphoma and non-Hodgkin lymphoma. Most non-Hodgkin lymphomas are B-cell lymphomas, that grow quickly (high-grade) or slowly (low-grade). There are 14 types of B-cell non-Hodgkin lymphomas. The others are T-cell lymphomas.

In some representative embodiments, the nucleic acid that may be used for the treatment of cancer is directed against a target gene, which is involved in the regulation of cell cycle. In some representative embodiments, the target gene may be Polo-like Kinase 1 (PLK), Cyclin D1, CHK1, Notch pathway genes.

According to some exemplary embodiments, the plurality of lipids of the lipid particles may be of natural or synthetic source and may be selected from, but not limited to: cationic lipids, phosphatidylethanolamines, ionized lipids, membrane stabilizing lipids, phospholipids, and the like, or combinations thereof. Each possibility represents a separate embodiment of the present invention.

According to some embodiments, the particle further comprises a targeting moiety connected to a component of the composition. According to some embodiments, the particle is conjugated to a targeting moiety. According to some embodiments, the targeting moiety may by conjugated to any one of the lipids included in the present particle.

According to some embodiments, the particles may be comprised of any one or more of the lipids of the present invention, a phospholipid (e.g. DSPC), a membrane stabilizing lipid (e.g. cholesterol), a PEG-lipid conjugate (e.g. DMG-PEG); at various mol:mol ratios, and further conjugated to a targeting moiety, wherein the targeting moiety is conjugated, linked or attached to any one of the particle's components.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

As referred to herein, the terms “nucleic acid”, “nucleic acid molecules” “oligonucleotide”, “polynucleotide”, and “nucleotide” may interchangeably be used herein. The terms are directed to polymers of deoxy ribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may include sense and antisense oligonucleotide or polynucleotide sequences of DNA or RNA. The DNA or RNA molecules may be, for example, but not limited to: complementary DNA (cDNA), genomic DNA, synthesized DNA, recombinant DNA, or a hybrid thereof or an RNA molecule such as, for example, mRNA, shRNA, siRNA, miRNA, Antisense RNA, and the like. Each possibility represents a separate embodiment of the present invention. The terms further include oligonucleotides composed of naturally occurring bases, sugars, and covalent inter nucleoside linkages, as well as oligonucleotides having non-naturally occurring portions, which function similarly to respective naturally occurring portions. The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “construct”, as used herein, refers to an artificially assembled or isolated nucleic acid molecule which may include one or more nucleic acid sequences, wherein the nucleic acid sequences may include coding sequences (that is, sequence which encodes an end product), regulatory sequences, non-coding sequences, or any combination thereof. The term construct includes, for example, vector but should not be seen as being limited thereto.

“Expression vector” refers to constructs that have the ability to incorporate and express heterologous nucleic acid fragments (such as, for example, DNA), in a foreign cell. In other words, an expression vector comprises nucleic acid sequences/fragments (such as DNA, mRNA, tRNA, rRNA), capable of being transcribed. Many prokaryotic and eukaryotic expression vectors are known and/or commercially available. Selection of appropriate expression vectors is within the knowledge of those having skill in the art. In some representative embodiments, the expression vector may encode for a double stranded RNA molecule in the target site.

The term “expression”, as used herein, refers to the production of a desired end-product molecule in a target cell. The end-product molecule may include, for example an RNA molecule; a peptide or a protein; and the like; or combinations thereof.

As used herein, the terms “introducing” and “transfection” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, and the like, or combinations thereof. The transfection of the cell may be performed on any type of cell, of any origin, such as, for example, human cells, animal cells, plant cells, virus cell, and the like. The cells may be selected from isolated cells, tissue cultured cells, cell lines, cells present within an organism body, and the like.

The term “treating” and “treatment” as used herein refers to abrogating, inhibiting, slowing or reversing the progression of a disease or condition, ameliorating clinical symptoms of a disease or condition or preventing the appearance of clinical symptoms of a disease or condition. The term “preventing” is defined herein as barring a subject from acquiring a disorder or disease or condition.

The term “treatment of cancer” is directed to include one or more of the following: a decrease in the rate of growth of the cancer (i.e. the cancer still grows but at a slower rate); cessation of growth of the cancerous growth, i.e., stasis of the tumor growth, and, the tumor diminishes or is reduced in size. The term also includes reduction in the number of metastases, reduction in the number of new metastases formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment, lengthening the time of diseases progression, tumor regression, and the like. In some embodiments, the cancer is a blood cancer.

The term “Leukocytes” is directed to white blood cells (WBCs), produced and derived from a multipotent, hematopoietic stem cell in the bone marrow. The white blood cells have nuclei, and types of white blood cells can be classified in into five main types, including, neutrophils, eosinophils, basophils, lymphocytes, and monocytes, based on functional or physical characteristics. The main types may be classified into subtypes. For example, lymphocytes include B cells, T cells, and NK cells. B-cells, for example, release antibodies and assist activation of T cells. T cells, for example, can be classified to several subtypes, including: T-helper cells (CD4+ Th) which activate and regulate T and B cells; cytotoxic T cells (CD8+) that can target and kill virus-infected cells and tumor cells; Gamma-delta T cells (γδ T cells) which can bridge between innate and adaptive immune responses and be involved in phagocytosis; and Regulatory (suppressor) T cells which modulate the immune system, maintain tolerance to self-antigens, and abrogate autoimmune conditions.

### EXAMPLES

### Example 1: Synthesis of Ionizable Lipids

Synthesis of DSL1-1

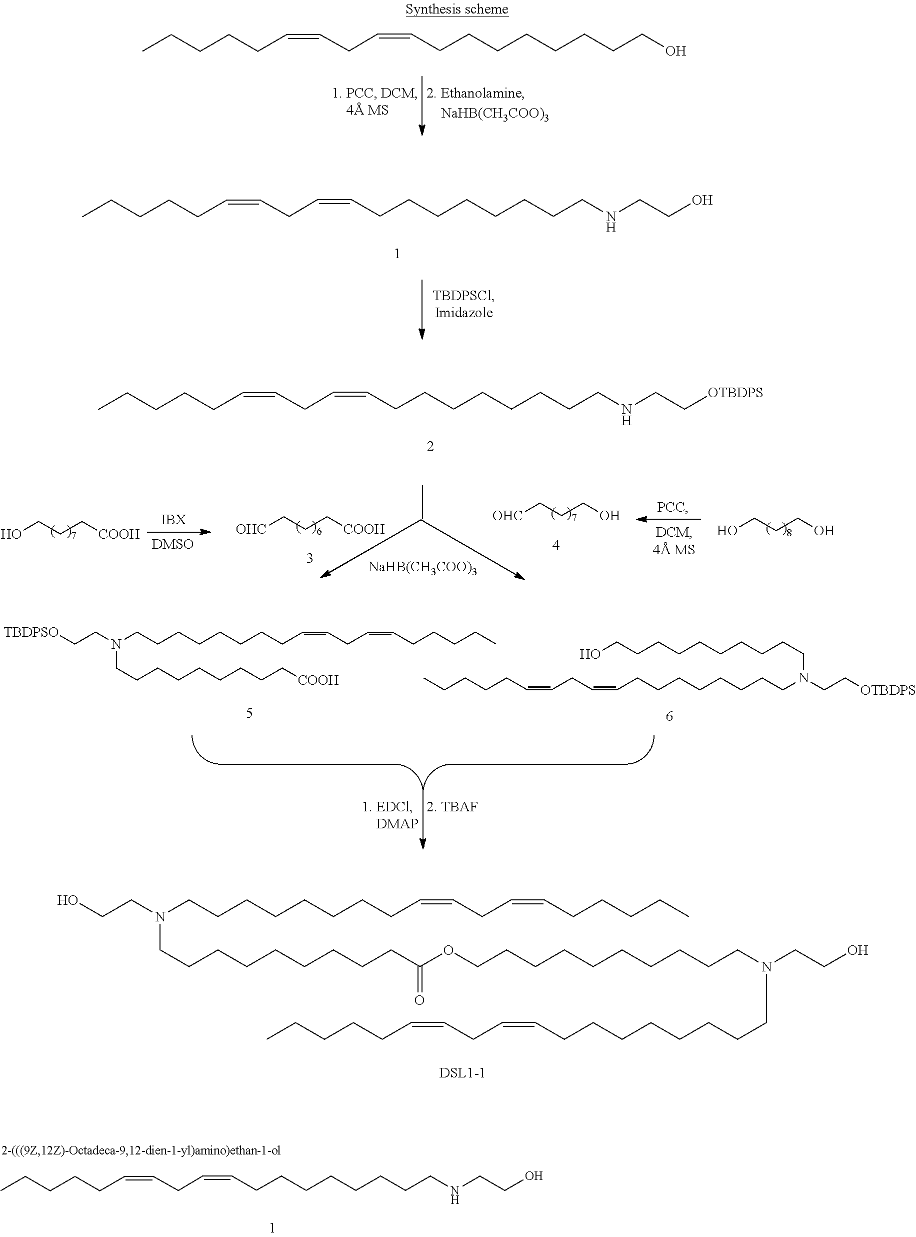

To a solution of linoleic alcohol (4.0 g, 15.0 mmol, 1 equiv.) in dry CH₂Cl₂ (80 mL), molecular sieves (4 Å MS) were added under argon atmosphere. Then PCC (6.4 g, 30.0 mmol, 2 equiv.) was added portion wise over a period of 10 min and stirred for 2 hr at room temperature. After completing the reaction filtered it through a silica gel pad using CH₂Cl₂ to remove PCC. The solvent was evaporated under reduced pressure to obtain the linoleic aldehyde 3.9 g (99%) as a colorless liquid.

Linoleic aldehyde (3.20 g, 12.12 mmol, 1 equiv.) and ethanolamine (0.89 ml, 14.54 mmol, 1.2 equiv.) were dissolved in dry CH₂Cl₂ (60 mL) under nitrogen atmosphere and stirred for 1 hr. at room temperature. Then sodium triacetoxyborohydride (5.10 g, 24.24 mmol, 2 equiv.) was added portion wise over a period of 15 min, and stirred it for 16 hr at the same temperature. Later, the reaction mixture was quenched with sat·NaHCO₃ solution followed by extract with CH₂Cl₂ (3 times). The organic layer was washed with brine solution and dried over anhydrous Na₂SO₄. The solvent was evaporated and the residue was purified by column chromatography using 0-10% MeOH in CHCl₃ to get 1 (2.0 g, 54%) as a pale yellowish liquid.

¹H NMR (400 MHz, CDCl3): δ 5.40-5.30 (4H, m), 3.64 (2H, t, J=5.2 Hz), 2.83-2.73 (4H, m), 2.62 (2H, t, J=7.2 Hz), 2.04 (4H, q, J=6.8 Hz), 1.54-1.43 (2H, m), 1.41-1.21 (16H, m), 0.88 (3H, t, J=6.8 HZ).

ESI-MS: m/z 310.5 [M+1]⁺.

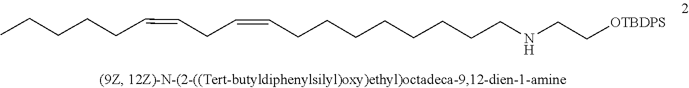

To a stirred solution of 1 (1.30 g, 4.2 mmol, 1 equiv.) and imidazole (628 mg, 9.24 mmol, 2.2 equiv.) in dry CH₂Cl₂ (40 mL), TBDPS-Cl (1.31 mL, 5.04 mmol, 1.2 equiv.) was added drop-wise under argon atmosphere and stirred it for overnight at room temperature. The reaction mixture was then poured in brain solution and extracted with CH₂Cl₂. The organic layer was dried over anhydrous Na₂SO₄ and concentrated under reduced pressure. The crude product was purified by column chromatography using 0-3% MeOH in CHCl₃ to afford TBDPS protected compound 2 in quantitative yield as a yellow color liquid.

¹H NMR (400 MHz, CDCl3): δ 7.69-7.63 (4H, m), 7.45-7.33 (6H, m), 5.44-5.28 (4H, m), 3.78 (2H, t, J=5.6 Hz), 2.77 (2H, t, J=6.4 Hz), 2.74 (2H, t, J=5.2 Hz), 2.59 (2H, t, J=7.2 Hz), 2.05 (4H, q, J=6.8 Hz), 1.53-1.42 (2H, m), 1.41-1.21 (16H, m), 1.05 (9H, s), 0.89 (3H, t, J=6.8 HZ)

ESI-MS: m/z 548.7 [M+1]⁺

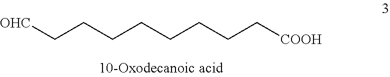 [SMILES: O=CCCCCCCCCC(=O)O]

To a solution of IBX (11.9 g, 45 wt. %, 19.15 mmol, 1.2 equiv.) in DMSO (40 mL), a solution of 10-Hydroxydecanoic acid (3.0 g, 15.96 mmol, 1.0 equiv.) in THF (20 mL) was added and stirred for 6 hr at room temperature. After that, the reaction was quenched with water (20 mL) and the precipitated solid was removed by filtration. The filtrate was diluted with water and extracted with diethyl ether (4×100 mL). The organic layer dried over anhydrous Na₂SO₄ and the solvent was removed on rotary evaporator. The crude product was purified by column chromatography using 0-20% ethyl acetate in hexane to obtain 10-oxodecanioc acid 3 (2.60 g, 87%) as a white solid.

¹H NMR (400 MHz, CDCl3): δ 9.75 (1H, t, J=2.0 Hz), 2.41 (2H, dt, J=7.2, 2.0 Hz), 2.33 (2H, t, J=7.6 Hz), 1.61 (4H, quint, J=7.2 Hz), 1.39-1.23 (8H, m).

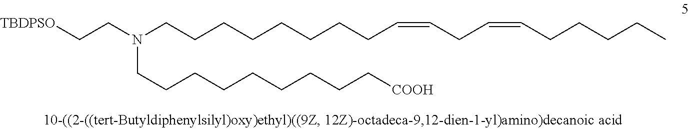

The compound 2 (700 mg, 1.27 mmol, 1.0 equiv.) and 10-oxodecanioc acid 3 (285 mg, 1.53 mmol, 1.2 equiv.) were dissolved in dry CH₂Cl₂ (30 mL) under argon atmosphere and stirred for 1 hr at room temperature. Then sodium triacetoxyborohydride (404 mg, 1.91 mmol, 1.5 equiv.) was added and stirred it for 24 hr at the same temperature. Later, the reaction was quenched with sat·NaHCO₃ solution followed by extract with CH₂Cl₂ (3 times). The organic layer was washed with brine solution and dried over anhydrous Na₂SO₄. The solvent was evaporated rotary evaporator and the residue was purified by column chromatography using 0-3% MeOH in CHCl₃ to provide 5 (778 mg, 85%) as a pale yellowish viscus liquid.

¹H NMR (400 MHz, CDCl3): δ 7.67-7.61 (4H, m), 7.46-7.34 (6H, m), 5.43-5.27 (4H, m), 3.86 (2H, t, J=5.6 Hz), 2.96 (2H, t, J=5.6 Hz), 2.85-2.66 (6H, m), 2.21 (2H, t, J=7.2 Hz), 2.11-1.96 (4H, m), 1.65-1.44 (6H, m), 1.42-1.13 (26H, m), 1.04 (9H, s), 0.88 (3H, t, J=6.8 Hz).

ESI-MS: m/z 718.9 [M+1]⁺; 716.9 [M−1]⁻

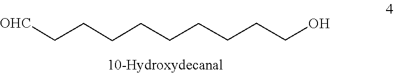 [SMILES: O=CCCCCCCCCCO]

1,10-Decanediol (1.0 g, 5.7 mmol, 1 equiv.) was dissolved in dry THF (40 mL) under argon atmosphere and molecular sieves (4 Å MS) were added. Then PCC (1.5 g, 6.9 mmol, 1.2 equiv.) was added portion wise to the reaction mixture over a period of 5 min and stirred for 2 hr at room temperature. After that, the reaction mixture was filtered through silica gel pad to remove PCC followed by wash with 30% ethyl acetate in hexane (2×100 mL). The solvent was evaporated under reduced pressure and the residue was purified by column chromatography using 5-15% ethyl acetate in hexane to obtain the 10-hydroxydecenal 4 (0.49 g, 49%) as a white solid.

¹H NMR (400 MHz, CDCl3): δ 9.77 (1H, t, J=2.0 Hz), 3.64 (2H, t, J=6.8 Hz), 2.43 (2H, dt, J=7.6, 2.0 Hz), 1.69-1.52 (4H, m), 1.41-1.25 (10H, m).

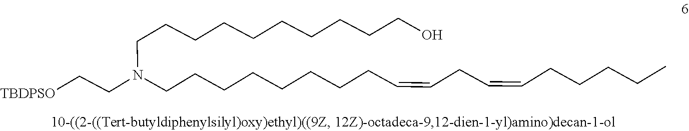

The compound 2 (1.8 g, 3.28 mmol, 1 equiv.) and 10-hydroxydecenal 4 (678 mg, 3.94 mmol, 1.2 equiv.) were dissolved in dry CH₂Cl₂ (50 mL) under argon atmosphere and stirred for 1 hr. at room temperature. Then sodium triacetoxyborohydride (1.38 g, 6.56 mmol, 2 equiv.) was added and stirred it for 24 hr at the same temperature. After that, the reaction was quenched with sat. NaHCO₃ solution followed by extract with CH₂Cl₂ (3×30 mL). The organic layer was washed with brine solution and dried over anhydrous Na₂SO₄. The solvent was evaporated on rotary evaporator and the residue was purified by column chromatography using 0-2% MeOH in CHCl₃ to obtain 6 (1.9 g, 82%) as a yellowish viscus liquid.

¹H NMR (400 MHz, CDCl3): δ 7.70-7.63 (4H, m), 7.45-7.33 (6H, m), 5.43-5.27 (4H, m), 3.85-3.68 (2H, br), 3.63 (2H, dt, J=6.8, 2.0 Hz), 2.77 (2H, t, J=6.4 Hz), 2.72-2.57 (2H, br), 2.53-2.32 (4H, br), 2.04 (4H, q, J=8.0 Hz) 1.65-1.47 (4H, m), 1.45-1.12 (30H, m), 1.05 (9H, s), 0.89 (3H, t, J=7.2 Hz).

ESI-MS: m/z 704.9 [M+1]⁺

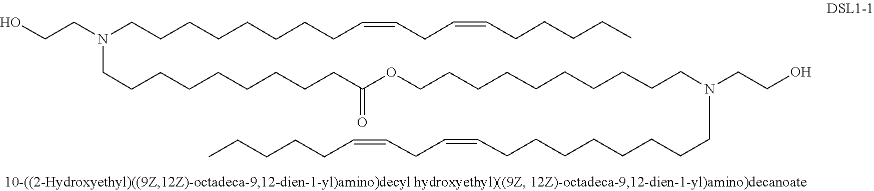 [SMILES: CCCCC/C=C\C/C=C\CCCCCCCCN(CCO)CCCCCCCCCCOC(=O)CCCCCCCCCN(CCO)CCCCCCCC/C=C\C/C=C\CCCCC]

The alcohol 6 (303 mg, 0.43 mmol, 1.0 equiv.), acid 5 (370 mg, 0.51 mmol, 1.2 equiv.), EDC·HCl (165 mg, 0.86 mmol, 2.0 equiv.) and DMAP (11 mg, 0.09 mmol, 0.2 equiv.) were dissolved in dry CH₂Cl₂ (15 mL) under nitrogen atmosphere and stirred for 24 hr at room temperature. Later, the reaction was quenched with sat. NaHCO₃ and extracted with CH₂Cl₂ (3 times). The organic portion was washed with brine solution and dried over anhydrous Na₂SO₄. The solvent was evaporated and the residue purified with a short silica gel column (2% IPA/CHCl₃) to get the desired product (417 mg, 69%). The obtained product (417 mg, 0.3 mmol, 1 equiv.) was dissolved in THF (5 mL), and TBAF (1.2 mL, 1.0 M in THF, 1.19 mmol, 4.0 equiv.) was added. The reaction was stirred for 3 hr at room temperature and quenched with sat. NH₄Cl, and extracted with 20% ethyl acetate in diethyl ether. Then the combined organic portion was washed with sat. NH₄Cl solution (3 times) to remove TBAF completely. The solvent was evaporated under reduced pressure and the residue purified by column chromatography using 0-15% isopropanol in chloroform to obtain the DSL1-1 (220 mg, 80%) as a yellow color viscus liquid.

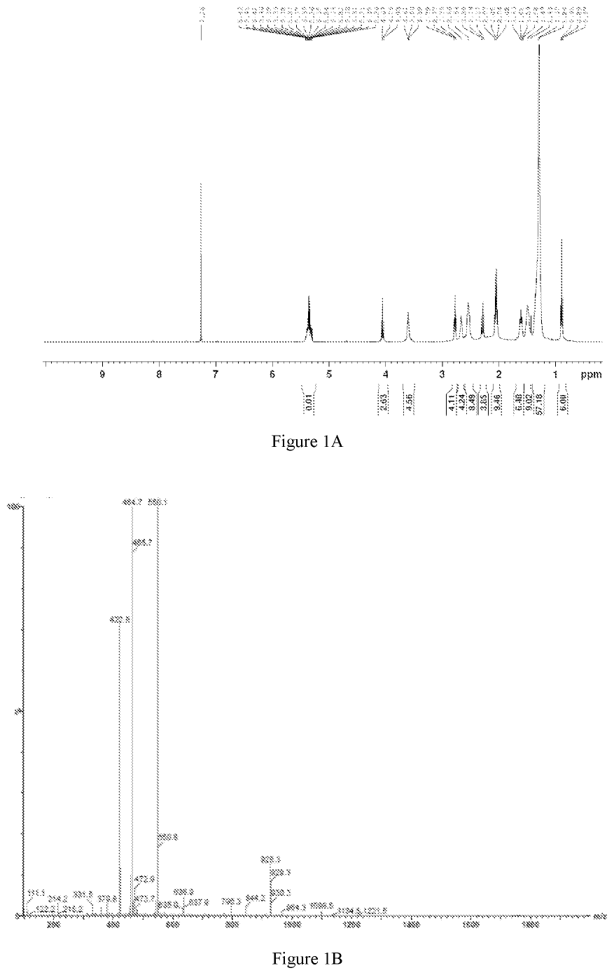

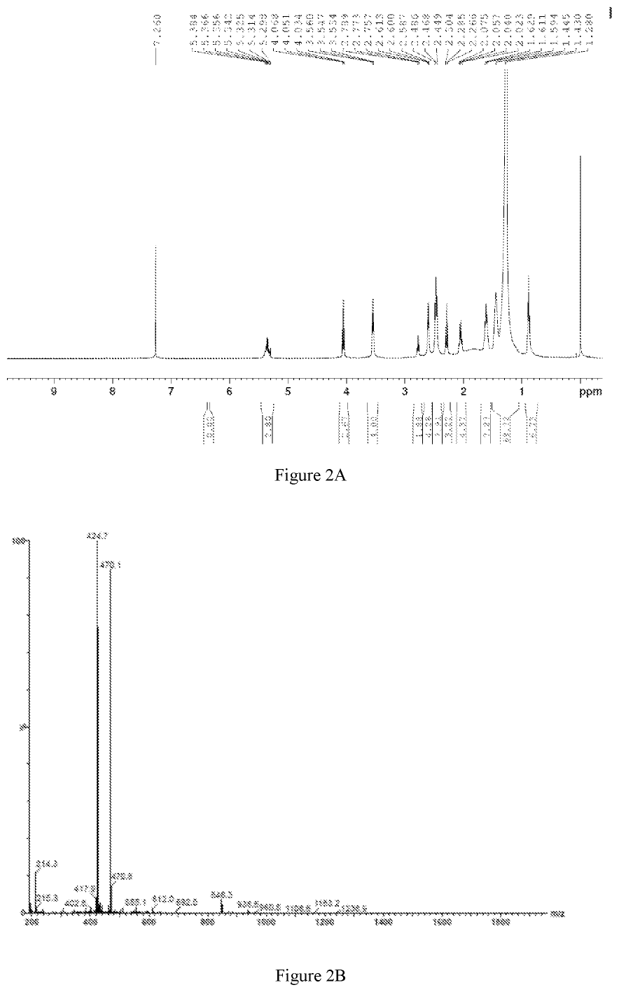

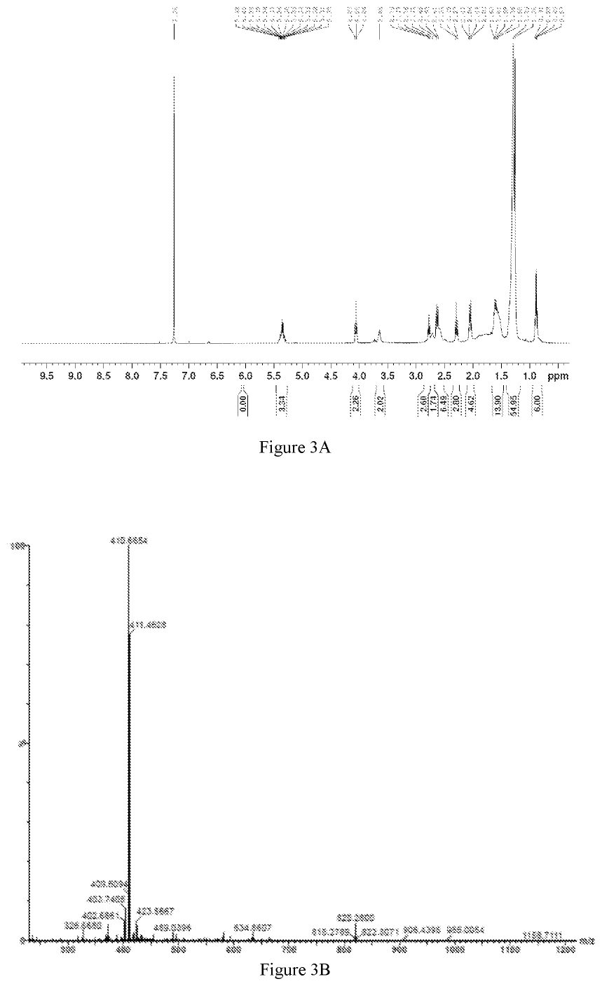

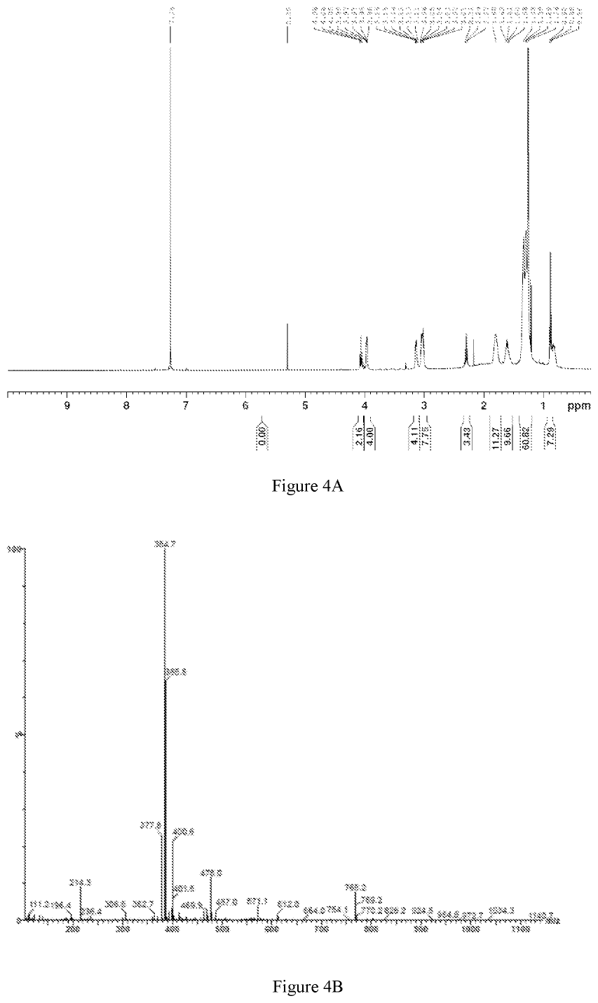

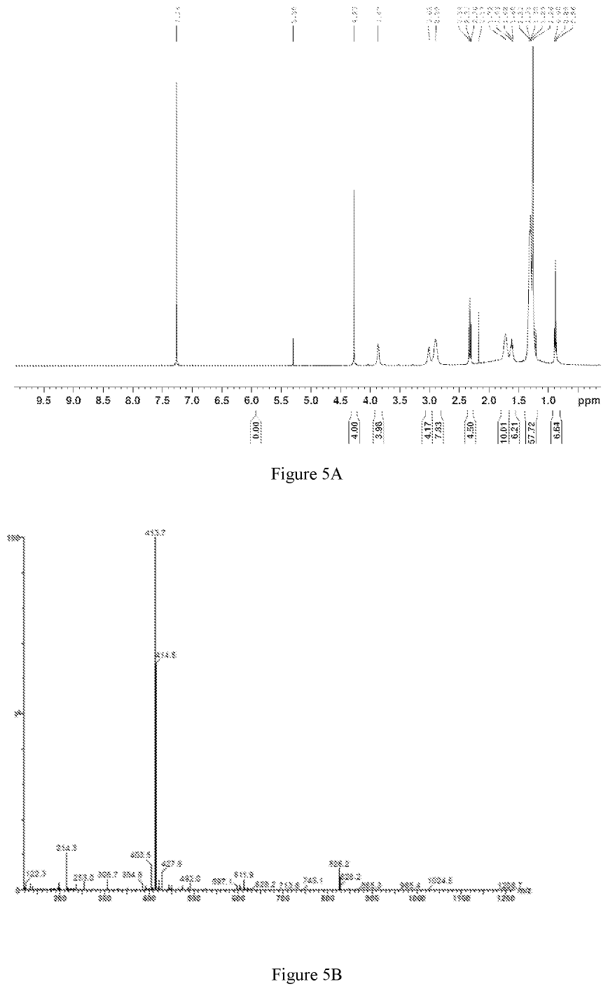

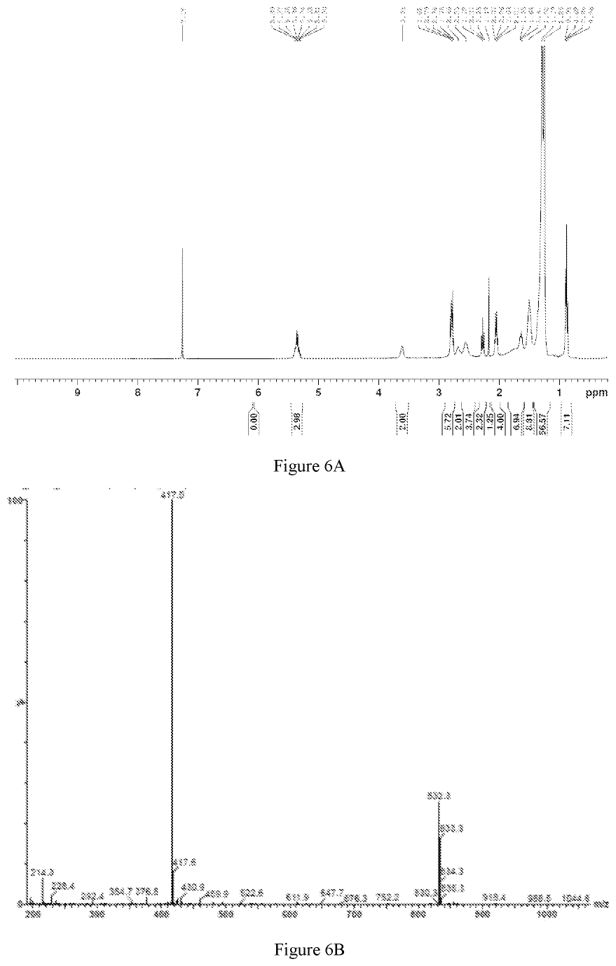

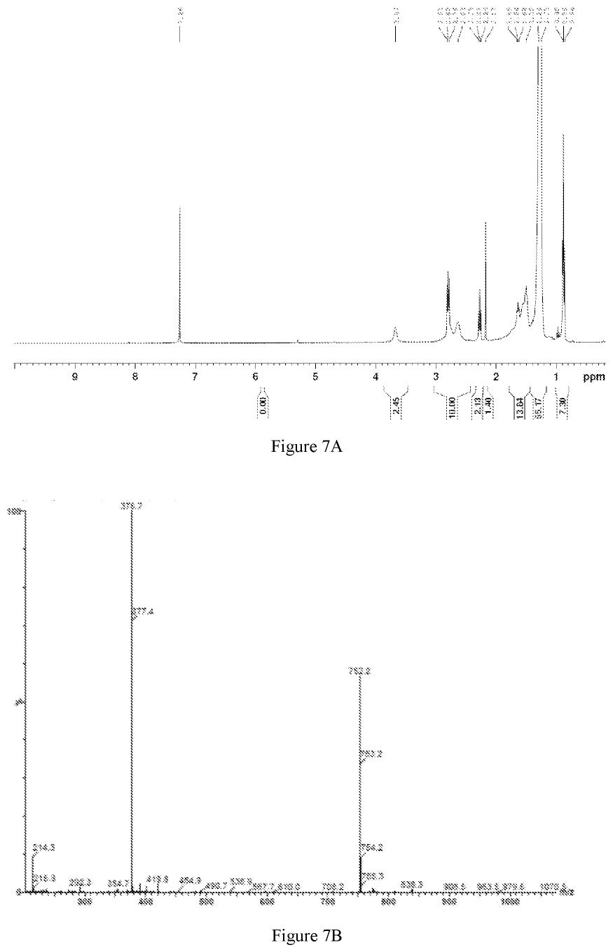

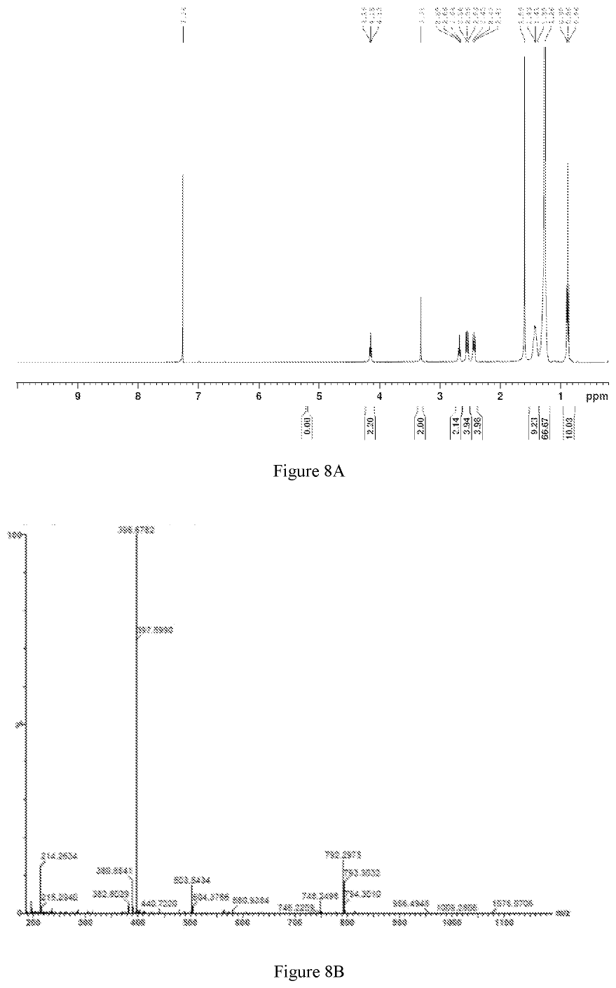

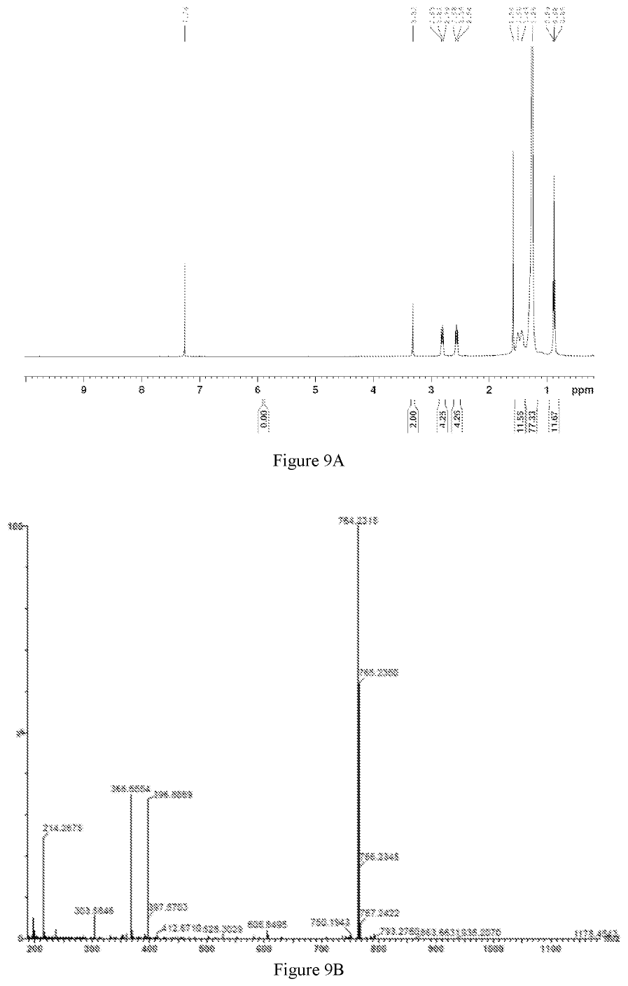

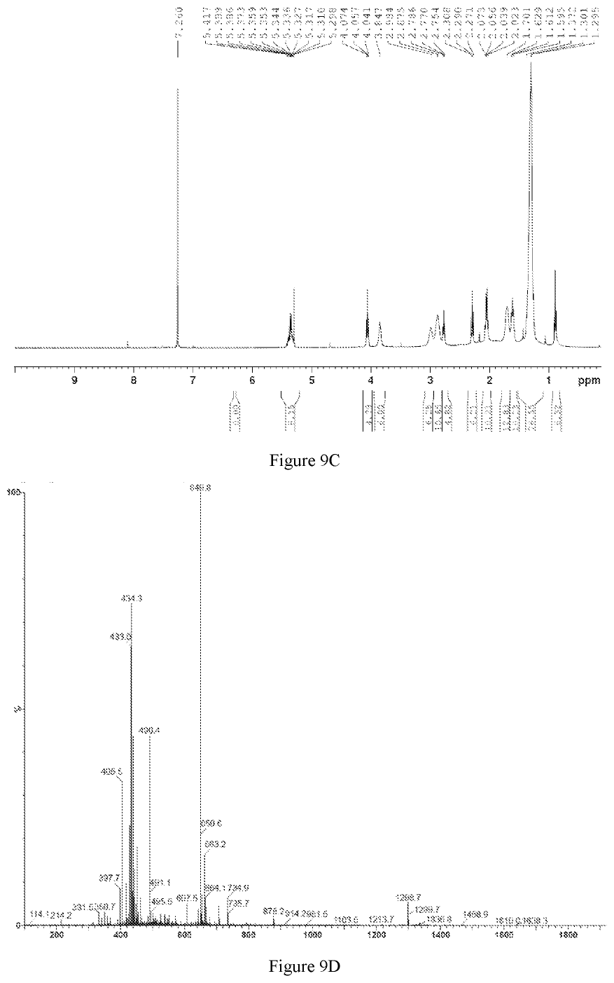

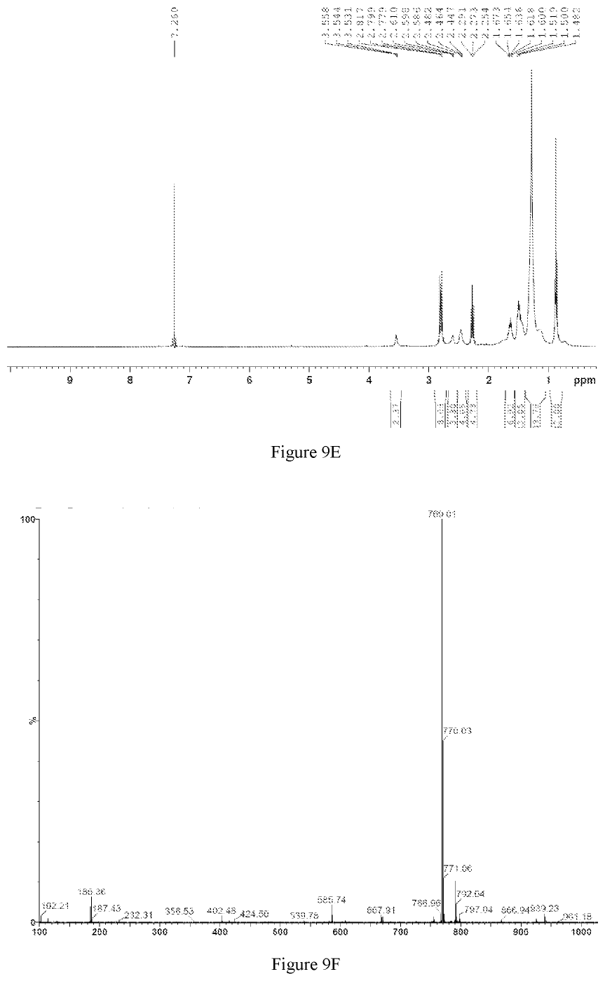

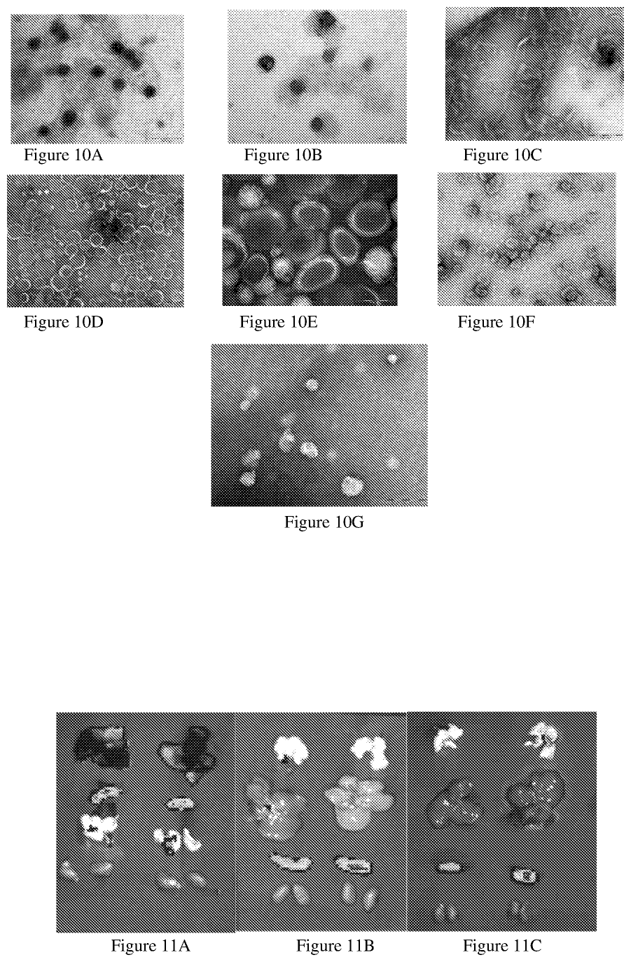

FIGS. 1A and 1B represent the ¹H NMR spectra and ESI-MS of DSL1-1, respectively.

¹H NMR (400 MHz, CDCl3): δ 5.45-5.26 (8H, m), 4.05 (2H, t, J=6.8 Hz), 3.60 (4H, t, J=4.8 Hz), 2.77 (4H, t, J=6.4 Hz), 2.71-2.62 (4H, br), 2.59-2.47 (8H, br), 2.28 (4H, t, J=7.6), 2.05 (8H, q, J=6.8 Hz), 1.60 (6H, quint, J=6.8 Hz), 1.55-1.42 (8H, m), 1.40-1.20 (50H, m), 0.89 (6H, t, J=7.2 Hz).

ESI-MS: m/z 928.3 [M+1]⁺; 464.7 [M/2+1]⁺

Synthesis of DSL1-2

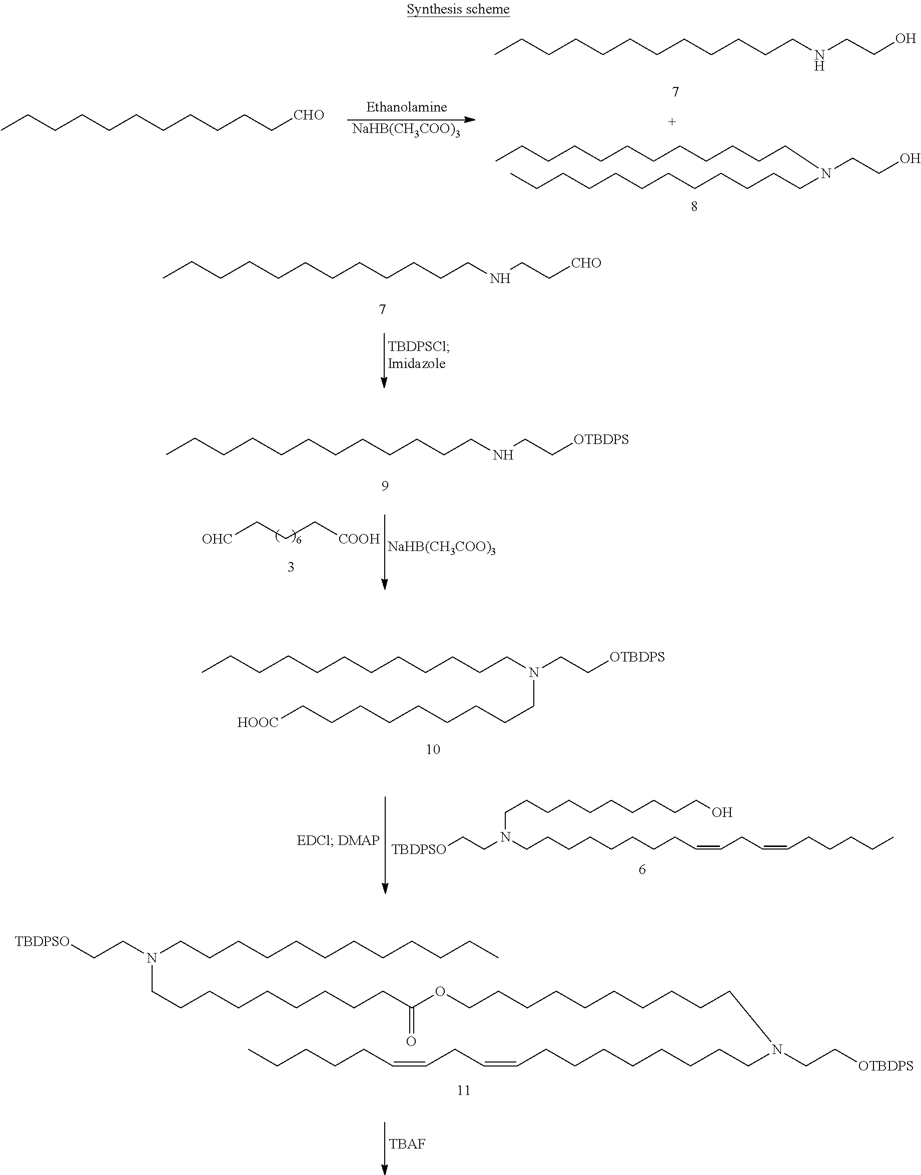

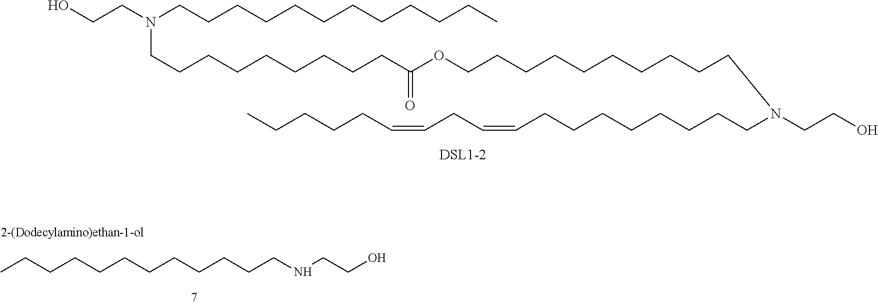 [SMILES: CCCCC/C=C\C/C=C\CCCCCCCCN(CCO)CCCCCCCCCCOC(=O)CCCCCCCCCN(CCO)CCCCCCCCCCCC ; CCCCCCCCCCCCNCCO]

To a solution of dodecanal (2.4 mL, 10.87 mmol, 1.0 equiv.) in dry CH₂Cl₂ (60 mL), ethanolamine (0.79 ml, 13.04 mmol, 1.2 equiv.) was added under nitrogen atmosphere and stirred for 1 hr. at room temperature. Then sodium triacetoxyborohydride (4.6 g, 21.74 mmol, 2.0 equiv.) was added portion wise over a period of 15 min and stirred for 24 hr at the same temperature. The reaction was quenched with sat·NaHCO₃ solution followed by extract with CH₂Cl₂ (3 times). The organic layer was washed with brine solution and dried over anhydrous Na₂SO₄. The solvent was evaporated and the residue was purified by column chromatography using 0-10% MeOH in CHCl₃ to obtain 7 (0.97 g, 40%) and 8 (0.45 g, 19%) as white solid and pale yellowish liquid respectively.

¹H NMR (400 MHz, CDCl3): δ 3.64 (2H, t, J=5.2 Hz), 3.08 (2H, br), 2.75 (2H, t, J=5.2 Hz), 2.60 (2H, t, J=7.2 Hz), 1.48 (2H, quint, J=7.2 Hz), 1.35-1.17 (18H, m), 0.86 (3H, t, J=7.2 Hz).

ESI-MS: m/z 230.4 [M+1]⁺

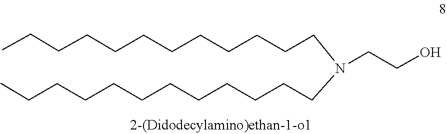 [SMILES: CCCCCCCCCCCCN(CCO)CCCCCCCCCCCC]

¹H NMR (400 MHz, CDCl3): δ 3.54 (2H, t, J=5.6 Hz), 2.60 (2H, t, J=5.2 Hz), 2.46 (4H, t,J10=7.6 Hz), 1.44 (4H, quint, J=6.8 Hz), 1.34-1.19 (36H, m), 0.88 (6H, t, J=7.2 Hz).

ESI-MS: m/z 398.7 [M+1]⁺

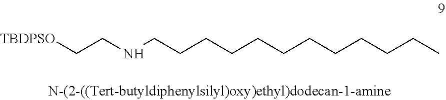

To a solution of 7 (4.0 g, 17.47 mmol, 1.0 equiv.) and imidazole (2.38 g, 34.94 mmol, 2.0 equiv.) in dry CH₂Cl₂ (100 mL), TBDPS-Cl (5.0 mL, 19.22 mmol, 1.1 equiv.) was added drop-wise over a period of 5 min under argon atmosphere and stirred for overnight at room temperature. Then the reaction mixture was poured in brain solution and extracted with CH₂Cl₂ (3 times). The organic portion was dried over anhydrous Na₂SO₄ and concentrated under reduced pressure. The crude product was purified by column chromatography using 0-2% MeOH in CHCl₃ to afford TBDPS protected compound 9 (7.73 g, 95%) as a pale yellowish liquid.

¹H NMR (400 MHz, CDCl3): δ 7.69-7.63 (4H, m), 7.45-7.34 (6H, m), 3.78 (2H, t, J=5.2 Hz), 2.74 (2H, t, J=5.2 Hz), 2.60 (2H, t, J=7.2 Hz), 1.49 (2H, quint, J=6.8 Hz), 1.36-1.18 (18H, m), 1.05 (9H, s), 0.88 (3H, t, J=6.8 Hz).

ESI-MS: m/z 468.7 [M+1]⁺

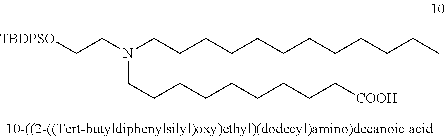

The compound 9 (5.03 g, 10.75 mmol, 1.0 equiv.) and 10-oxodecanioc acid 3 (2.4 g, 12.90 mmol, 1.2 equiv.) were dissolved in dry CH₂Cl₂ (180 mL) under argon atmosphere and stirred for 1 hr. at room temperature. Then sodium triacetoxyborohydride (3.4 g, 16.12 mmol, 1.5 equiv.) was added, and the reaction mixture was stirred for 24 hr at the same temperature. Later, the reaction was quenched with sat·NaHCO₃ solution followed by extract with CH₂Cl₂ (3 times). The organic layer was washed with brine solution and dried over anhydrous Na₂SO₄. The solvent was evaporated rotary evaporator and the residue was purified by column chromatography using 0-5% IPA in CHCl₃ to provide 10 (6.6 g, 97%) as a pale yellowish viscus liquid.

¹H NMR (400 MHz, CDCl3): δ 7.69-7.60 (4H, m), 7.46-7.33 (6H, m), 3.86 (2H, t, J=5.6 Hz), 2.95 (2H, t, J=5.6 Hz), 2.81-2.66 (4H, m), 2.21 (2H, t, J=7.2 Hz), 1.64-1.43 (6H, m), 1.36-1.13 (28H, m), 1.04 (9H, s), 0.87 (3H, t, J=6.8 Hz).

ESI-MS: m/z 638.9 [M+1]⁺; 636.8 [M−1]⁻

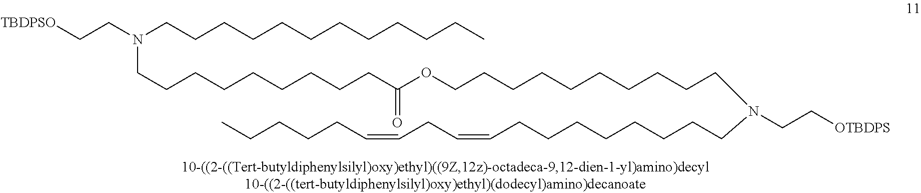

The alcohol 6 (338 mg, 0.48 mmol, 1.0 equiv.), acid 10 (460 mg, 0.72 mmol, 1.5 equiv.), EDC·HCl (183 mg, 0.96 mmol, 2.0 equiv.) and DMAP (12 mg, 0.01 mmol, 0.2 equiv.) were dissolved in dry CH₂Cl₂ (10 mL) under argon atmosphere and stirred for 24 hr at room temperature. Then the reaction was quenched with sat. NaHCO₃ and extracted with CH₂Cl₂ (3 times). The organic portion was washed with brine solution and dried over anhydrous Na₂SO₄. The solvent was evaporated and the residue purified by column chromatography using 0-2% IPA/CHCl₃ to obtain the desired product 11 (590 mg, 93%) as pale yellowish liquid.

¹H NMR (400 MHz, CDCl3): δ 7.71-7.64 (8H, m), 7.45-7.33 (12H, m), 5.44-5.23 (4H, m), 4.05 (2H, t, J=6.8 Hz), 3.70 (4H, t, J=6.8 Hz), 2.77 (2H, t, J=6.4 Hz), 2.61 (4H, t, J=6.8 Hz), 2.37 (8H, t, J=7.2 Hz), 2.28 (2H, t, J=7.6 Hz), 2.05 (4H, q, J=7.2 Hz), 1.72-1.52 (6H, m), 1.44-1.11 (62H, m), 1.05 (18H, s), 0.93-0.84 (6H, m).

ESI-MS: m/z 663.3 [M/2+1]⁺

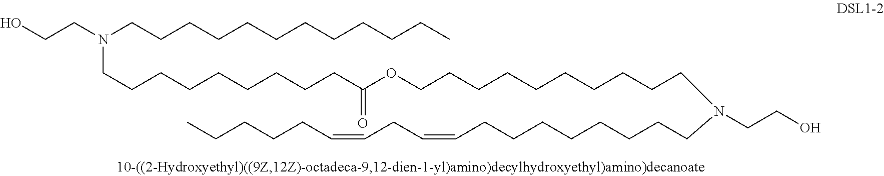 [SMILES: CCCCC/C=C\C/C=C\CCCCCCCCN(CCO)CCCCCCCCCCOC(=O)CCCCCCCCCN(CCO)CCCCCCCCCCCC]

To a solution of 11 (520 mg, 0.39 mmol, 1 equiv.) in dry THF (10 mL), TBAF (1.6 mL, 1.57 mmol, 4.0 equiv.) was added. The reaction was stirred for 3 hr at room temperature and quenched with sat. NH₄Cl and extracted with the mixture of ethyl acetate and diethyl ether (20:80). Then the combined organic portion was washed with sat. NH₄Cl solution (3 times) to remove TBAF completely. The solvent was evaporated and the residue purified by column chromatography using 0-15% isopropanol in chloroform to obtain the DSL1-2 (244 mg, 74%) as a yellow color viscus liquid.

FIGS. 2A and 2B represent the ¹H NMR spectra and ESI-MS of DSL1-2, respectively.

¹H NMR (400 MHz, CDCl3): δ 5.44-5.26 (4H, m), 4.05 (2H, t, J=6.4 Hz), 3.55 (4H, t, J=5.2 Hz), 2.77 (2H, t, J=6.4 Hz), 2.60 (4H, t, J=5.2 Hz), 2.47 (8H, t, J=7.2 Hz), 2.28 (2H, t, J=7.2 Hz), 2.05 (4H, q, J=6.8 Hz), 1.61 (6H, quint, J=6.8 Hz), 1.50-1.39 (8H, m), 1.38-1.13 (54H, m), 0.93-0.82 (6H, m).

ESI-MS: m/z 848.3 [M+1]⁺; 424.7 [M/2+1]⁺

Synthesis of DSL1-3

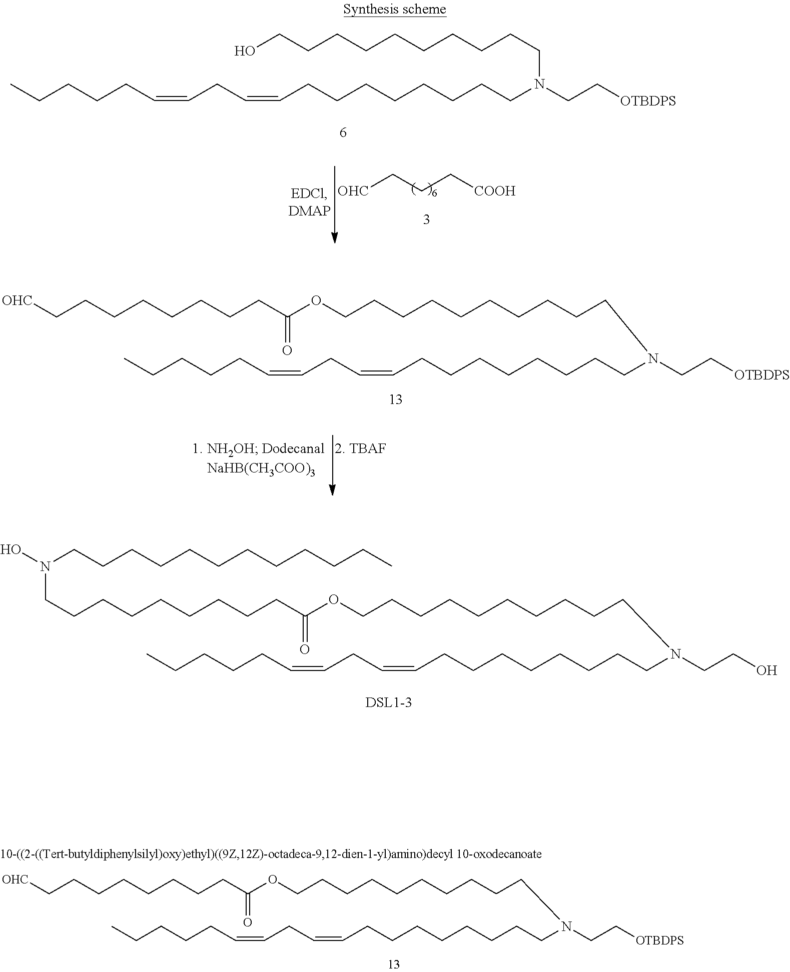

The alcohol 6 (512 mg, 0.73 mmol, 1.0 equiv.), 10-oxodecanioc acid 3 (202 mg, 1.09 mmol, 1.5 equiv.), EDC·HCl (277 mg, 1.45 mmol, 2.0 equiv.) and DMAP (18 mg, 0.14 mmol, 0.2 equiv.) were dissolved in dry CH₂Cl₂ (10 mL) under argon atmosphere and stirred for 24 hr at room temperature. The reaction mixture was quenched with sat. NaHCO₃ and extracted with CH₂Cl₂ (3 times). The organic portion was washed with brine solution and dried over anhydrous Na₂SO₄. The solvent was evaporated on rotary evaporator and the crude product was purified by column chromatography using 0-5% isopropanol in chloroform to obtain the 13 (564 mg, 89%) as a pale yellow color viscus liquid.

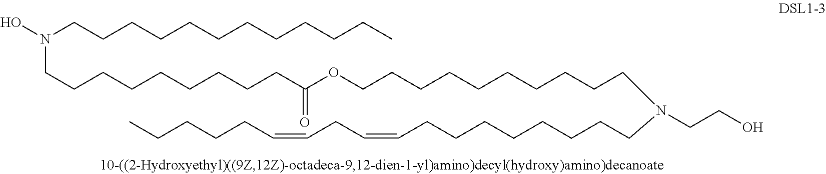 [SMILES: CCCCC/C=C\C/C=C\CCCCCCCCN(CCO)CCCCCCCCCCOC(=O)CCCCCCCCCN(O)CCCCCCCCCCCC]

To a suspension of hydroxylamine hydrochloride (34 mg, 0.48 mmol, 1.0 equiv.) in dry CH₂Cl₂ (5 mL), trimethylamine (67 μL, 0.48 mmol, 1.0 equiv.) was added under argon atmosphere and stirred for 5 min at room temperature. Later, a solution of aldehyde 13 (420 mg, 0.48 mmol, 1.0 equiv.) in dry CH₂Cl₂ (10 mL) was added drop wisely and stirred it for 2 hr. After that, the reaction mixture was diluted with another 10 mL of dry CH₂Cl₂, and sodium triacetoxyborohydride (150 mg, 0.72 mmol, 1.5 equiv.) was added portion wise over a period of 10 min and stirred for another 10 min. Then a solution of dodecanal (160 μl, 0.721 mmol, 1.5 equiv.) in dry CH₂Cl₂ (10 mL) was added drop wisely to the reaction mixture and stirred for another 10 min. Later the remaining amount of sodium triacetoxyborohydride (150 mg, 0.72 mmol, 1.5 equiv.) was added portion wise over a period of 15 min and left for the overnight stirring at room temperature under argon atmosphere. The reaction was quenched with sat·NaHCO₃ solution and extracted with CH₂Cl₂ (3 times). The solvent was evaporated on rotary evaporator and the crude product was dissolved in THF (10 mL) and TBAF (1.0 mL, 1.0 M in THF, 0.96 mmol, 2.0 equiv.) was added. The reaction was stirred for 3 hr at room temperature and quenched with sat. NH₄Cl, and extracted with 20% ethyl acetate in diethyl ether (3 times). The combined organic portion was washed with sat. NH₄Cl solution (3 times) to remove TBAF completely. The solvent was evaporated under reduced pressure and the residue purified by column chromatography using 0-5% isopropanol in chloroform to bestow the GS-198 (208 mg, 53%) as a pale yellowish semi solid. FIGS. 3A and 3B represent the ¹H NMR spectra and ESI-MS of DSL1-3, respectively.

¹H NMR (400 MHz, CDCl3): δ 5.44-5.27 (4H, m), 4.06 (2H, t, J=6.8 Hz), 3.69-3.59 (2H, br), 2.77 (2H, t, J=6.4 Hz), 2.74-2.67 (2H, br), 2.63 (4H, t, J=7.6 Hz), 2.67-2.51 (4H, br), 2.28 (2H, t, J=7.6 Hz), 2.05 (4H, q, J=6.8 Hz), 1.66-1.45 (14H, m), 1.38-1.13 (54H, m), 0.89 (3H, t, J=6.8 Hz), 0.88 (3H, t, J=6.8 Hz).

ESI-MS: m/z 820.28 [M+1]⁺; 410.68 [M/2+1]⁺

Synthesis of DSL1-4

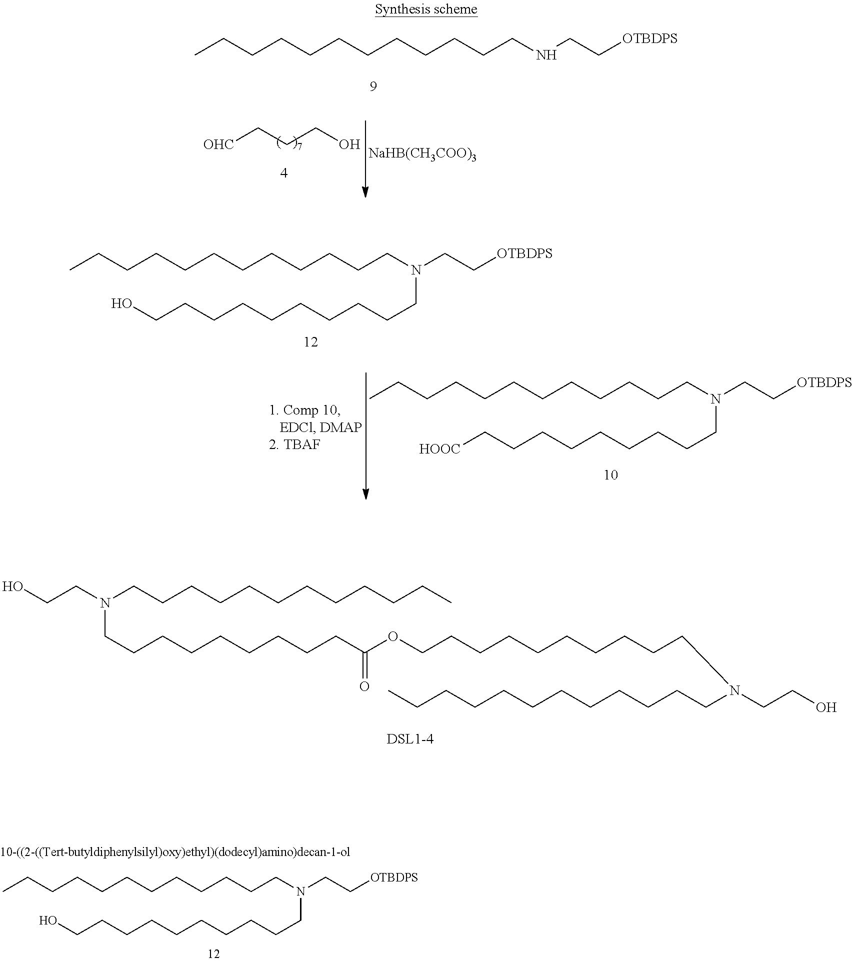

The compound 9 (982 mg, 2.1 mmol, 1.0 equiv.) and 10-hydroxydecanal 4 (470 mg, 2.73 mmol, 1.3 equiv.) were dissolved in dry dioxane (40 mL) under argon atmosphere and stirred for 1 hr. at room temperature. Then sodium triacetoxyborohydride (886 mg, 4.20 mmol, 2.0 equiv.) was added and stirred for 24 hr at the same temperature. The reaction mixture was quenched with sat·NaHCO₃ solution followed by extract with CH₂Cl₂ (3×30 mL). The organic layer was washed with brine solution and dried over anhydrous Na₂SO₄. The solvent was evaporated on rotary evaporator and the residue was purified by column chromatography using 0-2% MeOH in CHCl₃ to obtain 12 (1.21 g, 93%) as a yellowish viscus liquid.

¹H NMR (400 MHz, CDCl3): δ 7.71-7.60 (4H, m), 7.47-7.33 (6H, m), 3.90-3.70 (2H, br), 3.63 (2H, t, J=6.4 Hz), 2.92-2.30 (6H, m), 1.81-1.48 (6H, m), 1.39-1.13 (30H, m), 1.05 (9H, s), 0.88 (3H, t, J=7.2 Hz).

ESI-MS: m/z 624.9 [M+1]⁺

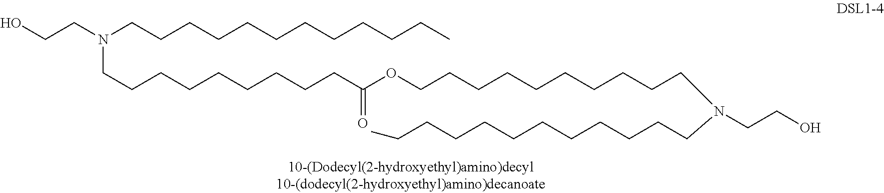 [SMILES: CCCCCCCCCCCCN(CCO)CCCCCCCCCCOC(=O)CCCCCCCCCN(CCO)CCCCCCCCCCCC]

The alcohol 12 (300 mg, 0.48 mmol, 1.0 equiv.), acid 10 (400 mg, 0.62 mmol, 1.3 equiv.), EDC·HCl (183 mg, 0.96 mmol, 2.0 equiv.) and DMAP (12 mg, 0.10 mmol, 0.2 equiv.) were dissolved in dry CH₂Cl₂ (10 mL) under argon atmosphere and stirred for 24 hr at room temperature. The reaction mixture was quenched with sat. NaHCO₃ and extracted with CH₂Cl₂ (3 times). The organic portion was washed with brine solution and dried over anhydrous Na₂SO₄. The solvent was evaporated on rotary evaporator and the crude product was dissolved in THF (5 mL) and TBAF (2.0 mL, 1.0 M in THF, 1.92 mmol, 4.0 equiv.) was added. The reaction was stirred for 3 hr at room temperature and quenched with sat. NH₄Cl, and extracted with 20% ethyl acetate in diethyl ether (3 times). The combined organic portion was washed with sat. NH₄Cl solution (3 times) to remove TBAF completely. The solvent was evaporated under reduced pressure and the residue purified by column chromatography using 0-12% isopropanol in chloroform to obtain the DSL1-4 (303 mg, 82%) as a pale yellowish viscus liquid.

FIGS. 4A and 4B represent the ¹H NMR spectra and ESI-MS of DSL1-4, respectively.

¹H NMR (400 MHz, CDCl3): δ 4.06 (2H, t, J=6.4 Hz), 3.99-3.92 (4H, m), 3.17-3.09 (4H, m), 3.08-2.97 (8H, m), 2.29 (2H, t, J=7.2 Hz), 1.88-1.71 (10H, m), 1.67-1.50 (8H, m), 1.40-1.18 (52H, m), 0.88 (6H, t, J=7.2 Hz).

ESI-MS: m/z 768.2 [M+1]⁺; 384.7 [M/2+1]⁺

Synthesis of DSL1-5

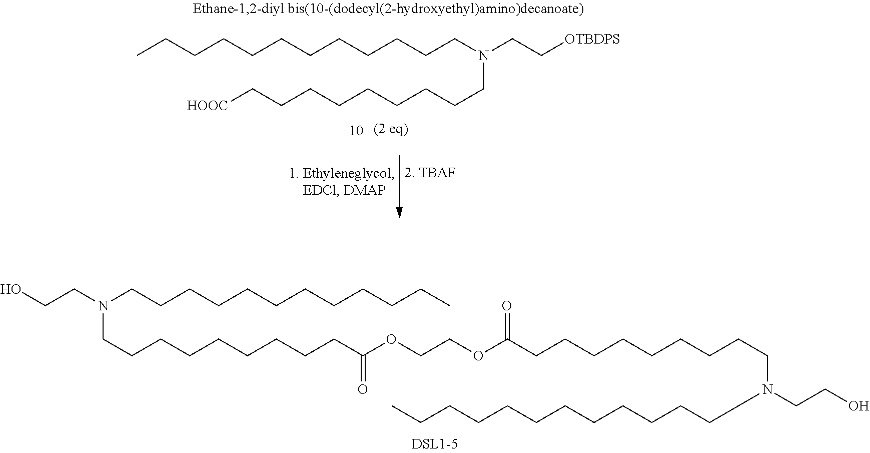

The acid 10 (700 mg, 1.1 mmol, 2.4 equiv.), EDC·HCl (351 mg, 1.84 mmol, 4.0 equiv.) and DMAP (17 mg, 0.14 mmol, 0.3 equiv.) were dissolved in dry CH₂Cl₂ (5 mL) under argon atmosphere and ethylene glycol (26 μL, 0.46 mmol, 1.0 equiv.) was added, and stirred for 24 hr at room temperature. Then the reaction mixture was quenched with sat. NaHCO₃ and extracted with CH₂Cl₂ (3 times). The organic portion was washed with brine solution and dried over anhydrous Na₂SO₄. The solvent was evaporated on rotary evaporator and the crude product was dissolved in THF (5 mL) and TBAF (1.85 mL, 1.0 M in THF, 1.85 mmol, 4.0 equiv.) was added. The reaction was stirred for 3 hr at room temperature and quenched with sat. NH₄Cl, and extracted with 20% ethyl acetate in diethyl ether (3 times). The combined organic portion was washed with sat. NH₄Cl solution (3 times) to remove TBAF completely. The solvent was evaporated under reduced pressure and the residue purified by column chromatography using 0-20% isopropanol in chloroform to obtain the DSL1-5 (280 mg, 74%) as a pale color viscus liquid.

FIGS. 5A and 5B represent the ¹H NMR spectra and ESI-MS of DSL1-5, respectively.

¹H NMR (400 MHz, CDCl3): δ 4.27 (4H, s), 3.93-3.78 (4H, br), 3.07-2.97 (4H, br), 2.96-2.82 (8H, br), 2.32 (4H, t, J=7.6 Hz), 1.80-1.66 (10H, m), 1.62 (8H, quint, J=7.2 Hz), 1.39-1.19 (50H, m), 0.88 (6H, t, J=6.8 Hz).

ESI-MS: m/z 826.2 [M+1]⁺; 413.7 [M/2+1]⁺

Synthesis of DSL2-1

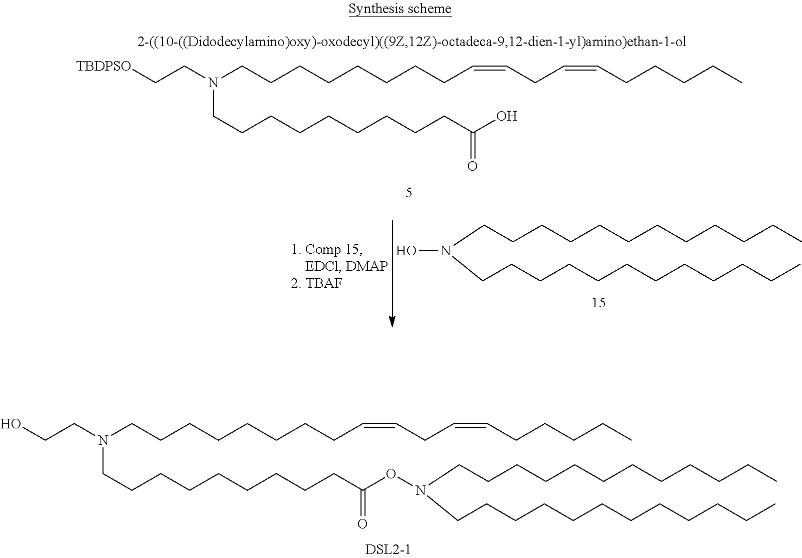

To a solution of acid 5 (400 mg, 0.56 mmol, 1.0 equiv.), EDC·HCl (212 mg, 1.11 mmol, 2.0 equiv.) and DMAP (14 mg, 0.011 mmol, 0.2 equiv.) in dry CH₂Cl₂ (10 mL), a solution of hydroxyl amine (226 mg, 0.61 mmol, 1.1 equiv.) in dry THF (5 mL) was added under argon atmosphere, and stirred for 24 hr at room temperature. Then the reaction was quenched with sat. NaHCO₃ and extracted with CH₂Cl₂ (3 times). The organic portion was washed with brine solution and dried over anhydrous Na₂SO₄. The solvent was evaporated on rotary evaporator and the crude product was dissolved in THF (5 mL) and TBAF (1.1 mL, 1.0 M in THF, 1.11 mmol, 2.0 equiv.) was added. The reaction was stirred for 3 hr at room temperature and quenched with sat. NH₄Cl, and extracted with 20% ethyl acetate in diethyl ether (3 times). The combined organic portion was washed with sat. NH₄Cl solution (3 times) to remove TBAF completely. The solvent was evaporated and the residue purified by column chromatography using 0-5% isopropanol in chloroform to obtain the GS-208 (315 mg, 68%) as a pale color liquid.

FIGS. 6A and 6B represent the ¹H NMR spectra and ESI-MS of DSL2-1, respectively.

¹H NMR (400 MHz, CDCl3): δ 5.44-5.27 (4H, m), 3.70-3.53 (2H, br), 2.79 (4H, t, J=8.0 Hz), 2.77 (2H, t, J=7.2 Hz), 2.74-2.63 (2H, br), 2.62-2.43 (4H, br), 2.27 (2H, t, J=7.6 Hz), 2.17 (1H, s), 2.05 (4H, q, J=6.8 Hz), 1.64 (6H, quint, J=7.2 Hz), 1.58-1.43 (8H, m), 1.35-1.18 (58H, m), 0.89 (3H, t, J=6.4 Hz), 0.88 (6H, t, J=6.8 Hz).

ESI-MS: m/z 832.3 [M+1]⁺; 417.0 [M/2+1]⁺

Synthesis of DSL2-2

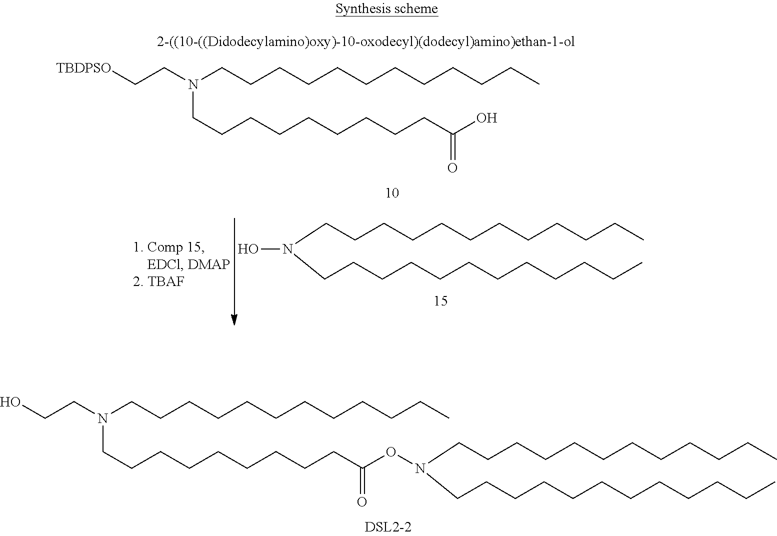

To a solution of acid 10 (460 mg, 0.72 mmol, 1.0 equiv.), EDC·HCl (275 mg, 1.44 mmol, 2.0 equiv.) and DMAP (17 mg, 0.144 mmol, 0.2 equiv.) in dry CH₂Cl₂ (10 mL), a solution of hydroxyl amine 15 (293 mg, 0.79 mmol, 1.1 equiv.) in dry THF (5 mL) was added under argon atmosphere, and stirred for 24 hr at room temperature. Then the reaction was quenched with sat. NaHCO₃ and extracted with CH₂Cl₂ (3 times). The organic portion was washed with brine solution and dried over anhydrous Na₂SO₄. The solvent was evaporated on rotary evaporator and the crude product was dissolved in THF (5 mL) and TBAF (1.5 mL, 1.0 M in THF, 1.44 mmol, 2.0 equiv.) was added. The reaction was stirred for 3 hr at room temperature and quenched with sat. NH₄Cl, and extracted with 20% ethyl acetate in diethyl ether (3 times). The combined organic portion was washed with sat. NH₄Cl solution (3 times) to remove TBAF completely. The solvent was evaporated and the residue purified by column chromatography using 0-5% isopropanol in chloroform to obtain the DSL2-2 (390 mg, 72%) as a pale yellowish liquid.

FIGS. 7A and 7B represent the ¹H NMR spectra and ESI-MS of DSL2-2, respectively.

¹H NMR (400 MHz, CDCl3): δ 3.79-3.57 (2H, br), 2.92-2.70 (2H, br), 2.79 (4H, t, J=7.6 Hz), 2.50-2.52 (4H, m), 2.27 (2H, t, J=7.6 Hz), 2.17 (1H, s), 1.79-1.43 (14H, m), 1.35-1.18 (60H, m), 0.88 (9H, t, J=7.2 Hz).

ESI-MS: m/z 752.2 [M+1]⁺; 376.7 [M/2+1]⁺

Synthesis of DSL4-1

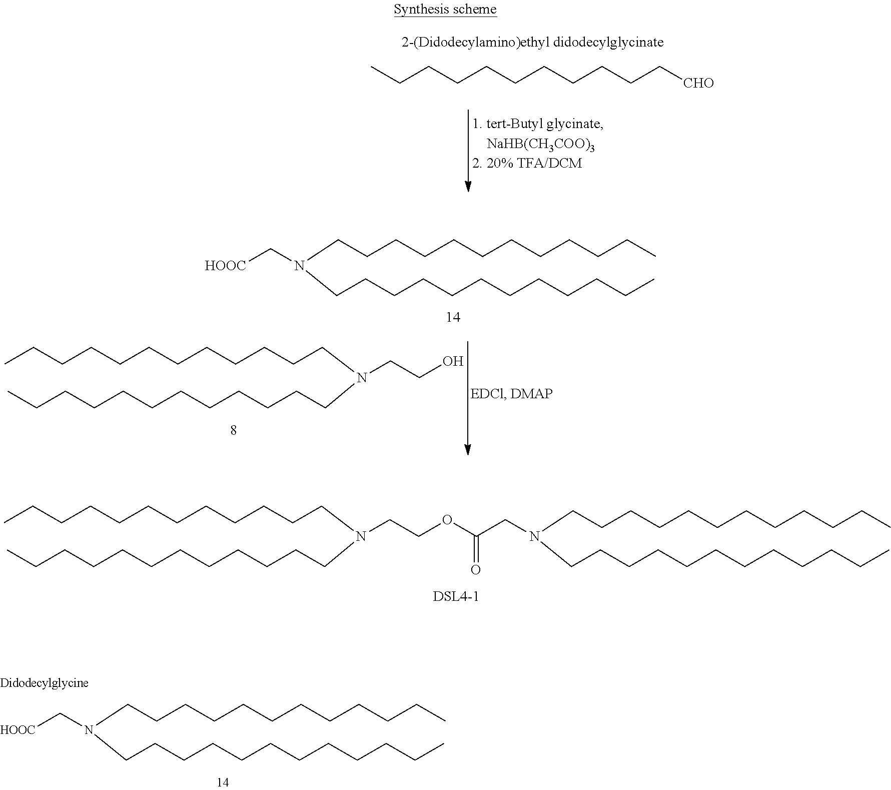 [SMILES: CCCCCCCCCCCC=O ; CCCCCCCCCCCCN(CCCCCCCCCCCC)CC(=O)O ; CCCCCCCCCCCCN(CCO)CCCCCCCCCCCC ; CCCCCCCCCCCCN(CCCCCCCCCCCC)CCOC(=O)CN(CCCCCCCCCCCC)CCCCCCCCCCCC ; CCCCCCCCCCCCN(CCCCCCCCCCCC)CC(=O)O]

To a suspension of tert-Butylglycinate hydrochloride (635 mg, 3.80 mmol, 0.5 equiv.) in dry CH₂Cl₂ (20 mL), trimethylamine (0.53 mL, 3.80 mmol, 0.5 equiv.) was added under argon atmosphere and stirred for 10 min at room temperature. Later, a solution of dodecanal (1.4 g, 7.60 mmol, 1.0 equiv.) in dry CH₂Cl₂ (10 mL) was added drop wisely and stirred for 2 hr. After that, the reaction mixture was diluted with another 20 mL of dry CH₂Cl₂, and sodium triacetoxyborohydride (2.40 g, 11.40 mmol, 1.5 equiv.) was added portion wise over a period of 15 min and stirred for 24 hr at room temperature. Later, the reaction was quenched with sat·NaHCO₃ solution and extracted with CH₂Cl₂. The solvent was evaporated on rotary evaporator and the crude product was dissolved in 20% TFA/CH₂Cl₂ (20 mL) and stirred for 4 hr at room temperature. After that, reaction was quenched with saturated·NaHCO₃ solution and extracted with CH₂Cl₂. The solvent was removed and the residue was purified by column chromatography using 0-15% isopropanol in chloroform to obtain didodecylglycine 14 (750 mg, 48%) as a white solid.

¹H NMR (400 MHz, CDCl3): δ 3.44 (2H, s), 3.01 (4H, t, J=8.0 Hz), 1.75-1.59 (4H, m), 1.40-1.18 (36H, m), 0.88 (6H, t, J=6.8 Hz).

ESI-MS: m/z 412.7 [M+1]⁺

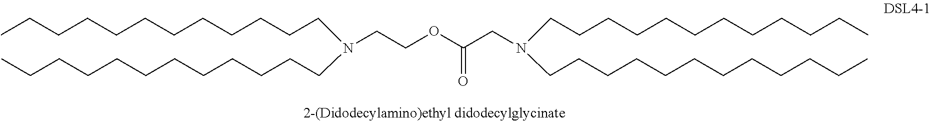 [SMILES: CCCCCCCCCCCCN(CCCCCCCCCCCC)CCOC(=O)CN(CCCCCCCCCCCC)CCCCCCCCCCCC]

To a solution of ethanol amine 8 (209 mg, 0.527 mmol, 1.0 equiv.), EDC·HCl (201 mg, 1.05 mmol, 2.0 equiv.) and DMAP (12 mg, 0.1 mmol, 0.2 equiv.) in dry CH₂Cl₂ (10 mL), a solution of acid 14 (260 mg, 0.63 mmol, 1.2 equiv.) in dry DMF (5 mL) was added under argon atmosphere, and stirred for 24 hr at room temperature. Then the reaction was quenched with sat. NaHCO₃ and extracted with ethyl acetate (3 times). The organic portion was washed with brine solution and dried over anhydrous Na₂SO₄. The solvent was evaporated on rotary evaporator and the residue was purified by column chromatography using 0-10% EtOAc in hexane to obtain DSL4-1 (310 mg, 75%) as a colorless liquid. FIGS. 8A and 8B represent the ¹H NMR spectra and ESI-MS of DSL4-1, respectively.

¹H NMR (400 MHz, CDCl3): δ 4.15 (2H, t, J=6.4 Hz), 3.31 (2H, s), 2.68 (2H, t, J=6.4 Hz), 2.54 (4H, t, J=8.0 Hz), 2.43 (4H, t, J=7.6 Hz), 1.50-1.36 (8H, m), 1.35-1.18 (72H, m), 0.88 (12H, t, J=7.2 Hz).

ESI-MS: m/z 792.3 [M+1]⁺; 396.7 [M/2+1]⁺

Synthesis of DSL4-2

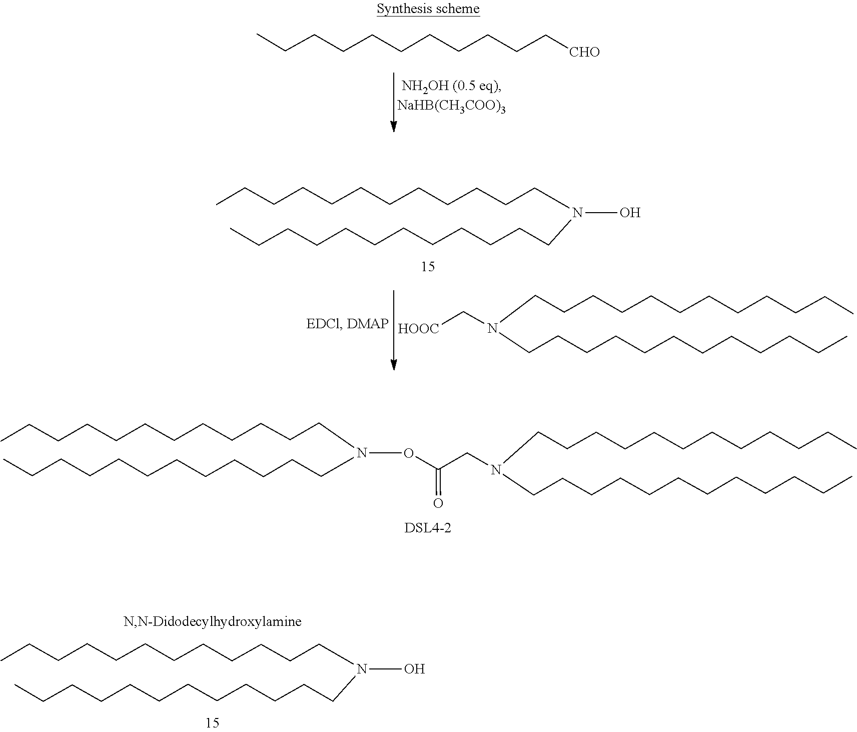

To a suspension of hydroxylamine hydrochloride (481 mg, 6.97 mmol, 0.4 equiv.) in dry CH₂Cl₂ (30 mL), trimethylamine (0.97 mL, 6.97 mmol, 0.4 equiv.) was added under argon atmosphere and stirred for 10 min at room temperature. Then, a solution of dodecanal (3.21 g, 17.44 mmol, 1.0 equiv.) in dry CH₂Cl₂ (20 mL) was added drop wisely to the reaction mixture and stirred for 2 hr. After that, the reaction mixture was diluted with another 30 mL of dry CH₂Cl₂ and sodium triacetoxyborohydride (5.52 g, 26.16 mmol, 1.5 equiv.) was added portion wise over a period of 15 min and stirred for 24 hr at room temperature. Later, the reaction was quenched with sat·NaHCO₃ solution. The product was precipitated and floated on the reaction mixture. The solvent dichloromethane which used in the reaction was removed by separating funnel, and the product was extracted with ethyl acetate from the aqueous portion. The solvent was evaporated and the product was purified by recrystallization using 20% EtOAc in hexane to obtain the desired hydroxyl amine 15 (2.13 g, 83%) as a white fluffy solid.

¹H NMR (400 MHz, CDCl3): δ 2.62 (4H, t, J=7.6 Hz), 1.68-1.47 (4H, m), 1.39-1.15 (36H, m), 0.88 (6H, t, J=6.8 Hz).

ESI-MS: m/z 370.7 [M+1]⁺

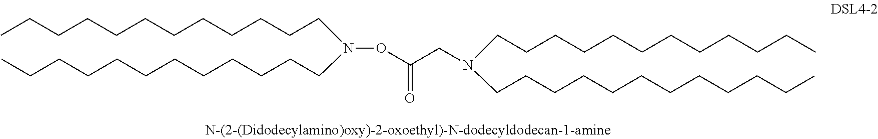 [SMILES: CCCCCCCCCCCCN(CCCCCCCCCCCC)CC(=O)ON(CCCCCCCCCCCC)CCCCCCCCCCCC]

To a solution of EDC·HCl (216 mg, 1.13 mmol, 2.0 equiv.) and DMAP (13 mg, 0.11 mmol, 0.2 equiv.) in dry CH₂Cl₂ (10 mL), solution of hydroxyl amine 15 (209 mg, 0.567 mmol, 1.0 equiv.) in dry THF (5 mL) and solution of acid 14 (280 mg, 0.68 mmol, 1.2 equiv.) in dry DMF (5 mL) were added under argon atmosphere, and stirred for 24 hr at room temperature. Then the reaction was quenched with sat. NaHCO₃ and extracted with ethyl acetate (3 times). The organic portion was washed with brine solution and dried over anhydrous Na₂SO₄. The solvent was evaporated on rotary evaporator and the crude product was purified by column chromatography using 0-5% EtOAc in hexane to obtain the DSL4-2 (334 mg, 79%) as a low melting white solid.

FIGS. 9A and 9B represent the ¹H NMR spectra and ESI-MS of DSL4-2, respectively.

¹H NMR (400 MHz, CDCl3): δ 3.32 (2H, s), 2.81 (4H, t, J=8.0 Hz), 2.56 (4H, t, J=8.0 Hz), 1.55-1.38 (8H, m), 1.37-1.14 (72H, m), 0.88 (12H, t, J=7.2 Hz).

ESI-MS: m/z 764.2 [M+1]⁺

Synthesis of DSL3c-1

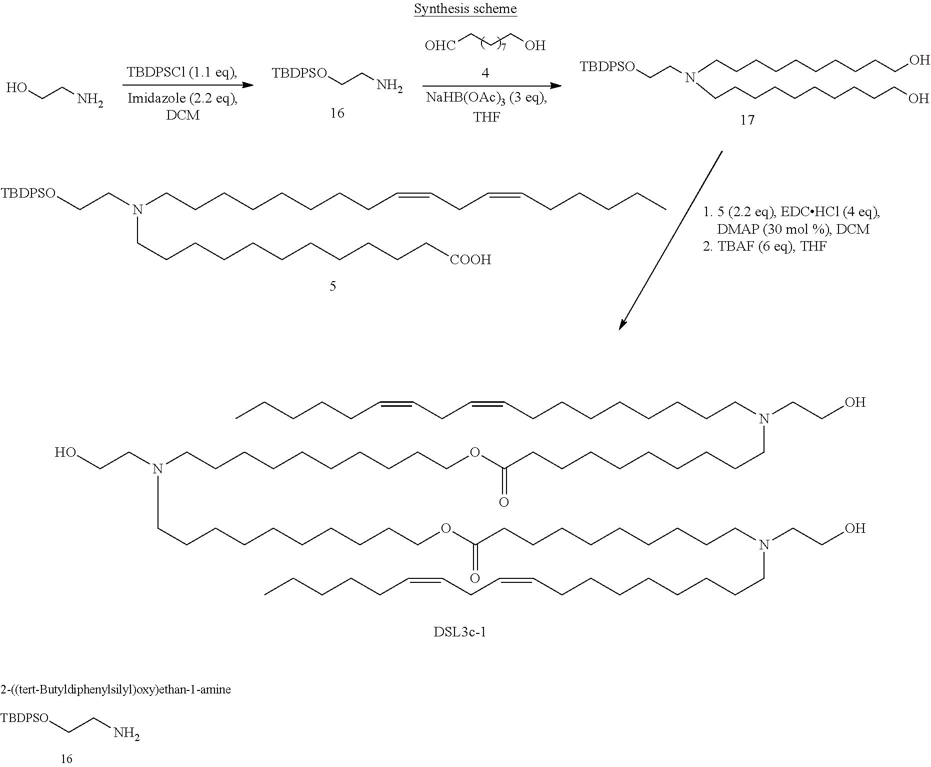

To a solution of ethanolamine (0.3 mL, 4.92 mmol, 1 equiv.) and imidazole (736 mg, 10.82 mmol, 2.2 equiv.) in dry CH₂Cl₂ (30 mL), TBDPS-Cl (1.40 mL, 5.41 mmol, 1.1 equiv.) was added drop-wise under argon atmosphere and stirred it for overnight at room temperature. Then the reaction mixture was poured in brain solution and extracted with CH₂Cl₂. The organic layer was dried over anhydrous Na₂SO₄ and concentrated under reduced pressure. The crude product was purified by column chromatography using 0-5% MeOH in CHCl₃ to afford TBDPS protected ethanolamine 16 in quantitative yield as a pale yellowish liquid.

¹H NMR (400 MHz, CDCl₃): δ 7.74-7.63 (4H, m), 7.47-7.34 (6H, m), 3.68 (2H, t, J=5.6 Hz), 2.81 (2H, t, J=5.2 Hz), 1.06 (9H, s).

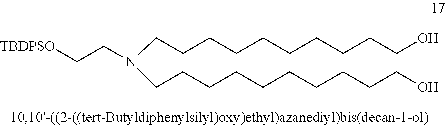

TBDPS protected ethanolamine 16 (226 mg, 0.75 mmol, 1.0 equiv.) and 10-hydroxydecanal 4 (260 mg, 1.51 mmol, 2.0 equiv.) were dissolved in dry THF (30 mL) under nitrogen atmosphere and stirred for 2 hr at room temperature. Then sodium triacetoxyborohydride (480 mg, 2.27 mmol, 3.0 equiv.) was added portion wise over a period of 15 min and stirred for 24 hr at the same temperature. Later the reaction was quenched with sat. NaHCO₃ solution followed by extract with ethyl acetate (3 times). The organic layer was washed with brine solution and dried over anhydrous Na₂SO₄. The solvent was evaporated on rotary evaporator and the residue was purified by column chromatography using 0-10% MeOH in CHCl₃ to provide 17 (450 mg, 98%) as a pale yellowish liquid.

¹H NMR (400 MHz, CDCl₃): δ 7.72-7.62 (4H, m), 7.45-7.32 (6H, m), 3.72 (2H, t, J=6.4 Hz), 3.63 (4H, t, J=6.4 Hz), 2.72-2.55 (2H, br), 2.50-2.31 (4H, br), 5.56 (8H, quint, J=6.8 Hz), 1.45-1.12 (24H, m) 1.04 (9H, s).

ESI-MS: m/z 612.9 [M+1]⁺

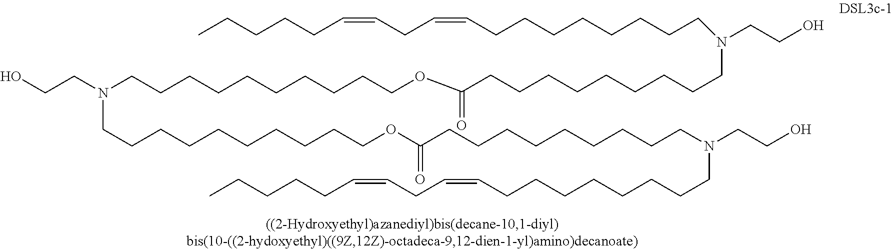 [SMILES: CCCCC/C=C\C/C=C\CCCCCCCCN(CCO)CCCCCCCCCC(=O)OCCCCCCCCCCN(CCO)CCCCCCCCCCOC(=O)CCCCCCCCCN(CCO)CCCCCCCC/C=C\C/C=C\CCCCC]

The diol 17 (162 mg, 0.26 mmol, 1.0 equiv.), acid 5 (420 mg, 0.58 mmol, 2.2 equiv.), EDC·HCl (202 mg, 1.1 mmol, 4.0 equiv.) and DMAP (10 mg, 0.08 mmol, 0.3 equiv.) were dissolved in dry CH₂Cl₂ (10 mL) under nitrogen atmosphere and stirred for 24 hr at room temperature. Later, the reaction was quenched with sat. NaHCO₃ and extracted with CH₂Cl₂ (3 times). The organic portion was washed with brine solution and dried over anhydrous Na₂SO₄. The solvent was evaporated on rotary evaporator and the crude product was dissolved in THF (5 mL) and TBAF (1.6 mL, 1.0 M in THF, 1.56 mmol, 6.0 equiv.) was added. The reaction was stirred for 5 hr at room temperature and quenched with sat. NH₄Cl, and extracted with 30% ethyl acetate in diethyl ether. Then the combined organic portion was washed with sat. NH₄Cl solution (3 times) to remove TBAF completely. The solvent was evaporated under reduced pressure and the residue purified by column chromatography using 0-10% methanol in chloroform to obtain the DSL3c-1 (195 mg, 58%) as a yellowish viscus liquid.

¹H NMR (400 MHz, CDCl₃): δ 5.44-5.25 (8H, m), 4.06 (4H, t, J=6.8 Hz), 3.92-2.78 (6H, br), 3.09-2.94 (6H, br), 2.94-2.80 (12H, br), 2.77 (4H, t, J=6.4 Hz), 2.29 (4H, t, J=7.6 Hz), 2.05 (8H, q, J=6.8 Hz), 1.78-1.65 (12H, br), 1.61 (8H, quint, J=6.8 Hz), 1.42-1.18 (76H, m), 0.89 (6H, t, J=6.8 Hz).

ESI-MS: m/z 12.98.7 [M+1]⁺; 649.8 [M/2+1]⁺; 433.0 [M/3+1]⁺.

Synthesis of DSL1-50

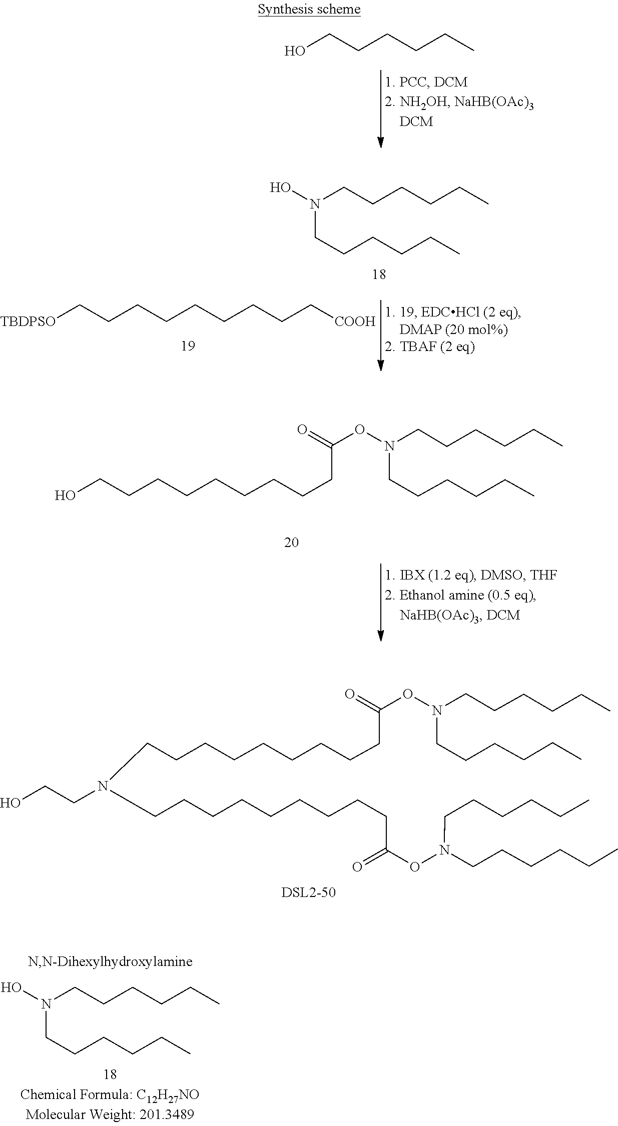

To a solution of hexanol (4.0 g, 39.2 mmol, 1 equiv.) in dry CH₂Cl₂ (100 mL), molecular sieves (4 Å MS) were added under argon atmosphere. Then PCC (12.64 g, 58.8 mmol, 1.5 equiv.) was added portion wise over a period of 10 min and stirred for 2 hr at room temperature. After completing the reaction filtered it through a silica gel pad using CH₂Cl₂ to remove PCC. The solvent was evaporated on rotary evaporator with low vacuum to get a solution of hexanal (20 mL). The aldehyde solution was dried over anhydrous sodium sulfate and subjected to the next step.

To a suspension of hydroxylamine hydrochloride (871 mg, 11.76 mmol, 0.3 equiv.) in dry CH₂C₁₂ (20 mL), dry trimethylamine (1.6 mL, 11.76 mmol, 0.3 equiv.) was added under argon atmosphere and stirred for 10 min at room temperature. Then, a solution of hexanal in dry CH₂Cl₂ (30 mL) was added drop wisely and stirred for 2 hr. After that, the reaction mixture was diluted with another 50 mL of dry CH₂Cl₂ and sodium triacetoxyborohydride (7.4 g, 35.3 mmol, 0.9 equiv.) was added portion wise and left for the overnight stirring at room temperature. Later, the reaction was quenched with sat·NaHCO₃ solution followed by extract with CH₂Cl₂ (3 times). The organic layer was washed with brine solution and dried over anhydrous Na₂SO₄. The solvent was evaporated and the residue was purified by column chromatography using 0-10% EtOAc in Hexane to obtain N,N-dihexylhydroxylamine 18 (2.2 g, 91%).

¹H NMR (400 MHz, CDCl₃): δ 2.64 (4H, t, J=7.6 Hz), 1.58 (4H, quint, J=7.2 Hz), 1.40-1.18 (12H, m), 0.87 (6H, t, J=6.8 Hz).

ESI-MS: m/z 202.4 [M+1]⁺

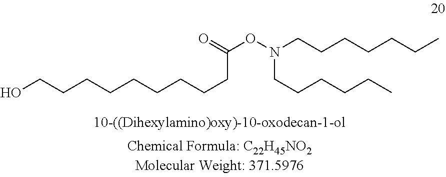 [SMILES: CCCCCCCN(CCCCCC)OC(=O)CCCCCCCCCO]

The above hydroxylamine 18 (500 mg, 2.49 mmol, 1.0 equiv.), 10-((tert-butyldiphenylsilyl)oxy)decanoic acid 19 (1.27 g, 2.98 mmol, 1.2 equiv.), EDC·HCl (950 mg, 4.97 mmol, 2.0 equiv.) and DMAP (60 mg, 0.50 mmol, 0.2 equiv.) were dissolved in dry CH₂Cl₂ (20 mL) under argon atmosphere and left for the overnight stirring at room temperature. Then the reaction was quenched with sat. NaHCO₃ followed by extract with CH₂Cl₂ (3 times) and washed with brine solution and dried over with anhydrous Na₂SO₄. The solvent was evaporated on rotary evaporator and the crude product was dissolved in THF (10 mL) and TBAF (5.0 mL, 1.0 M in THF, 4.97 mmol, 2.0 equiv.) was added. The reaction was stirred for 3 hr at room temperature and quenched with sat. NH₄Cl, and extracted with ethyl acetate (3 times). The solvent was evaporated and the residue purified by column chromatography using 0-5% EtOAc in hexane to obtain the desired alcohol 20 (721 mg, 78%) as a colorless liquid.

¹H NMR (400 MHz, CDCl₃): δ 3.63 (2H, q, J=4.4 Hz), 2.80 (4H, t, J=7.6 Hz), 2.27 (2H, t, J=7.6 Hz), 1.71-1.44 (8H, m), 1.41-1.17 (22H, m), 0.87 (6H, t, J=6.8 Hz).

ESI-MS: m/z 394.5 [M+Na]⁺

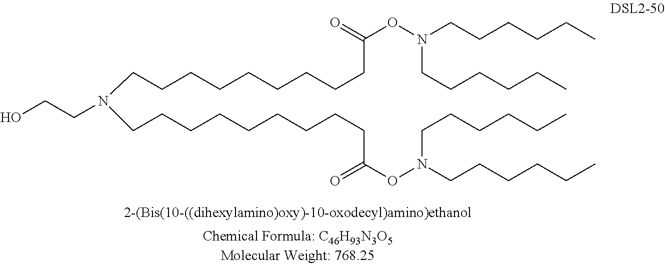 [SMILES: CCCCCCN(CCCCCC)OC(=O)CCCCCCCCCN(CCO)CCCCCCCCCC(=O)ON(CCCCCC)CCCCCC]

To a solution of IBX (704 mg, 45 wt. %, 1.13 mmol, 1.2 equiv.) in DMSO (6 mL), a solution of a solution of alcohol 20 (350 mg, 0.94 mmol, 1.0 equiv.) in THF (6 mL) was added and stirred for hr at room temperature. After that, the reaction was quenched with water (10 mL) and the precipitated solid was removed by filtration. The filtrate was diluted with water and extracted with diethyl ether (4×10 mL). The organic layer dried over anhydrous Na₂SO₄ and the solvent was removed on rotary evaporator. The crude product was dried and subject to the next step. The crude product was dissolved in dry CH₂Cl₂ (20 mL) and ethanolamine (27 μL, 0.45 mmol, 0.475 equiv.) was added under argon atmosphere and stirred for 2 hr. at room temperature. Then sodium triacetoxyborohydride (298 mg, 1.41 mmol, 1.5 equiv.) was added portion wise and stirred for 24 hr at the same temperature. The reaction was quenched with sat·NaHCO₃ solution followed by extract with CH₂Cl₂ (3 times). The organic layer was washed with brine solution and dried over anhydrous Na₂SO₄. The solvent was evaporated and the residue was purified by column chromatography using 0-6% IPA in CHCl₃ to obtain DSL2-50 (195 mg, 57%) as colorless liquid.

¹H NMR (400 MHz, CDCl₃): δ 3.54 (2H, t, J=5.2 Hz), 2.80 (8H, t, J=7.6 Hz), 2.60 (2H, t, J=5.2 Hz), 2.46 (4H, t, J=7.2 Hz), 2.27 (4H, t, J=7.7.2 Hz), 1.64 (6H, quint, J=7.6 Hz), 1.56-1.38 (10H, m), 1.41-1.17 (44H, m), 0.87 (12H, t, J=6.8 Hz).

ESI-MS: m/z 769.01 [M+1]⁺.

Synthesis of DSL1-49

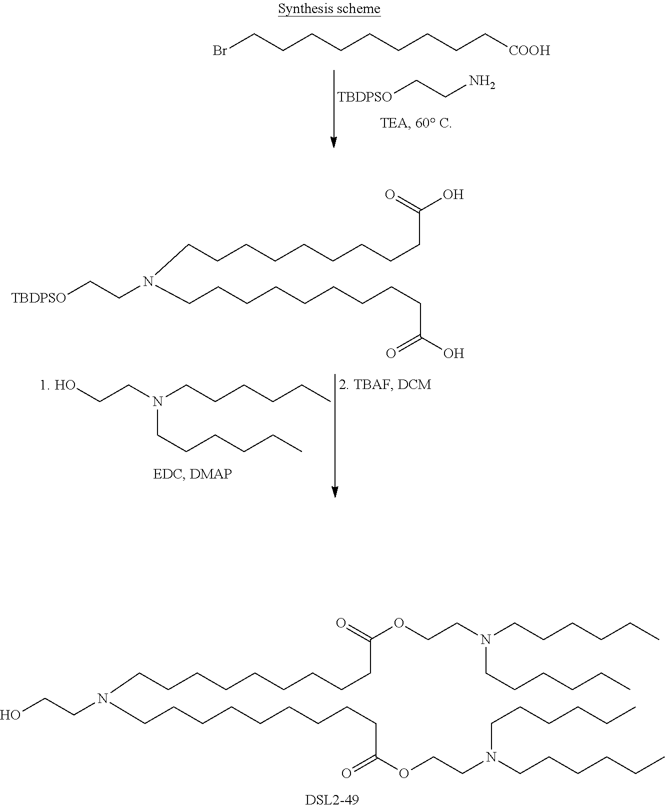

¹H NMR (400 MHz, CDCl3): δ 4.34 (4H, t, J=5.2 Hz), δ 3.54 (2H, t, J=5.2 Hz), 2.80 (12H, t, J=7.6 Hz), 2.60 (2H, t, J=5.2 Hz), 2.46 (4H, t, J=7.2 Hz), 2.27 (4H, t, J=7.7.2 Hz), 1.64 (6H, quint, J=7.6 Hz), 1.56-1.38 (10H, m), 1.41-1.17 (44H, m), 0.87 (12H, t, J=6.8 Hz).

ESI-MS: m/z 825.4 [M+1]⁺.

### Example 2: Preparation and Characterization of LNPs Comprising Ionizable Lipids

Various LNPs comprising different ionizable lipid were prepared by using microfluidic micro mixture (Precision NanoSystems, Vancouver, BC) device. Briefly, one volume of lipid mixtures (ionizable lipid, DSPC, Cholesterol and DMG-PEG at 30:18:49.5:2.5 mole ratios) in ethanol and three volumes of mRNA containing acetate buffer solutions were mixed through the micromixer at a combined flow rate of 12 mL/min. The resultant mixture was dialyzed against PBS (pH 7.4) for 16 hours to remove ethanol.

The prepared LNPs comprising different ionizable lipids were characterized using transmission electron microscopy (TEM) analysis (FIGS. 10A-10G), dynamic light scattering (DLS) and zeta potential measurements. Table 1 represents the measured hydrodynamic diameter (Z-ave; d.nm), polydispersity index (PDI) and zeta potential (mV) of various LNPs. The resultant LNPs were spherical and uniformly distributed with a hydrodynamic diameter size of ˜50-260 nm, depending on the ionizable lipid used. Additionally, LNP surface potentials were negative.

TABLE 1
Physico-chemical characterization of LNPs
made of different ionizable lipids

| | Z-ave (d · nm) | PDI | Zeta (mV) |
|---|---|---|---|
| DSL1-1 | 187.3 | 0.23 | −6.37 |
| DSL1-2 | 197.2 | 0.17 | −7.52 |
| DSL1-3 | 164.8 | 0.17 | −3.90 |
| DSL1-4 | 188.7 | 0.13 | −1.43 |
| DSL1-5 | 190.4 | 0.16 | 1.31 |
| DSL2-1 | 49.2 | 0.24 | −6.97 |
| DSL2-2 | 50.8 | 0.23 | −5.31 |
| DSL4-2 | 72.4 | 0.64 | −5.00 |
| DSL3c-1 | 261.9 | 0.45 | −2.68 |
| DSL2-49 | 63.9 | 0.07 | −5.65 |
| DSL2-50 | 80.3 | 0.04 | −2.68 |

### Example 3: In-Vivo Delivery of mRNA Encoding Luciferase Using LNPs

LNPs comprising DSL1-1, DSL1-2, DSL1-5, DSL1-4, DSL4-2, DSL1-3, DSL2-1, DSL3c-1 DSL2-2, DSL2-49, or DSL2-50, (as described in Example 2), were encapsulated with luciferase-encoding mRNA (mLUC-LNPs) prepared by microfluidic mixing device as mentioned above.

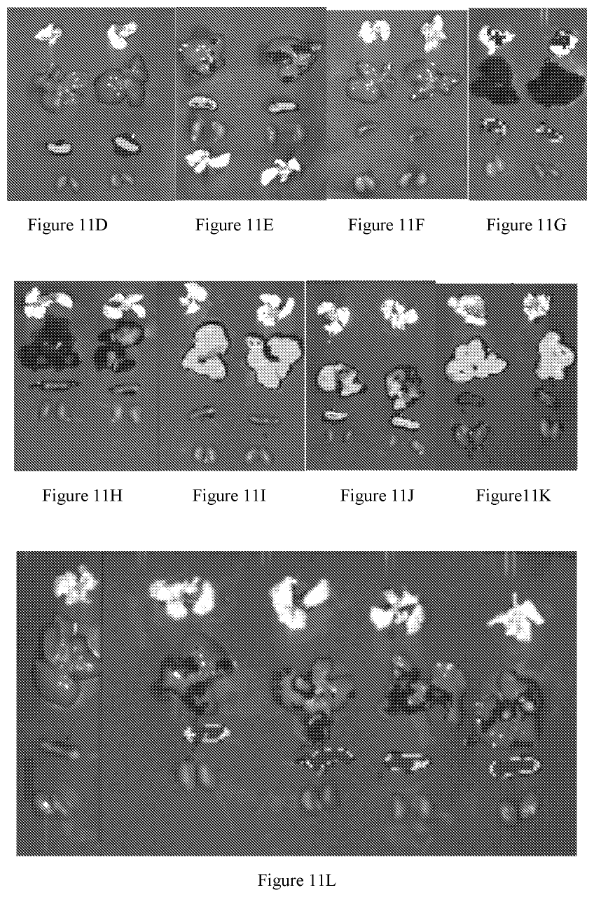

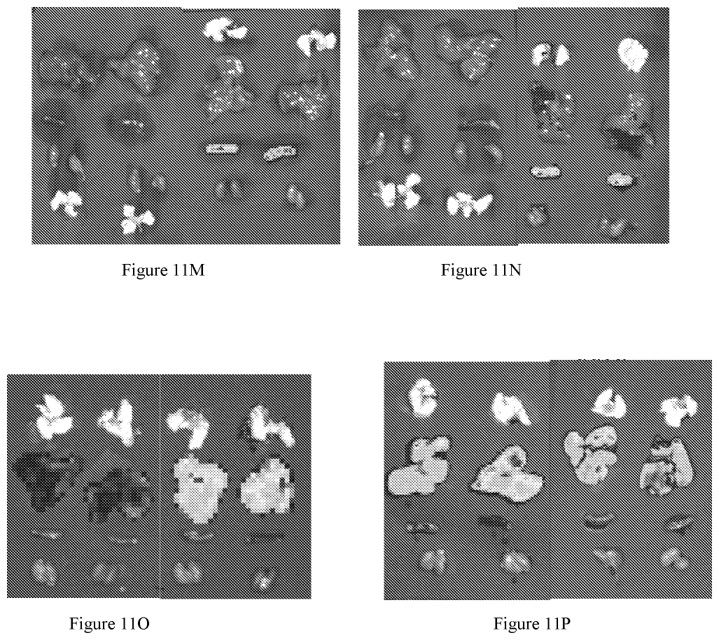

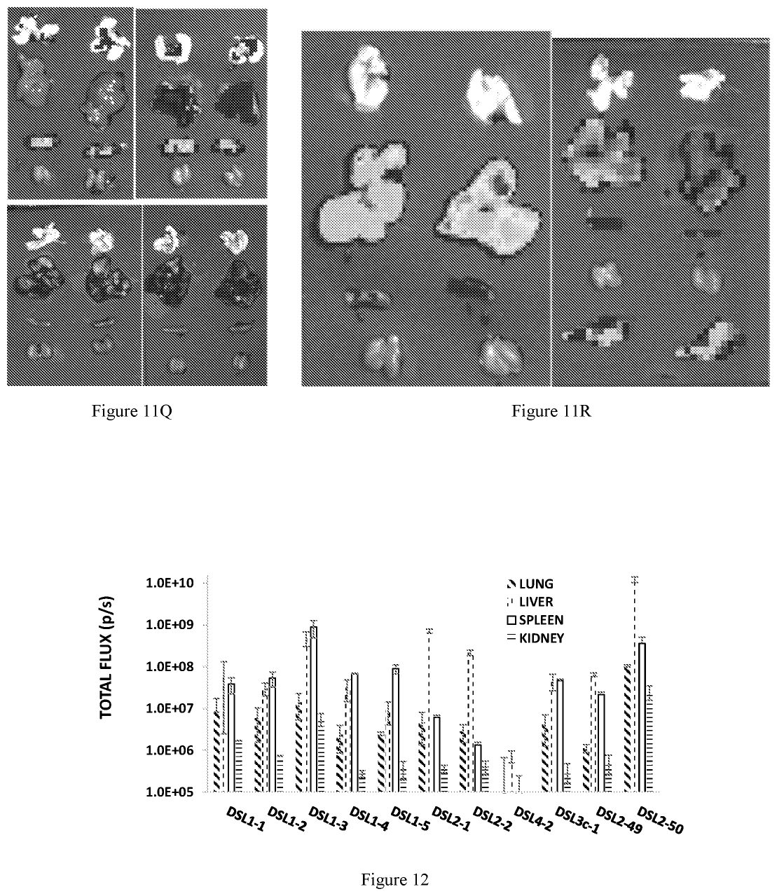

Mice were administered intravenously (IV) with the different mLUC-LNPs at 0.5 mg/kg dose. Mice were sacrificed 7 hr post administration. Then, organs were isolated and luciferase expression was analyzed by IVIS (FIGS. 11A-11K). Luciferase expression in the lung, liver, spleen and kidney were further quantified (FIG. 12).

As can be seen in FIGS. 11A-11K, the bio-distribution of LNPs was different depending on type of LNPs. LNPs comprising DSL2-1 and DSL2-2 mostly taken-up by the liver, whereas DSL1-1 and DSL3c-1 LNPs distributed to both liver and spleen. However, LNPs comprising of DSL1-3, DSL1-4 and DSL1-5 were preferentially taken up by spleen and DSL4-2 LNPs has no effect. Representative bar graphs for the amount luciferase expression are shown in FIG. 12.

In addition, DSL1-3 LNPs comprising different amounts of PEG (1.5 mol % or 2.5 mol %) were compared for bio-distribution. Mice treated and sacrificed; organs prepared for luciferase expression analysis as detailed above. As shown in FIG. 11L, there was no significant different observed in LNP distribution and expression between two formulations. Next, as shown in FIGS. 11M-11P, mRNA-LNPs composed of DSL1-4, 1-5, 2-2 or 2-50 lipids were compared to DSPC or DOPE as co-lipids. Interestingly, DSL1-4 & 1-5 LNPs with DOPE as co-lipid loses its luciferase activity compared to DSPC containing LNPs. However, enhanced luciferase activity observed in the liver for DOPE containing DSL2-2 & 2-50 LNPs compared to DSPC counterparts. Tables 2-5 detail the physio-chemical properties of LNPs made of either DOPE or DSPC (the measured hydrodynamic diameter (Z-ave; d.nm), polydispersity index (PDI)).

TABLE 2
DSL1-4 LNPs

| | Z-ave (d · nm) | PDI |
|---|---|---|
| DOPE | 92.3 | 0.1 |
| DSPC | 190.9 | 0.15 |

TABLE 3
DSL1-5 LNPs

| | Z-ave (d · nm) | PDI |
|---|---|---|
| DOPE | 52.4 | 0.19 |
| DSPC | 189.9 | 0.11 |

TABLE 4
DSL2-2 LNPs

| | Z-ave (d · nm) | PDI |
|---|---|---|
| DOPE | 72.5 | 0.27 |
| DSPC | 50.8 | 0.23 |

TABLE 5
DSL2-2 LNPs

| | Z-ave (d · nm) | PDI |
|---|---|---|
| DOPE | 88.5 | 0.08 |
| DSPC | 80.3 | 0.04 |

The effect of ionizable lipid amount on bio-distribution was further tested. mRNA-LNPs composed of DSL 1-5 or 2-50 lipid with different mole ratios (30 or 40%) administered IV in to the mice. As shown in FIG. 11Q, increasing amount of DSL1-5 lipid (40%) in the LNPs altering the bio-distribution from the spleen to the liver, whereas for DSL2-50 lipid no significant changes in bio-distribution were observed. The effect of different administration routes on LNP bio-distribution was further evaluated. As shown in FIG. 11R, intravenous (IV) administration of DSL2-50 containing mRNA-LNPs distributing to the liver (second row of both panels) mainly. Interestingly, intramuscular (IM) administration of the same LNPs also reaching the liver apart from the injection site of the muscle (bottom row, right panel).

### Example 4: In-Vivo Delivery of mRNA Encoding Cre Recombinase Using LNPs

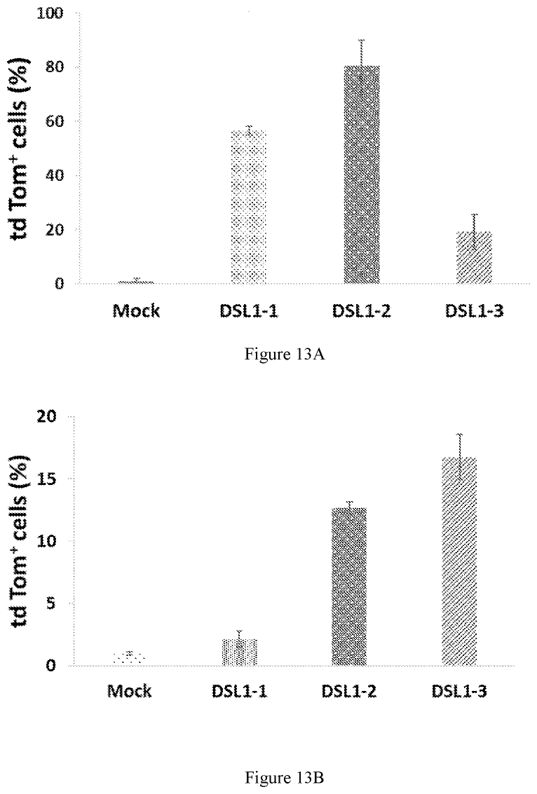

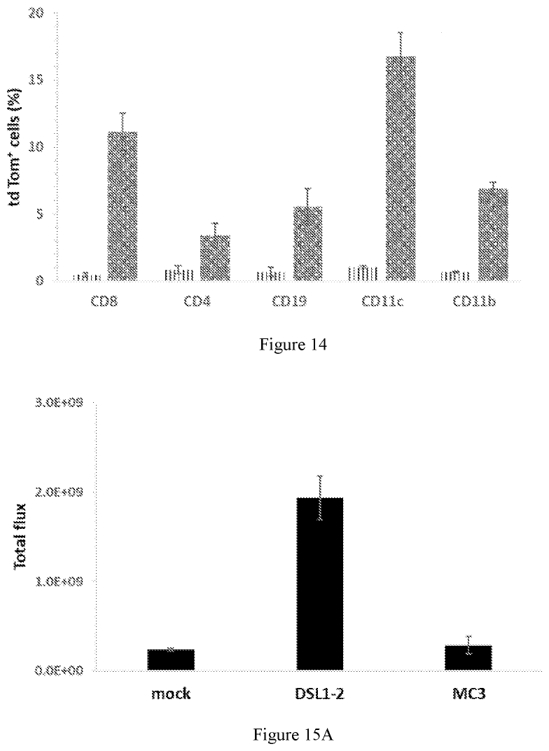

LNPs comprising DSL1-1, DSL1-2 or DSL1-3 (as described in Example 2), encapsulated with Cre recombinase encoding mRNA (mCRE-LNPs) were synthesized by Nanoassemblr as mentioned above. Ai9 mice that express robust tdTomato fluorescence following cre-mediated recombination were injected intravenously with the various mCRE-LNPs at 0.5 mg/kg dose. Mice were sacrificed 72 hr post administration and peritoneal macrophages and spleenocytes were isolated and analyzed by FACS for tdTomato expression (FIGS. 13 and 14).

As shown in FIG. 13A, about 90% of peritoneal macrophages expressed tdTomato in mice treated with DSL1-2 LNPs compared to LNPs comprising DSL1-1 (˜60%) and DSL1-3 (˜20%). However, DSL1-3 LNPs efficiently taken up by CD11c positive splenic dendritic cells (˜18%) compared to LNPs comprising DSL1-1 (˜3%) and DSL1-2 (˜12%).

### Example 5: Biodistribution of DSL1-2 LNPs Compared to Dlin-MC3-DMA LNPs

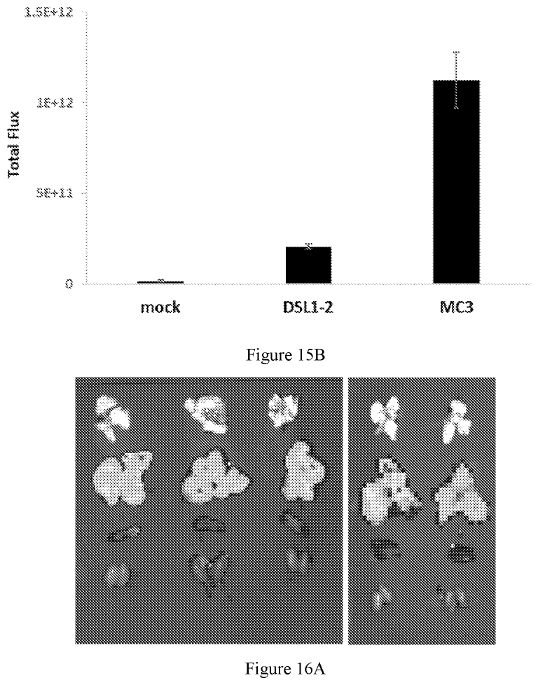

Ai9 mice that express robust tdTomato fluorescence following cre-mediated recombination were injected intravenously with either Dlin-MC3-DMA or DSL1-2 mCRE-LNPs at 0.5 mg/kg dose. Mice were sacrificed 72 hr post administration followed by spleen and liver isolation and analyzed by IVIS for tdTomato expression. As can be seen in FIGS. 15A and 15B, FDA approved ionizable lipid Dlin-MC3-DMA accumulated in the liver, whereas lipid DSL1-2 accumulated in the spleen.

### Example 6: Stability and Safety of mRNA-LNPs

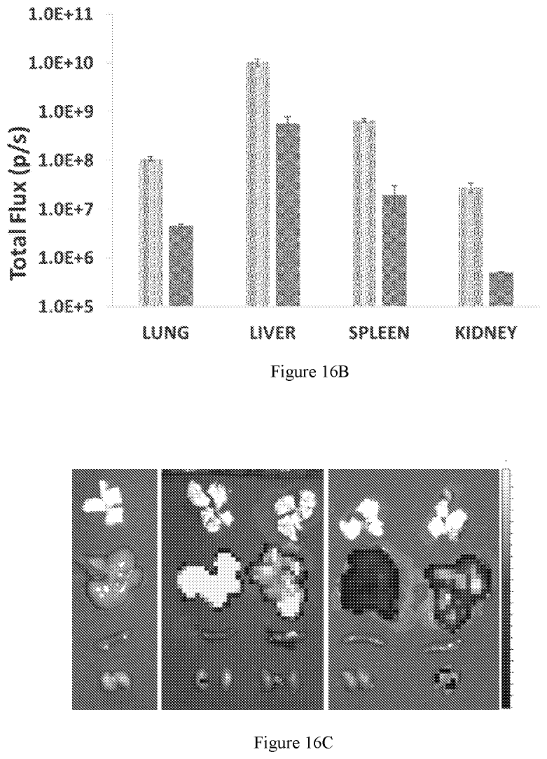

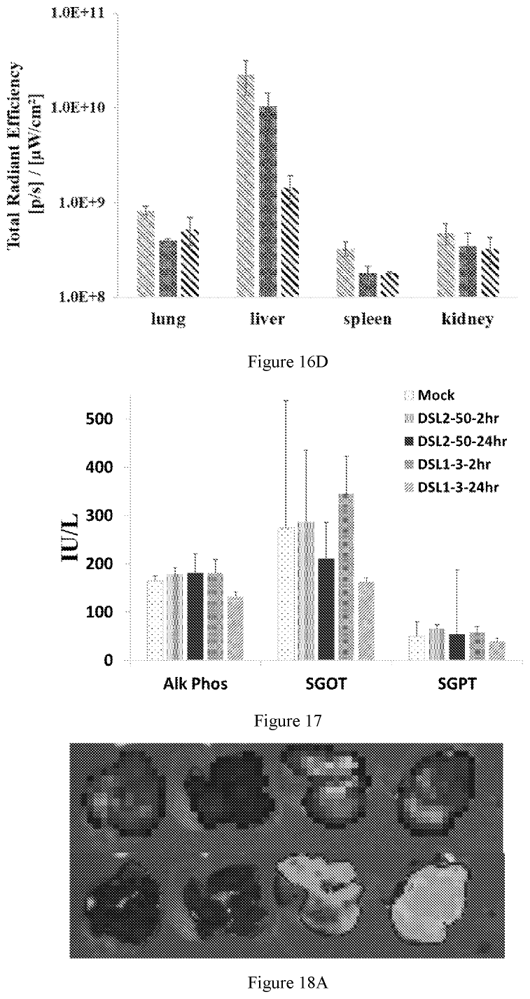

The stability of mRNA-LNPs is challenging and cost effective as COVID-19 mRNA vaccine need freezing conditions for long term storage and delivery. Hence, the stability of DSL2-50 containing mRNA-LNPs stored at 2-8° C. was tested. DSL2-50 LNPs were encapsulated either mLuc or mCherry and stored for 210 days (7 months) and 90 days (3 months) respectively. Interestingly, luciferase LNPs are stable even after 7 months of preparation as the LNPs retain its luciferase activity in the liver as shown in the FIG. 16A. In support to these results, as shown in table 6, the size and distribution of the LNPs did not change compared to freshly prepared LNPs. On the other hand, mCherry LNPs are also stable for 3 months. As shown in FIG. 16B, the expression levels of red fluorescent protein in the liver was similar to freshly prepared LNPs. In support to these results, as shown in table 7, the size of the LNPs slightly increased after 3 months compared to freshly prepared LNPs.

TABLE 6
Physico-chemical characterization of mLuc-LNPs

| | Z-ave (d · nm) | PDI |
|---|---|---|
| Day 0 | 80.3 | 0.04 |
| Day 210 | 96.0 | 0.19 |

TABLE 7
Physico-chemical characterization of mCherry-LNPs

| | Z-ave (d · nm) | PDI |
|---|---|---|
| Day 0 | 88.5 | 0.05 |
| Day 90 | 101.9 | 0.09 |

Safety is an important limitation for clinical development of LNPs. Therefore, mRNA-LNPs system made of DSL2-50 and DSL1-3 lipids were tested and evaluated for the toxicity in mice. As shown FIG. 17, LNPs were not toxic, as the levels of liver enzymes did not affect compared to untreated mice.

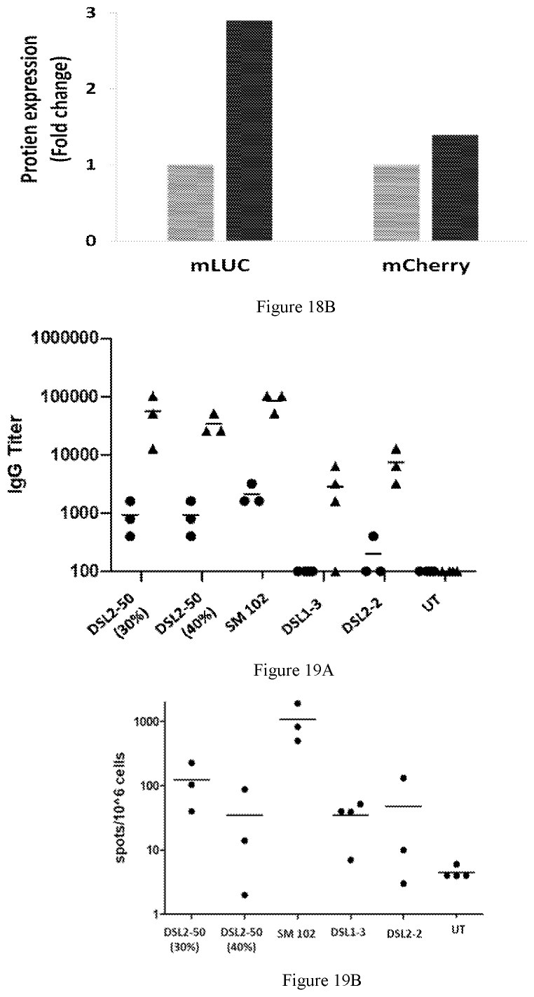

DSL2-50 efficiently deliver two types of mRNA's encapsulated in single LNP system. As shown in FIGS. 18A-B, the amount of Luciferase and mCherry expression were high in mice administered with DSL2-50 LNPs compared to Moderna's covid-19 vaccine lipid SM102-LNPs.

### Example 7: Evaluation of mRNA-LNPs for COVID-19 Vaccine Delivery

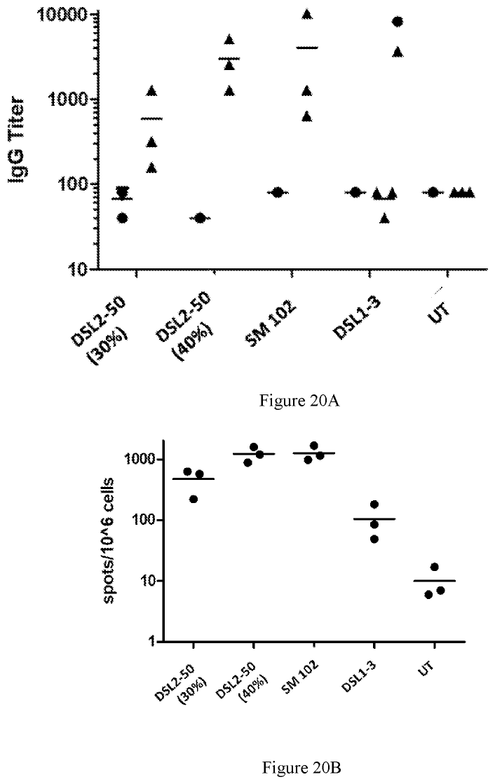

SARS-CoV-2 spike (S) protein consists of S1, including receptor-binding domain (RBD), which specifically recognizes the ACE2 receptor and plays a crucial role in mediating viral entry into cells, and S2 subunits. To evaluate the efficiency of our LNP system for vaccine delivery, mRNA against human Fc conjugated RBD (RBD-hFc mRNA) was chosen. LNPs encapsulated RBD-mRNA administered IM in to BALB/c mice (FIG. 19) and C57BL6 mice (FIG. 20) on day ‘0’ and a booster dose on day ‘21’. Serum was collected on day ‘21’ (pre boost) and 3 weeks after the booster dose (post boost) and analyzed for humoral response. As shown in FIGS. 19A & 20A, pre-boost humoral response (antibody response) against SARS-CoV-2 was limited, however a robust antibody response similar to Moderna's COVID-19 vaccine lipid SM 102 LNPs was observed in DSL2-50 LNPs. The cellular response (SARS-CoV-2 specific T-cell response) was measured by ELISPOT assay for IFN-γ response. As shown in FIG. 19B, the cellular response in BALB/c mice were low compared to SM102 LNPs. However, in C57BL6 mice treated with DSL2-50 LNPs a robust cellular response was observed (FIG. 20B).

The foregoing examples of the related art and limitations related therewith are intended to be illustrative and not exclusive. Other limitations of the related art will become apparent to those of skill in the art upon a reading of the specification and a study of the figures.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1-54. (canceled)

55. A lipid represented by the structure of Formula (II): 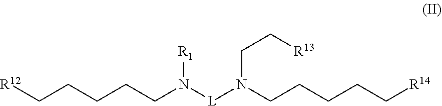
wherein
R¹ is selected from the group consisting of: OH and C₁₋₃ alkyl-OH
R¹³ is selected from the group consisting of: OH, C₁₋₃ alkyl-OH, C₄₋₁₄ alkyl and C₄₋₁₄ alkenyl;
R¹² is selected from the group consisting of: C₁₋₁₃ alkyl; C₂₋₁₅ alkenyl, C₁₋₆ alkyl-CO₂—C₀₋₃ alkylene-N(C₁₋₈ alkyl)₂ and C₁₋₆ alkyl-CO₂— C₀₋₃ alkylene-NH—C₁₋₈ alkyl;
R¹⁴ is selected from the group consisting of: C₁₋₁₃ alkyl; C₂₋₁₅ alkenyl and 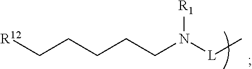
each L is an alkylene ester linker represented by: L^{a}-X^{a}-L^{b};
X^{a} is selected from the group consisting of: —O₂C—, —CO₂—C₂₋₄ alkylene-O₂C—, O₂C—C₂₋₄ alkylene-O₂C—, —CO₂—C₂₋₄ alkylene-CO₂—, and O₂C—C₂₋₄ alkylene-CO₂—;
L^{a} is selected from the group consisting of: C₁₋₃ alkylene, C₄₋₁₂ alkylene, C₂₋₁₀ alkenylene and absent; and
L^{b} is selected from the group consisting of: C₁₋₃ alkylene, C₂₋₁₀ alkenylene and C₄₋₁₂ alkylene.

56. The lipid according to claim 55, wherein R¹ is selected from the group consisting of: OH, C₂₋₃ alkyl-OH, and C₈₋₁₄ alkyl.

57. The lipid according to claim 55, wherein R¹³ is selected from the group consisting of: OH, CH₂—OH, and C₄₋₁₂ alkyl.

58. The lipid according to claim 55, wherein R¹² is selected from the group consisting of: C₉₋₁₅ alkenyl, C₅₋₁₁ alkyl, C₃₋₆ alkyl-CO₂—C₀₋₂ alkylene-N(C₂₋₈ alkyl)₂.

59. The lipid according to claim 55, wherein R¹⁴ is selected from the group consisting of: C₉₋₁₅ alkenyl, C₁₋₉ alkyl and

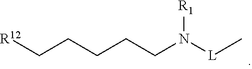

60. The lipid according to claim 55, wherein X^{a} is selected from the group consisting of: —O₂C—and —CO₂—C₂₋₄ alkylene-O₂C—.

61. The lipid according to claim 55, wherein L^{a} is absent or selected from the group consisting of: C₂₋₃ alkylene and C₆₋₁₁ alkylene.

62. The lipid according to claim 55, wherein L^{b} is selected from the group consisting of: C₁₋₃ alkylene and C₄₋₁₂ alkylene.

63. The lipid according to claim 55, which is selected from the group consisting of

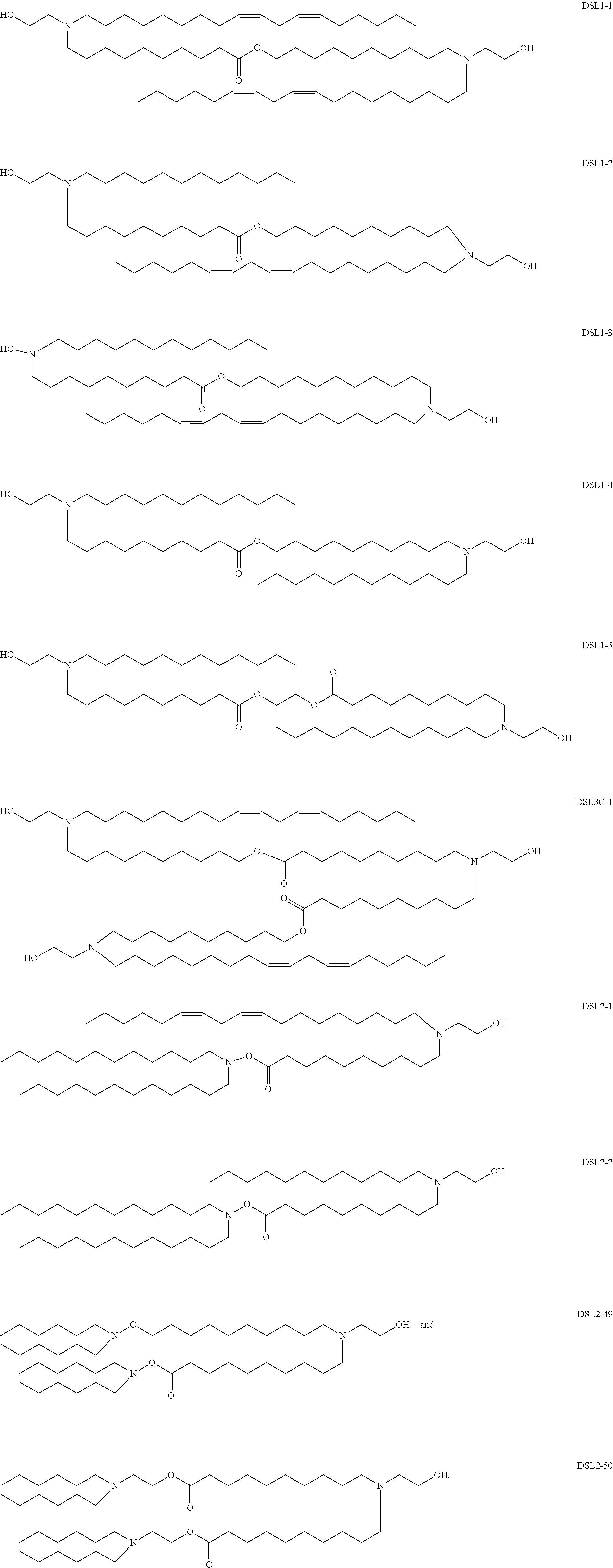

64. A lipid selected from the group consisting of:

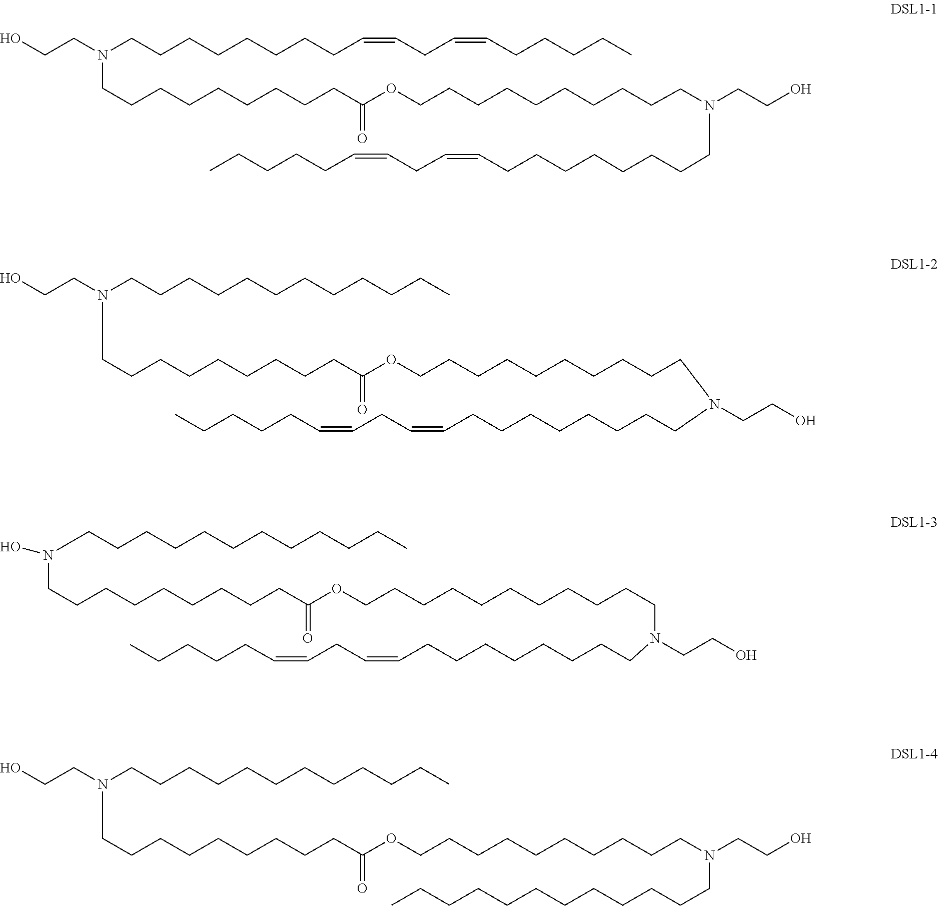 [SMILES: CCCCC/C=C\C/C=C\CCCCCCCCN(CCO)CCCCCCCCCCOC(=O)CCCCCCCCCN(CCO)CCCCCCCC/C=C\C/C=C\CCCCC ; CCCCC/C=C\C/C=C\CCCCCCCCN(CCO)CCCCCCCCCCOC(=O)CCCCCCCCCN(CCO)CCCCCCCCCCCC ; CCCCC/C=C\C/C=C\CCCCCCCCN(CCO)CCCCCCCCCCCOC(=O)CCCCCCCCCN(O)CCCCCCCCCCCC ; CCCCCCCCCCCCN(CCO)CCCCCCCCCCOC(=O)CCCCCCCCCN(CCO)CCCCCCCCCCCC]

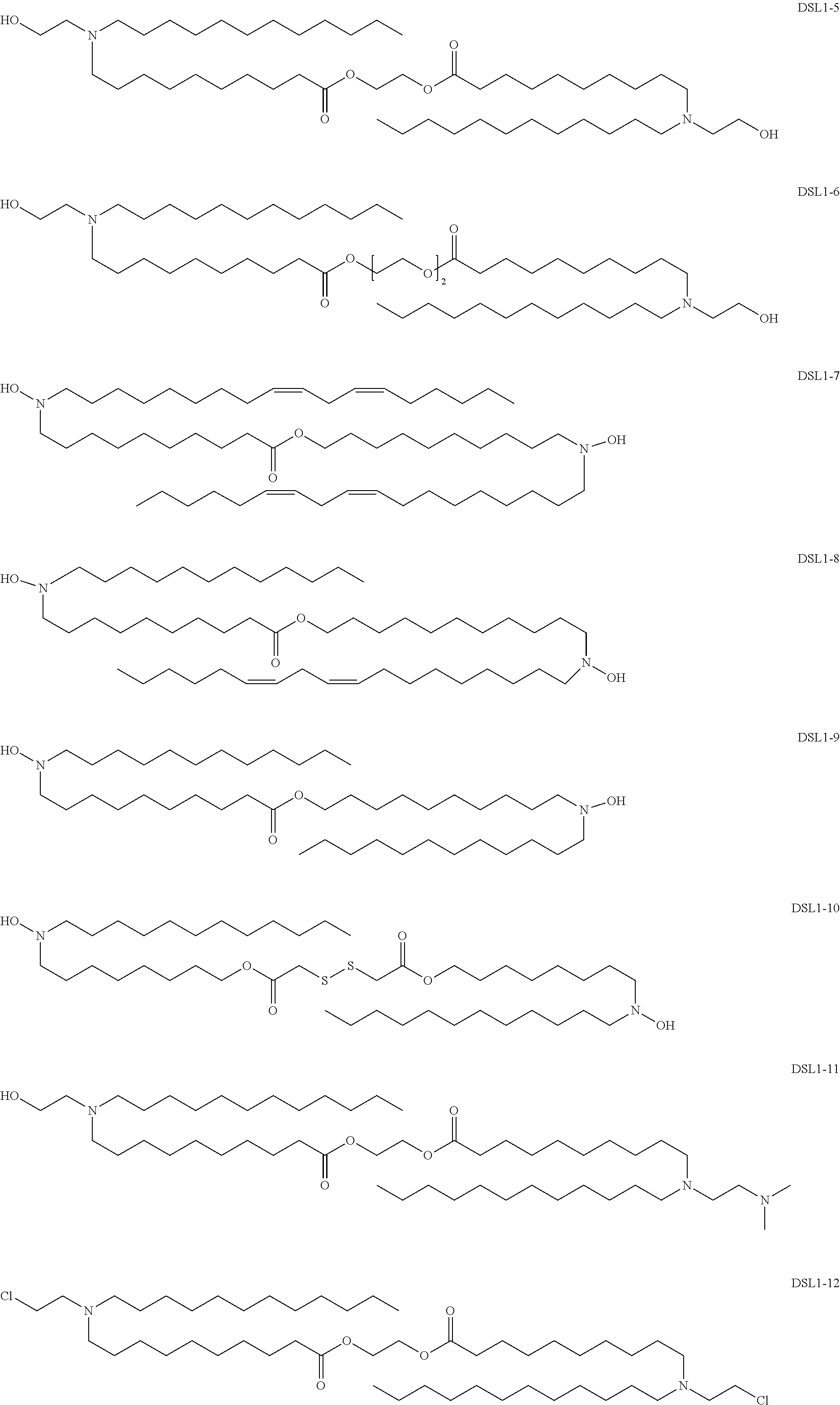

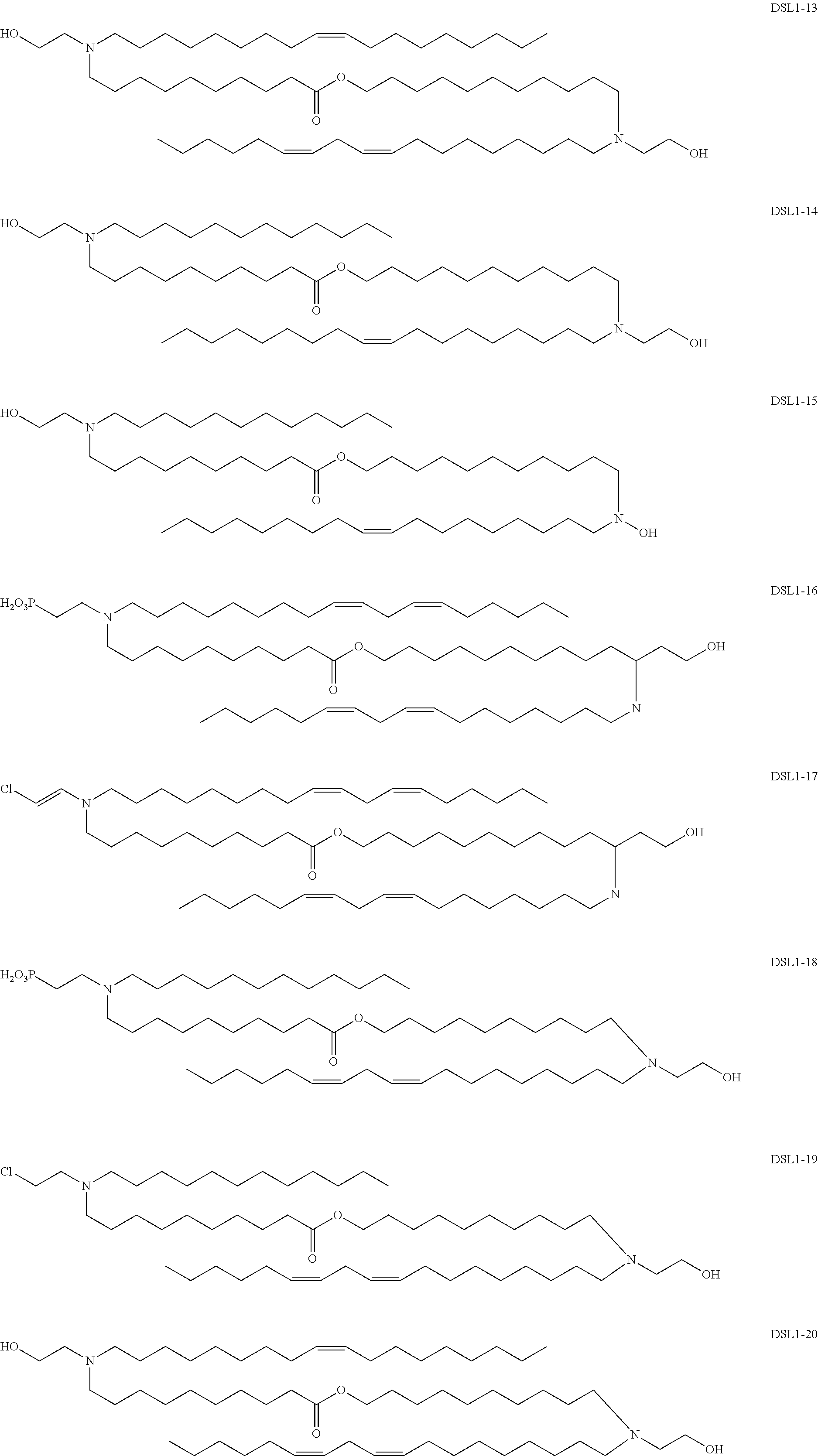

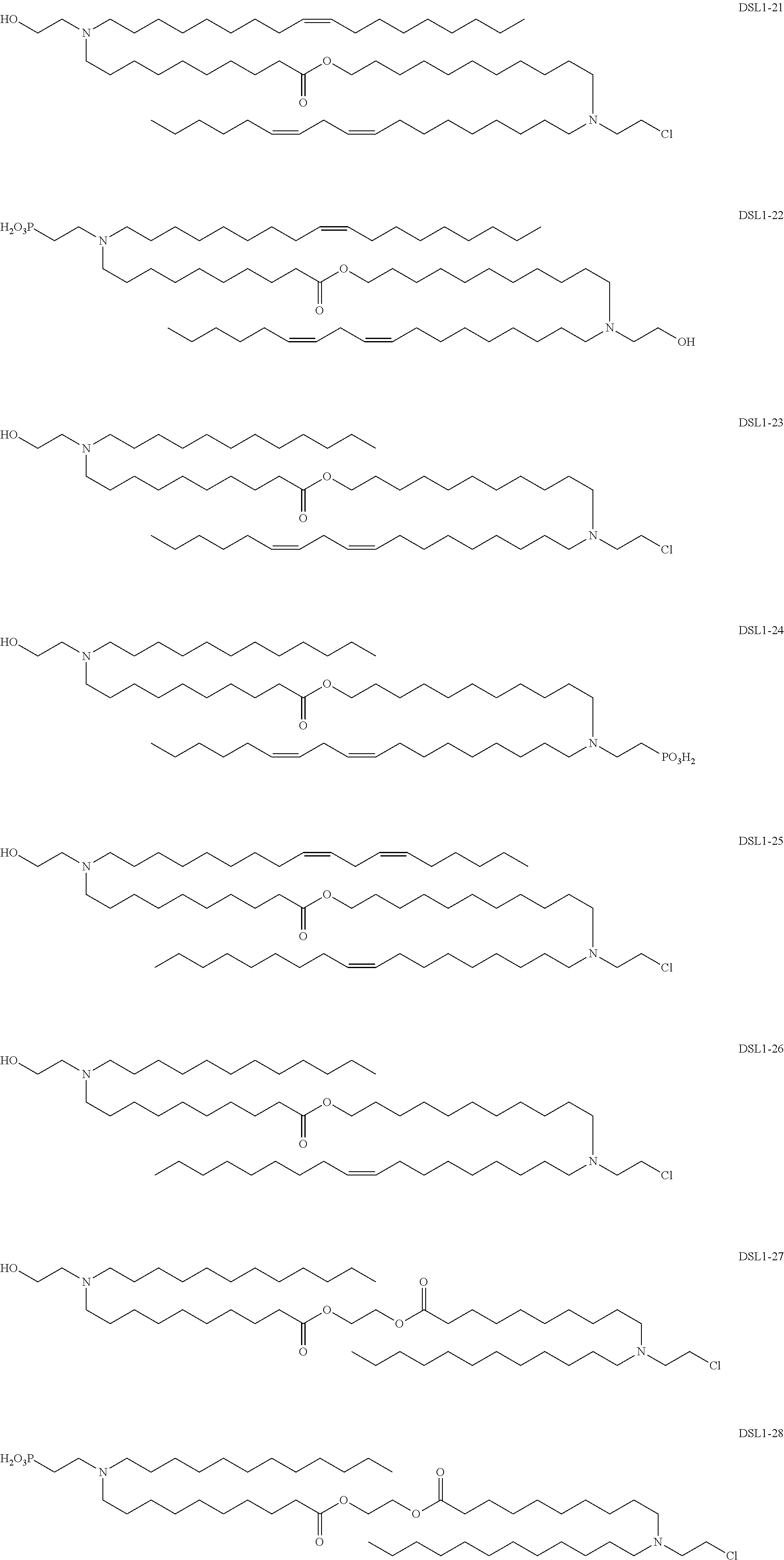

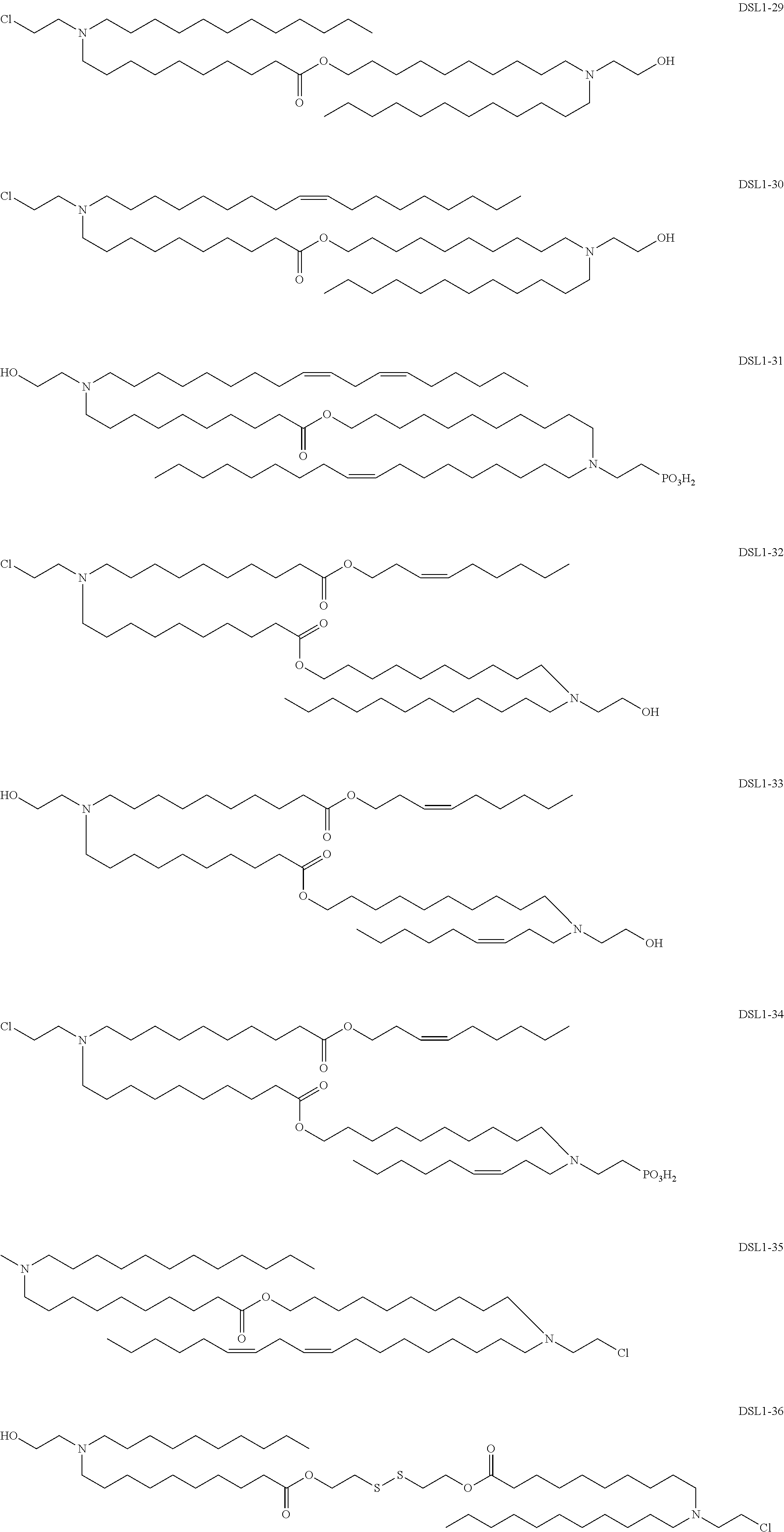

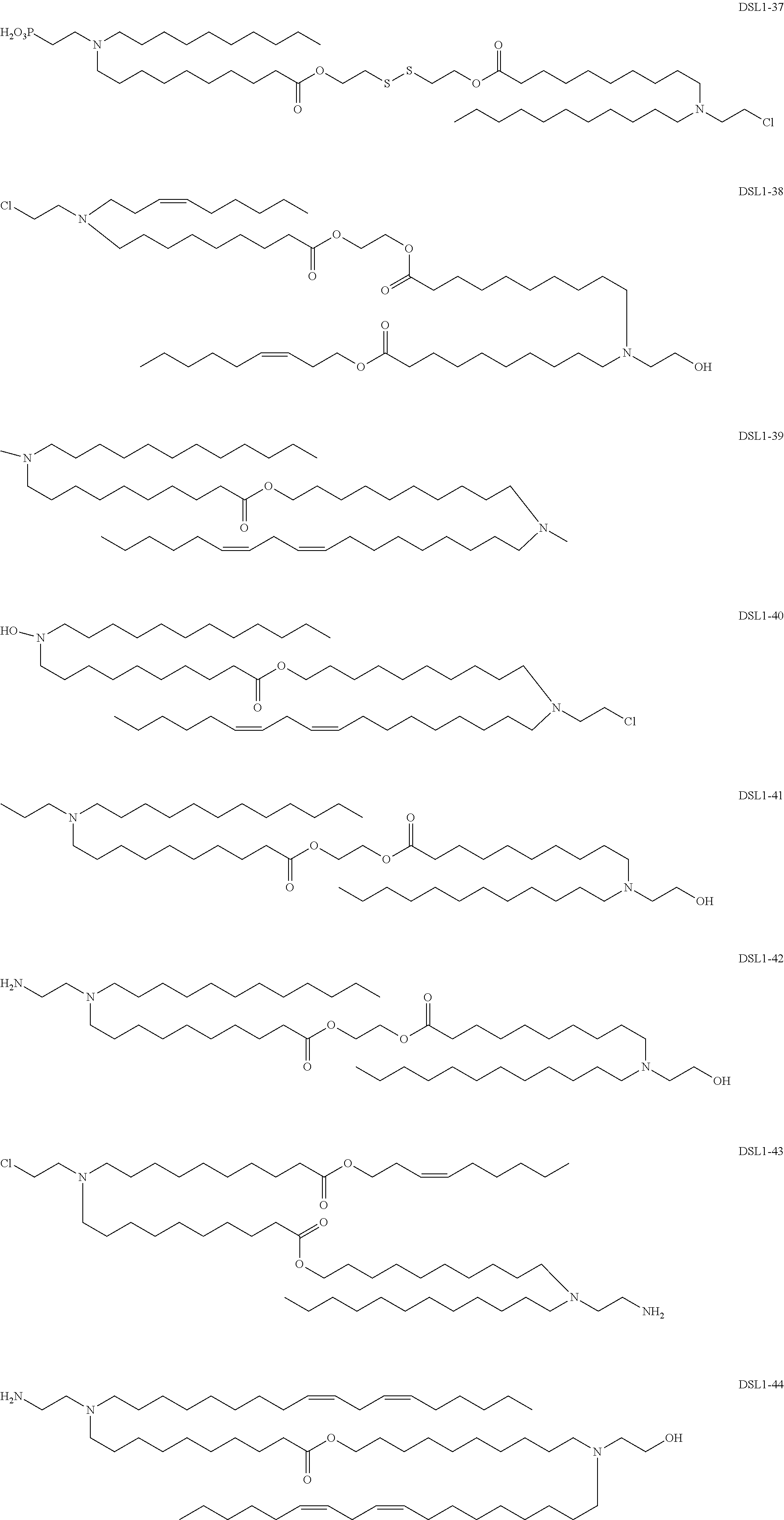

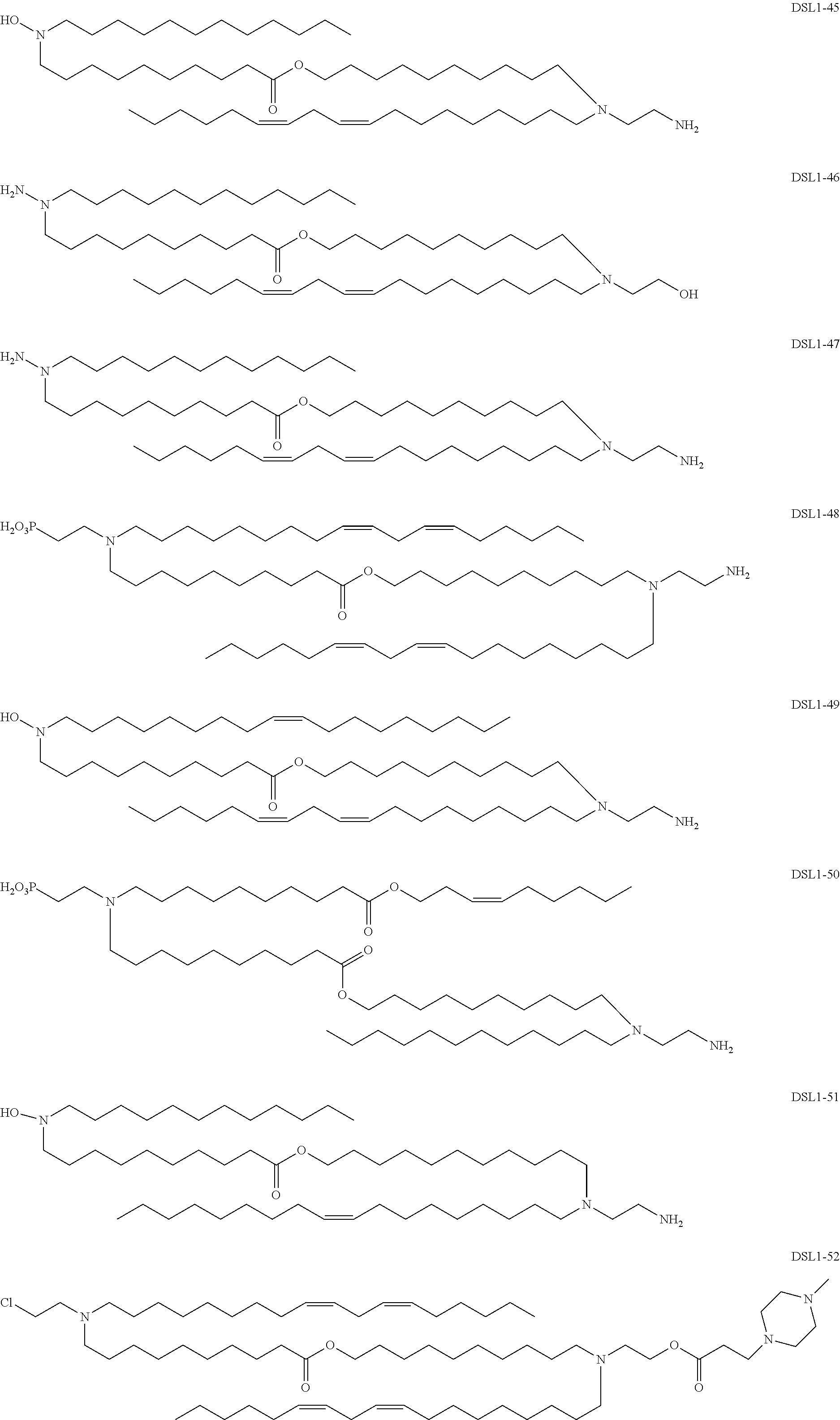

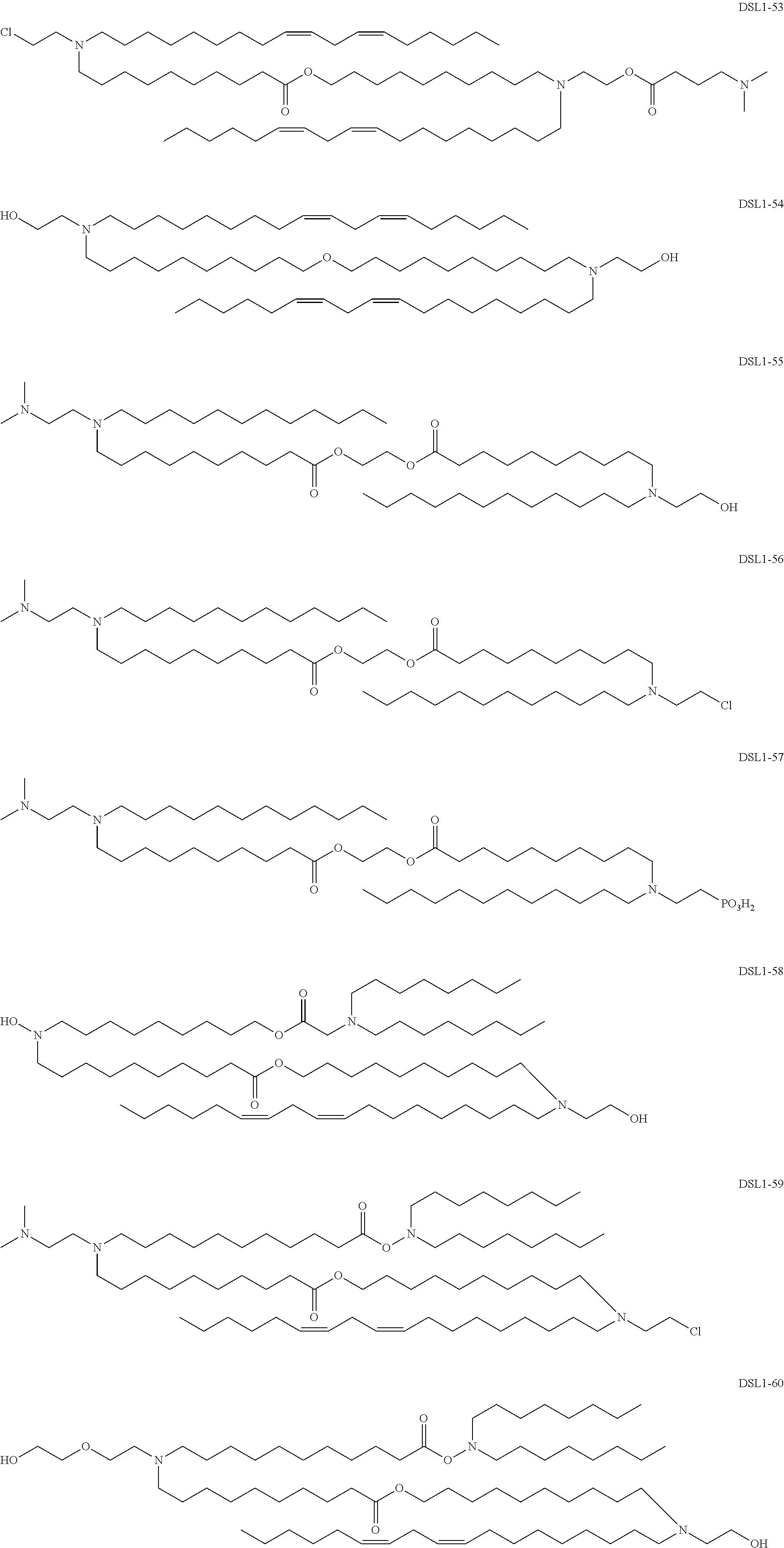

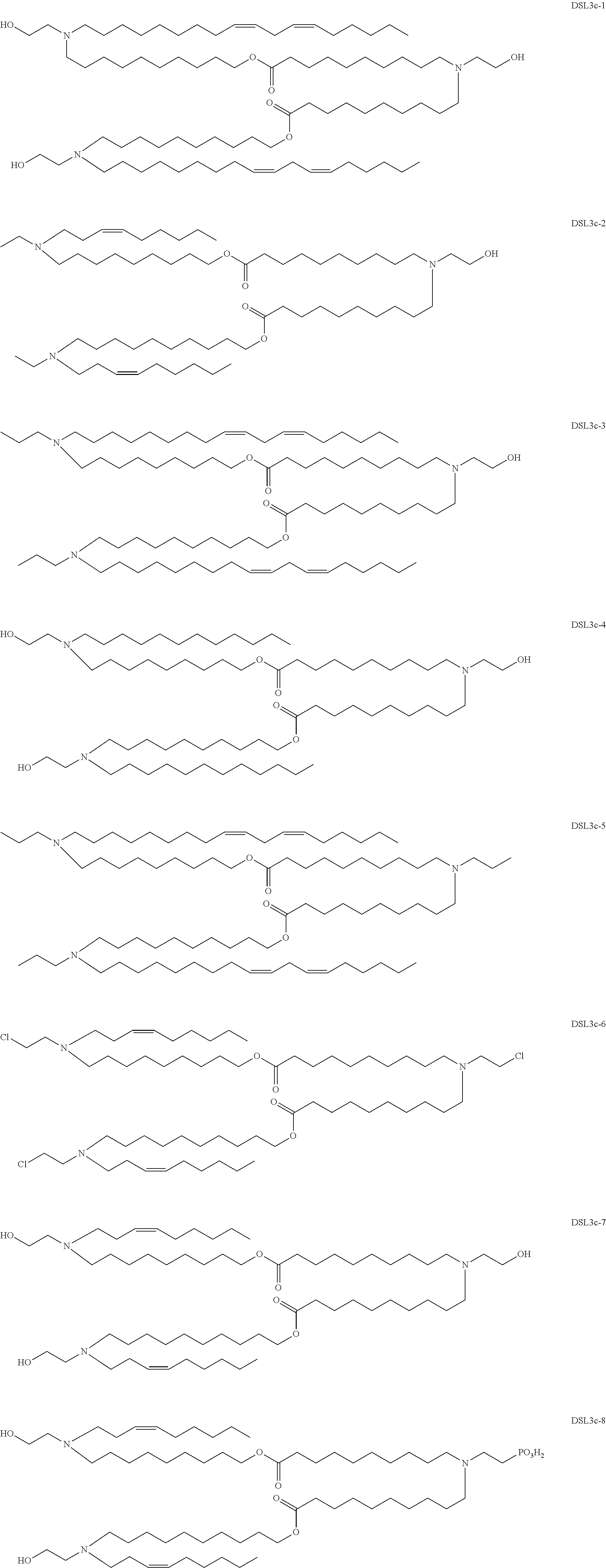

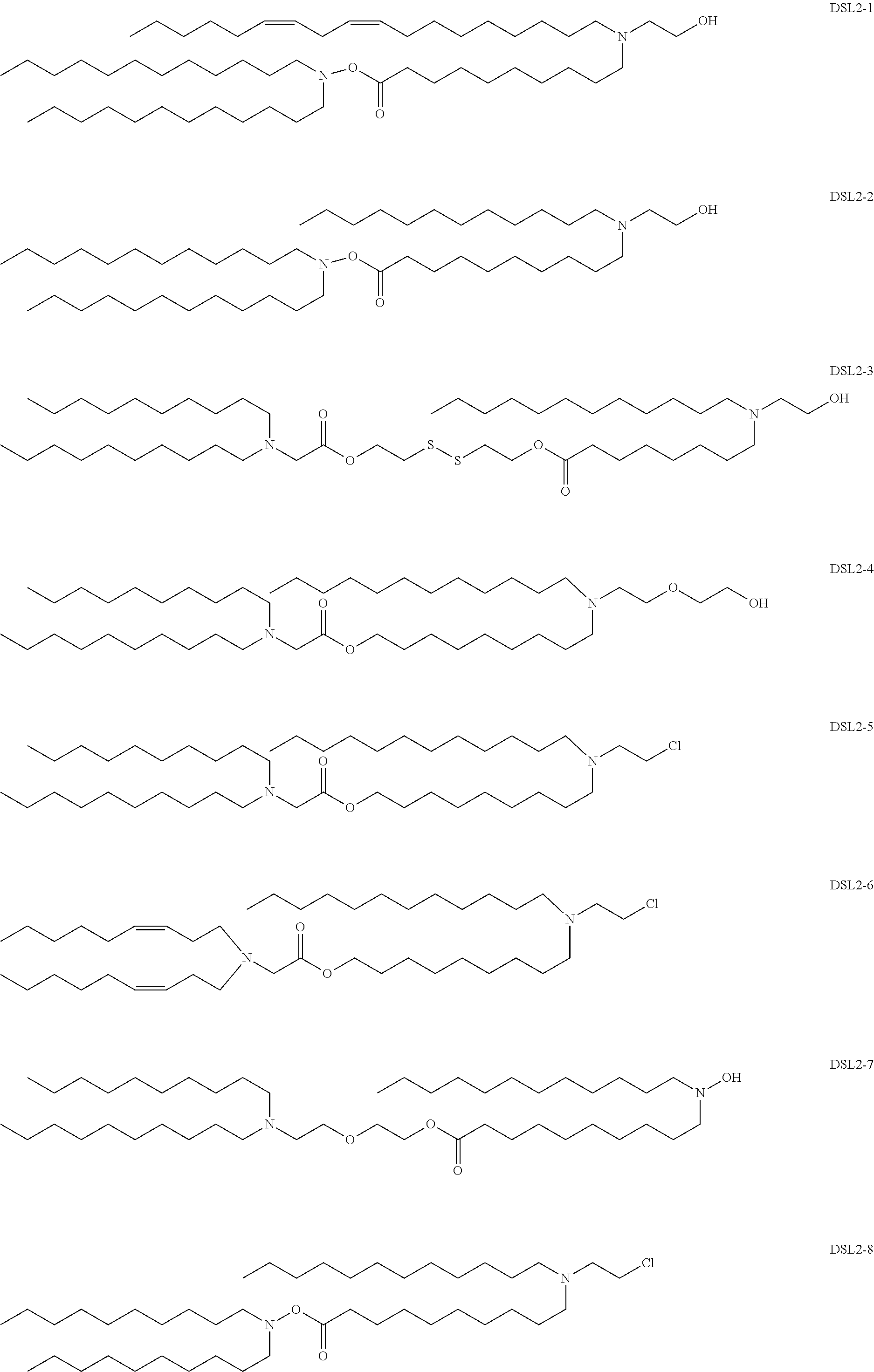

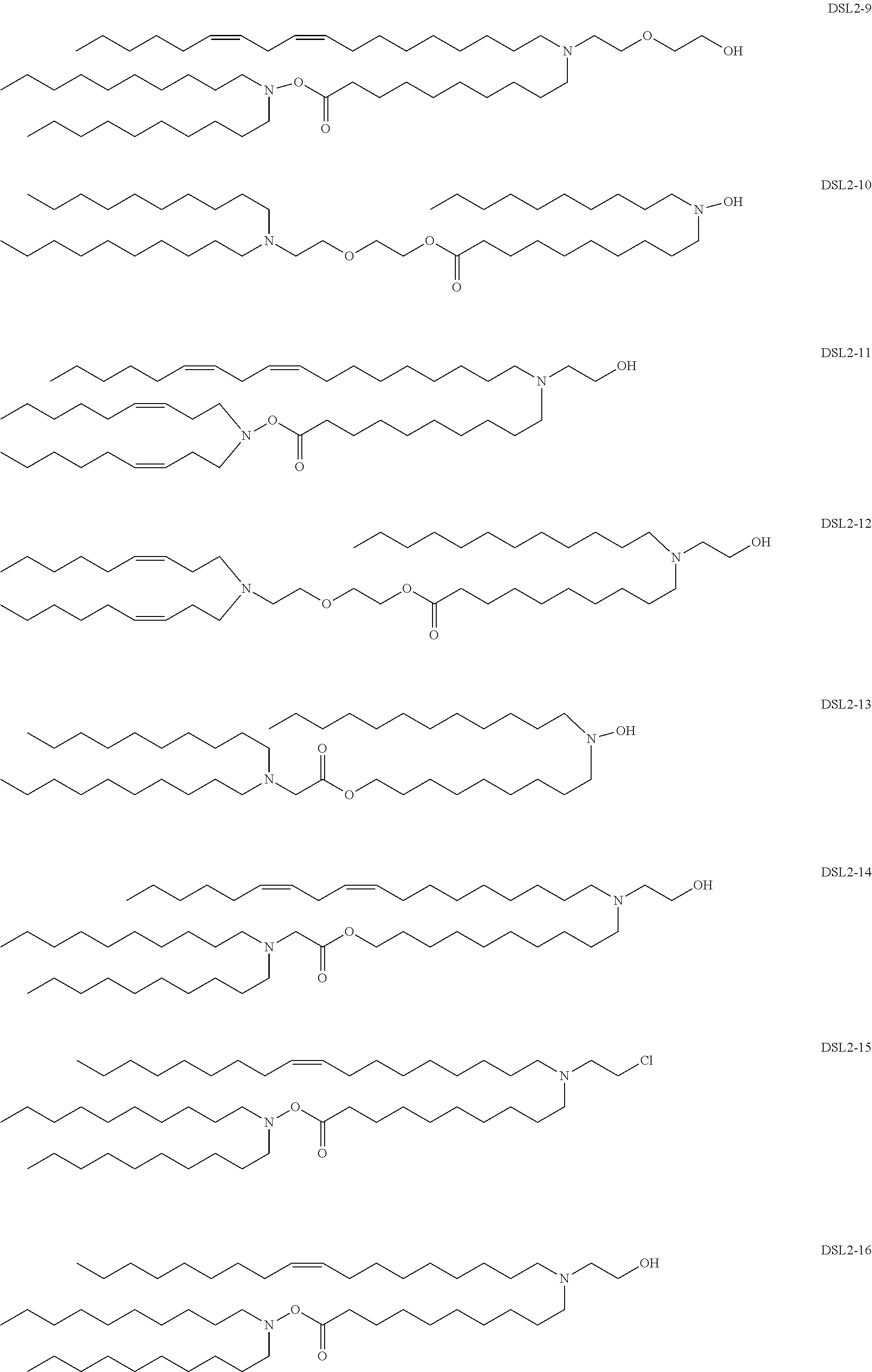

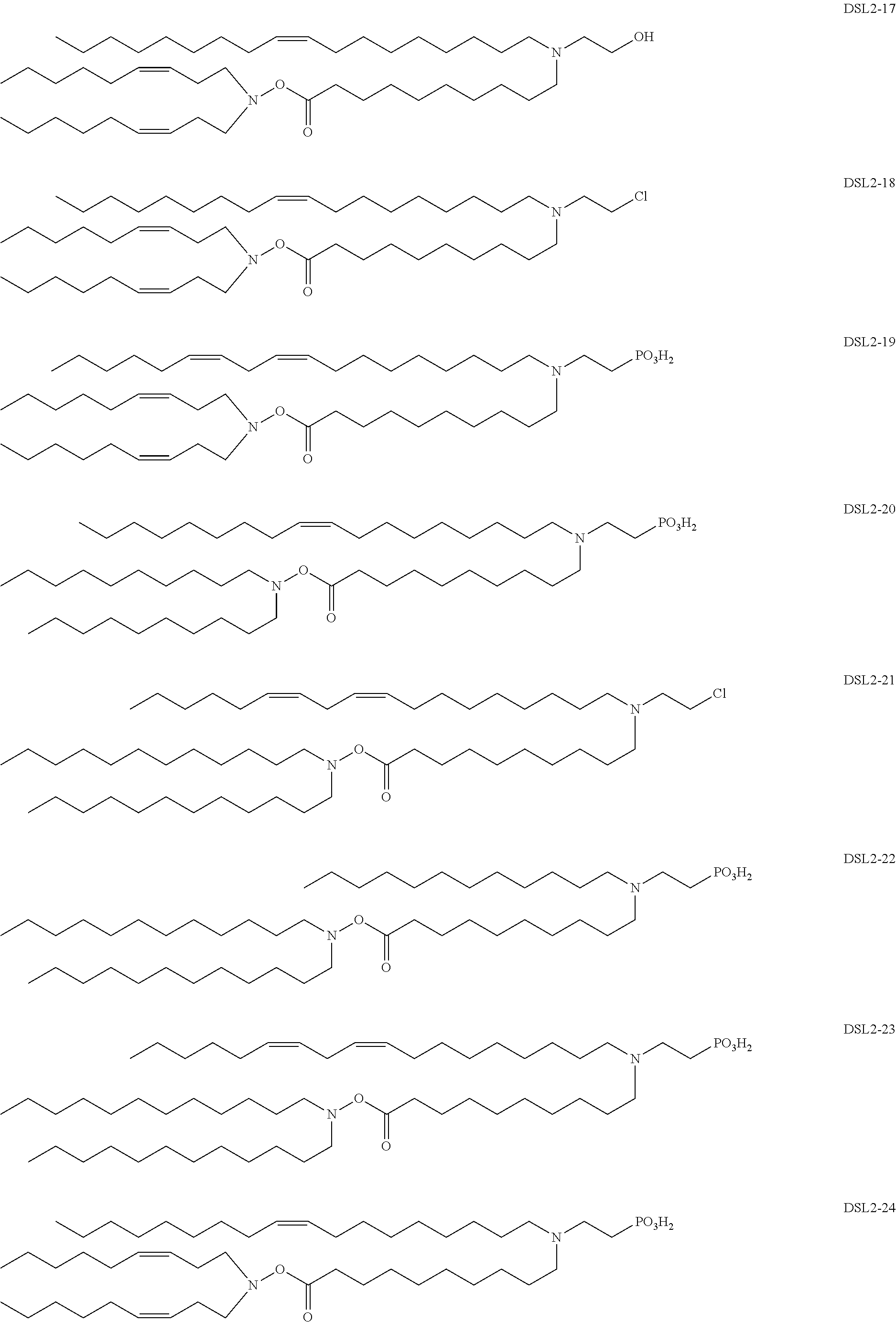

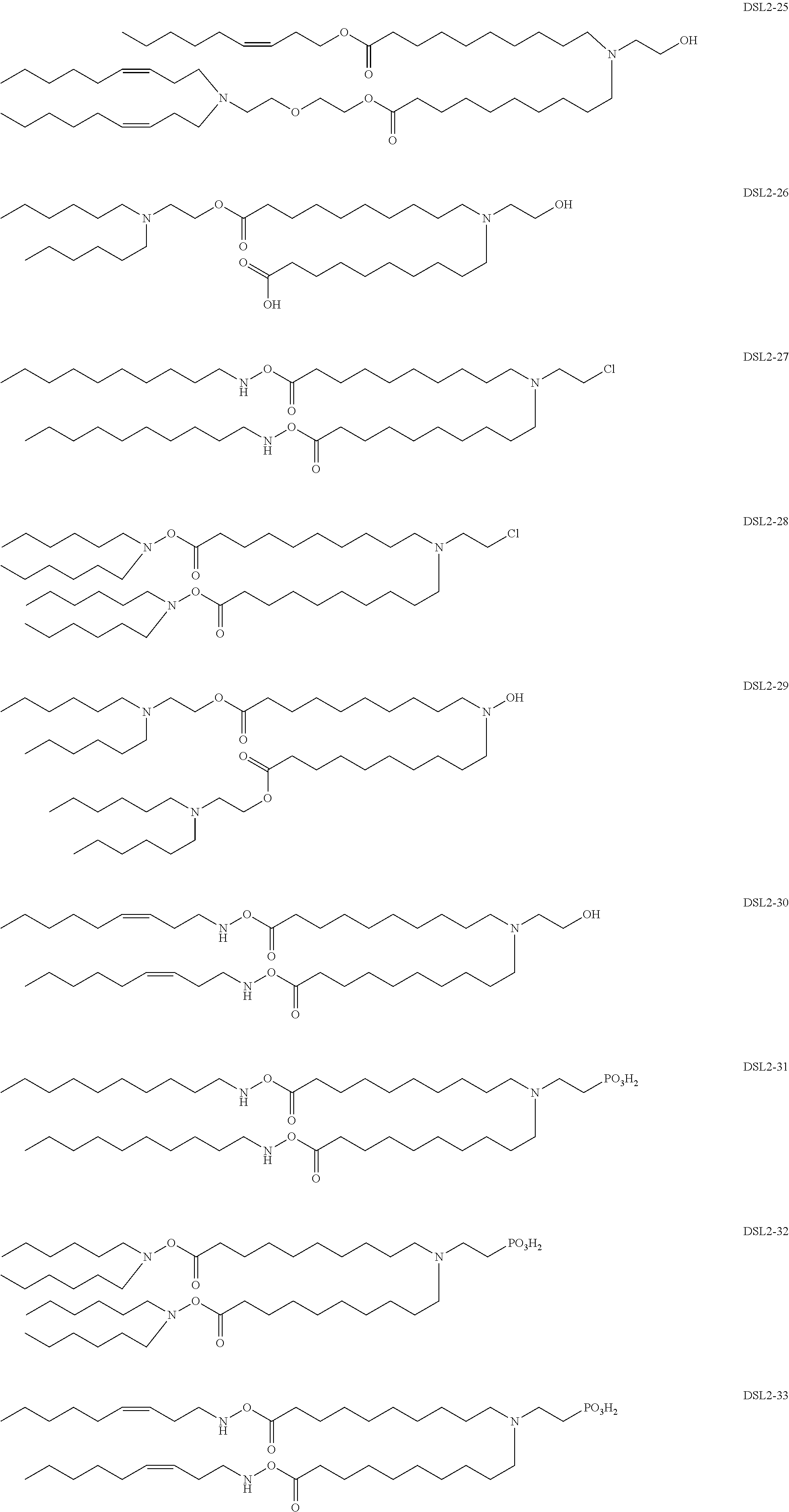

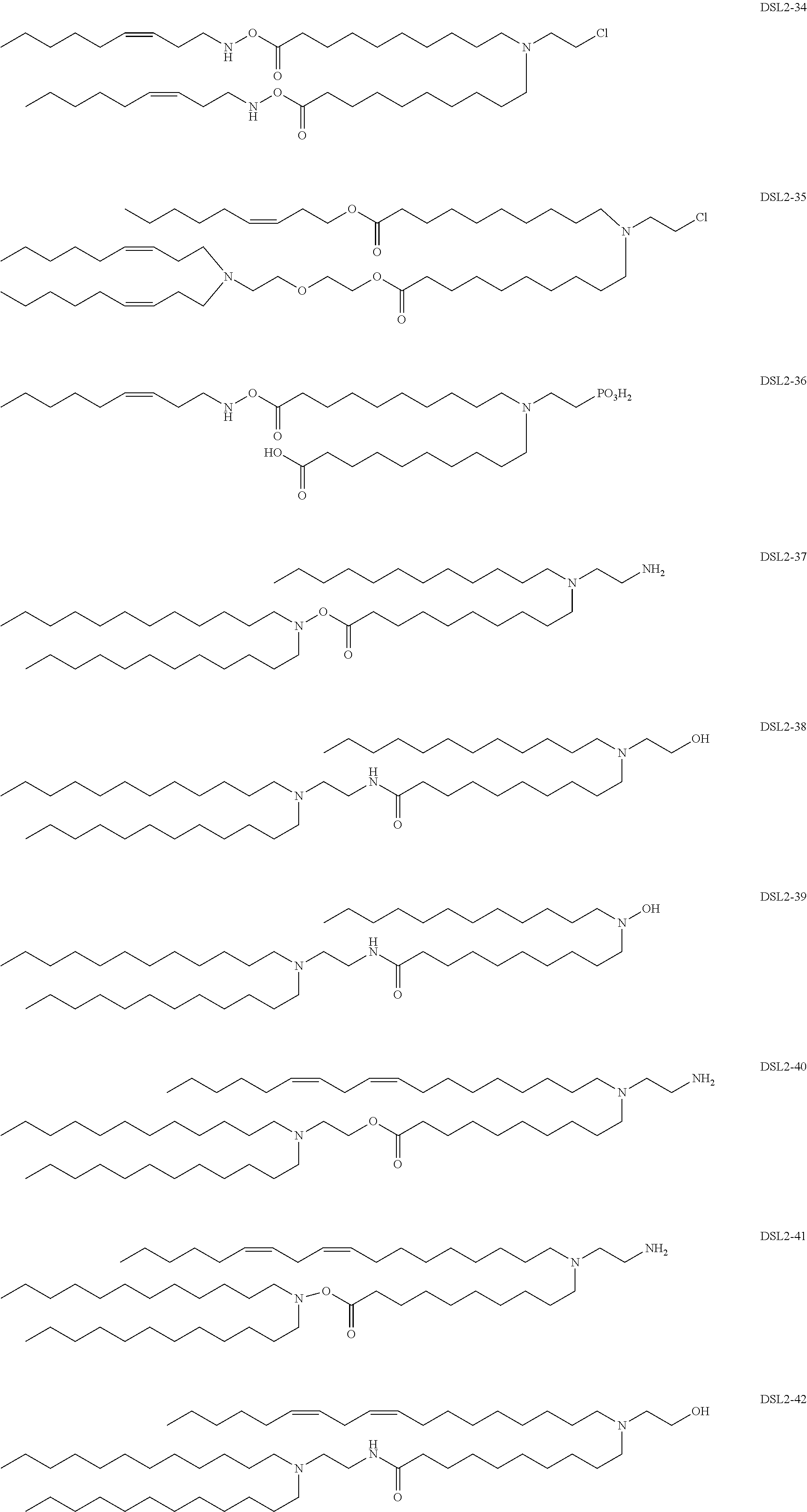

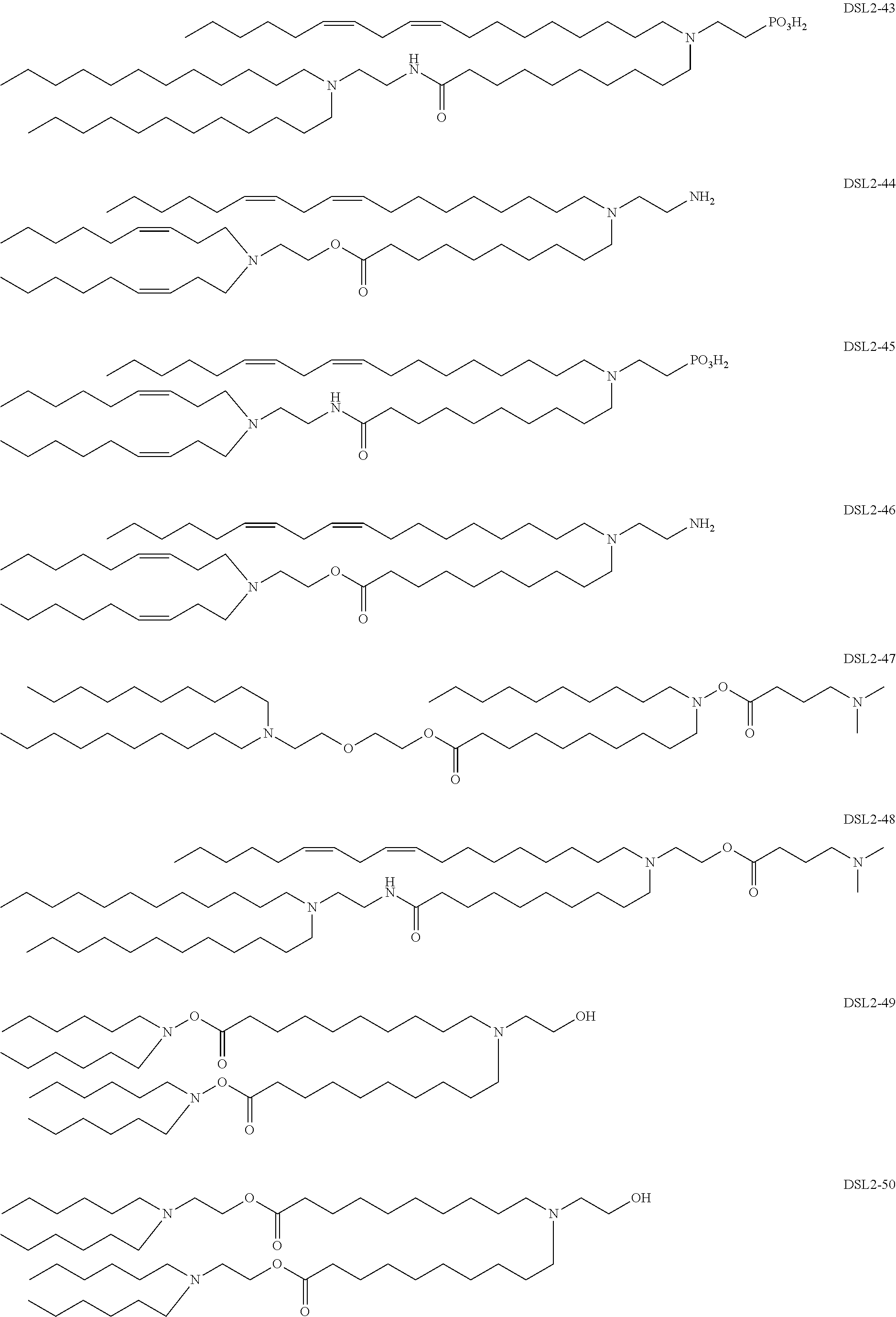

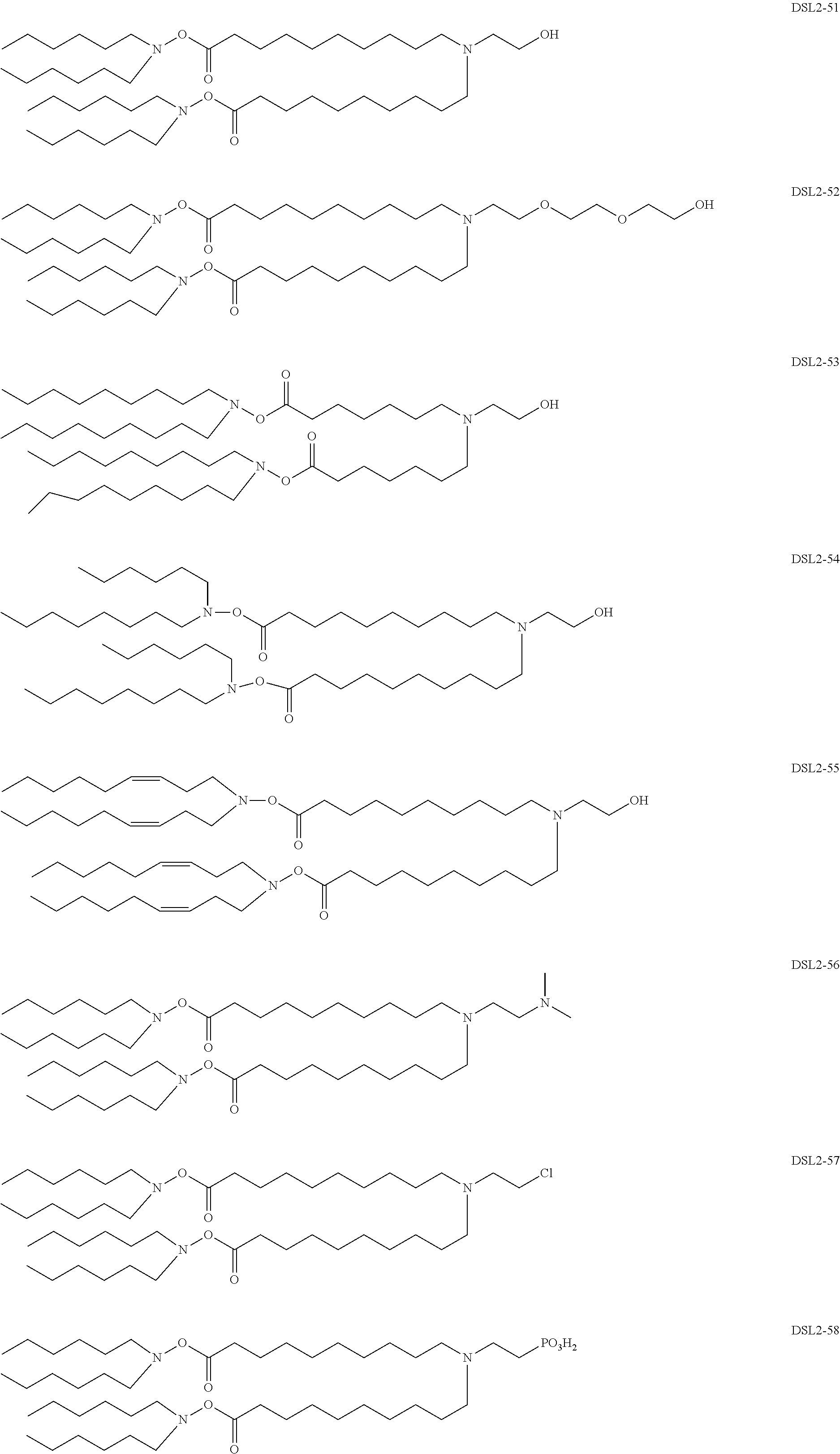

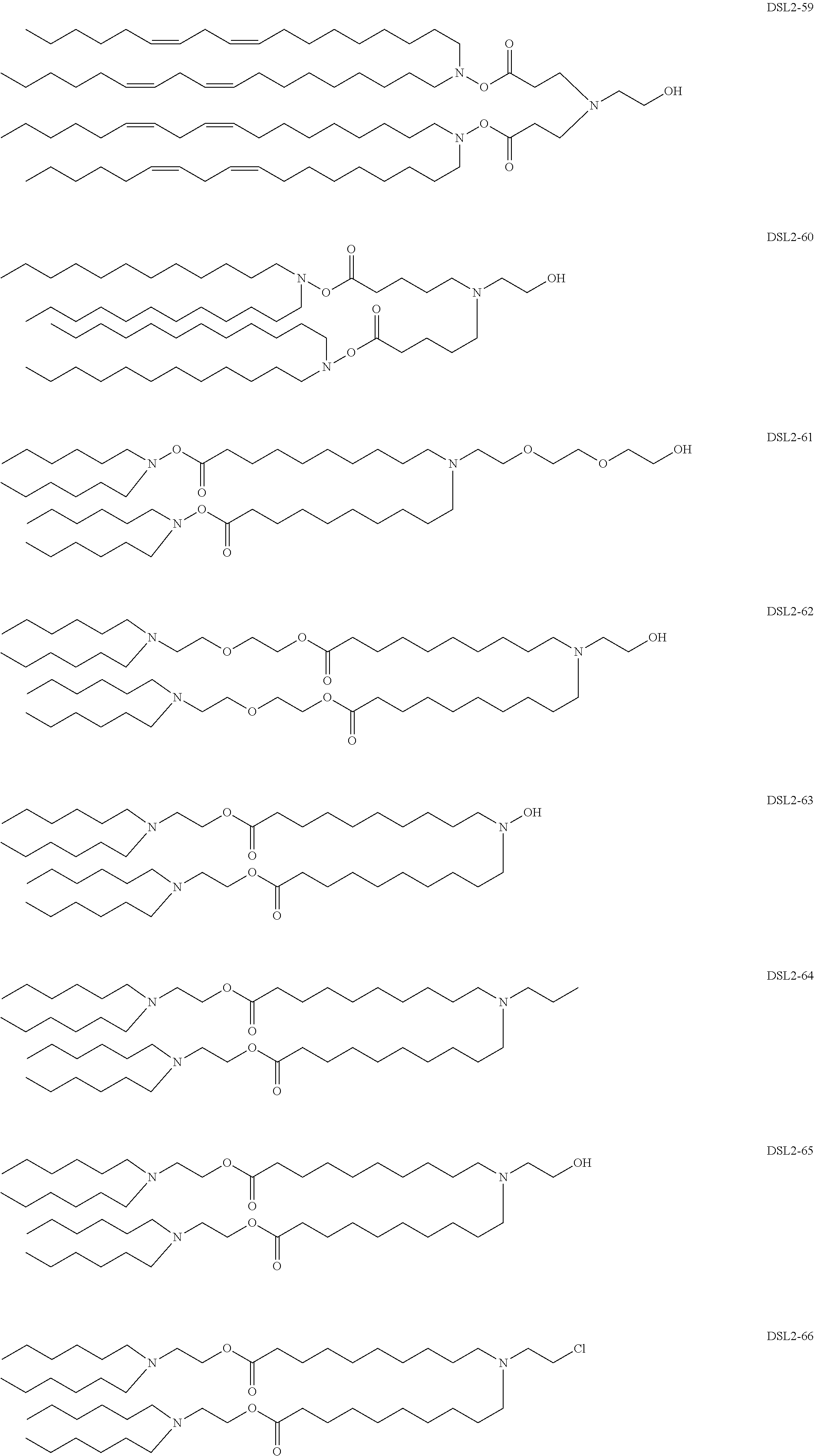

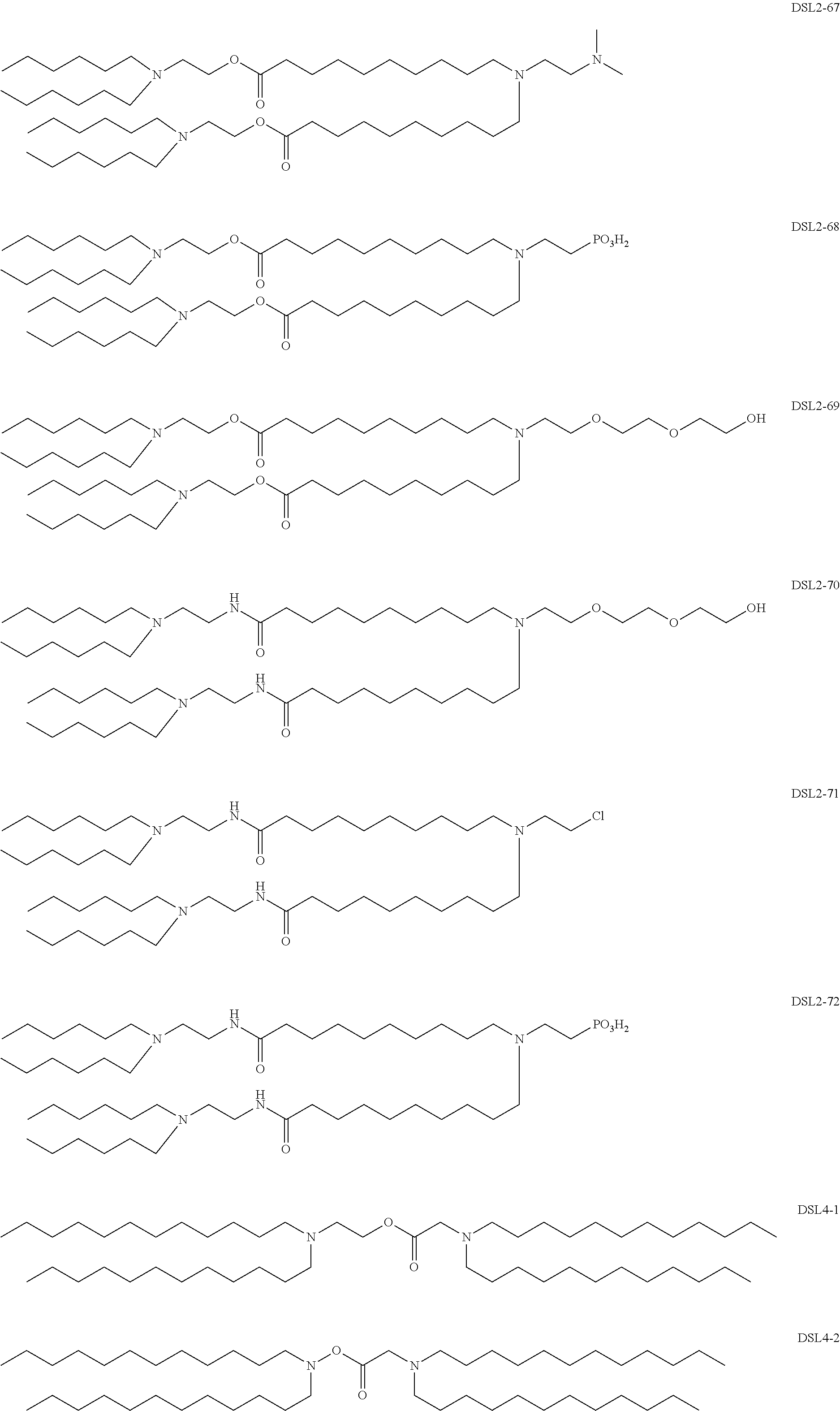

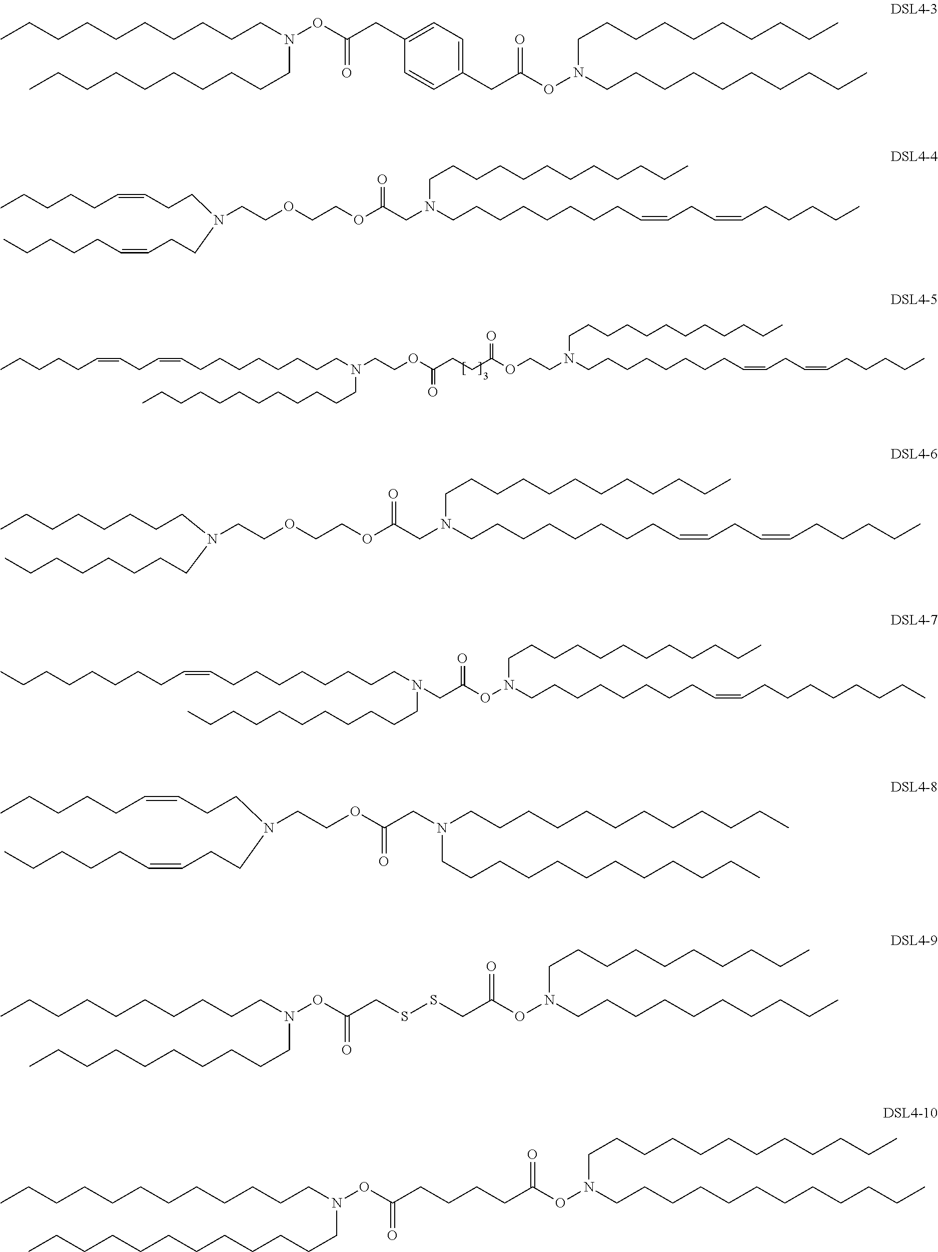

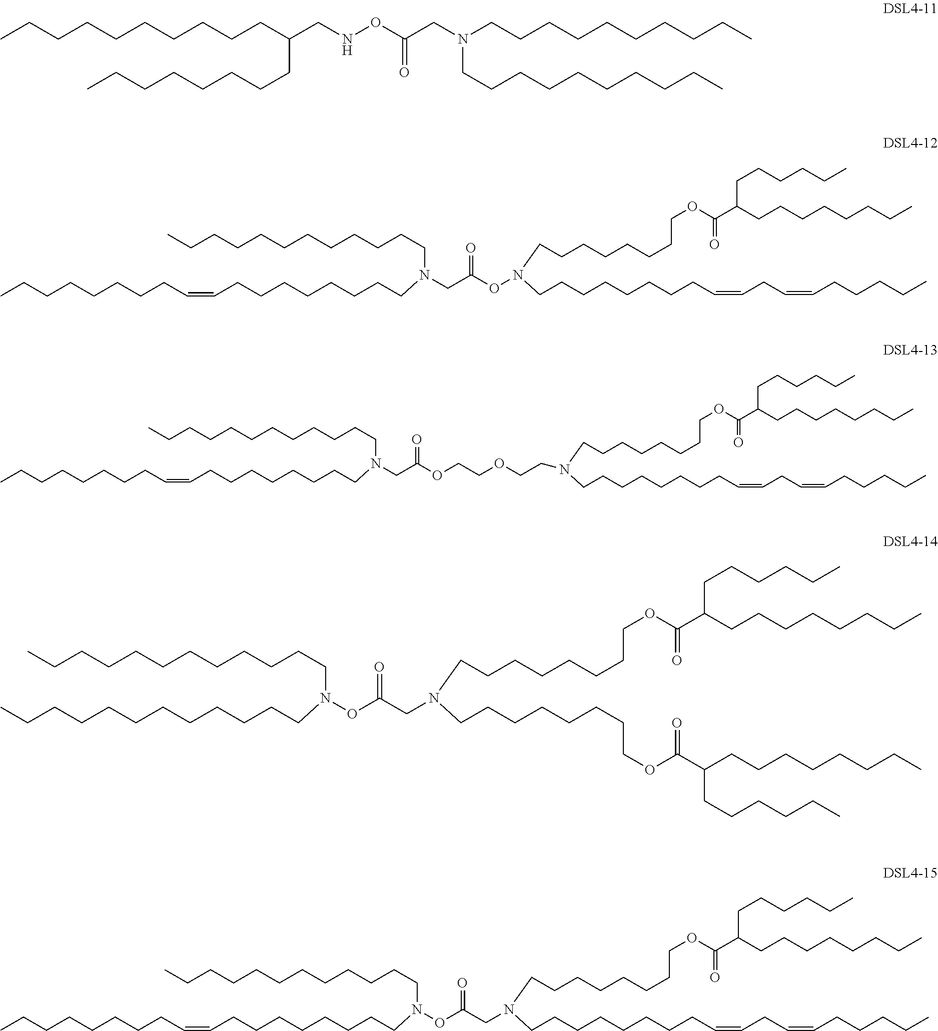

65. The lipid according to claim 64, which is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54, DSL1-55, DSL1-56, DSL1-57, DSL1-58, DSL1-59, DSL1-60, DSL3c-1, DSL3c-2, DSL3c-3, DSL3c-4, DSL3c-5, DSL3c-6, DSL3c-7 and DSL3c-8.

66. The lipid according to claim 64, which is selected from the group consisting of: DSL1-1, DSL1-2, DSL1-3, DSL1-4, DSL1-5, DSL1-6, DSL1-7, DSL1-8, DSL1-9, DSL1-10, DSL1-11, DSL1-12, DSL1-13, DSL1-14, DSL1-15, DSL1-16, DSL1-17, DSL1-18, DSL1-19, DSL1-20, DSL1-21, DSL1-22, DSL1-23, DSL1-24, DSL1-25, DSL1-26, DSL1-27, DSL1-28, DSL1-29, DSL1-30, DSL1-31, DSL1-32, DSL1-33, DSL1-34, DSL1-35, DSL1-36, DSL1-37, DSL1-38, DSL1-39, DSL1-40, DSL1-41, DSL1-42, DSL1-43, DSL1-4,4 DSL1-45, DSL1-46, DSL1-47, DSL1-48, DSL1-49, DSL1-50, DSL1-51, DSL1-52, DSL1-53, DSL1-54 DSL1-55, DSL1-56, DSL1-57, DSL1-58, DSL1-59 and DSL1-60.

67. The lipid according to claim 64, which is selected from the group consisting of: DSL3c-1, DSL3c-2, DSL3c-3, DSL3c-4, DSL3c-5, DSL3c-6, DSL3c-7 and DSL3c-8.

68. The lipid according to claim 64, which is selected from the group consisting of: DSL2-1, DSL2-2, DSL2-3, DSL2-4, DSL2-5, DSL2-6, DSL2-7, DSL2-8, DSL2-9, DSL2-10, DSL2-11, DSL2-12, DSL2-13, DSL2-14, DSL2-15, DSL2-16, DSL2-17, DSL2-18, DSL2-19, DSL2-20, DSL2-21, DSL2-22, DSL2-23, DSL2-24, DSL2-25, DSL2-26, DSL2-27, DSL2-28, DSL2-29, DSL2-30, DSL2-31, DSL2-32, DSL2-33, DSL2-34, DSL2-35, DSL2-36, DSL2-37, DSL2-38, DSL2-39, DSL2-40, DSL2-41, DSL2-42, DSL2-43, DSL2-44, DSL2-45, DSL2-46, DSL2-47, DSL2-48, DSL2-49, DSL2-50, DSL2-51, DSL2-52, DSL2-53, DSL2-54, DSL2-55, DSL2-56, DSL2-57, DSL2-58, DSL2-59, DSL2-60, DSL2-61, DSL2-62, DSL2-63, DSL2-64, DSL2-65, DSL2-66, DSL2-67, DSL2-68, DSL2-69, DSL2-70, DSL2-71 and DSL2-72.

69. The lipid according to claim 64, which is selected from the group consisting of: DSL4-1, DSL4-2, DSL4-3, DSL4-4, DSL4-5, DSL4-6, DSL4-7, DSL4-8, DSL4-9, DSL4-10, DSL4-11, DSL4-12, DSL4-13, DSL4-14 and DSL4-15.

70. The lipid according to claim 64, which is selected from the group consisting of: DSL1-1, DSL1-2, DSL1-3, DSL1-4, DSL1-5, DSL2-1, DSL2-2, DSL4-1, DSL4-2, DSL3c-1, DSL2-49 and DSL2-50.

71. A particle comprising the lipid according to claim 55 and a membrane stabilizing lipid, wherein the membrane stabilizing lipid is selected from the group consisting of cholesterol, phospholipids, cephalins, sphingolipids and glycoglycerolipids.

72. The particle according to claim 71, further comprising a nucleic acid encapsulated within a particle comprising the lipid, wherein the nucleic acid is selected from the group consisting of small interfering RNA (siRNA), micro RNA (miRNA), antisense oligo nucleotides, messenger RNA (mRNA), ribozymes, pDNA, CRISPR mRNA, gRNA, circular RNA and immune stimulating nucleic acids.

73. A method of gene silencing, comprising the step of contacting a cell with a composition comprising a plurality of particles according to claim 71 and a pharmaceutically acceptable carrier, diluent or excipient.

74. A method of treating a leukocyte associated condition, the method comprising the step of administering to a subject in need thereof a composition comprising a plurality of particles according to claim 71 and a pharmaceutically acceptable carrier, diluent or excipient.
